(19) United States
(12) Patent Application Publication
(10) Pub. No.: US 2026/0055103 A1
(43) Pub. Date: Feb. 26, 2026

(54) 2,3,4,9-TETRAHYDRO-1H-PYRIDO[3,4-B]INDOLE DERIVATIVES AS ESTROGEN RECEPTOR MODULATORS FOR THE TREATMENT OF CANCER

(71) Applicant: Olema Pharmaceuticals, Inc., San Francisco, CA (US)

(72) Inventors: Brian R. Hearn, Moraga, CA (US); Rupa S. Shetty, San Francisco, CA (US); David C. Myles, Berkeley, CA (US); Dirk A. Heerding, Bend, OR (US)

(21) Appl. No.: 19/104,872

(22) PCT Filed: Aug. 18, 2023
(86) PCT No.: PCT/US2023/030598

§ 371 Date: Feb. 19, 2025

Related U.S. Application Data
(60) Provisional application No. 63/399,273, filed on Aug. 19, 2022.

(51) Int. Cl. C07D 471/04; A61K 31/437; A61K 31/4545; A61K 31/496

(52) U.S. Cl. CPC C07D 471/04; A61K 31/437; A61K 31/4545; A61K 31/496

(57) ABSTRACT

The present disclosure provides 2,3,4,9-tetrahydro-lH-pyrido[3,4-b]indole derivatives of formula I as estrogen receptor modulators for the treatment of cancer.

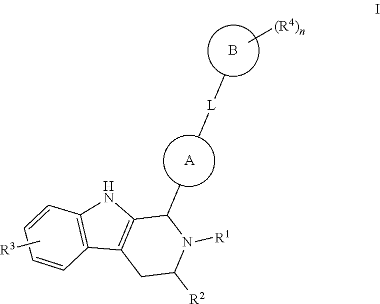

### RELATED APPLICATIONS

This application claims priority to and benefit of U.S. Application No. 63/399,273, filed Aug. 19, 2022, the entire contents of which are hereby incorporated by reference.

### BACKGROUND

The estrogen receptor (ER) plays important roles in various diseases, disorders, and conditions, such as cancers, including breast cancers, menopause-related conditions or symptoms, and osteoporosis. An, K-C. Asian Spine J 10(4); 787-91 (2016 August). About 70% of human breast cancers are hormone dependent and ER-positive. Lumachi, et al., Curr. Med. Chem., 20(5):596-604 (2013). A variety of treatments have been developed to target the estrogen receptor and/or its activities.

### SUMMARY

Selective estrogen receptor modulators (SERMs) or degraders (SERDs) are a particularly useful or promising tools for such therapy. For example, an estrogen receptor modulator that acts as an agonist (or partial agonist) in bone tissue may be useful for treating osteoporosis, e.g., in post-menopausal women. Further, an estrogen receptor modulator that acts as an antagonist in breast tissue may be useful for treating breast cancer. In some instances, the same estrogen receptor modulator may be used in both scenarios.

In some embodiments, the present disclosure provides compounds that are estrogen receptor modulators. In some embodiments, provided compounds are estrogen receptor agonists, e.g., as defined herein. In some embodiments, provided compounds are estrogen receptor antagonists, e.g., as defined herein.

Additionally, there remains a need for anti-estrogen agents that can completely inhibit estrogen receptors, including those coded for by both wild-type and mutant versions (e.g., those containing activating mutations) of the gene encoding Estrogen Receptor-alpha (ERα), Estrogen Receptor 1 (ESR1). The estrogen receptor is a tripartite protein comprising two distinct transcriptional activation functions (AF1 and AF2). Complete anti-estrogen activity requires inactivation of both AF1 and AF2. Activating mutations in the gene that codes for estrogen receptor 1 allows for activation of both AF1 and AF2 even in the absence of estrogen.

Many patients develop resistance to certain therapies that target the estrogen receptor (ER) over time. Certain first line therapies for treating ER-associated diseases, disorders, or conditions, are found to exhibit agonistic activity in conjunction with their antagonistic properties. Fulvestrant, in contrast, is the only approved therapy that exhibits complete anti-estrogenic activity, but is not orally bioavailable, and must be administered parenterally.

In some embodiments, the present disclosure provides certain compounds and compositions that are complete estrogen receptor antagonists, and therefore do not suffer from the deficiencies found in previous therapies.

Additionally, in some embodiments, provided compounds may be orally bioavailable.

In some embodiments, the present disclosure provides an estrogen receptor modulator (e.g., an estrogen receptor agonist, an estrogen receptor antagonist, and/or a complete estrogen receptor antagonist) that is a compound of Formula I.

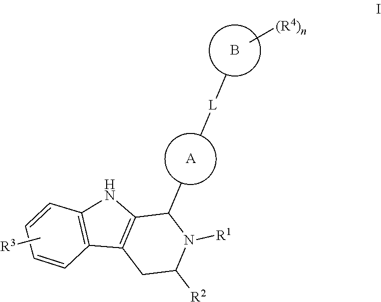

or a pharmaceutically acceptable salt thereof, wherein A, B, L, n, R¹, R², R³, and R⁴ are as defined herein.

In some embodiments, the present disclosure provides methods of treating a disease, disorder, or condition associated with an estrogen receptor. In some embodiments, the present disclosure provides a methods of treating a disease, disorder, or condition associated with a mutation of an estrogen receptor.

In some embodiments, the present disclosure provides methods of treating a cancer. In some embodiments, the present disclosure provides methods of treating a cancer comprising administering a compound of Formula I or a pharmaceutically acceptable salt thereof, optionally in combination with an anti-cancer agent.

In some embodiments, the present disclosure provides methods of preventing recurrence of a cancer. In some embodiments, the present disclosure provides methods of preventing recurrence of a cancer comprising administering a compound of Formula I or a pharmaceutically acceptable salt thereof, optionally in combination with an anti-cancer agent.

In some embodiments, the present disclosure provides methods of treating osteoporosis, e.g., in post-menopausal women. In some embodiments, the present disclosure provides methods of treating osteoporosis comprising administering a compound of Formula I or a pharmaceutically acceptable salt thereof.

In some embodiments, the present disclosure provides methods of treating one or more menopausal symptoms or conditions. In some embodiments, the present disclosure provides methods of treating one or more menopausal symptoms comprising administering a compound of Formula I or a pharmaceutically acceptable salt thereof.

### DETAILED DESCRIPTION OF CERTAIN EMBODIMENTS

The present disclosure provides compounds and compositions useful as complete estrogen receptor modulators (e.g., estrogen receptor agonists, estrogen receptor antagonists, and/or complete estrogen receptor antagonists). In some embodiments, such compounds include those of the formulae described herein, or a pharmaceutically acceptable salt thereof, wherein each variable is as defined and described herein.

### Compounds and Definitions

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19, 18%, 17%, 16%, 15%, 14%, 13%, 12%, 1%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition followed by, after an interval of time, administration of a second or subsequent dose of a pharmaceutical composition.

Agonist: As used herein, the term “agonist” generally refers to an agent whose presence or level correlates with elevated level or activity of a target, as compared with that observed absent the agent (or with the agent at a different level). In some embodiments, an agonist is one whose presence or level correlates with a target level or activity that is comparable to or greater than a particular reference level or activity (e.g., that observed under appropriate reference conditions, such as presence of a known agonist, e.g., a positive control). In some embodiments, an agonist may be a direct agonist in that it exerts its influence directly on (e.g., interacts directly with) the target; in some embodiments, an agonist may be an indirect agonist in that it exerts its influence indirectly (e.g., by acting on, such as interacting with, a regulator of the target, or with some other component or entity.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The term “alkylene” is refers to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” when appended to certain groups herein are intended to refer to a bifunctional moiety of said group. For example, “-ene”, when appended to “cyclopropyl” becomes “cyclopropylene” and is intended to refer to a bifunctional cyclopropyl group, e.g.,

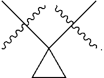 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Aryl: The term “aryl” refers to monocyclic and bicyclic ring systems having a total of six to fourteen ring members (e.g., C₆-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

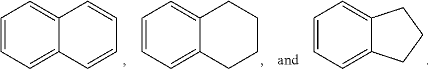

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₅₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated monocyclic or polycyclic ring system of about 3 to about 10 ring carbon atoms. Exemplary monocyclic cycloalkyl rings include cyclopropyl, cyclobutyl, cyclopentyl, cyclohexyl, and cycloheptyl.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π-electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 6- to 10-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 6- to 11-membered bridged heterocyclic ring having one, two, or three bridging atoms).

Modulator: The term “modulator,” as used herein, refers to a compound (e.g., a small molecule) that can alter the activity of another molecule (e.g., a protein). For example, in some embodiments, a modulator can cause an increase or decrease in the magnitude of a certain activity of a type of molecule as compared to the magnitude of the activity in the absence of the modulator. For example, a modulator can be an agonist or an antagonist of a particular target, as those terms are defined herein. For example, in some embodiments, a modulator is an agonist. In some embodiments, a modulator is an antagonist.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

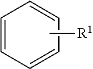

refers to at least

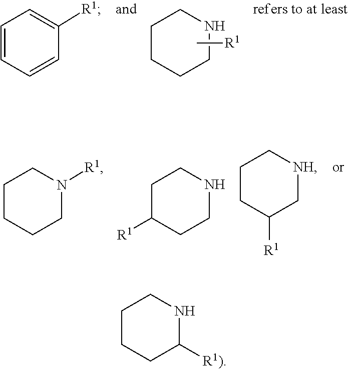

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; —SiR^{◯}₃; —C₁₋₄ straight or branched alkylene)O—N(R^{◯})₂; or —(C₁₋₄ straight or branched alkylene)C(O)O—N(R^{◯})₂, wherein each R◯ may be substituted as defined below and is independently hydrogen, CL-6 aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R◯ (or the ring formed by taking two independent occurrences of R◯ together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR●₃, —C(O)SR^{●}, —C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol [symbol] , as used herein, refers to a point of attachment between two atoms. Additionally or alternatively, the symbol [symbol] refers to a point of attachment ring in a spirocyclic manner.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Estrogen Receptor Modulators

In some embodiments, the present disclosure provides compounds that are estrogen receptor modulators. In some embodiments, provided compounds are estrogen receptor agonists. As used herein, an “estrogen receptor agonist” refers to a compound or composition that produces an agonistic effect when contacting the estrogen receptor of a subject or biological sample. In some embodiments, an estrogen receptor agonist is characterized by having (i) at least 80% increase in the E2-normalized signal in the AP assay (agonist mode) of Example 189 and (ii) no more than 80% reduction in the E2-normalized signal in the AP assay (antagonist mode) of Example 189. In some embodiments, provided compounds are estrogen receptor antagonists. As used herein, an “estrogen receptor antagonist” refers to a compound or composition that produces an antagonistic effect when contacting the estrogen receptor of a subject or biological sample. In some embodiments, an estrogen receptor antagonist is characterized by having:
1. (i) between 10% and 80% increase in the E2-normalized signal in the AP assay (agonist mode) of Example 189 and (ii) between 10% and 80% reduction in the E2-normalized signal in the AP assay (antagonist mode) of Example 189;
2. (i) a pIC₅₀ greater than 6.4 and at least a 10% reduction in the E2-normalized signal in the AP assay (antagonist mode) of Example 189; and (ii) no more than 10% increase in E2-normalized signal in the AP assay (agonist mode) of Example 189; or
3. (i) a pIC₅₀ greater than 7.5 and at least a 10% reduction in the E2-normalized signal in the AP assay (antagonist mode) of Example 189; and (ii) no more than 10% increase in E2-normalized signal in the AP assay (agonist mode) of Example 189.

In some embodiments, the present disclosure provides compounds that are complete estrogen receptor (ER) antagonists. As used herein, a “complete estrogen receptor antagonist” refers to a compound or composition that produces an antagonistic effect when contacting the estrogen receptor of a subject or biological sample, with minimal agonistic effect (e.g., with no or substantially no agonistic effect). Complete estrogen receptor antagonism is determined according to methods described herein, for example in Example 189. In some embodiments, a complete estrogen receptor antagonist is characterized by having (i) a pIC₅₀ greater than 6.4 and at least a 10% reduction in the E2-normalized signal in the AP assay (antagonist mode) of Example 189; and (ii) no more than 10% increase in E2-normalized signal in the AP assay (agonist mode) of Example 189. In some embodiments, a complete estrogen receptor antagonist is characterized by having (i) a pIC₅₀ greater than 7.5 and at least a 10% reduction in the E2-normalized signal in the AP assay (antagonist mode) of Example 189; and (ii) no more than 10% increase in E2-normalized signal in the AP assay (agonist mode) Example 189. In some embodiments, a complete estrogen receptor antagonist is an agent (e.g., a small molecule compound) that shows ER antagonism and no or substantially no ER agonism in one or more of ERα protein level assays, MCF-7 cell line assays, Ishikawa cell line assays (measuring wild type ER and certain mutants including mutants lacking AF1 and/or AF2 domains), and rodent uterine weight gain assays. See, generally, WO 2017/059139. Alternatively or additionally, in some embodiments, a complete estrogen receptor antagonist has three characteristics: it (1) inhibits both activating function 1 (AF1) and activating function 2 (AF2), as complete anti-estrogen activity requires inactivation of both AF1 and AF2; (2) promotes ER degradation; and (3) lacks the partial ER agonist activity observed with certain other agents. Without being bound by theory, it is understood that complete inhibition of both AF1 and AF2 is required for complete estrogen receptor activity, activating mutations in the gene that codes for estrogen receptor 1 allows for activation of both AF1 and AF2 even in the absence of estrogen.

In some embodiments, the present disclosure provides a compound of Formula I:

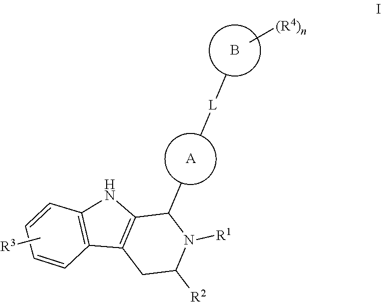

or a pharmaceutically acceptable salt thereof, wherein:
A is an optionally substituted 5-membered heteroaryl comprising 1-3 heteroatoms selected from N, O, and S, wherein at least 1 heteroatom is S or O;
L is a covalent bond or an optionally substituted bivalent group selected from C₁-C₆ aliphatic, -L^{a}-C₀-C₅ aliphatic-, and —C₁-C₅ aliphatic-L^{a}-, wherein L^{a} is selected from —S—, —SO—, —SO₂—, and —N(R^{a})—;
B is selected from 3- to 12-membered heterocyclyl comprising 1-3 heteroatoms selected from N, O, and S and C₃-C₆ cycloaliphatic;
R¹ is selected from hydrogen and optionally substituted C₁-C₆ aliphatic;
R² is selected from hydrogen and optionally substituted C₁-C₆ aliphatic;
R³ is selected from hydrogen, halogen, —CN, —OR^{a}, —C(O)R^{a}, —C(O)OR^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)OR^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group;
each R⁴ is independently oxo, halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S;
each R^{a} is independently selected from hydrogen and optionally substituted C₁-C₆ aliphatic; and n is 0 to 5.

As defined generally above, A is an optionally substituted 5-membered heteroaryl comprising 1-3 heteroatoms selected from N, O, and S, wherein at least 1 heteroatom is S or O. In some embodiments, A is 5-membered heteroaryl comprising 1-3 heteroatoms selected from N, O, and S, wherein at least 1 heteroatom is S or O, optionally substituted with halogen, —(CH₂)₀₋₄R^{◯}, or —(CH₂)₀₋₄OR^{◯}. In some embodiments, A is 5-membered heteroaryl comprising 1-3 heteroatoms selected from N, O, and S, wherein at least 1 heteroatom is S or O, optionally substituted with halogen or C₁-C₆ aliphatic. In some embodiments, A is optionally substituted thiophenyl or thiazolyl. In some embodiments, A is unsubstituted thiophenyl or thiazolyl. In some embodiments, A is thiophenyl or thiazolyl substituted with halogen, —(CH₂)₀₋₄R^{◯}, or —(CH₂)₀₋₄OR^{◯}. In some embodiments, A is thiophenyl or thiazolyl substituted with halogen. In some embodiment, A is optionally substituted thiophenyl. In some embodiments, A is unsubstituted thiophenyl. In some embodiments, A is thiophenyl substituted with halogen, —(CH₂)₀₋₄R^{◯}, or —(CH₂)₀₋₄OR^{◯}. In some embodiments, A is thiophenyl substituted with halogen. In some embodiments, A is optionally substituted thiazolyl. In some embodiments, A is unsubstituted thiazolyl. In some embodiments, A is thiazolyl substituted with halogen, —(CH₂)₀₋₄R^{◯}, or —(CH₂)₀₋₄OR^{◯}. In some embodiments, A is thiazolyl substituted with halogen.

In some embodiments, A is selected from:

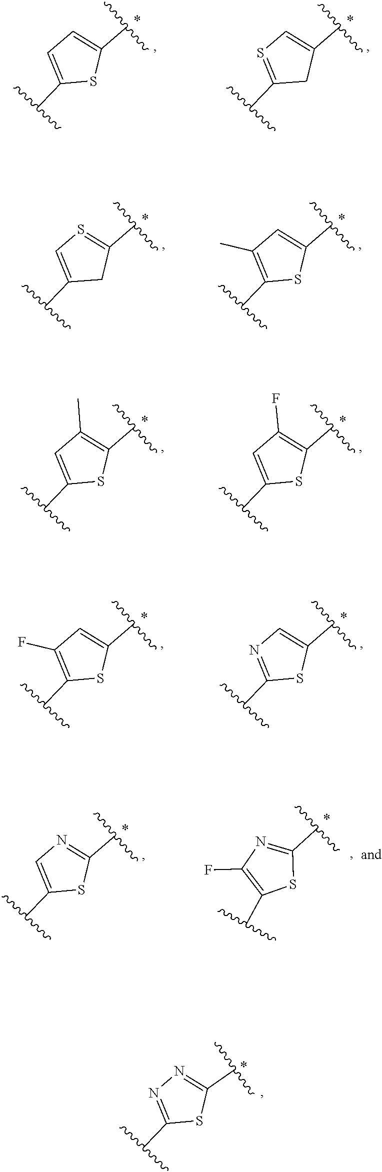

wherein * represents a point of attachment to moiety L. In some embodiments, A is selected from

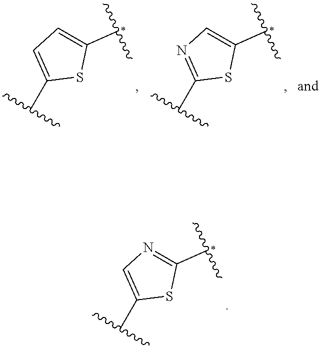 [SMILES: *c1ccc(*)s1 ; *c1cnc(*)s1 ; *c1cnc(*)s1]

In some embodiments, A is

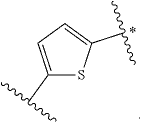 [SMILES: *c1ccc(*)s1]

In some embodiments, A is

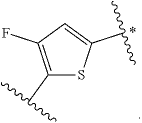 [SMILES: *c1cc(F)c(*)s1]

In some embodiments, A is

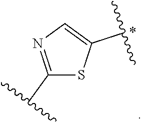 [SMILES: *c1cnc(*)s1]

In some embodiments, A is

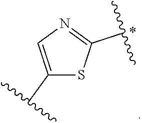 [SMILES: *c1cnc(*)s1]

In some embodiments, A is

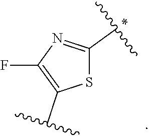 [SMILES: *c1nc(F)c(*)s1]

As described generally above, L is a covalent bond or an optionally substituted bivalent group selected from C₁-C₆ aliphatic, -L^{a}-C₀-C₅ aliphatic-, and —C₁-C₅ aliphatic-L^{a}-, wherein L^{a} is selected from —S—, —SO—, —SO₂—, and —N(R^{a})—. It will be appreciated that the point of attachment of L is indicated by the direction in which its definition is written, such that the leftmost atom is attached to A and the rightmost atom is attached to B. For example, when L is —S—CH₂—, L is attached to A via the sulfur atom and to B via the carbon atom.

In some embodiments, L is a covalent bond.

In some embodiments, L is optionally substituted C₁-C₆ aliphatic. In some embodiments, L is —(CH₂)₁₋₆—. In some embodiments, L is —CH₂—, —CH₂—CH₂—, —CH₂—CH₂—, —CH₂—CH₂—CH₂—, —CH₂—CH₂—CH₂—CH₂—, —CH₂—CH₂—CH₂—CH₂—CH₂—, or —CH₂—CH₂—CH₂—CH₂—CH₂—CH₂—. In some embodiments, L is —CH₂—.

In some embodiments, L is optionally substituted -L^{a}-C₀-C₅ aliphatic-. In some embodiments, L is —S—C₀-C₅ alipahtic-, —SO—C₀-C₅ aliphatic-, —SO₂—C₀-C₅ aliphatic-, or —N(R^{a})—C₀-C₅ aliphatic-. In some embodiments, L is —S—C₀-C₅ aliphatic-. In some embodiments, L is —SO—C₀-C₅ aliphatic-. In some embodiments, L is —SO₂—C₀-C₅ aliphatic-. In some embodiments, L is —N(R^{a})—C₀-C₅ aliphatic-. In some embodiments, L is L^{a}. In some embodiments, L is —S—, —SO—, —SO₂—, or —N(R^{a})—. In some embodiments, L is —S—. In some embodiments, L is —SO—. In some embodiments, L is —SO₂—. In some embodiments, L is —N(R^{a})—. In some embodiments, L is —N(H)—.

In some embodiments, L is selected from a covalent bond, —CH₂—, —CH₂—CH₂—, —CH₂—CH₂—CH₂—, —S—, and —SO₂—. In some embodiments, L is selected from a covalent bond, —CH₂—, —CH₂—CH₂—, —CH₂—CH₂—CH₂—, —S—, and —N(H)—.

As described generally above, B is selected from 3- to 12-membered heterocyclyl comprising 1-3 heteroatoms selected from N, O, and S and C₃-C₆ cycloaliphatic.

In some embodiments, B is selected from 3- to 6-membered heterocyclyl comprising 1-3 heteroatoms selected from N, O, and S and C₃₋₆ cycloaliphatic. In some embodiments, B is selected from 4- to 6-membered heterocyclyl comprising 1-3 heteroatoms selected from N, O, and S and C₃₋₆ cycloaliphatic.

In some embodiments, B is 3- to 6-membered heterocyclyl comprising 1-3 heteroatoms selected from N, O, and S. In some embodiments, B is 4- to 6-membered heterocyclyl comprising 1-3 heteroatoms selected from N, O, and S. In some embodiments, B is 4- to 5-membered heterocyclyl comprising 1-3 heteroatoms selected from N, O, and S. In some embodiments, B is 3-membered heterocyclyl comprising 1 heteroatom selected from N, O, and S. In some embodiments, B is 4-membered heterocyclyl comprising 1-2 heteroatoms selected from N, O, and S. In some embodiments, B is 5-membered heterocyclyl comprising 1-3 heteroatoms selected from N, O, and S. In some embodiments, B is 6-membered heterocyclyl comprising 1-3 heteroatoms selected from N, O, and S. In some embodiments, B is azetidinyl, pyrrolidinyl, piperidinyl, or piperazinyl. In some embodiments, B is azetidinyl or pyrrolidinyl. In some embodiments, B is azetidinyl. In some embodiments, B is pyrrolidinyl. In some embodiments, B is selected from:

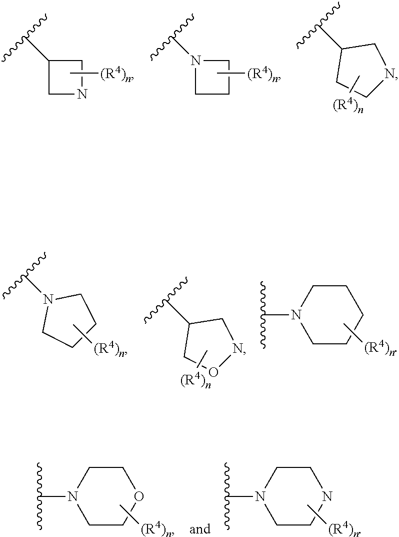

In some embodiments, B is 6- to 12-membered bicyclic fused or spirocyclic heterocyclyl. In some embodiments, B is 6- to 8-membered bicyclic fused or spirocyclic heterocyclyl. In some embodiments, B is 6- to 12-membered bicyclic fused heterocyclyl. In some embodiments, B is 6- to 8-membered bicyclic fused heterocyclyl. In some embodiments, B is 6- to 12-membered bicyclic spirocyclic heterocyclyl. In some embodiments, B is 6- to 8-membered bicyclic spirocyclic heterocyclyl. In some embodiments, B is selected from:

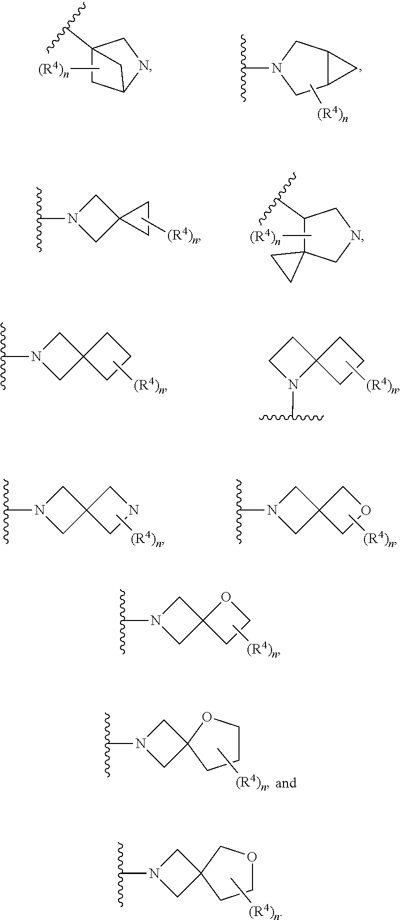

In some embodiments, B is C₃-C₆ cycloaliphatic. In some embodiments, B is cyclopropyl, cyclobutyl, cyclopentyl, or cyclohexyl.

As described generally above, each R⁴ is independently oxo, halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S.

In some embodiments, each R⁴ is independently halogen or optionally substituted C₁₋₆ aliphatic.

In some embodiments, R⁴ is oxo.

In some embodiments, R⁴ is halogen. In some embodiments, R⁴ is fluoro.

In some embodiments, R⁴ is —CN.

In some embodiments, R⁴ is —OR^{a}. In some embodiments, R⁴ is —OH. In some embodiments, R⁴ is —O—C₁-C₆ aliphatic.

In some embodiments, R⁴ is —N(R^{a})₂. In some embodiments, R⁴ is —N(H)(R^{a}). In some embodiments, R⁴ is —NH₂. In some embodiments, R⁴ is —N(H)C₁-C₆ aliphatic. In some embodiments, R⁴ is —N(C₁-C₆ aliphatic)₂. In some embodiments, R⁴ is —N(H)CH₃, —N(H)CH₂CH₃, —N(CH₂CH₃)₂, —N(H)CH₂CH₂CH₃, —N(CH₃)₂, —N(CH₃)CH₂CH₃, or —N(CH₃)CH₂CH₂CH₃.

In some embodiments, R⁴ is —C(O)R^{a}. In some embodiments, R⁴ is C(O)—C₁-C₆ aliphatic optionally substituted with —(CH₂)₀₋₄OR^{◯}. In some embodiments, R⁴ is —C(O)CH₃, —C(O)CH₂OCH₃, or —C(O)CH₂CH₂OCH₃.

In some embodiments, R⁴ is —OC(O)R^{a}. In some embodiments, R⁴ is —OC(O)—C₁-C₆ aliphatic.

In some embodiments, R⁴ is —C(O)₂R^{a}. In some embodiments, R⁴ is —C(O)OH. In some embodiments, R⁴ is —C(O)₂—C₁-C₆ aliphatic.

In some embodiments, R⁴ is —C(O)N(R^{a})₂. In some embodiments, R⁴ is —C(O)N(H)R^{a}. In some embodiments, R⁴ is —C(O)NH₂. In some embodiments, R⁴ is —C(O)N(H)C₁-C₆ aliphatic.

In some embodiments, R⁴ is —N(R^{a})C(O)R^{a}. In some embodiments, R⁴ is —N(H)C(O)R^{a}. In some embodiments, R⁴ is —N(H)C(O)(C₁-C₆ aliphatic optionally substituted with —(CH₂)₀₋₄OR^{◯}). In some embodiments, R⁴ is —N(H)C(O)CH₂OH or —N(H)C(O)CH₂OCH₃.

In some embodiments, R⁴ is an optionally substituted C₁-C₆ aliphatic. In some embodiments, R⁴ is C₁-C₆ aliphatic optionally substituted with halogen, —(CH₂)₀₋₄R^{◯}, —(CH₂)₀₋₄OR^{◯}, —O(CH₂)₀₋₄R^{◯}, —CN, —(CH₂)₀₋₄N(R^{◯})₂, or phenyl. In some embodiments, R⁴ is C₁-C₆ aliphatic substituted with halogen, —OH, —OCH₃, —CN, or 3- to 6-membered saturated, partially unsaturated, or aryl ring having 0-4 heteroatoms independently selected from nitrogen, oxygen, or sulfur. In some embodiments, R⁴ is C₁-C₆ aliphatic optionally substituted with halogen. In some embodiments, R⁴ is C₁-C₃ alkyl optionally substituted with halogen. In some embodiments, R⁴ is methyl, ethyl, propyl, butyl, pentyl, hexyl, —CH₂F, —CH₂CH₂F, —CH₂CHF₂, —CH₂CH₂CHF₂, —CH₂CH₂CF₃, —CH₂CH₂CH₂OH, —CH₂CH₂CH₂OCH₃, —CH₂CH₂CH₂F, —CH₂CH₂CH(CH₃)₂, —CH₂CH₂Ph, —CH₂CH₂OCH₃, —CH₂CH₂OCH₂CH₃, —CH₂CH₂CH₂OCH₂CH₃, —CH₂C≡CH, —CH₂C≡CHCH₃, —CH₂CN, —CH₂CH₂CN, —CH₂CH₂CH₂OCF₃,

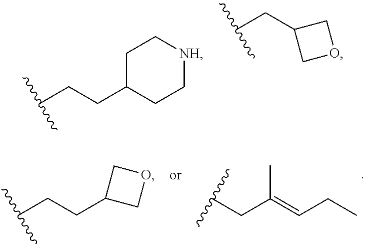

In some embodiments, R⁴ is ethyl, propyl, —CH₂CH₂CH₂F, —CH₂CH₂CHF₂, —CH₂CH₂CH₂OCH₃, —CH₂C≡CH, or

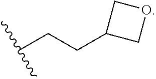 [SMILES: *CCC1COC1]

In some embodiments, R⁴ is methyl, ethyl, propyl, —CH₂F, —CH₂CH₂F, —CH₂CH₂CH₂F, —CH₂CH₂CHF₂, —CH₂CH₂CF₃, —CH₂CH₂CH₂OCH₃, —CH₂C≡CH, —CH₂CH₂CN, or

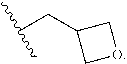 [SMILES: *CC1COC1]

In some embodiments, R⁴ is —CH₂F or —CH₂CH₂CH₂F.

In some embodiments, R⁴ is an optionally substituted 3- to 12-membered heterocyclyl comprising 1 to 3 heteroatoms selected from N, O, and S. In some embodiments, R⁴ is optionally substituted 3- to 6-membered heterocyclyl comprising 1 to 3 heteroatoms selected from N, O, and S. In some embodiments, R⁴ is optionally substituted azetidinyl.

In some embodiments, R⁴ is oxo, —OH, —OCH₃, fluoro, methyl, ethyl, propyl, isopropyl, butyl, pentyl, hexyl, —CH₂F, —CH₂CH₂F, —CH₂CHF₂, —CH₂CH₂CHF₂, —CH₂CH₂CF₃, —CH₂CH₂CH₂OH, —CH₂CH₂CH₂OCH₃, —CH₂CH₂CH₂F, —CH₂CH₂CH(CH₃)₂, —CH₂CH₂-Ph, —C(CH₃)₂—OH, —CH₂OCH₃, —CH₂CH₂OCH₃, —CH₂CH₂OCH₂CH₃, —CH₂CH₂CH₂OCH₂CH₃, —CH₂C≡CH, —CH₂C≡CHCH₃, —CH₂CN, —CH₂CH₂CN, —CH₂CH₂CH₂OCF₃, —NHCH₂CH₃, —NHCH₂CH₂CH₃, —NHCH₂CH₂CH₃, —NHCH₃, —N(CH₃)CH₂CH₃, —N(CH₃)CH₂CH₂CH₃, —N(CH₃)₂, —NHC(O)CH₂OCH₃, —NHC(O)CH₂CH₂OCH₃, —NHC(O)CH₂OH, —C(O)CF₃,

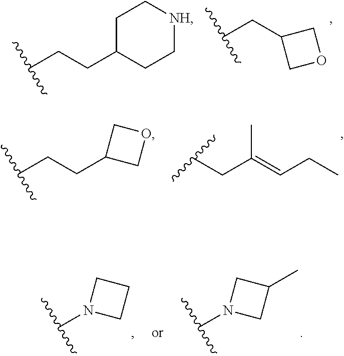

In some embodiments, R⁴ is fluoro, ethyl, propyl, —CH₂CH₂CH₂F, —CH₂CH₂CHF₂, —CH₂CH₂CH₂OCH₃, —CH₂C≡CH, or

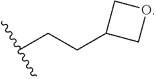 [SMILES: *CCC1COC1]

In some embodiments, R⁴ is fluoro, methyl, ethyl, propyl, —CH₂F, —CH₂CH₂F, —CH₂CH₂CH₂F, —CH₂CH₂CHF₂, —CH₂CH₂CF₃, —CH₂CH₂CH₂OCH₃, —CH₂C≡CH, —CH₂CH₂CN, or

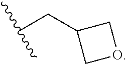 [SMILES: *CC1COC1]

In some embodiments, R⁴ is —CH₂F or —CH₂CH₂CH₂F.

In some embodiments, a moiety:

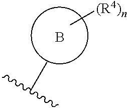

is a moiety selected from:

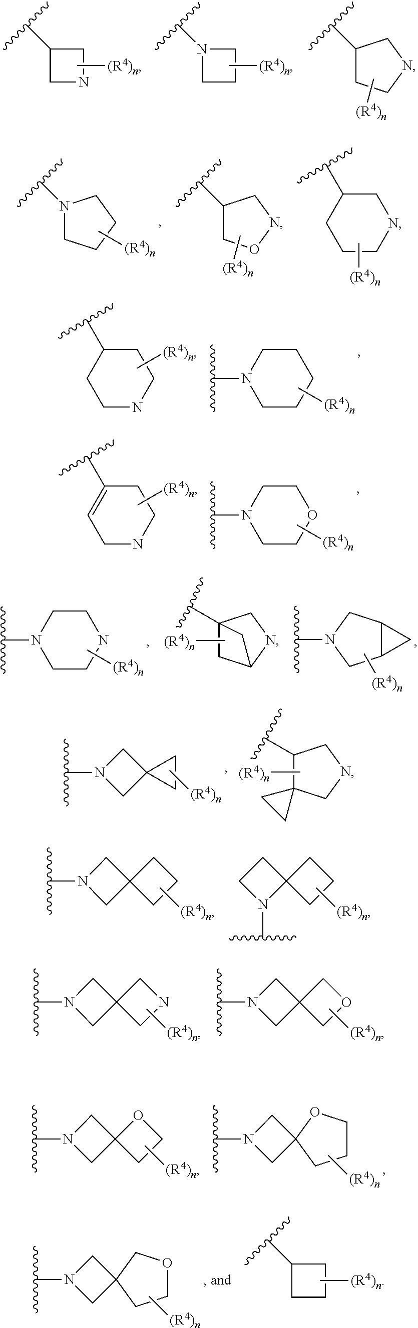

In some embodiments, a moiety:

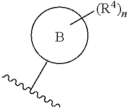

is a moiety selected from:

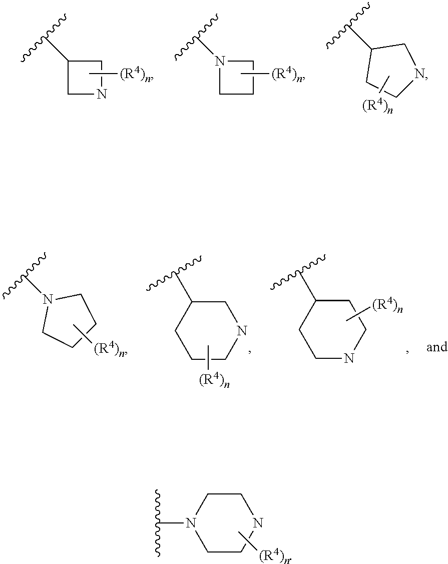

In some embodiments, a moiety:

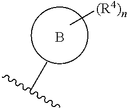

is a moiety selected from:

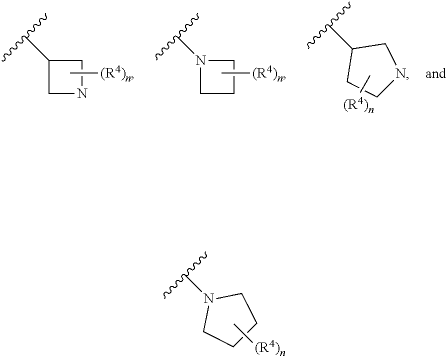

In some embodiments, a moiety:

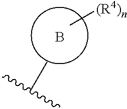

is a moiety selected from:

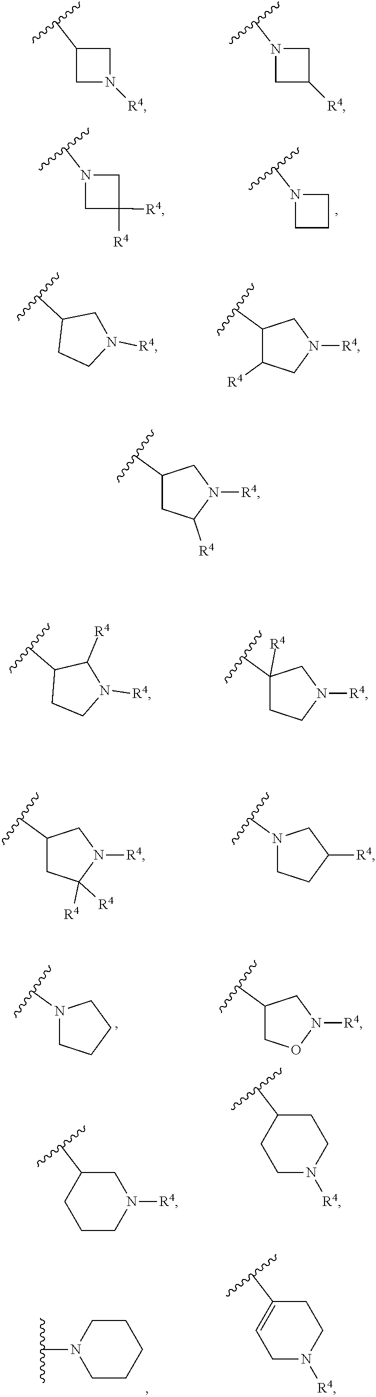

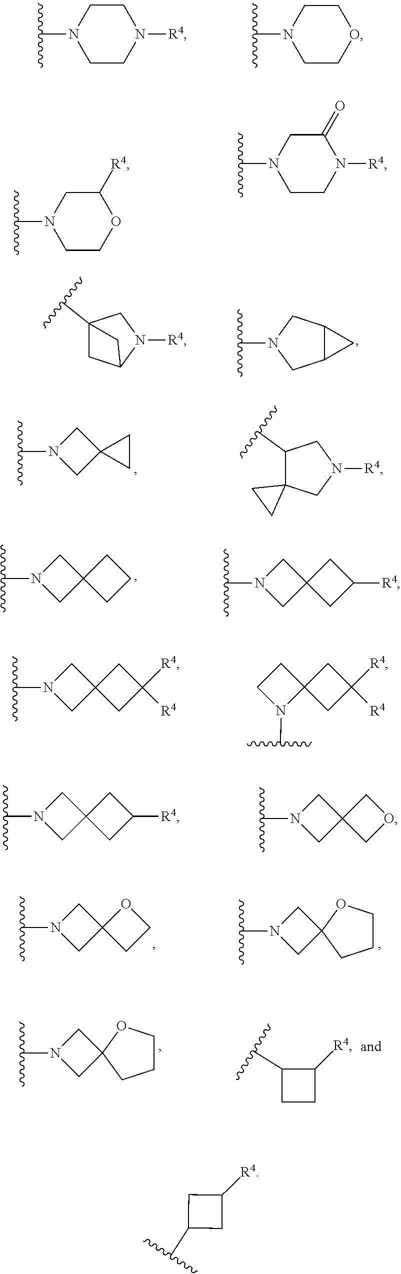

In some embodiments, a moiety:

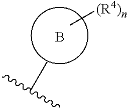

is a moiety selected from:

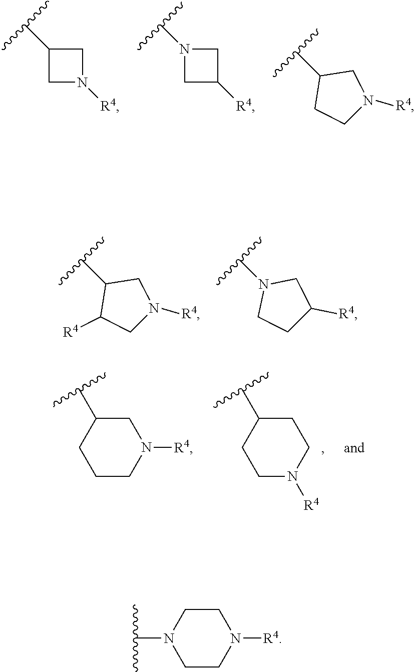

In some embodiments, a moiety:

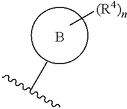

is a moiety selected from:

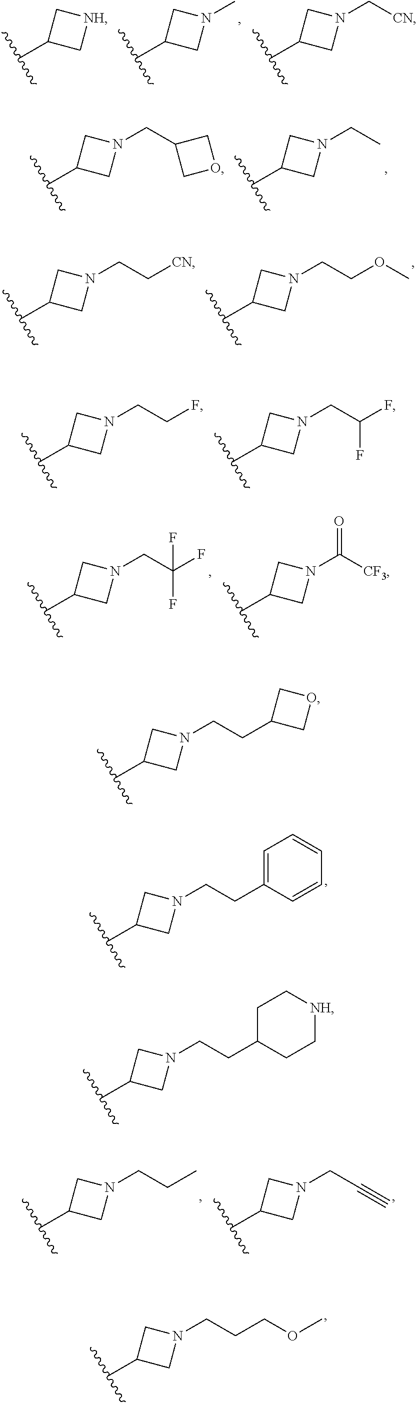

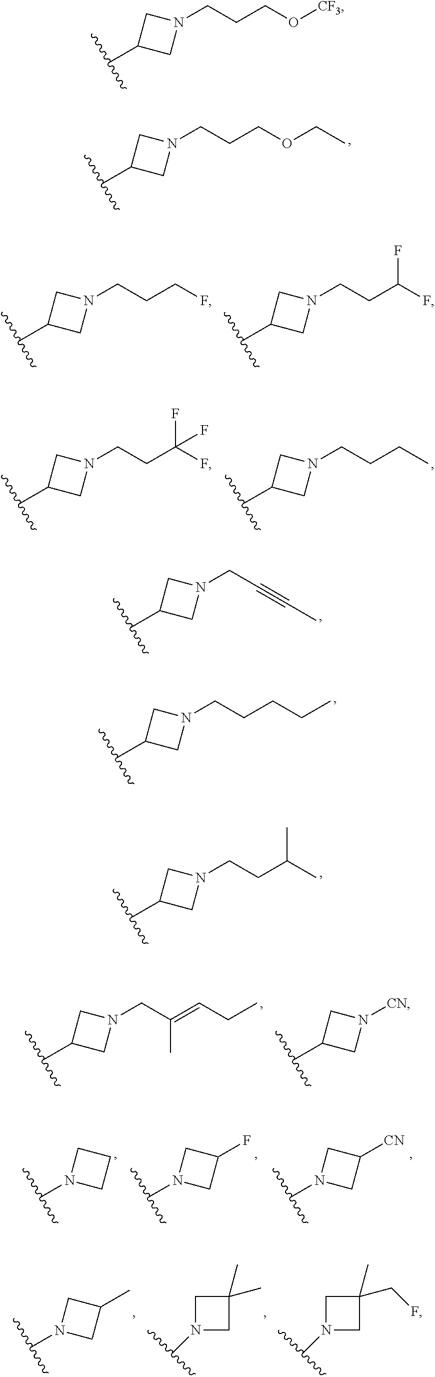

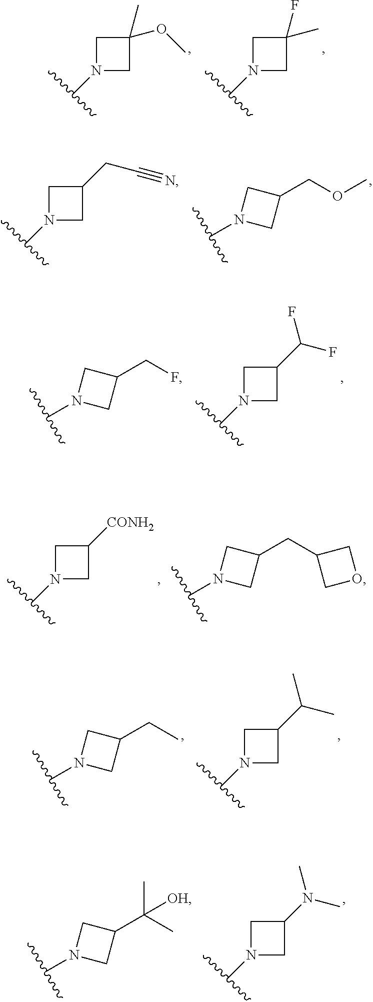

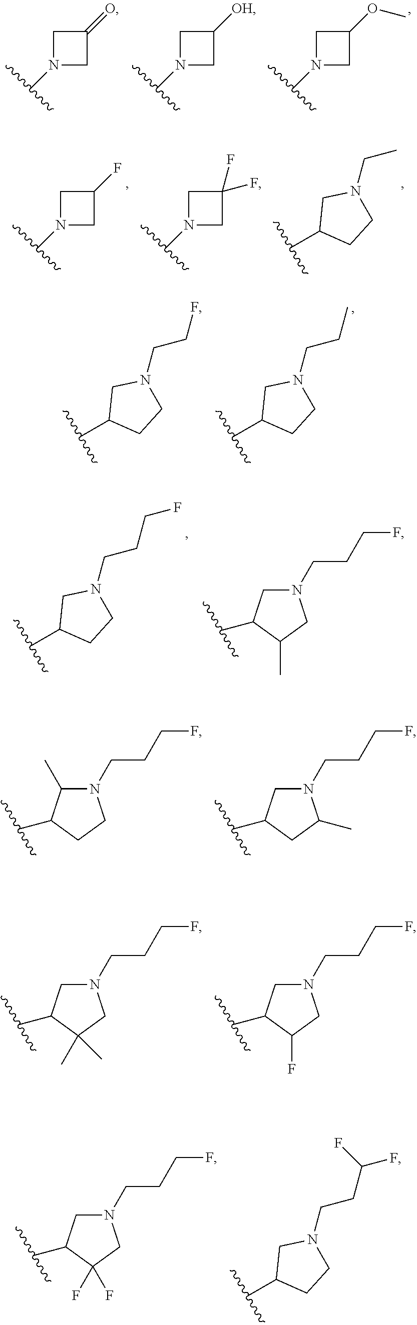

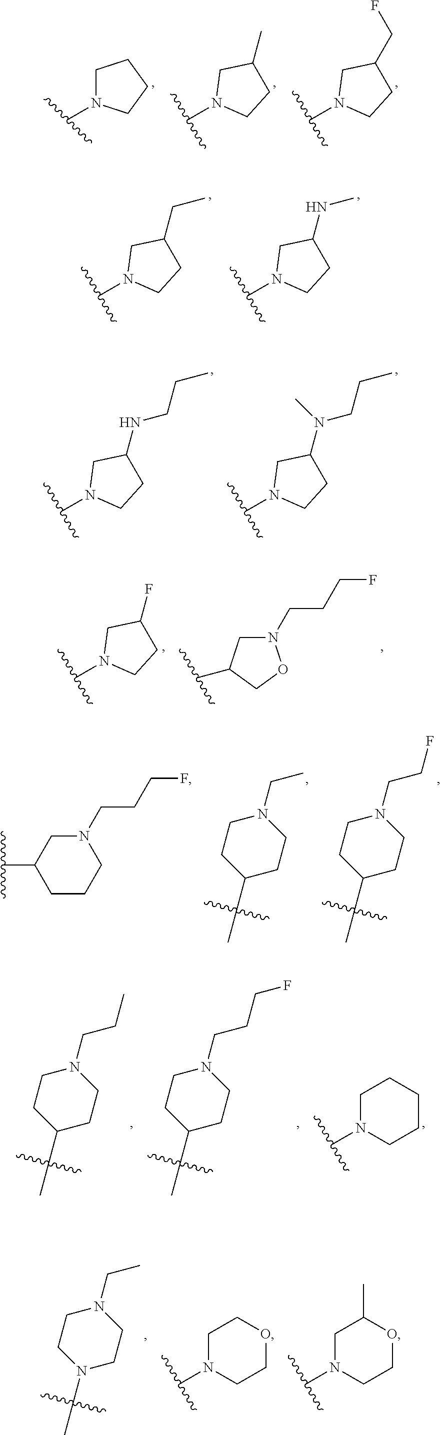

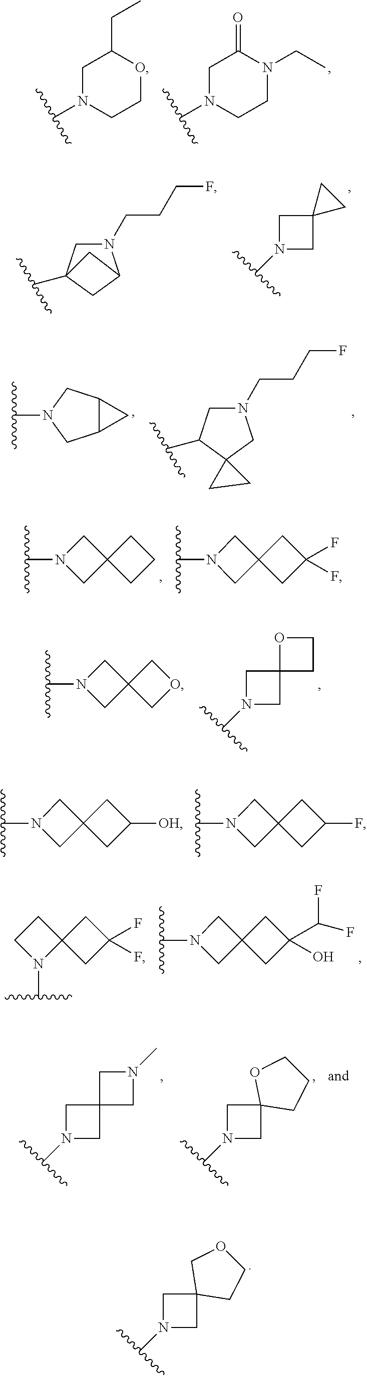

In some embodiments, a moiety:

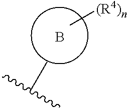

is a moiety selected from:

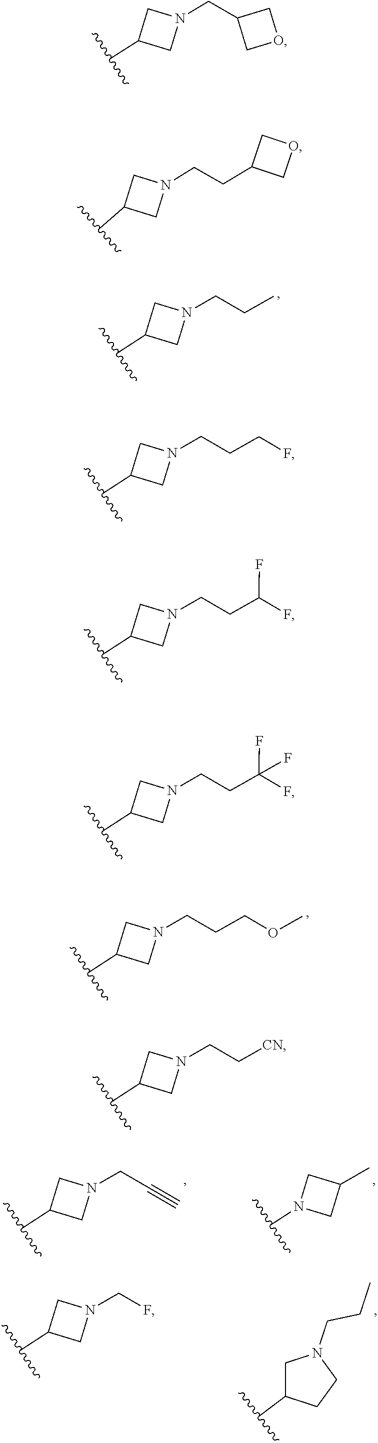

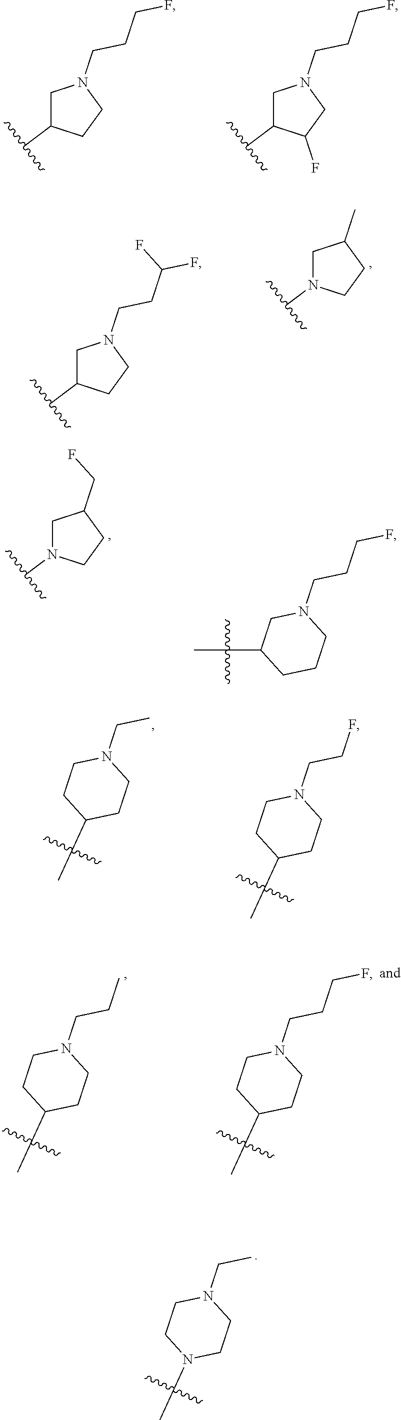

In some embodiments, a moiety:

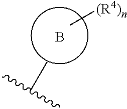

is a moiety selected from:

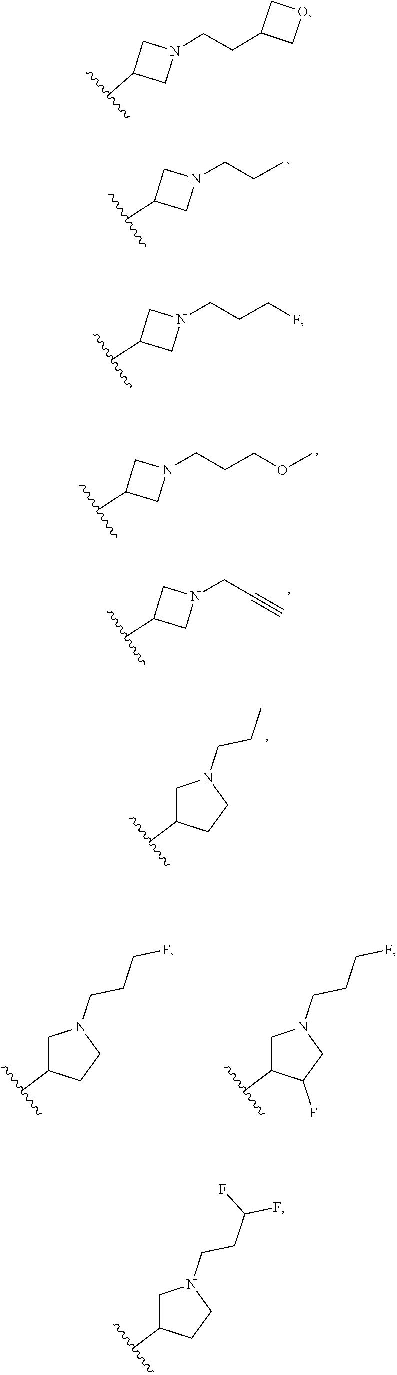

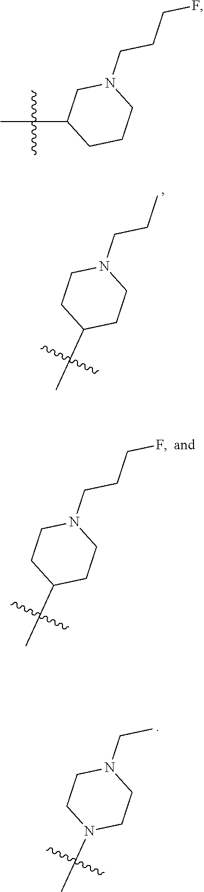

In some embodiments, a moiety:

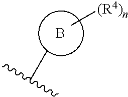

is a moiety selected from:

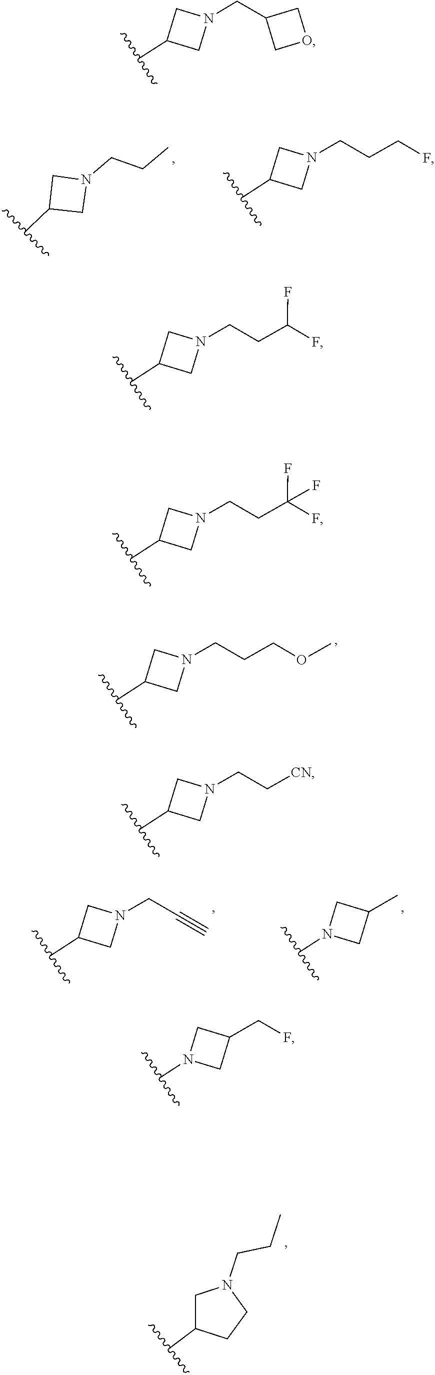

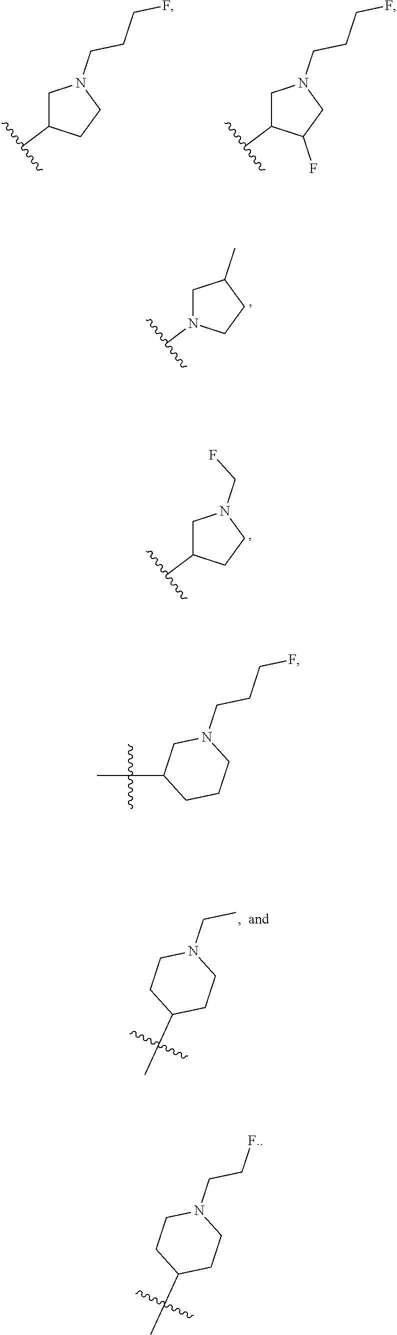

In some embodiments, a moiety:

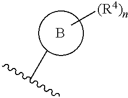

is a moiety selected from:

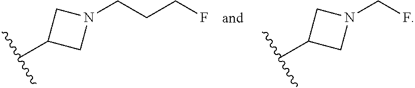 [SMILES: *C1CN(CCCF)C1 ; "and" ; *C1CN(CF)C1]

As described generally above, R¹ is selected from hydrogen and optionally substituted C₁-C₆ aliphatic. In some embodiments, R¹ is hydrogen. In some embodiments, R¹ is C₁-C₆ aliphatic optionally substituted with halogen, —(CH₂)₀₋₄R^{◯}, —(CH₂)₀₋₄OR^{◯}, or —(CH₂)₀₋₄Ph. In some embodiments, R¹ is C₁-C₆ aliphatic optionally substituted with halogen or —OH.

In some embodiments, R¹ is selected from:

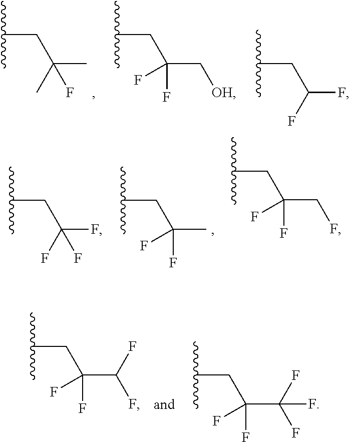

In some embodiments, R¹ is selected from:

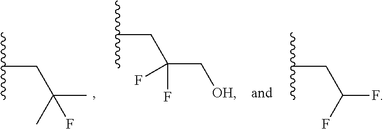

As described generally above, R² is selected from hydrogen and optionally substituted C₁-C₆ aliphatic. In some embodiments, R² is hydrogen. In some embodiments, R² is C₁-C₆ aliphatic. In some embodiments, R² is methyl, ethyl, propyl, butyl, pentyl, or hexyl. In some embodiments, R² is methyl.

As described generally above, R³ is selected from hydrogen, halogen, —CN, —OR^{a}, —C(O)R^{a}, —C(O)₂R^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)₂R^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group. In some embodiments, R³ is hydrogen.

As described generally above, n is 0-5. In some embodiments, n is 0. In some embodiments, n is 1, 2, 3, 4, or 5. In some embodiments, n is 1. In some embodiments, n is 2. In some embodiments, n is 3. In some embodiments, n is 4. In some embodiments, n is 5.

In some embodiments, a compound of Formula I is a compound of Formula II:

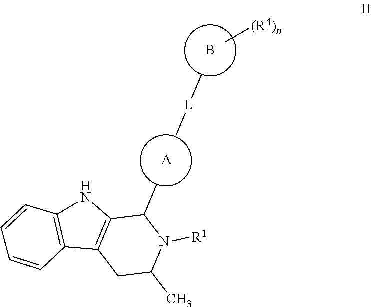

or a pharmaceutically acceptable salt thereof, wherein A, B, L, n, R¹, and R⁴ are as defined above for Formula I and described in classes and subclasses herein, both singly and in combination.

In some embodiments, a compound of Formula I is a compound of Formula II-a:

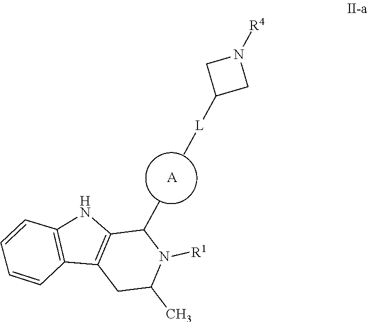

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-b:

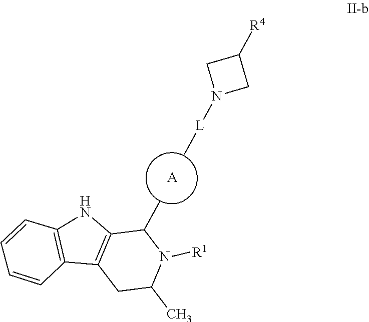

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-c:

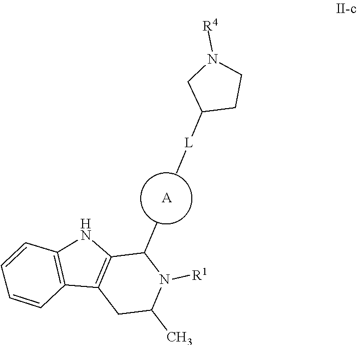

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-d:

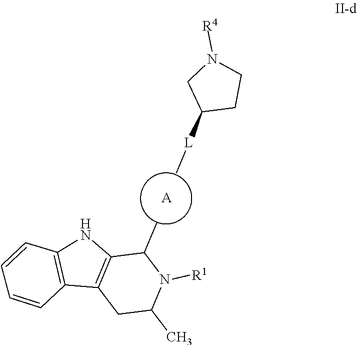

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-e:

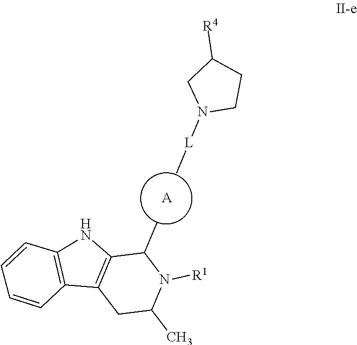

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-f:

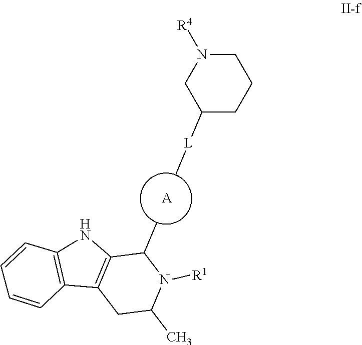

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-g:

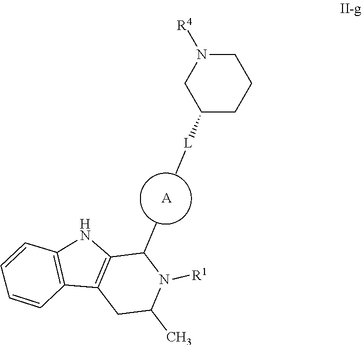

or a pharmaceutically acceptable salt thereof, wherein A, L, R¹, and R⁴ are as defined above for Formula I and described in classes and subclasses herein, both singly and in combination.

In some embodiments a compound of Formula I is a compound of Formula II-h:

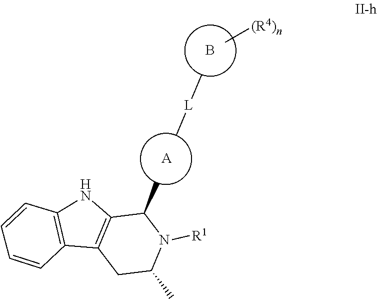

or a pharmaceutically acceptable salt thereof, wherein A, B, L, n, R¹, and R⁴ are as defined above for Formula I and described in classes and subclasses herein, both singly and in combination.

In some embodiments, a compound of Formula I is a compound of Formula III:

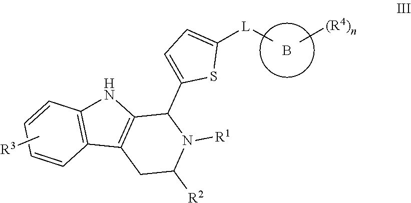

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula IV:

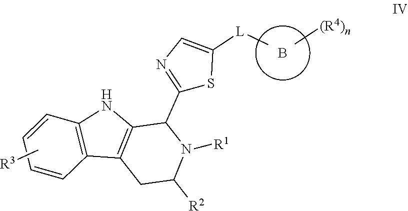

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula V:

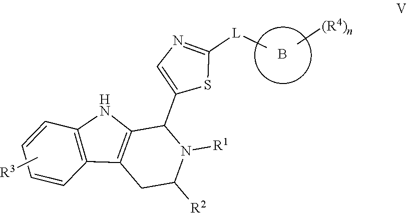

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula VI:

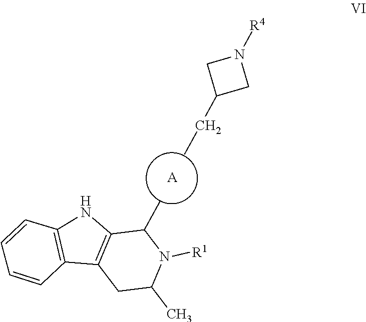

or a pharmaceutically acceptable salt thereof, wherein A, R¹, and R⁴ are as defined above for Formula I and described in classes and subclasses herein, both singly and in combination.

In some embodiments, the present disclosure provides compounds selected from Table 1, or a pharmaceutically acceptable salt thereof:

TABLE 1

| Compound Structure | Compound No. |
|---|---|
| 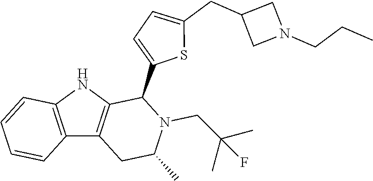 [SMILES: CCCN1CC(Cc2ccc([C@@H]3c4[nH]c5ccccc5c4C[C@@H](C)N3CC(C)(C)F)s2)C1] | I-1 |
| 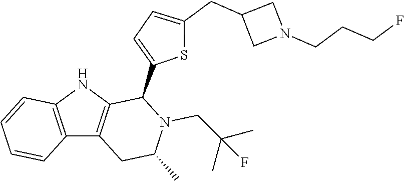 [SMILES: C[C@@H]1Cc2c([nH]c3ccccc23)[C@@H](c2ccc(CC3CN(CCCF)C3)s2)N1CC(C)(C)F] | I-2 |
| 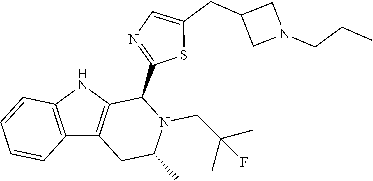 [SMILES: CCCN1CC(Cc2cnc([C@@H]3c4[nH]c5ccccc5c4C[C@@H](C)N3CC(C)(C)F)s2)C1] | I-3 |
| 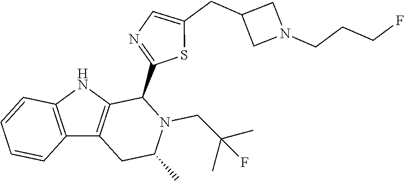 [SMILES: C[C@@H]1Cc2c([nH]c3ccccc23)[C@@H](c2ncc(CC3CN(CCCF)C3)s2)N1CC(C)(C)F] | I-4 |
| 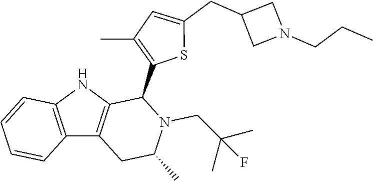 [SMILES: CCCN1CC(Cc2cc(C)c([C@@H]3c4[nH]c5ccccc5c4C[C@@H](C)N3CC(C)(C)F)s2)C1] | I-5 |
| 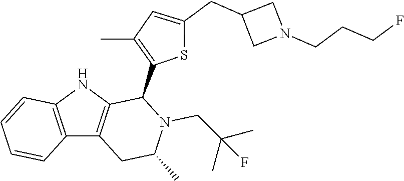 [SMILES: Cc1cc(CC2CN(CCCF)C2)sc1[C@@H]1c2[nH]c3ccccc3c2C[C@@H](C)N1CC(C)(C)F] | I-6 |
| 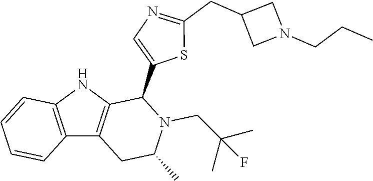 [SMILES: CCCN1CC(Cc2ncc([C@@H]3c4[nH]c5ccccc5c4C[C@@H](C)N3CC(C)(C)F)s2)C1] | I-7 |
| 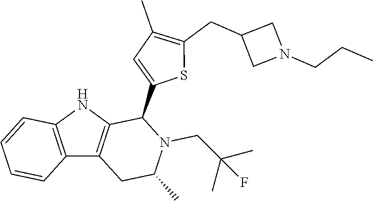 [SMILES: CCCN1CC(Cc2sc([C@@H]3c4[nH]c5ccccc5c4C[C@@H](C)N3CC(C)(C)F)cc2C)C1] | I-8 |
| 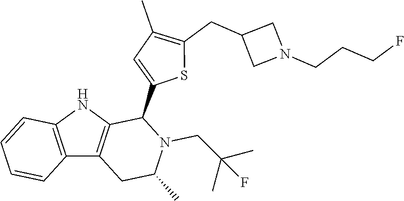 [SMILES: Cc1cc([C@@H]2c3[nH]c4ccccc4c3C[C@@H](C)N2CC(C)(C)F)sc1CC1CN(CCCF)C1] | I-9 |
| 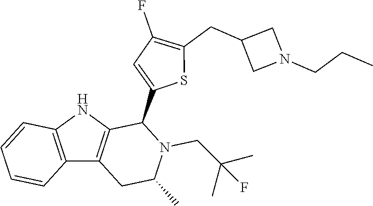 [SMILES: CCCN1CC(Cc2sc([C@@H]3c4[nH]c5ccccc5c4C[C@@H](C)N3CC(C)(C)F)cc2F)C1] | I-10 |
| 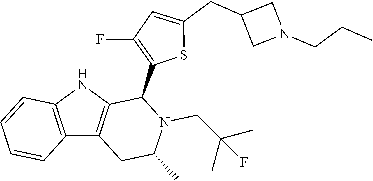 [SMILES: CCCN1CC(Cc2cc(F)c([C@@H]3c4[nH]c5ccccc5c4C[C@@H](C)N3CC(C)(C)F)s2)C1] | I-11 |
| 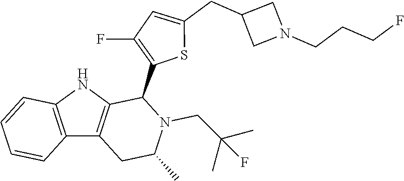 [SMILES: C[C@@H]1Cc2c([nH]c3ccccc23)[C@@H](c2sc(CC3CN(CCCF)C3)cc2F)N1CC(C)(C)F] | I-12 |
| 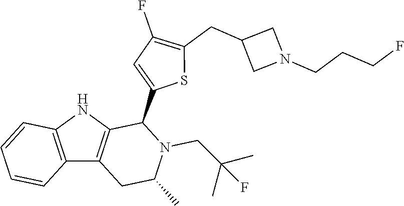 [SMILES: C[C@@H]1Cc2c([nH]c3ccccc23)[C@@H](c2cc(F)c(CC3CN(CCCF)C3)s2)N1CC(C)(C)F] | I-13 |
| 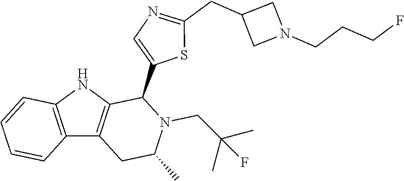 [SMILES: C[C@@H]1Cc2c([nH]c3ccccc23)[C@@H](c2cnc(CC3CN(CCCF)C3)s2)N1CC(C)(C)F] | I-14 |
| 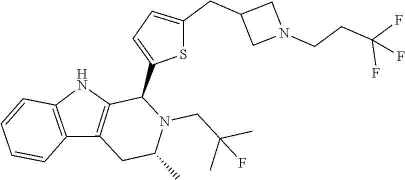 [SMILES: C[C@@H]1Cc2c([nH]c3ccccc23)[C@@H](c2ccc(CC3CN(CCC(F)(F)F)C3)s2)N1CC(C)(C)F] | I-15 |
| 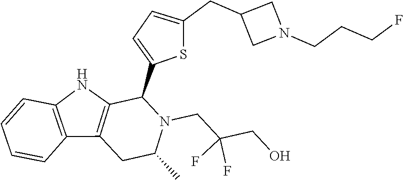 [SMILES: C[C@@H]1Cc2c([nH]c3ccccc23)[C@@H](c2ccc(CC3CN(CCCF)C3)s2)N1CC(F)(F)CO] | I-16 |
| 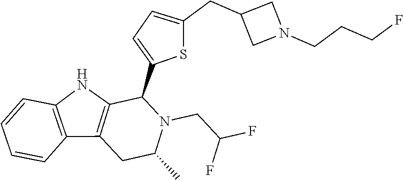 [SMILES: C[C@@H]1Cc2c([nH]c3ccccc23)[C@@H](c2ccc(CC3CN(CCCF)C3)s2)N1CC(F)F] | I-17 |
| 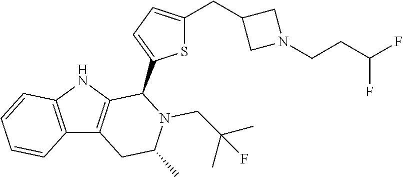 [SMILES: C[C@@H]1Cc2c([nH]c3ccccc23)[C@@H](c2ccc(CC3CN(CCC(F)F)C3)s2)N1CC(C)(C)F] | I-18 |
| 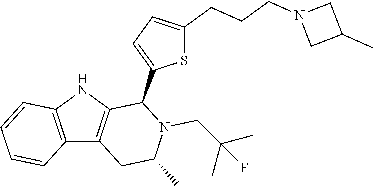 [SMILES: CC1CN(CCCc2ccc([C@@H]3c4[nH]c5ccccc5c4C[C@@H](C)N3CC(C)(C)F)s2)C1] | I-19 |
| 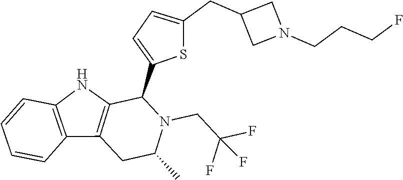 [SMILES: C[C@@H]1Cc2c([nH]c3ccccc23)[C@@H](c2ccc(CC3CN(CCCF)C3)s2)N1CC(F)(F)F] | I-20 |
| 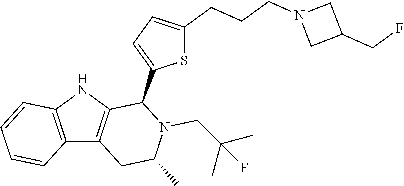 [SMILES: C[C@@H]1Cc2c([nH]c3ccccc23)[C@@H](c2ccc(CCCN3CC(CF)C3)s2)N1CC(C)(C)F] | I-21 |
| 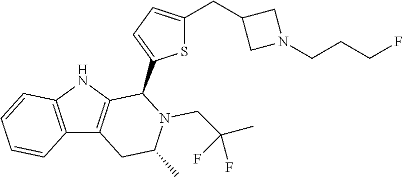 [SMILES: C[C@@H]1Cc2c([nH]c3ccccc23)[C@@H](c2ccc(CC3CN(CCCF)C3)s2)N1CC(C)(F)F] | I-22 |
| 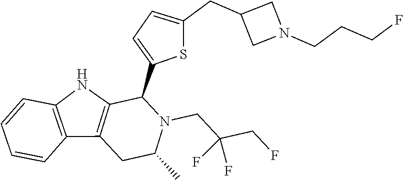 [SMILES: C[C@@H]1Cc2c([nH]c3ccccc23)[C@@H](c2ccc(CC3CN(CCCF)C3)s2)N1CC(F)(F)CF] | I-23 |
| 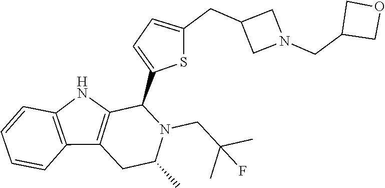 [SMILES: C[C@@H]1Cc2c([nH]c3ccccc23)[C@@H](c2ccc(CC3CN(CC4COC4)C3)s2)N1CC(C)(C)F] | I-24 |
| 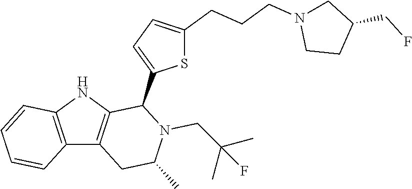 [SMILES: C[C@@H]1Cc2c([nH]c3ccccc23)[C@@H](c2ccc(CCCN3CC[C@@H](CF)C3)s2)N1CC(C)(C)F] | I-25 |
| 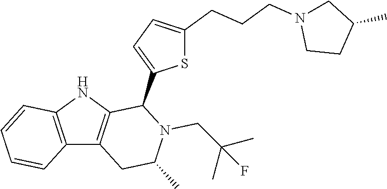 [SMILES: C[C@@H]1CCN(CCCc2ccc([C@@H]3c4[nH]c5ccccc5c4C[C@@H](C)N3CC(C)(C)F)s2)C1] | I-26 |
| 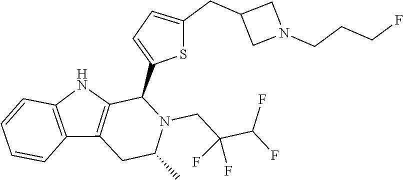 [SMILES: C[C@@H]1Cc2c([nH]c3ccccc23)[C@@H](c2ccc(CC3CN(CCCF)C3)s2)N1CC(F)(F)C(F)F] | I-27 |
| 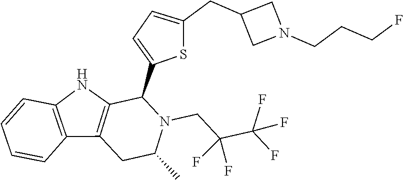 [SMILES: C[C@@H]1Cc2c([nH]c3ccccc23)[C@@H](c2ccc(CC3CN(CCCF)C3)s2)N1CC(F)(F)C(F)(F)F] | I-28 |
| 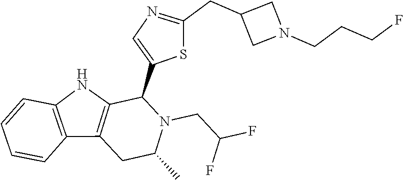 [SMILES: C[C@@H]1Cc2c([nH]c3ccccc23)[C@@H](c2cnc(CC3CN(CCCF)C3)s2)N1CC(F)F] | I-29 |
| 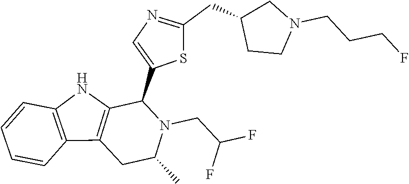 [SMILES: C[C@@H]1Cc2c([nH]c3ccccc23)[C@@H](c2cnc(C[C@H]3CCN(CCCF)C3)s2)N1CC(F)F] | I-30 |
| 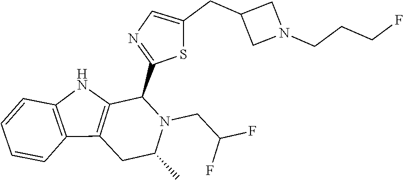 [SMILES: C[C@@H]1Cc2c([nH]c3ccccc23)[C@@H](c2ncc(CC3CN(CCCF)C3)s2)N1CC(F)F] | I-31 |
| 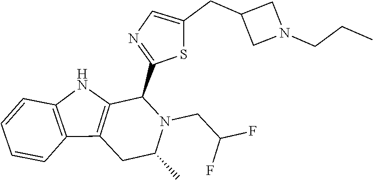 [SMILES: CCCN1CC(Cc2cnc([C@@H]3c4[nH]c5ccccc5c4C[C@@H](C)N3CC(F)F)s2)C1] | I-32 |
| 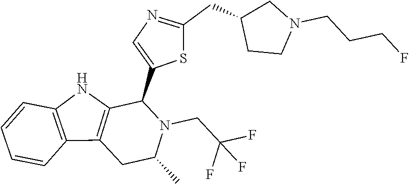 [SMILES: C[C@@H]1Cc2c([nH]c3ccccc23)[C@@H](c2cnc(C[C@H]3CCN(CCCF)C3)s2)N1CC(F)(F)F] | I-33 |
| 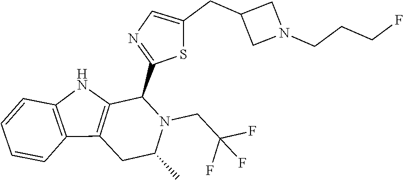 [SMILES: C[C@@H]1Cc2c([nH]c3ccccc23)[C@@H](c2ncc(CC3CN(CCCF)C3)s2)N1CC(F)(F)F] | I-34 |
| 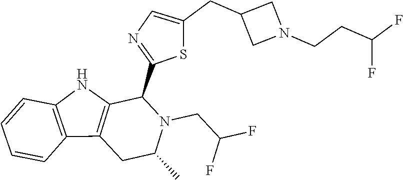 [SMILES: C[C@@H]1Cc2c([nH]c3ccccc23)[C@@H](c2ncc(CC3CN(CCC(F)F)C3)s2)N1CC(F)F] | I-35 |
| 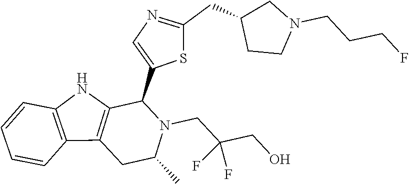 [SMILES: C[C@@H]1Cc2c([nH]c3ccccc23)[C@@H](c2cnc(C[C@H]3CCN(CCCF)C3)s2)N1CC(F)(F)CO] | I-36 |
| 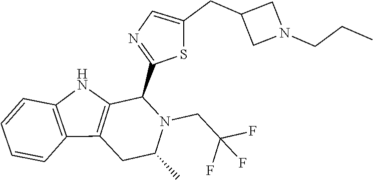 [SMILES: CCCN1CC(Cc2cnc([C@@H]3c4[nH]c5ccccc5c4C[C@@H](C)N3CC(F)(F)F)s2)C1] | I-37 |
| 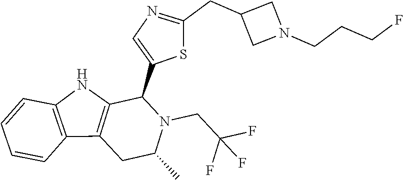 [SMILES: C[C@@H]1Cc2c([nH]c3ccccc23)[C@@H](c2cnc(CC3CN(CCCF)C3)s2)N1CC(F)(F)F] | I-38 |
| 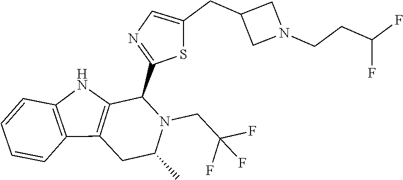 [SMILES: C[C@@H]1Cc2c([nH]c3ccccc23)[C@@H](c2ncc(CC3CN(CCC(F)F)C3)s2)N1CC(F)(F)F] | I-39 |
| 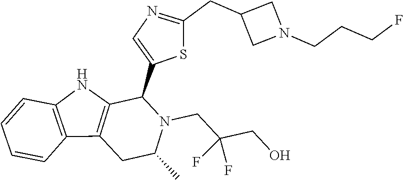 [SMILES: C[C@@H]1Cc2c([nH]c3ccccc23)[C@@H](c2cnc(CC3CN(CCCF)C3)s2)N1CC(F)(F)CO] | I-40 |
| 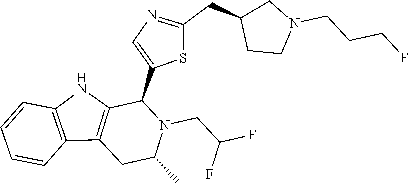 [SMILES: C[C@@H]1Cc2c([nH]c3ccccc23)[C@@H](c2cnc(C[C@@H]3CCN(CCCF)C3)s2)N1CC(F)F] | I-41 |
| 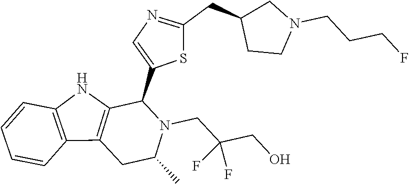 [SMILES: C[C@@H]1Cc2c([nH]c3ccccc23)[C@@H](c2cnc(C[C@@H]3CCN(CCCF)C3)s2)N1CC(F)(F)CO] | I-42 |
| 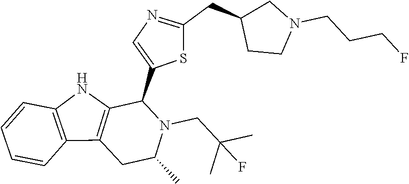 [SMILES: C[C@@H]1Cc2c([nH]c3ccccc23)[C@@H](c2cnc(C[C@@H]3CCN(CCCF)C3)s2)N1CC(C)(C)F] | I-43 |
| 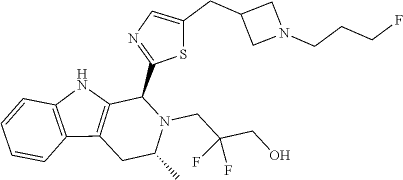 [SMILES: C[C@@H]1Cc2c([nH]c3ccccc23)[C@@H](c2ncc(CC3CN(CCCF)C3)s2)N1CC(F)(F)CO] | I-44 |
| 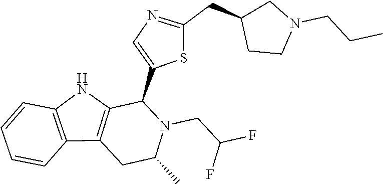 [SMILES: CCCN1CC[C@@H](Cc2ncc([C@@H]3c4[nH]c5ccccc5c4C[C@@H](C)N3CC(F)F)s2)C1] | I-45 |
| 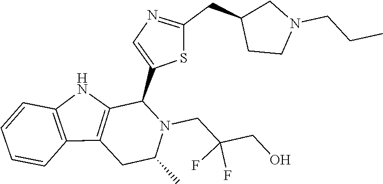 [SMILES: CCCN1CC[C@@H](Cc2ncc([C@@H]3c4[nH]c5ccccc5c4C[C@@H](C)N3CC(F)(F)CO)s2)C1] | I-46 |
| 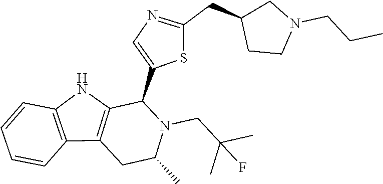 [SMILES: CCCN1CC[C@@H](Cc2ncc([C@@H]3c4[nH]c5ccccc5c4C[C@@H](C)N3CC(C)(C)F)s2)C1] | I-47 |
| 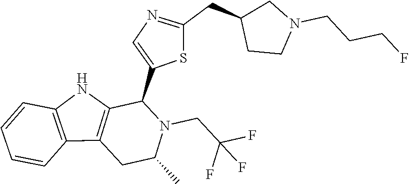 [SMILES: C[C@@H]1Cc2c([nH]c3ccccc23)[C@@H](c2cnc(C[C@@H]3CCN(CCCF)C3)s2)N1CC(F)(F)F] | I-48 |
| 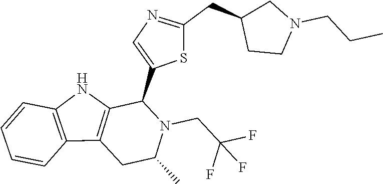 [SMILES: CCCN1CC[C@@H](Cc2ncc([C@@H]3c4[nH]c5ccccc5c4C[C@@H](C)N3CC(F)(F)F)s2)C1] | I-49 |
| 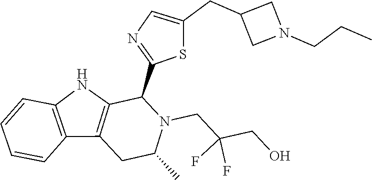 [SMILES: CCCN1CC(Cc2cnc([C@@H]3c4[nH]c5ccccc5c4C[C@@H](C)N3CC(F)(F)CO)s2)C1] | I-50 |
| 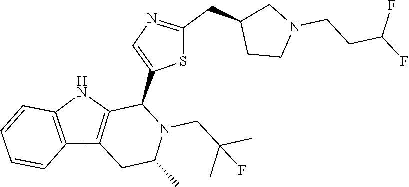 [SMILES: C[C@@H]1Cc2c([nH]c3ccccc23)[C@@H](c2cnc(C[C@@H]3CCN(CCC(F)F)C3)s2)N1CC(C)(C)F] | I-51 |
| 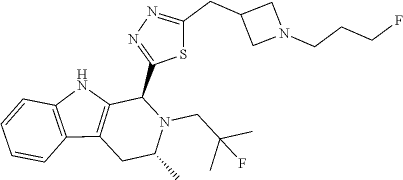 [SMILES: C[C@@H]1Cc2c([nH]c3ccccc23)[C@@H](c2nnc(CC3CN(CCCF)C3)s2)N1CC(C)(C)F] | I-52 |
| 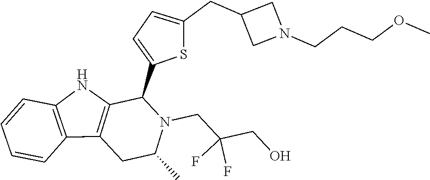 [SMILES: COCCCN1CC(Cc2ccc([C@@H]3c4[nH]c5ccccc5c4C[C@@H](C)N3CC(F)(F)CO)s2)C1] | I-53 |
| 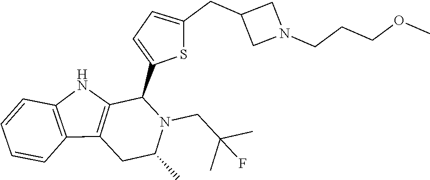 [SMILES: COCCCN1CC(Cc2ccc([C@@H]3c4[nH]c5ccccc5c4C[C@@H](C)N3CC(C)(C)F)s2)C1] | I-54 |
| 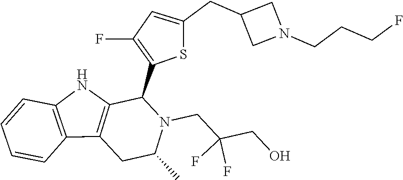 [SMILES: C[C@@H]1Cc2c([nH]c3ccccc23)[C@@H](c2sc(CC3CN(CCCF)C3)cc2F)N1CC(F)(F)CO] | I-55 |
| 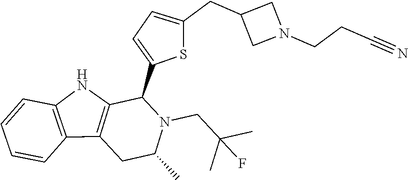 [SMILES: C[C@@H]1Cc2c([nH]c3ccccc23)[C@@H](c2ccc(CC3CN(CCC#N)C3)s2)N1CC(C)(C)F] | I-56 |
| 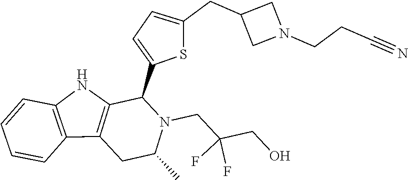 [SMILES: C[C@@H]1Cc2c([nH]c3ccccc23)[C@@H](c2ccc(CC3CN(CCC#N)C3)s2)N1CC(F)(F)CO] | I-57 |
| 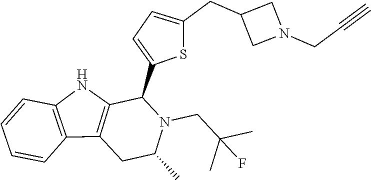 [SMILES: C#CCN1CC(Cc2ccc([C@@H]3c4[nH]c5ccccc5c4C[C@@H](C)N3CC(C)(C)F)s2)C1] | I-58 |
| 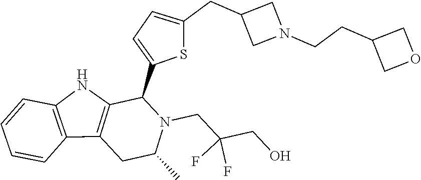 [SMILES: C[C@@H]1Cc2c([nH]c3ccccc23)[C@@H](c2ccc(CC3CN(CCC4COC4)C3)s2)N1CC(F)(F)CO] | I-59 |
| 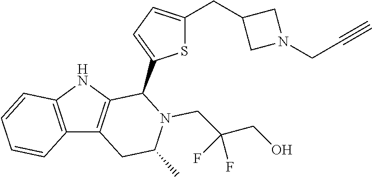 [SMILES: C#CCN1CC(Cc2ccc([C@@H]3c4[nH]c5ccccc5c4C[C@@H](C)N3CC(F)(F)CO)s2)C1] | I-60 |
| 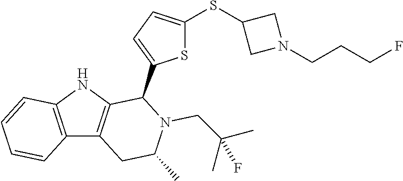 | I-61 |
| 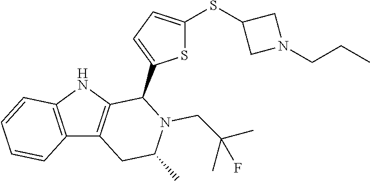 [SMILES: CCCN1CC(Sc2ccc([C@@H]3c4[nH]c5ccccc5c4C[C@@H](C)N3CC(C)(C)F)s2)C1] | I-62 |
| 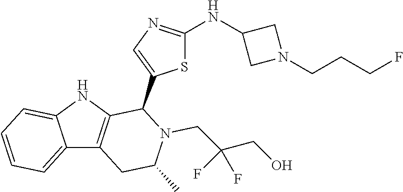 [SMILES: C[C@@H]1Cc2c([nH]c3ccccc23)[C@@H](c2cnc(NC3CN(CCCF)C3)s2)N1CC(F)(F)CO] | I-63 |
| 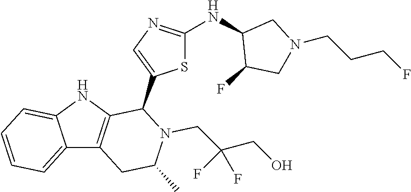 [SMILES: C[C@@H]1Cc2c([nH]c3ccccc23)[C@@H](c2cnc(N[C@H]3CN(CCCF)C[C@H]3F)s2)N1CC(F)(F)CO] | I-64 |
| 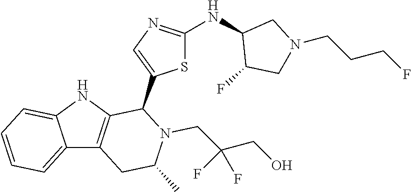 [SMILES: C[C@@H]1Cc2c([nH]c3ccccc23)[C@@H](c2cnc(N[C@H]3CN(CCCF)C[C@@H]3F)s2)N1CC(F)(F)CO] | I-65 |
| 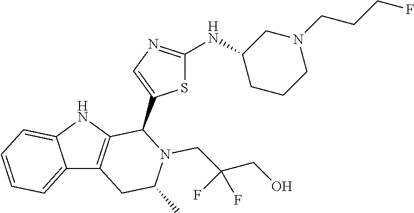 [SMILES: C[C@@H]1Cc2c([nH]c3ccccc23)[C@@H](c2cnc(N[C@H]3CCCN(CCCF)C3)s2)N1CC(F)(F)CO] | I-66 |
| 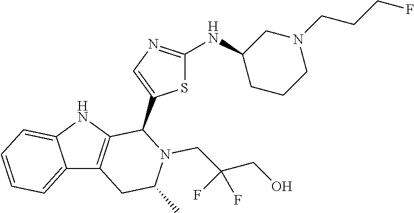 [SMILES: C[C@@H]1Cc2c([nH]c3ccccc23)[C@@H](c2cnc(N[C@@H]3CCCN(CCCF)C3)s2)N1CC(F)(F)CO] | I-67 |
| 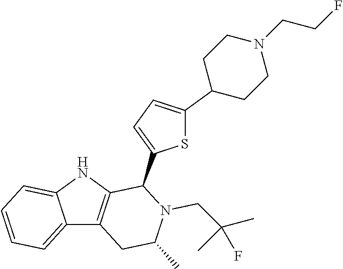 [SMILES: C[C@@H]1Cc2c([nH]c3ccccc23)[C@@H](c2ccc(C3CCN(CCF)CC3)s2)N1CC(C)(C)F] | I-68 |
| 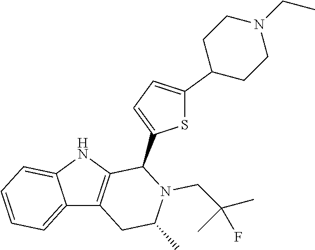 [SMILES: CCN1CCC(c2ccc([C@@H]3c4[nH]c5ccccc5c4C[C@@H](C)N3CC(C)(C)F)s2)CC1] | I-69 |
| 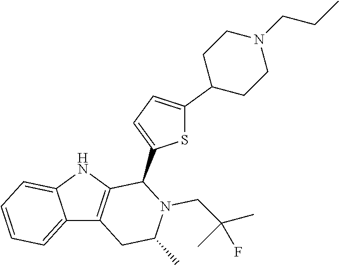 [SMILES: CCCN1CCC(c2ccc([C@@H]3c4[nH]c5ccccc5c4C[C@@H](C)N3CC(C)(C)F)s2)CC1] | I-70 |
| 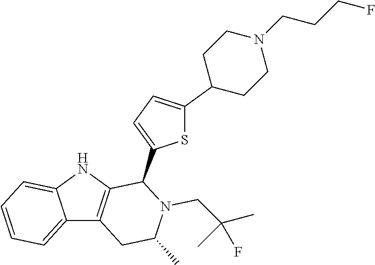 [SMILES: C[C@@H]1Cc2c([nH]c3ccccc23)[C@@H](c2ccc(C3CCN(CCCF)CC3)s2)N1CC(C)(C)F] | I-71 |
| 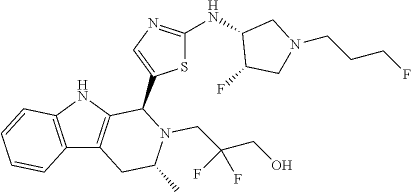 [SMILES: C[C@@H]1Cc2c([nH]c3ccccc23)[C@@H](c2cnc(N[C@@H]3CN(CCCF)C[C@@H]3F)s2)N1CC(F)(F)CO] | I-72 |
| 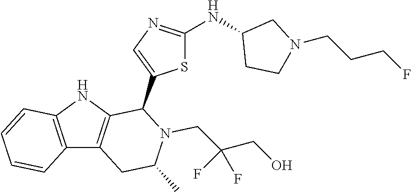 [SMILES: C[C@@H]1Cc2c([nH]c3ccccc23)[C@@H](c2cnc(N[C@H]3CCN(CCCF)C3)s2)N1CC(F)(F)CO] | I-73 |
| 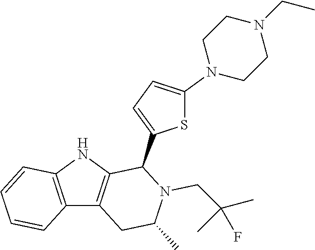 [SMILES: CCN1CCN(c2ccc([C@@H]3c4[nH]c5ccccc5c4C[C@@H](C)N3CC(C)(C)F)s2)CC1] | I-74 |

In some embodiments, the present disclosure provides a compound of Table 2:

TABLE 2

| Compound No. | Compound Name |
|---|---|
| I-1 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl}methyl)-1-propylazetidine |
| I-2 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl}methyl)-1-(3-fluoropropyl)azetidine |
| I-3 | 2-[(1S,3R)-2-(2-fluoro-2-methylpropyl)-3-methyl-1H,2H,3H,4H,9H- |
| | pyrido[3,4-b]indol-1-yl]-5-[(1-propylazetidin-3-yl)methyl]-1,3-thiazole |
| I-4 | 2-[(1S,3R)-2-(2-fluoro-2-methylpropyl)-3-methyl-1H,2H,3H,4H,9H- |
| | pyrido[3,4-b]indol-1-yl]-5-{[1-(3-fluoropropyl)azetidin-3-yl]methyl}-1,3- |
| | thiazole |
| I-5 | 2-((1S,3R)-2-(2-fluoro-2-methylpropyl)-3-methyl-2,3,4,9-tetrahydro-1H- |
| | pyrido[3,4-b]indol-1-yl)-5-((1-(3-fluoropropyl)azetidin-3-yl)methyl)thiazole |
| I-6 | 3-({5-[(1S,3R)-2-(2-fluoro-2-methylpropyl)-3-methyl-1H,2H,3H,4H,9H- |
| | pyrido[3,4-b]indol-1-yl]-4-methylthiophen-2-yl}methyl)-1-(3- |
| | fluoropropyl)azetidine |
| I-7 | 5-((1S,3R)-2-(2-fluoro-2-methylpropyl)-3-methyl-2,3,4,9-tetrahydro-1H- |
| | pyrido[3,4-b]indol-1-yl)-2-((1-propylazetidin-3-yl)methyl)thiazole |
| I-8 | (1S,3R)-2-(2-fluoro-2-methylpropyl)-3-methyl-1-(4-methyl-5-((1- |
| | propylazetidin-3-yl)methyl)thiophen-2-yl)-2,3,4,9-tetrahydro-1H-pyrido[3,4- |
| | b]indole |
| I-9 | (1S,3R)-2-(2-fluoro-2-methylpropyl)-1-(5-((1-(3-fluoropropyl)azetidin-3- |
| | yl)methyl)-4-methylthiophen-2-yl)-3-methyl-2,3,4,9-tetrahydro-1H-pyrido[3,4- |
| | b]indole |
| I-10 | (1S,3R)-2-(2-fluoro-2-methylpropyl)-1-(4-fluoro-5-((1-propylazetidin-3- |
| | yl)methyl)thiophen-2-yl)-3-methyl-2,3,4,9-tetrahydro-1H-pyrido[3,4-b]indole |
| I-11 | 3-({4-fluoro-5-[(1S,3R)-2-(2-fluoro-2-methylpropyl)-3-methyl- |
| | 1H,2H,3H,4H,9H-pyrido[3,4-b]indol-1-yl]thiophen-2-yl}methyl)-1- |
| | propylazetidine |
| I-12 | 3-({4-fluoro-5-[(1S,3R)-2-(2-fluoro-2-methylpropyl)-3-methyl- |
| | 1H,2H,3H,4H,9H-pyrido[3,4-b]indol-1-yl]thiophen-2-yl}methyl)-1-(3- |
| | fluoropropyl)azetidine |
| I-13 | (1S,3R)-2-(2-fluoro-2-methylpropyl)-1-(4-fluoro-5-((1-(3- |
| | fluoropropyl)azetidin-3-yl)methyl)thiophen-2-yl)-3-methyl-2,3,4,9-tetrahydro- |
| | 1H-pyrido[3,4-b]indole |
| I-14 | 5-[(1S,3R)-2-(2-fluoro-2-methylpropyl)-3-methyl-1H,2H,3H,4H,9H- |
| | pyrido[3,4-b]indol-1-yl]-2-{[1-(3-fluoropropyl)azetidin-3-yl]methyl}-1,3- |
| | thiazole |
| I-15 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl}methyl)-1-(3,3,3- |
| | trifluoropropyl)azetidine |
| I-16 | 2,2-difluoro-3-[(1S,3R)-1-(5-{[1-(3-fluoropropyl)azetidin-3- |
| | yl]methyl}thiophen-2-yl)-3-methyl-1H,2H,3H,4H,9H-pyrido[3,4-b]indol-2- |
| | yl]propan-1-ol |
| I-17 | 3-({5-[(1S,3R)-2-(2,2-difluoroethyl)-3-methyl-1H,2H,3H,4H,9H-pyrido[3,4- |
| | b]indol-1-yl]thiophen-2-yl}methyl)-1-(3-fluoropropyl)azetidine |
| I-18 | 1-(3,3-difluoropropyl)-3-({5-[(1S,3R)-2-(2-fluoro-2-methylpropyl)-3-methyl- |
| | 1H,2H,3H,4H,9H-pyrido[3,4-b]indol-1-yl]thiophen-2-yl}methyl)azetidine |
| I-19 | 1-(3-{5-[(1S,3R)-2-(2-fluoro-2-methylpropyl)-3-methyl-1H,2H,3H,4H,9H- |
| | pyrido[3,4-b]indol-1-yl]thiophen-2-yl}propyl)-3-methylazetidine |
| I-20 | 1-(3-fluoropropyl)-3-({5-[(1S,3R)-3-methyl-2-(2,2,2-trifluoroethyl)- |
| | 1H,2H,3H,4H,9H-pyrido[3,4-b]indol-1-yl]thiophen-2-yl}methyl)azetidine |
| I-21 | 1-(3-{5-[(1S,3R)-2-(2-fluoro-2-methylpropyl)-3-methyl-1H,2H,3H,4H,9H- |
| | pyrido[3,4-b]indol-1-yl]thiophen-2-yl} propyl)-3-(fluoromethyl)azetidine |
| I-22 | 3-({5-[(1S,3R)-2-(2,2-difluoropropyl)-3-methyl-1H,2H,3H,4H,9H-pyrido[3,4- |
| | b]indol-1-yl]thiophen-2-yl}methyl)-1-(3-fluoropropyl)azetidine |
| I-23 | (1S,3R)-1-(5-((1-(3-fluoropropyl)azetidin-3-yl)methyl)thiophen-2-yl)-3- |
| | methyl-2-(2,2,3-trifluoropropyl)-2,3,4,9-tetrahydro-1H-pyrido[3,4-b ]indole |
| I-24 | (1S,3R)-2-(2-fluoro-2-methylpropyl)-3-methyl-1-(5-((1-(oxetan-3- |
| | ylmethyl)azetidin-3-yl)methyl)thiophen-2-yl)-2,3,4,9-tetrahydro-1H- |
| | pyrido[3,4-b]indole |
| I-25 | (1S,3R)-2-(2-fluoro-2-methylpropyl)-1-(5-(3-((R)-3-(fluoromethyl)pyrrolidin- |
| | 1-yl)propyl)thiophen-2-yl)-3-methyl-2,3,4,9-tetrahydro-1H-pyrido[3,4-b]indole |
| I-26 | (3R)-1-(3-{5-[(1S,3R)-2-(2-fluoro-2-methylpropyl)-3-methyl- |
| | 1H,2H,3H,4H,9H-pyrido[3,4-b]indol-1-yl]thiophen-2-yl}propyl)-3- |
| | methylpyrrolidine |
| I-27 | (1S,3R)-1-(5-((1-(3-fluoropropyl)azetidin-3-yl)methyl)thiophen-2-yl)-3- |
| | methyl-2-(2,2,3,3-tetrafluoropropyl)-2,3,4,9-tetrahydro-1H-pyrido[3,4-b]indole |
| I-28 | (1S,3R)-1-(5-((1-(3-fluoropropyl)azetidin-3-yl)methyl)thiophen-2-yl)-3- |
| | methyl-2-(2,2,3,3,3-pentafluoropropyl)-2,3,4,9-tetrahydro-1H-pyrido[3,4- |
| | b]indole |
| I-29 | 5-[(1S,3R)-2-(2,2-difluoroethyl)-3-methyl-1,3,4,9-tetrahydropyrido[3,4- |
| | b]indol-1-yl]-2-[1-(3-fluoropropyl)azetidin-3-yl]methyl]thiazole |
| I-30 | 5-((1S,3R)-2-(2,2-difluoroethyl)-3-methyl-2,3,4,9-tetrahydro-1H-pyrido[3,4- |
| | b]indol-1-yl)-2-(((R)-1-(3-fluoropropyl)pyrrolidin-3-yl)methyl)thiazole |
| I-31 | 2-[(1S,3R)-2-(2,2-difluoroethyl)-3-methyl-1,3,4,9-tetrahydropyrido[3,4- |
| | b]indol-1-yl]-5-[1-(3-fluoropropyl)azetidin-3-yl]methyl]thiazole |
| I-32 | 2-[(1S,3R)-2-(2,2-difluoroethyl)-3-methyl-1,3,4,9-tetrahydropyrido[3,4- |
| | b]indol-1-yl]-5-[(1-propylazetidin-3-yl)methyl]thiazole |
| I-33 | 2-(((R)-1-(3-fluoropropyl)pyrrolidin-3-yl)methyl)-5-((1S,3R)-3-methyl-2- |
| | (2,2,2-trifluoroethyl)-2,3,4,9-tetrahydro-1H-pyrido[3,4-b]indol-1-yl)thiazole |
| I-34 | 5-[[1-(3-fluoropropyl)azetidin-3-yl]methyl]-2-[(1S,3R)-3-methyl-2-(2,2,2- |
| | trifluoroethyl)-1,3,4,9-tetrahydropyrido[3,4-b]indol-1-yl]thiazole |
| I-35 | 2-[(1S,3R)-2-(2,2-difluoroethyl)-3-methyl-1,3,4,9-tetrahydropyrido[3,4- |
| | b]indol-1-yl]-5-[[1-(3,3-difluoropropyl)azetidin-3-yl]methyl]thiazole |
| I-36 | 2,2-difluoro-3-[(1S,3R)-1-[2-[[(3R)-1-(3-fluoropropyl)pyrrolidin-3- |
| | yl]methyl]thiazol-5-yl]-3-methyl-1,3,4,9-tetrahydropyrido[3,4-b]indol-2- |
| | yl]propan-1-ol |
| I-37 | 2-[(1S,3R)-3-methyl-2-(2,2,2-trifluoroethyl)-1,3,4,9-tetrahydropyrido[3,4- |
| | b]indol-1-yl]-5-[(1-propylazetidin-3-yl)methyl]thiazole |
| I-38 | 2-[[1-(3-fluoropropyl)azetidin-3-yl]methyl]-5-[(1S,3R)-3-methyl-2-(2,2,2- |
| | trifluoroethyl)-1,3,4,9-tetrahydropyrido[3,4-b]indol-1-yl]thiazole |
| I-39 | 5-[[1-(3,3-difluoropropyl)azetidin-3-yl]methyl]-2-[(1S,3R)-3-methyl-2-(2,2,2- |
| | trifluoroethyl)-1,3,4,9-tetrahydropyrido[3,4-b]indol-1-yl]thiazole |
| I-40 | 2,2-difluoro-3-[(1S,3R)-1-[2-[[1-(3-fluoropropyl)azetidin-3-yl]methyl]thiazol- |
| | 5-yl]-3-methyl-1,3,4,9-tetrahydropyrido[3,4-b]indol-2-yl]propan-1-ol |
| I-41 | 5-[(1S,3R)-2-(2,2-difluoroethyl)-3-methyl-1,3,4,9-tetrahydropyrido[3,4- |
| | b]indol-1-yl]-2-[[(3S)-1-(3-fluoropropyl)pyrrolidin-3-yl]methyl]thiazole |
| I-42 | 2,2-difluoro-3-[(1S,3R)-1-[2-[(3S)-1-(3-fluoropropyl)pyrrolidin-3- |
| | yl]methyl]thiazol-5-yl]-3-methyl-1,3,4,9-tetrahydropyrido[3,4-b]indol-2- |
| | yl]propan-1-ol |
| I-43 | 5-[(1S,3R)-2-(2-fluoro-2-methyl-propyl)-3-methyl-1,3,4,9- |
| | tetrahydropyrido[3,4-b]indol-1-yl]-2-[(3S)-1-(3-fluoropropyl)pyrrolidin-3- |
| | yl]methyl]thiazole |
| I-44 | 2,2-difluoro-3-[(1S,3R)-1-[5-[[1-(3-fluoropropyl)azetidin-3-yl]methyl]thiazol- |
| | 2-yl]-3-methyl-1,3,4,9-tetrahydropyrido[3,4-b]indol-2-yl]propan-1-ol |
| I-45 | 5-[(1S,3R)-2-(2,2-difluoroethyl)-3-methyl-1,3,4,9-tetrahydropyrido[3,4- |
| | b]indol-1-yl]-2-[[(3S)-1-propylpyrrolidin-3-yl]methyl]thiazole |
| I-46 | 2,2-difluoro-3-((1S,3R)-3-methyl-1-(2-(((S)-1-propylpyrrolidin-3- |
| | yl)methyl)thiazol-5-yl)-1,3,4,9-tetrahydro-2H-pyrido[3,4-b]indol-2-yl)propan- |
| | 1-ol |
| I-47 | 5-[(1S,3R)-2-(2-fluoro-2-methyl-propyl)-3-methyl-1,3,4,9- |
| | tetrahydropyrido[3,4-b]indol-1-yl]-2-[(3S)-1-propylpyrrolidin-3- |
| | yl]methyl]thiazole |
| I-48 | 2-[[(3S)-1-(3-fluoropropyl)pyrrolidin-3-yl]methyl]-5-[(1S,3R)-3-methyl-2- |
| | (2,2,2-trifluoroethyl)-1,3,4,9-tetrahydropyrido[3,4-b]indol-1-yl]thiazole |
| I-49 | 5-[(1S,3R)-3-methyl-2-(2,2,2-trifluoroethyl)-1,3,4,9-tetrahydropyrido[3,4- |
| | b]indol-1-yl]-2-[[(3S)-1-propylpyrrolidin-3-yl]methyl]thiazole |
| I-50 | 2,2-difluoro-3-[(1S,3R)-3-methyl-1-[5-[(1-propylazetidin-3-yl)methyl]thiazol- |
| | 2-yl]-1,3,4,9-tetrahydropyrido[3,4-b]indol-2-yl]propan-1-ol |
| I-51 | 2-[[(3S)-1-(3,3-difluoropropyl)pyrrolidin-3-yl]methyl]-5-[(1S,3R)-2-(2-fluoro- |
| | 2-methyl-propyl)-3-methyl-1,3,4,9-tetrahydropyrido[3,4-b]indol-1-yl]thiazole |
| I-52 | 2-((1S,3R)-2-(2-fluoro-2-methylpropyl)-3-methyl-2,3,4,9-tetrahydro-1H- |
| | pyrido[3,4-b]indol-1-yl)-5-((1-(3-fluoropropyl)azetidin-3-yl)methyl)-1,3,4- |
| | thiadiazole |
| I-53 | 2,2-difluoro-3-[(1S,3R)-1-[5-[[1-(3-methoxypropyl)azetidin-3-yl]methyl]-2- |
| | thienyl]-3-methyl-1,3,4,9-tetrahydropyrido[3,4-b]indol-2-yl]propan-1-ol |
| I-54 | (1S,3R)-2-(2-fluoro-2-methyl-propyl)-1-[5-[[1-(3-methoxypropyl)azetidin-3- |
| | yl]methyl]-2-thienyl]-3-methyl-1,3,4,9-tetrahydropyrido[3,4-b]indole |
| I-55 | 2,2-difluoro-3-[(1S,3R)-1-[3-fluoro-5-[[1-(3-fluoropropyl)azetidin-3- |
| | yl]methyl]-2-thienyl]-3-methyl-1,3,4,9-tetrahydropyrido[3,4-b]indol-2- |
| | yl]propan-1-ol |
| I-56 | 3-[3-[[5-[(1S,3R)-2-(2-fluoro-2-methyl-propyl)-3-methyl-1,3,4,9- |
| | tetrahydropyrido[3,4-b]indol-1-yl]-2-thienyl]methyl]azetidin-1- |
| | yl]propanenitrile |
| I-57 | 3-[3-[5-[(1S,3R)-2-(2,2-difluoro-3-hydroxy-propyl)-3-methyl-1,3,4,9- |
| | tetrahydropyrido[3,4-b]indol-1-yl]-2-thienyl]methyl]azetidin-1- |
| | yl]propanenitrile |
| I-58 | (1S,3R)-2-(2-fluoro-2-methyl-propyl)-3-methyl-1-[5-[(1-prop-2-ynylazetidin-3- |
| | yl)methyl]-2-thienyl]-1,3,4,9-tetrahydropyrido[3,4-b]indole |
| I-59 | 2,2-difluoro-3-((1S,3R)-3-methyl-1-(5-((1-(2-(oxetan-3-yl)ethyl)azetidin-3- |
| | yl)methyl)thiophen-2-yl)-1,3,4,9-tetrahydro-2H-pyrido[3,4-b]indol-2- |
| | yl)propan-1-ol |
| I-60 | 2,2-difluoro-3-[(1S,3R)-3-methyl-1-[5-[(1-prop-2-ynylazetidin-3-yl)methyl]-2- |
| | thienyl]-1,3,4,9-tetrahydropyrido[3,4-b]indol-2-yl]propan-1-ol |
| I-61 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl} sulfanyl)-1-(3-fluoropropyl)azetidine |
| I-62 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl}sulfanyl)-1-propylazetidine |
| I-63 | 2,2-difluoro-3-[(1S,3R)-1-[2-[[1-(3-fluoropropyl)azetidin-3-yl]amino]thiazol-5- |
| | yl]-3-methyl-1,3,4,9-tetrahydropyrido[3,4-b]indol-2-yl]propan-1-ol |
| I-64 | 2,2-difluoro-3-[(1S,3R)-1-[2-[[(3S,4R)-4-fluoro-1-(3-fluoropropyl)pyrrolidin-3- |
| | yl]amino]thiazol-5-yl]-3-methyl-1,3,4,9-tetrahydropyrido[3,4-b]indol-2- |
| | yl]propan-1-ol |
| I-65 | 2,2-difluoro-3-((1S,3R)-1-(2-(((3S,4S)-4-fluoro-1-(3-fluoropropyl)pyrrolidin-3- |
| | yl)amino)thiazol-5-yl)-3-methyl-1,3,4,9-tetrahydro-2H-pyrido[3,4-b]indol-2- |
| | yl)propan-1-ol |
| I-66 | 2,2-difluoro-3-[(1S,3R)-1-[2-[(3S)-1-(3-fluoropropyl)-3- |
| | piperidyl]amino]thiazol-5-yl]-3-methyl-1,3,4,9-tetrahydropyrido[3,4-b]indol-2- |
| | yl]propan-1-ol |
| I-67 | 2,2-difluoro-3-((1S,3R)-1-(2-(((R)-1-(3-fluoropropyl)piperidin-3- |
| | yl)amino)thiazol-5-yl)-3-methyl-1,3,4,9-tetrahydro-2H-pyrido[3,4-b]indol-2- |
| | yl)propan-1-ol |
| I-68 | (1S,3R)-1-[5-[1-(2-fluoroethyl)-4-piperidyl]-2-thienyl]-2-(2-fluoro-2-methyl- |
| | propyl)-3-methyl-1,3,4,9-tetrahydropyrido[3,4-b]indole |
| I-69 | (1S,3R)-1-(5-(1-ethylpiperidin-4-yl)thiophen-2-yl)-2-(2-fluoro-2- |
| | methylpropyl)-3-methyl-2,3,4,9-tetrahydro-1H-pyrido[3,4-b]indole |
| I-70 | (1S,3R)-2-(2-fluoro-2-methyl-propyl)-3-methyl-1-[5-(1-propyl-4-piperidyl)-2- |
| | thienyl]-1,3,4,9-tetrahydropyrido[3,4-b]indole |
| I-71 | (1S,3R)-2-(2-fluoro-2-methyl-propyl)-1-[5-[1-(3-fluoropropyl)-4-piperidyl]-2- |
| | thienyl]-3-methyl-1,3,4,9-tetrahydropyrido[3,4-b]indole |
| I-72 | 2,2-difluoro-3-[(1S,3R)-1-[2-[[(3R,4S)-4-fluoro-1-(3-fluoropropyl)pyrrolidin-3- |
| | yl]amino]thiazol-5-yl]-3-methyl-1,3,4,9-tetrahydropyrido[3,4-b]indol-2- |
| | yl]propan-1-ol |
| I-73 | 2,2-difluoro-3-[(1S,3R)-1-[2-[[(3S)-1-(3-fluoropropyl)pyrrolidin-3- |
| | yl]amino]thiazol-5-yl]-3-methyl-1,3,4,9-tetrahydropyrido[3,4-b]indol-2- |
| | yl]propan-1-ol |
| I-74 | (1S,3R)-1-[5-(4-ethylpiperazin-1-yl)-2-thienyl]-2-(2-fluoro-2-methyl-propyl)-3- |
| | methyl-1,3,4,9-tetrahydropyrido[3,4-b]indole |

In some embodiments, the present disclosure provides a compound selected from Table 3, or a pharmaceutically acceptable salt thereof:

TABLE 3

Compound No.
I-3
I-4
I-36
I-45
I-47
I-49
I-51
I-54
I-58
I-59
I-65
I-67
I-70
I-71
I-74

In some embodiments, a compound provided herein is an agonist, and is selected from Table 3.

In some embodiments, the present disclosure provides a compound selected from Table 4, or a pharmaceutically acceptable salt thereof:

TABLE 4

Compound No.
I-1
I-2
I-5
I-6
I-7
I-8
I-9
I-10
I-11
I-12
I-13
I-14
I-15
I-16
I-17
I-18
I-19
I-20
I-21
I-22
I-23
I-24
I-25
I-26
I-27
I-28
I-29
I-30
I-31
I-32
I-33
I-34
I-35
I-37
I-38
I-39
I-40
I-41
I-42
I-43
I-44
I-46
I-48
I-50
I-52
I-53
I-55
I-56
I-57
I-60
I-61
I-62
I-63
I-64
I-66
I-68
I-69
I-72
I-73

In some embodiments, a compound provided herein is an antagonist, and is selected from Table 4.

In some embodiments, the present disclosure provides a compound selected from Table 5, or a pharmaceutically acceptable salt thereof:

TABLE 5

Compound No.
I-16
I-17
I-21
I-29
I-40
I-55

In some embodiments, a compound provided herein is an antagonist, and is selected from Table 5.

In some embodiments, provided compounds are provided and/or utilized in a salt form (e.g., a pharmaceutically acceptable salt form). Reference to a compound provided herein is understood to include reference to salts thereof, unless otherwise indicated.

It will be appreciated that throughout the present disclosure, unless otherwise indicated, reference to a compound of Formula I is intended to also include Formulae I-VI, and compound species of such formulas disclosed herein.

### Preparing Provided Compounds

Provided compounds may generally be made by the processes described in the ensuing schemes and examples. In some embodiments, provided compounds are prepared according to Schemes 1-5:

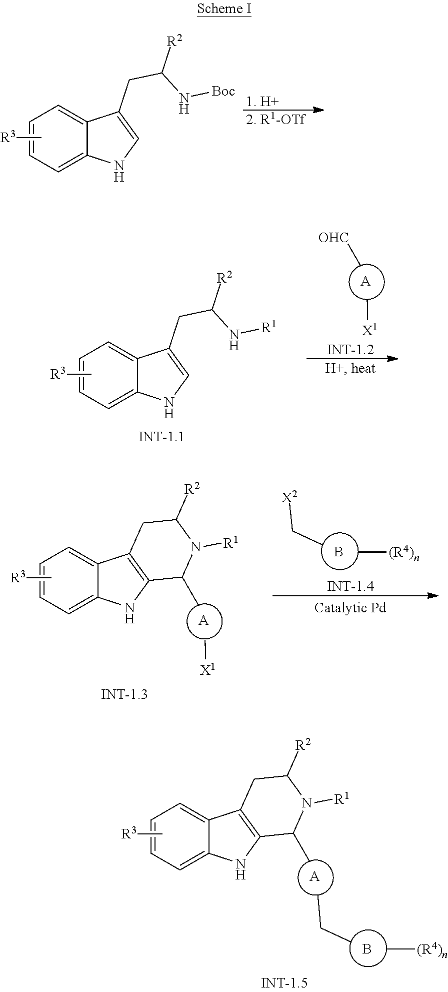

In some embodiments, a compound INT-1.3 is provided by Pictet-Spengler reaction, wherein a functionalized N-alkyl-alpha-methyl tryptamine (INT-1.1) is contacted with a carbaldehyde (INT-1.2) in the presence of an acid and heat, wherein A, R¹, R², and R³ are as defined in classes and subclasses herein with respect to Formula I both singly and in combination, and X¹ is halogen. In some embodiments, INT-1.1 is provided after deprotection (e.g., with acid) and alkylation of a N-Boc-alpha-methyl tryptamine compound, as shown in Scheme 1. In some embodiments, a compound INT-1.5 is provided by a Suzuki reaction, wherein INT-1.3 is contacted with INT-1.4 (wherein B, R⁴, and n are as described in classes and subclasses herein with respect to Formula I both singly and in combination, and X² is -BPin or —BF₃) in the presence of catalytic palladium.

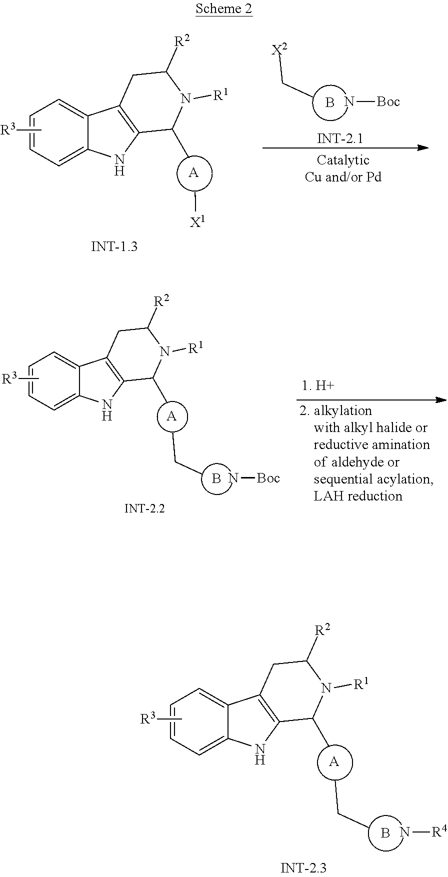

In some embodiments, a compound INT-2.1 (wherein B is as described in classes and subclasses herein with respect to Formula I both singly and in combination, and X² is -BPin or —BF₃) is provided by Suzuki or Negishi conditions comprising contacting a compound INT-1.3 with a compound INT-2.1 in the presence of catalytic Cu or Pd. In some embodiments, a compound INT-2.3 is provided by contacting a compound INT-2.2 with an acid to provide a reaction product, followed by (i) alkylation, (ii) reductive amination, or (iii) acylation and reduction of the reaction product to yield INT-2.3.

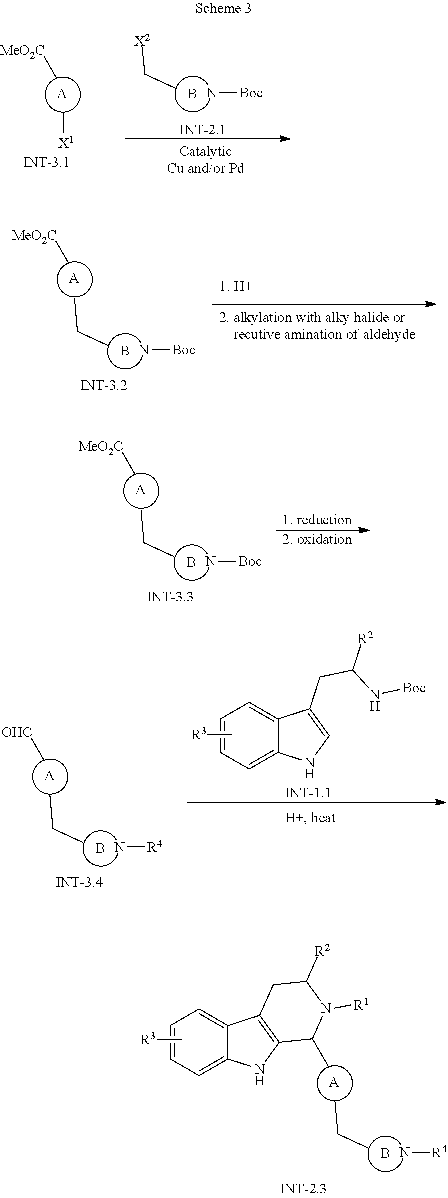

In some embodiments, a compound INT-3.2 is provided under Suzuki or Negishi reaction conditions between INT-3.1 (wherein A is as described in classes and subclasses herein with respect to Formula I both singly and in combination, and X¹ is halogen) and INT-2.1. In some embodiments, a compound INT-3.3 is provided by contacting a compound INT-3.2 with an acid to provide a free amine reaction product, which is modified by S_{N}² alkylation or reductive amination (wherein R⁴ is as described in classes and subclasses herein with respect to Formula I both singly and in combination). In some embodiments, a compound INT-3.4 is prepared by a reduction-oxidation sequence of compound INT-3.3, converting an ester to an aldehyde. In some embodiments, a compound INT-2.3 is provided by contacting INT-3.4 with N-alkyl-alpha-methyl tryptamine (INT-1.1) under Pictet-Spengler reaction conditions (e.g., in the presence of an acid and heat) to provide INT-2.3.

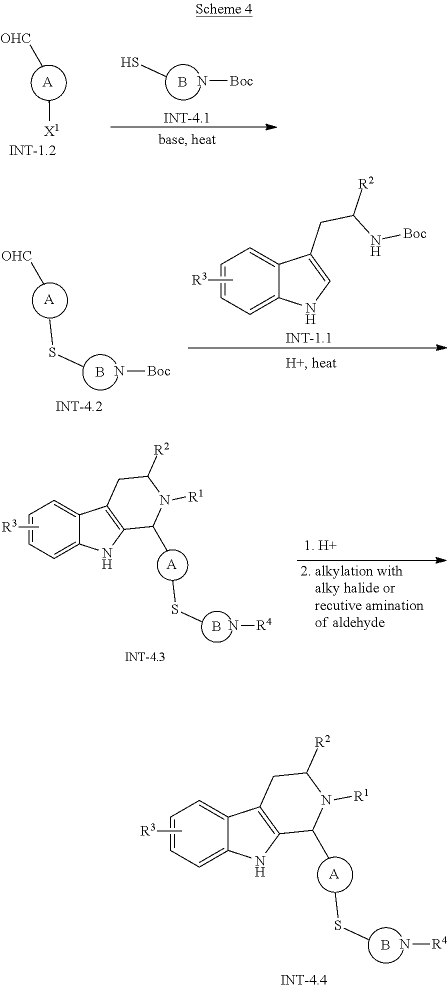

In some embodiments, a compound INT-4.2 is provided by contacting INT-1.2 with INT-4.1 in the presence of a base and heat, wherein B is as described in classes and subclasses herein with respect to Formula I both singly and in combination, and X¹ is halogen. In some embodiments, a compound INT-4.3 is provided by contacting a compound INT-4.2 with N-alkyl-alpha-methyl tryptamine (INT-1.1) in the presence of an acid and heat (wherein R¹, R², and R³ are each described in classes and subclasses herein with respect to Formula I both singly and in combination). In some embodiments, a compound INT-4.4 is provided by contacting a compound INT-4.3 with an acid, followed by (i) alkylation with an alkyl halide or (ii) reductive amination of an aldehyde to provide INT-4.4, wherein R⁴ is described in classes and subclasses herein with respect to Formula I.

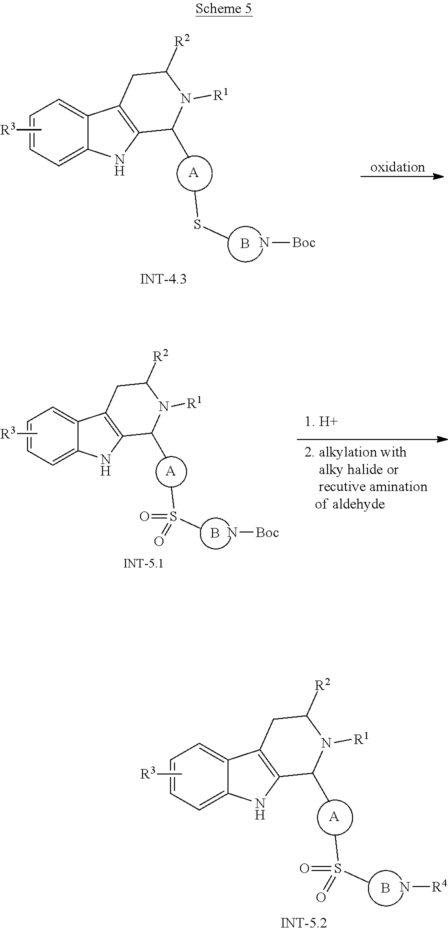

In some embodiments, a compound INT-5.1 is provided by oxidation of a compound INT-4.3, wherein A, B, R¹, R², and R³ are as described in classes and subclasses herein with respect to Formula I both singly and in combination. In some embodiments, a compound INT-5.2 is provided by contacting a compound INT-5.1 with an acid to provide a free amine reaction product. The free amine reaction product is subjected to (i) alkylation or (ii) reductive amination to provide a compound INT-5.2, wherein R⁴ is as described in classes and subclasses herein with respect to Formula I both singly and in combination.

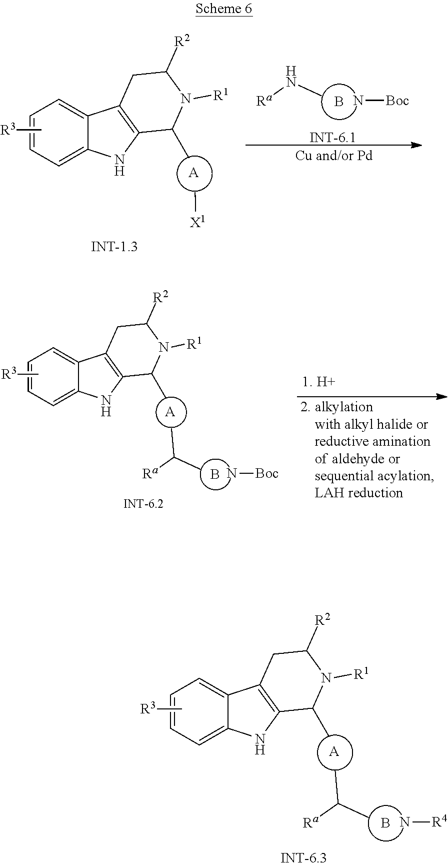

In some embodiments, a compound INT-6.2 (wherein A, B, R¹, R², R³, and R^{a} are as described in classes and subclasses herein with respect to Formula I both singly and in combination) is provided using Buchwald conditions, comprising contacting a compound INT-1.3 (wherein X¹ is halogen) with a compound INT-6.1 in the presence of catalytic Pd. Other C—N bond forming reactions, including SNAr, may provide alternative routes to INT-6.2. In some embodiments, a compound INT-6.2 is provided by contacting a compound INT-6.2 with an acid to provide a reaction product, followed by (i) alkylation, (ii) reductive amination, or (iii) acylation of the reaction product to yield INT-6.3 (wherein R⁴ is as described in classes and subclasses herein with respect to Formula I both singly and in combination).

### Uses, Formulation, and Administration

The present disclosure provides uses for compounds and compositions described herein. In some embodiments, provided compounds and compositions are useful in medicine (e.g., as therapy). In some embodiments, provided compounds and compositions are useful in research as, for example, analytical tools and/or control compounds in biological assays.

### Pharmaceutically Acceptable Compositions

In some embodiments, the present disclosure provides a composition comprising a compound described herein or a pharmaceutically acceptable salt thereof and a pharmaceutically acceptable carrier, adjuvant, or vehicle. In some embodiments, the amount of compound in compositions described herein is such that it is effective to measurably induce degradation of a target in a biological sample or in a patient. In some embodiments, a composition described herein is formulated for administration to a patient in need of such composition. In some embodiments, a composition described herein is formulated for oral administration to a patient.

Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injections forms, deport forms, as well as injectable and infusible solutions. Methods of preparing pharmaceutical compositions are well known in the art.

In some embodiments, provided compounds are formulated in a unit dosage form for ease of administration and uniformity of dosage. A unit dosage form may be, for example, a liquid pharmaceutical composition containing a predetermined quantity of one or more active agents, a solid pharmaceutical composition (e.g., a tablet, a capsule, or the like) containing a predetermined quantity of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein. For example, compositions of the present disclosure may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

### Estrogen Receptor-Associated Diseases and Disorders

The estrogen receptor (“ER”) is involved in a variety of biological processes, relating, for example, to development of the female reproductive system, maintenance of bone mass, protection of cardiovascular and/or central nervous system components, etc. (see, for example, Pearce & Jordan Crit. Rev. Onc/Hem 50:3, 2004; Heldring Phys. Rev. 87:905, 2007). The ER has been implicated in a variety of cancers. In many tumors that express the estrogen receptor (i.e., ER⁺ tumors), active ERα signaling has been demonstrated to drive cell proliferation (although ERβ signaling has been reported to be able to achieve tumor suppressor effects; see, for example, Nilsson & Gustafson Clin. Pharmacol. Ther. 89:44, 2011). Typically, tumors (e.g., breast tumors) with as few as 1% of cells staining positive for ER are classified as “ER⁺”. Therapies targeting the ER are standard of care for many patients with ER⁺ tumors (see, for example, Cardoso et al Annals Onc. <https://doi.org/10.1093/announc/mdmx036>, 2017; Rugo et al. J. Clin. Oncol. 34:3069, 2016; Senkus et al Annal Onc. 26:v8, 2015; Sareddy & Vadlamudi Clin. J Nat. Med, 13:801, 2015). For early stage breast cancer patients, for example, recommended therapy typically involves tumor resection, followed by ER-targeted therapy (e.g., as discussed below). For advanced breast cancer, including metastatic breast cancer, ER-targeted therapy is the mainstay.

Given the importance of ER signaling in many cancers, as well as in certain cardiovascular, inflammatory, and neurodegenerative diseases, significant effort has been invested in developing therapeutic agents and modalities that target the ER. There is some fluidity/flexibility in terminology that has been used to describe ER-targeting agents, but a variety of agents, with different mechanisms, have been developed and/or studied.

For example, some ER-targeting agents are designed and/or documented to reduce levels of estrogen (i.e., 17β estradiol) production. In other embodiments, some ER-targeting agents are designed and/or documented to increase levels of estrogen production.

Some ER-targeting agents are designed and/or documented to bind directly to the ER; in some cases, such agents compete with estrogen for binding to the ER and/or interfere with the allosteric changes that estrogen binding would naturally produce. Often, the term “antiestrogen” is used to refer to agents that bind to the ER, and sometimes is specifically used to indicate those agents that compete with estrogen for ER binding.

The term “selective estrogen receptor modulator, “SERM”, has been used to refer to compounds that are designed and/or documented to alter some aspect of ER activity. Some writings refer to “SERMs” as representing a particular type of anti-estrogens; other writings, however, use the term “SERM” more generally, to refer to a compound that specifically impacts some feature of ER (particularly ERα) expression and/or activity.

The term “selective estrogen receptor degrader” (“SERD”) has been used to refer to compounds that are designed and/or documented to trigger or enhance degradation of the ER. In many instances, if presence of a compound correlates with reduced level of ER, the compound may be referred to as a SERD. Some writings classify compounds either as SERMs or as SERDs; others refer to SERDs as a particular type, or species, of compounds that are SERMs.

Regardless of mechanism of action of a particular agent, clinical experience thus far has revealed that incomplete effects (e.g., within an individual patient and/or across patient populations) and/or development of resistance remain a problem.

Among other things, presence or development of certain ER mutations has been reported to impact effectiveness of various ER-targeted therapies (see, for example, Jeselsohn et al Nature Rev. Clin. Onc. 12, 573, 2015; Gelsomino et al. Breast Cancer Res. Treat 157:253, 2016; Toy et al. 2013). Some particularly problematic mutations are those that “activate” one or more aspects of ER expression and/or function; some activating mutations have been reported that can render the ER ligand-independent (i.e., constitutively active). For example, particular mutations in the ER ligand binding domain, including D538G and Y537S, have been demonstrated to constitutively activate the ER; other mutations including deletions and/or fusions that remove the ligand binding domain, can have similar effects (see, for example, Li et al. Cell Repts 4:1116, 2013; Veeraraghavan et al Breast Cancer Research and Treatment 158, 219-232, 2016; Veeraraghavan, et al. Nature Comms 5:4577, 2014). Some reports have indicated that as many as 50% of women with metastatic breast cancer may have activating ER mutations detectible in circulating tumor DNA.

### Estrogen Receptor Antagonists

In some embodiments, compounds provided herein are estrogen receptor antagonists. As used herein, an “estrogen receptor antagonist” refers to a compound or composition that produces an antagonistic effect when contacting the estrogen receptor of a subject or biological sample. In some embodiments, an estrogen receptor antagonist is characterized by having:
1. (i) between 10% and 80% increase in the E2-normalized signal in the AP assay (agonist mode) of Example 189 and (ii) between 10% and 80% reduction in the E2-normalized signal in the AP assay (antagonist mode) of Example 189;
2. (i) a pIC₅₀ greater than 6.4 and at least a 10% reduction in the E2-normalized signal in the AP assay (antagonist mode) of Example 189; and (ii) no more than 10% increase in E2-normalized signal in the AP assay (agonist mode) of Example 189; or
3. (i) a pIC₅₀ greater than 7.5 and at least a 10% reduction in the E2-normalized signal in the AP assay (antagonist mode) of Example 189; and (ii) no more than 10% increase in E2-normalized signal in the AP assay (agonist mode) of Example 189.

In some embodiments, compounds provided herein are complete estrogen receptor antagonists. As described herein, a complete estrogen receptor antagonist (a “CERAN”) is one that (1) inhibits both AF1 and AF2, and in particular inhibits AF1 activity that remains present in constitutively active ER mutants; (2) promotes ER degradation; and (3) lacks the partial ER agonist activity observed with certain other agents. The present disclosure further appreciates that many previous therapies, including for example, ARN-810, AZD9496, tamoxifen, and others, are less effective than CERANs at least in part because they only partially antagonize ER, and specifically because they inhibit activation of AF2 but not AF1. In some embodiments, an estrogen receptor antagonist is characterized by having (i) a pIC₅₀ greater than 6.4 and at least a 10% reduction in the E2-normalized signal in the AP assay (antagonist mode) of Example 189; and (ii) no more than 10% increase in E2-normalized signal in the AP assay (agonist mode) of Example 189. In some embodiments, an estrogen receptor antagonist is characterized by having (i) a pIC₅₀ greater than 7.5 and at least a 10% reduction in the E2-normalized signal in the AP assay (antagonist mode) of Example 189; and (ii) no more than 10% increase in E2-normalized signal in the AP assay (agonist mode) of Example 189.

### Estrogen Receptor Agonists

In some embodiments, compounds provided herein are estrogen receptor agonists. As used herein, an “estrogen receptor agonist” refers to a compound or composition that produces an agonistic effect when contacting the estrogen receptor of a subject or biological sample. In some embodiments, an estrogen receptor agonist is characterized by (i) at least 80% increase in the E2-normalized signal in the AP assay (agonist mode) of Example 189 and (ii) no more than 80% reduction in the E2-normalized signal in the AP assay (antagonist mode) of Example 189.

### Diseases, Disorders, and Conditions

The present disclosure encompasses the insight that provided compounds have a number of uses, including treatment of an ER-associated disorder (e.g., an ER-associated cancer, such as breast cancer, osteoporosis, or menopause symptoms), detection, and/or characterization of certain tumors. In some embodiments, a disease, disorder, or condition is a cancer. In some embodiments, a disease, disorder, or condition is associated with a mutation in an estrogen receptor.

In some embodiments, provided compounds are useful for treating a disorder associated with increased ER activity (e.g., an ER-associated cancer such as breast cancer). In some embodiments, provided estrogen receptor antagonists (e.g., complete estrogen receptor antagonists) are useful for treating such disorders.

In some embodiments, provided compounds are useful for treating a disorder associated with decreased ER activity (e.g., menopause-related conditions or symptoms, or osteoporosis). In some embodiments, provided estrogen receptor agonists are useful for treating such disorders. Other uses of estrogen receptor agonists exist; see, e.g., Harrison, R. F. and Bonnar, J., Pharmac. Ther., 1980, 11, 451-67.

In some embodiments, the present disclosure provides a method of treating a disorder mediated by an estrogen receptor in a subject, comprising administering to the subject a therapeutically effective amount of a compound described herein.

In some embodiments, a disorder is selected from the group consisting of breast cancer, ovarian cancer, endometrial cancer, vaginal cancer, lung cancer, bone cancer, uterine cancer, and endometriosis. In some embodiments, a disorder is breast cancer. In some embodiments, a disorder is ovarian cancer. In some embodiments, a disorder is endometrial cancer. In some embodiments, a disorder is vaginal cancer. In some embodiments, a disorder is lung cancer. In some embodiments, a disorder is bone cancer. In some embodiments, a disorder is uterine cancer. In some embodiments, a disorder is endometriosis.

In some embodiments, the present disclosure provides a method of treating a disorder associated with a mutation of an estrogen receptor in a subject, comprising administering to the subject a therapeutically effective amount of a compound described herein. In some embodiments, such a disorder is selected from the group consisting of breast cancer, ovarian cancer, endometrial cancer, vaginal cancer, lung cancer, bone cancer, uterine cancer, and endometriosis. In some embodiments, a disorder is breast cancer. In some embodiments, a disorder is ovarian cancer. In some embodiments, a disorder is endometrial cancer. In some embodiments, a disorder is vaginal cancer. In some embodiments, a disorder is lung cancer. In some embodiments, a disorder is bone cancer. In some embodiments, a disorder is uterine cancer. In some embodiments, a disorder is endometriosis.

In some embodiments, a method of treating a disorder in a subject described herein comprises administering to the subject a compound described herein in combination with or alternation with an anti-cancer agent. In some embodiments, an anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

In some embodiments, an anti-cancer agent is a HER2 inhibitor. In some embodiments, a HER2 inhibitor is selected from tucatinib, trastuzumab, pertuzumab, ado-trastuzumab, trastuzumab emtansine, ado-trastuzumab emtansine, trastuzumab deruxtecan pertuzumab, lapatinib, and neratinib.

In some embodiments, an anti-cancer agent is an mTOR inhibitor. In some embodiments, an mTOR inhibitor is selected from everolimus, sirolimus, temsirolimus, and LY3023414.

In some embodiments, an anti-cancer agent is a CDK4/6 inhibitor. In some embodiments, a CDK4/6 inhibitor is selected from palbociclib, abemaciclib, ribociclib, lerociclib, trilaciclib, and SHR6390.

In some embodiments, an anti-cancer agent is a PI3 kinase inhibitor. In some embodiments, a PI3 kinase inhibitor is selected from perifosine, CAL101, BEZ235, XL147, XL765, GDC-0941, and IPI-145.

In some embodiments, a PI3 kinase inhibitor is a PIK3CA inhibitor. In some embodiments, a PIK3CA inhibitor is selected from alpelisib, taselisib, and LY3023414.

In some embodiments, an anti-cancer agent is an aromatase inhibitor. In some embodiments, an aromatase inhibitor is selected from aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione.

In some embodiments, an anti-cancer agent is an antibody to or inhibitor of PD-1, PD-L1 or CTLA-4.

In some embodiments, an anti-cancer agent is an antibody to or inhibitor of EGFR, PGFR, or IGFR. In some embodiments, an anti-cancer agent is erlotinib or gefitinib.

In some embodiments, a method described herein comprises administering a compound reported herein in combination or in alternation with an estrogen receptor antagonist or a partial estrogen receptor antagonist.

In some embodiments, the present disclosure provides a method of preventing recurrence of a cancer in a subject comprising administering to the subject a compound described herein. In some embodiments, a cancer is selected from breast cancer, ovarian cancer, endometrial cancer, vaginal cancer, lung cancer, bone cancer, and uterine cancer. In some embodiments, a compound described herein is administered as an adjunctive therapy after or instead of chemotherapy, radiation, or surgery. In some embodiments, a compound is administered after surgery. In some embodiments, a compound is administered prior to surgery. In some embodiments, a cancer is a breast cancer that has progressed in the presence of endocrine or aromatase therapy.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Example 1: 2-Fluoro-2-methylpropan-1-ol

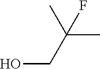

To a flask equipped with a mechanical stirrer, nitrogen inlet, and thermocouple was added methyl 2-fluoro-2-methylpropanoate (28.63 g, 238.4 mmol, 1.0 equiv) in diethyl ether (560 mL). The solution was cooled to 0° C. and treated with lithium aluminum hydride (9.05 g, 238.4 mmol, 1.0 equiv) over the course of 20 min while maintaining the temperature below 10° C. The reaction was stirred at 0° C. for one hour. Water (9.1 mL), 15% sodium hydroxide solution (9.1 mL) and water (17.8 mL) were sequentially added at 0° C. The mixture was vigorously stirred for 15 min at 0° C., then allowed to warm to room temperature and stirred for additional 15 min. Magnesium sulfate (15 g) was added to the resulting white cloudy suspension which was stirred for an additional 10 min. The mixture was filtered and the solid was washed with diethyl ether (2×50 mL). The solvent was removed under reduced pressure to give the title compound as a colorless liquid (20.54 g, 94% yield). ¹H-NMR (400 MHz, CDCl₃) δ=3.59-3.52 (dd, J=6.7 Hz, J=13.6 Hz, 2H), 3.47 (s, 1H), 1.39 (s, 3H), 1.34 (s, 3H)

### Example 2: 2-Fluoro-2-methylpropyl trifluoromethanesulfonate

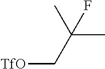

A mixture of compound 2-fluoro-2-methylpropan-1-ol (30.0 g, 325.7 mmol) and triethylamine (42.9 g, 59.1 mL, 423.8 mmol, 1.3 equiv) in MTBE (300 mL) was cooled to −20° C. Trifluoromethanesulfonic anhydride (110.3 g, 65.7 mL, 390.8 mmol, 1.2 equiv) was added via an addition funnel while maintaining the temperature below −5° C. After stirring at −10° C. for one hour, the reaction was cooled to −20° C., diluted with MTBE (150 mL) and 1M HCl. The mixture was warmed to room temperature and stirred for additional 10 min. The layers were separated, and the organic layer was washed with saturated sodium bicarbonate solution (2×150 mL), saturated brine (250 mL), dried over magnesium sulfate (7 g), filtered, and concentrated under reduced pressure to give the title compound as a colorless liquid (36.1 g, 49% yield). ¹H-NMR (300 MHz, CDCl₃) δ=4.43-4.37 (dd, J=6.7 Hz, J=13.6 Hz, 2H), 1.51 (s, 3H), 1.46 (s, 3H)

### Example 3: (R)-1-(1H-Indol-3-yl)propan-2-amine

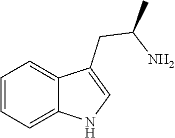

Trifluoroacetic acid (51.9 g, 34.9 mL, 455.6 mmol, 5.0 equiv) was slowly added to a stirred solution of tert-butyl (R)-(1-(1H-indol-3-yl)propan-2-yl)carbamate (25 g, 91.1 mmol, 1.0 equiv) in dichloromethane (250 mL) at room temperature. After stirring for 16 h, the pH was adjusted to 13 with 10% sodium hydroxide. The mixture was extracted with dichloromethane (3×100 mL). The combined organic layers were concentrated under reduced pressure to give the title compound as a brown solid, which was used subsequently. LCMS: m/z=175.2 (M+H)⁺

### Example 4: (R)—N-(1-(1H-indol-3-yl)propan-2-yl)-2-fluoro-2-methylpropan-1-amine

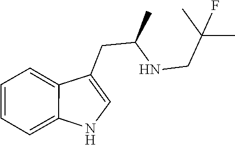 [SMILES: C[C@H](Cc1c[nH]c2ccccc12)NCC(C)(C)F]

N,N-Diisopropylethylamine (17.1 g, 22.8 mL, 132.1 mmol, 1.45 equiv) was added at room temperature to a mixture of (R)-1-(1H-indol-3-yl)propan-2-amine (15.9 g, 91.1 mmol, 1.0 equiv) and 2-fluoro-2-methylpropyl trifluoromethanesulfonate (20.4 g, 91.1 mmol, 1.0 equiv) in 1,4-dioxane (180 mL). After heating at 75° C. for 16 h, the reaction was cooled to room temperature, and diluted with water (180 mL) and MTBE (70 mL). The layers were separated, and the aqueous layer was extracted with MTBE (3×70 mL). The combined organic layers were washed with water (150 mL), saturated brine (150 mL), dried over magnesium sulfate (8 g) and filtered. The filtrate was cooled to 0° C. and carefully treated with 5-6 N HCl in 2-propanol (59 mL, 3.0 equiv). After stirring for 30 min, the suspension was filtered and the solid was triturated with 10% 2-propanol in MTBE (150 mL) to give the hydrochloride salt of the title compound as a white solid. The solid was suspended in 2-propanol (15 mL) and carefully treated with 15% sodium hydroxide (180 mL). The mixture was extracted with MTBE (3×150 mL). The combined organic layers were washed with saturated brine (150 mL), dried over magnesium sulfate (5 g), filtered and concentrated under reduced pressure to give the title compound as a brown solid (13.5 g, 60% yield).

¹H NMR (400 MHz, CDCl₃) δ=8.09 (br s, 1H) 7.62-7.60 (d, 1H), 7.35-7.33 (d, 1H), 7.20-7.09 (m, 2H), 7.02 (s, 1H), 3.02-2.91 (m, 1H), 2.87-281 (m, 1H), 2.79-2.69 (m, 3H), 149 (br s, 1H), 1.37 (s, 3H), 1.30 (s, 3H), 1.11-1.09 (d, J 6.7 Hz, 3H); ¹⁹F NMR: (376 MHz, CDCl₃) δ=−144.1; LCMS: m/z=249.2 (M+H)⁺

### Example 5: (R)—N-(1-(1H-Indol-3-yl)propan-2-yl)-3-((tert-butyldiphenylsilyl)oxy)-2,2-difluoropropan-1-amine

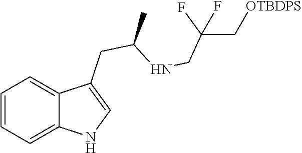

N,N-Diisopropylethylamine (3.5 mL, 20.3 mmol, 1.45 equiv) was added at room temperature to a mixture of (R)-1-(1H-indol-3-yl)propan-2-amine (2.44 g, 14.0 mmol, 1.0 equiv) and 3-((tert-butyldiphenylsilyl)oxy)-2,2-difluoropropyl trifluoromethanesulfonate (6.75 g, 14.0 mmol, 1.0 equiv) in 1,4-dioxane (27 mL). After heating at 85° C. for 16 h, the reaction was cooled to room temperature and diluted with water (30 mL) and methyl tert-butyl ether (30 mL). The layers were separated, and the aqueous layer was extracted with methyl tert-butyl ether (2×30 mL). The combined organic layers were washed with water (50 mL), saturated brine (50 mL), dried over magnesium sulfate, filtered and concentrated under reduced pressure. The residue was purified on an Interchim automated chromatography system (Biotage® Sfar Silica HC High Capacity 20 μm 350 g silica gel column), eluting with a gradient of 0 to 50% ethyl acetate in heptanes with 1% trimethylamine to give the title compound (3.9 g, 55% yield) as a brown oil. LCMS: m/z=507.3 [M+H]⁺.

### Example 6: (R)—N-(2,2-Difluoroethyl)-1-(1H-indol-3-yl)propan-2-amine

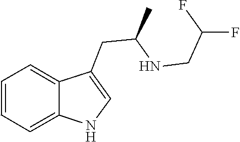 [SMILES: C[C@H](Cc1c[nH]c2ccccc12)NCC(F)F]

N,N-Diisopropylethylamine (3.75 mL, 21.8 mmol, 1.45 equiv) was added at room temperature to a mixture of (R)-1-(1H-indol-3-yl)propan-2-amine (2.61 g, 15.0 mmol, 1.0 equiv) and 2,2-difluoroethyl trifluoromethanesulfonate (3.21 g, 15.0 mmol, 1.0 equiv) in 1,4-dioxane (30 mL). After heating at 85° C. for 16 h, the reaction was cooled to room temperature and diluted with water (30 mL) and methyl tert-butyl ether (30 mL). The layers were separated, and the aqueous layer was extracted with methyl tert-butyl ether (2×30 mL). The combined organic layers were washed with water (50 mL), saturated brine (50 mL), dried over magnesium sulfate, filtered and concentrated under reduced pressure. The residue was purified on an Interchim automated chromatography system (Sorbtech 330 g silica gel column), eluting with a gradient of 0 to 70% ethyl acetate in heptanes with 1% trimethylamine to give the title compound (2.4 g, 67% yield) as a brown oil. LCMS: m/z=239.1 [M+H]⁺.

### Example 7: (R)-1-(1H-Indol-3-yl)-N-(2,2,2-trifluoromethyl)propan-2-amine

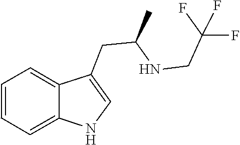 [SMILES: C[C@H](Cc1c[nH]c2ccccc12)NCC(F)(F)F]

N,N-Diisopropylethylamine (3.75 mL, 21.8 mmol, 1.45 equiv) was added at room temperature to a mixture of (R)-1-(1H-indol-3-yl)propan-2-amine (2.61 g, 15.0 mmol, 1.0 equiv) and 2,2,2-trifluoroethyl trifluoromethanesulfonate (3.48 g, 15.0 mmol, 1.0 equiv) in 1,4-dioxane (30 mL). After heating at 85° C. for 16 h, the reaction was cooled to room temperature and diluted with water (30 mL) and methyl tert-butyl ether (30 mL). The layers were separated, and the aqueous layer was extracted with methyl tert-butyl ether (2×30 mL). The combined organic layers were washed with water (50 mL), saturated brine (50 mL), dried over magnesium sulfate, filtered and concentrated under reduced pressure. The residue was purified on an Interchim automated chromatography system (Sorbtech 330 g silica gel column), eluting with a gradient of 0 to 70% ethyl acetate in heptanes with 1% trimethylamine to give the title compound (3.2 g, 83% yield) as a brown oil. LCMS: m/z=257.1 [M+H]⁺.

### Example 8: (R)—N-(1-(1H-Indol-3-yl)propan-2-yl)-2,2-difluoropropan-1-amine)

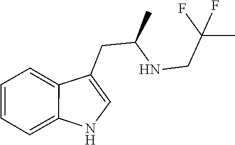 [SMILES: C[C@H](Cc1c[nH]c2ccccc12)NCC(C)(F)F]

N,N-Diisopropylethylamine (2.65 mL, 15.4.1 mmol, 1.45 equiv) was added at room temperature to a mixture of (R)-1-(1H-indol-3-yl)propan-2-amine (1.86 g, 10.6 mmol, 1.0 equiv) and 2,2-difluoropropyl trifluoromethanesulfonate (2.42 g, 10.6 mmol, 1.0 equiv) in 1,4-dioxane (18 mL). After heating at 75° C. for 16 h, the reaction was cooled to room temperature, and diluted with water (50 mL) and MTBE (50 mL). The layers were separated, and the aqueous layer was extracted with MTBE (3×40 mL). The combined organic layers were washed with water (50 mL), saturated brine (50 mL), dried over magnesium sulfate (5 g) and filtered. The filtrate was cooled to 0° C. and carefully treated with 5-6 N HCl in 2-propanol (6.4 mL, 3.0 equiv). After stirring for 16 h, the suspension was filtered and the solid was triturated with 10% 2-propanol in MTBE (30 mL) to give the hydrochloride salt of the title compound as a white solid. The solid was suspended in 15% sodium hydroxide (50 mL). The mixture was extracted with MTBE (3×50 mL). The combined organic layers were washed with saturated brine (50 mL), dried over magnesium sulfate (5 g), filtered and concentrated under reduced pressure to give the title compound as a brown solid (2.16 g, 81% yield). ¹H NMR (400 MHz, CDCl₃) δ=8.02 (br s, 1H), 7.62 (d, J=7.9 Hz, 1H), 7.37 (td, J=0.9, 8.1 Hz, 1H). 7.21 (dt, J=1.2, 7.5 Hz, 1H), 7.15-7.10 (m, 1H), 7.04 (d, J=2.2 Hz, 1H), 3.09 (sxt, J=6.4 Hz, 1H), 3.02-2.88 (m, 2H), 2.87-2.78 (m, 2H), 1.64-1.54 (m, 3H), 1.37 (br s, 1H), 1.12 (d, J=6.2 Hz, 3H); LCMS: m/z=253.1 [M+H]⁺.

### Example 9: (1S,3R)-1-(5-Bromothiophen-2-yl)-2-(2-fluoro-2-methylpropyl)-3-methyl-2,3,4,9-tetrahydro-1H-pyrido[3,4-b]indole

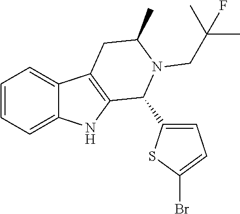 [SMILES: C[C@@H]1Cc2c([nH]c3ccccc23)[C@@H](c2ccc(Br)s2)N1CC(C)(C)F]

5-Bromothiophene-2-carbaldehyde (6.4 g, 33.5 mmol, 1.0 equiv) was added at room temperature to a solution of (R)—N-(1-(1H-indol-3-yl)propan-2-yl)-2-fluoro-2-methylpropan-1-amine (10 g, 40.2 mmol, 1.2 equiv) and acetic acid (3.8 mL, 67 mmol, 2.0 equiv) in toluene (30 mL). After heating at 80° C. for 3 h, the reaction was cooled to room temperature and diluted with saturated sodium bicarbonate (100 mL). The layers were separated, and the aqueous layer was extracted with ethyl acetate (3×50 mL). The combined organic layers were washed with saturated brine (50 mL), dried over magnesium sulfate (10 g), filtered and concentrated under reduced pressure. The crude yellow oil was purified on an Interchim automated chromatography system (Biotage Sfar Silica HC 20 μm, 250 g), eluting with a gradient of 0 to 20% ethyl acetate in heptanes to give the title compound (8.07 g, 57% yield) as a yellow solid. LCMS: m/z=422.3 [M+H]⁺.

### Example 10: 3-(Fluoromethyl)-1-(3-(4,4,5,5-tetramethyl-1,3,2-dioxaborolan-2-yl)propyl)azetidine

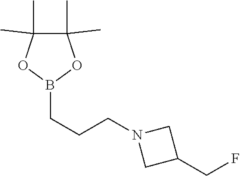 [SMILES: CC1(C)OB(CCCN2CC(CF)C2)OC1(C)C]

2-(3-Bromopropyl)-4,4,5,5-tetramethyl-1,3,2-dioxaborolane (0.58 mL, 2.8 mmol, 1.0 equiv) was added to a mixture of 3-(fluoromethyl)azetidine hydrochloride (0.449 g, 3.58 mmol, 1.3 equiv) and potassium carbonate (1.14 g, 8.25 mmol, 3.0 equiv) in acetonitrile (6.9 mL) in a 40 mL vial. The reaction was stirred at room temperature for 2 days and was monitored by ¹H-NMR. The reaction mixture was filtered and the solid was washed with ethyl acetate (20 mL). The filtrate was concentrated under reduced pressure and the residue was diluted with ethyl acetate (10 mL) and filtered a second time. The resulting filtrate was concentrated under reduced pressure to give the title compound (0.649 g, 92% yield) as a pale-yellow oil.

### Example 11: (1S,3R)-2-(2-Fluoro-2-methylpropyl)-1-(5-(3-(3-(fluoromethyl)azetidin-1-yl)propyl)thiophen-2-yl)-3-methyl-2,3,4,9-tetrahydro-1H-pyrido[3,4-b]indole

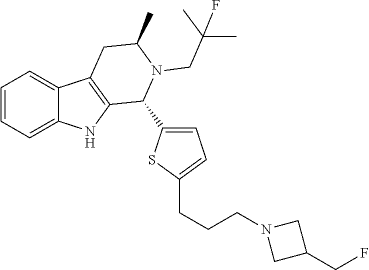 [SMILES: C[C@@H]1Cc2c([nH]c3ccccc23)[C@@H](c2ccc(CCCN3CC(CF)C3)s2)N1CC(C)(C)F]

3-(Fluoromethyl)-1-(3-(4,4,5,5-tetramethyl-1,3,2-dioxaborolan-2-yl)propyl)azetidine (0.611 g, 2.38 mmol, 2.0 equiv), palladium acetate (0.045 g, 0.20 mmol, 17 mol %), RuPhos (0.182 g, 0.393 mmol, 33 mol %), potassium carbonate (0.821 g, 5.95 mmol, 5 equiv) and (1S,3R)-1-(5-bromothiophen-2-yl)-2-(2-fluoro-2-methylpropyl)-3-methyl-2,3,4,9-tetrahydro-1H-pyrido[3,4-b]indole (0.500 g, 1.19 mmol, 1.0 equiv) were added to a 40 mL vial. Toluene (8 mL) and water (4 mL) were added to the reaction flask, which was sparged with nitrogen for 1-2 min. The reaction was heated at 90° C. for 18 h. The reaction was cooled to room temperature then transferred to a separatory funnel with dichloromethane (30 mL) and water (30 mL). The layers were separated, and the aqueous layer was extracted with dichloromethane (2×30 mL). The combined organic extracts were dried over sodium sulfate, filtered and concentrated under reduced pressure onto Celite® (5 g). The material was purified on a Biotage automated chromatography system (28 g, Biotage 50 μm KP-amino-d column), eluting with a gradient of 0 to 6% ethyl acetate in dichloromethane. No separation was obtained, and material was absorbed (0.60 g) onto Celite® (5 g). The material was purified on a Biotage automated chromatography system (28 g, Biotage 50 μm KP-amino-d column), eluting with a gradient of 0 to 50% ethyl acetate in heptanes to give the title compound (70 mg, ˜50% HPLC purity). This material was absorbed onto Celite® (2 g) and was purified on a Biotage automated chromatography system (5.5 g, Redigold C18 column), eluting with a gradient of 0 to 100% methanol in water to give the title compound (17 mg, 3% yield) as an off-white solid. ¹H NMR (400 MHz, CDCl₃) δ=7.83 (br s, 1H), 7.52 (d, J=7.7 Hz, 1H), 7.33 (d, J=7.6 Hz, 1H), 7.17 (dt, J=1.2, 7.5 Hz, 1H), 7.14-7.09 (m, 1H), 6.52 (t, J=3.5 Hz, 2H), 5.22 (br s, 1H), 4.56 (d, J=5.9 Hz, 1H), 4.44 (d, J=5.9 Hz, 1H), 3.46 (br s, 1H), 3.37-3.30 (m, 2H), 2.99 (t, J=6.9 Hz, 2H), 2.86-2.73 (m, 3H), 2.69-2.42 (m, 6H), 1.72-1.64 (m, 2H), 1.55-1.47 (m, 3H), 1.37-1.30 (m, 3H), 1.15 (d, J=6.7 Hz, 3H); LCMS: m/z=472.3 [M+H]⁺.

### Example 12: (R)-3-Methyl-1-(3-(4,4,5,5-tetramethyl-1, 3,2-dioxaborolan-2-yl)propyl)pyrrolidine

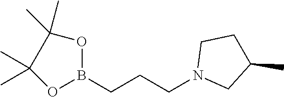

2-(3-Bromopropyl)-4,4,5,5-tetramethyl-1,3,2-dioxaborolane (0.33 mL, 1.6 mmol, 1.0 equiv) was added to a mixture of (R)-3-methylpyrrolidine hydrochloride (0.250 g, 2.06 mmol, 1.3 equiv) and potassium carbonate (0.655 g, 4.74 mmol, 3.0 equiv) in acetonitrile (4.0 mL) in a 40 mL vial. The reaction was stirred at room temperature for 2 days and was monitored by ¹H-NMR. The reaction was filtered and the solid was washed with ethyl acetate (15 mL). The resulting filtrate was concentrated under reduced pressure to give the title compound (0.311 g, 78% yield) as a colorless oil.

### Example 13: (1S,3R)-2-(2-Fluoro-2-methylpropyl)-3-methyl-1-(5-(3-((R)-3-methylpyrrolidin-1-yl)propyl)thiophen-2-yl)-2,3,4,9-tetrahydro-1H-pyrido[3,4-b]indole

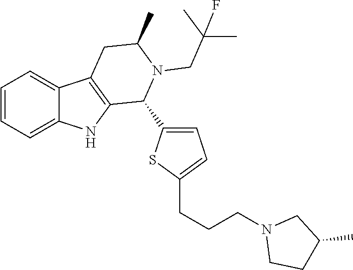 [SMILES: C[C@@H]1CCN(CCCc2ccc([C@@H]3c4[nH]c5ccccc5c4C[C@@H](C)N3CC(C)(C)F)s2)C1]

(R)-3-Methyl-1-(3-(4,4,5,5-tetramethyl-1,3,2-dioxaborolan-2-yl)propyl)pyrrolidine (0.311 g, 1.23 mmol, 2.0 equiv), palladium acetate (0.023 g, 0.10 mmol, 17 mol %), RuPhos (0.094 g, 0.203 mmol, 33 mol %), potassium carbonate (0.424 g, 3.07 mmol, 5 equiv) and (1S,3R)-1-(5-bromothiophen-2-yl)-2-(2-fluoro-2-methylpropyl)-3-methyl-2,3,4,9-tetrahydro-1H-pyrido[3,4-b]indole (0.259 g, 0.614 mmol, 1.0 equiv) were added to a 40 mL vial. Toluene (4.1 mL) and water (2 mL) were added to the reaction flask, which was sparged with nitrogen for 10 min. The reaction was heated at 90° C. for 18 h. The reaction was cooled to room temperature then transferred to a separatory funnel with dichloromethane (15 mL) and water (15 mL). The layers were separated, and the aqueous layer was extracted with dichloromethane (2×15 mL). The combined organic extracts were dried over sodium sulfate, filtered and concentrated under reduced pressure onto Celite® (3 g). The material was purified on a Biotage automated chromatography system (28 g, Biotage 50 μm KP-amino-d column), eluting with a gradient of 0 to 50% ethyl acetate in heptanes to give the title compound (90 mg, ˜85% HPLC purity), which contained an unknown amount of compound (R)-3-methyl-1-(3-(4,4,5,5-tetramethyl-1,3,2-dioxaborolan-2-yl)propyl)pyrrolidine. This material was absorbed onto Celite® (2 g) and was purified on a Biotage automated chromatography system (15 g, Redigold C18 column), eluting with a gradient of 0 to 95% methanol in water. The material was dried under vacuum at room temperature for 3 h to give the title compound (20 mg, 6.8% yield) as a white foam. ¹H NMR (400 MHz, CDCl₃) δ=7.84 (br s, 1H), 7.53 (d, J=7.7 Hz, 1H), 7.34 (d, J=7.9 Hz, 1H), 7.18 (dt, J=1.3, 7.5 Hz, 1H), 7.15-7.09 (m, 1H), 6.54 (s, 2H), 5.23 (br s, 1H), 3.47 (br s, 1H), 2.88-2.75 (m, 3H), 2.74-2.58 (m, 4H), 2.57-2.40 (m, 4H), 2.25 (qd, J=6.9, 16.1 Hz, 1H), 2.07-1.95 (m, 2H), 1.86 (quin, J=7.7 Hz, 2H), 1.58-1.46 (m, 3H), 1.38-1.29 (m, 3H), 1.16 (d, J=6.7 Hz, 3H), 1.03 (d, J=6.8 Hz, 3H); LCMS: m/z=468.3 [M+H]⁺.

### Example 14: 3-Methyl-1-(3-(trifluoro-λ⁴-boraneyl)propyl)azetidine, potassium salt

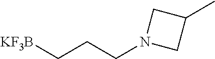

A 4.5 M solution of potassium hydrogen difluoride (1.0 mL, 4.5 mmol, 4.5 equiv) was added to 3-methyl-1-(3-(4,4,5,5-tetramethyl-1,3,2-dioxaborolan-2-yl)propyl)azetidine (0.24 g, 1.0 mmol, 1.0 equiv) in methanol (5.0 mL) in a 40 mL vial. The reaction was stirred at room temperature for 3 h. The reaction was concentrated under reduced pressure. The resulting residue was dissolved in 1 to 1 mixture of methanol and water (12 mL), followed by removal of the solvents under reduced pressure. This protocol was repeated another 3 times, after which no pinacol was detected by ¹H-NMR analysis. The residue was then suspended in acetone (15 mL) and the undissolved solids were filtered off. The filtrate was concentrated under reduced pressure to give the tile compound (0.160 g, 72% yield) as an off-white semi-solid, which was used subsequently.

### Example 15: (1S,3R)-2-(2-Fluoro-2-methylpropyl)-3-methyl-1-(5-(3-(3-methylazetidin-1-yl)propyl)thiophen-2-yl)-2,3,4,9-tetrahydro-1H-pyrido[3,4-b]indole

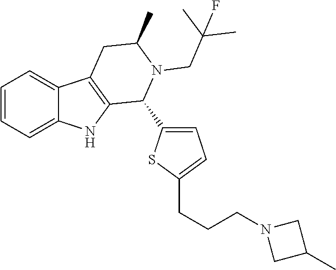 [SMILES: CC1CN(CCCc2ccc([C@@H]3c4[nH]c5ccccc5c4C[C@@H](C)N3CC(C)(C)F)s2)C1]

3-Methyl-1-(3-(trifluoro-2-boraneyl)propyl)azetidine, potassium salt (0.333 g, 1.42 mmol, 2.0 equiv), palladium acetate (0.027 g, 0.121 mmol, 17 mol %), RuPhos (0.109 g, 0.234 mmol, 33 mol %), potassium carbonate (0.500 g, 3.60 mmol, 5 equiv) and (1S,3R)-1-(5-bromothiophen-2-yl)-2-(2-fluoro-2-methylpropyl)-3-methyl-2,3,4,9-tetrahydro-1H-pyrido[3,4-b]indole (0.300 g, 0.712 mmol, 1.0 equiv) were added to a 40 mL vial. Toluene (4.8 mL) and water (2.3 mL) were added to the reaction flask, which was sparged with nitrogen for 1-2 min. The reaction was heated at 90° C. for 18 h. The reaction was cooled to room temperature then transferred to a separatory funnel with dichloromethane (20 mL) and water (20 mL). The layers were separated, and the aqueous layer was extracted with dichloromethane (2×20 mL). The combined organic extracts were dried over sodium sulfate, filtered and concentrated under reduced pressure. The material was purified on a Biotage automated chromatography system (11 g, Biotage 50 m KP-amino-d column), eluting with a gradient of 0 to 3% ethyl acetate in dichloromethane to give the title compound (30 mg, 45% HPLC). The material was absorbed onto Celite® (1 g) and was purified on a Biotage automated chromatography system (11 g, Biotage 50 μm KP-amino-d column), eluting with a gradient of 0 to 50% ethyl acetate in heptanes. The material was lyophilized from a 9 to 1 mixture of acetonitrile and water for 20 h to give the title compound (7.1 mg, 2.1% yield) as a white solid. ¹H NMR (400 MHz, CDCl₃) δ=7.91 (br s, 1H), 7.51 (d, J=7.7 Hz, 1H), 7.33 (d, J=7.6 Hz, 1H), 7.17 (dt, J=1.3, 7.5 Hz, 1H), 7.15-7.07 (m, 1H), 6.52 (t, J=3.9 Hz, 2H), 5.22 (br s, 1H), 3.59 (br t, J=7.1 Hz, 2H), 3.46 (br s, 1H), 2.89-2.72 (m, 4H), 2.71-2.46 (m, 7H), 1.73 (quin, J=7.5 Hz, 2H), 1.56-1.45 (m, 3H), 1.37-1.31 (m, 3H), 1.15 (d, J=6.7 Hz, 6H); LCMS: m/z=454.3 [M+H]⁺.

### Example 16: tert-Butyl-3-((5-((1S,3R)-2-(2-fluoro-2-methylpropy)-3-methyl-2,3,4,9-tetrahydro-1H-pyrido[3,4-b]indol-1-yl)thiophen-2-yl)methyl)azetidine-1-carboxylate

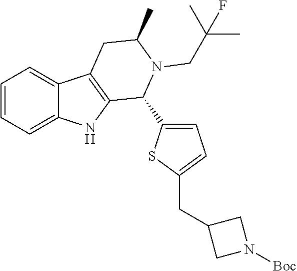 [SMILES: C[C@@H]1Cc2c([nH]c3ccccc23)[C@@H](c2ccc(CC3CN(C(=O)OC(C)(C)C)C3)s2)N1CC(C)(C)F]

A mixture of (1S,3R)-1-(5-bromothiophen-2-yl)-2-(2-fluoro-2-methylpropyl)-3-methyl-2,3,4,9-tetrahydro-1H-pyrido[3,4-b]indole (2.0 g, 4.7 mmol, 1.0 equiv), potassium (1-Boc-azetidin-3-yl)methyltrifluoroborate (1.6 g, 5.7 mmol, 1.2 equiv), toluene (32 mL) and water (16 mL) was sparged with nitrogen for 30 min. Potassium carbonate (2.0 g, 14.2 mmol, 3.0 equiv), RuPhos (0.44 g, 0.94 mmol, 0.2 equiv) and palladium acetate (0.11 g, 0.47 mml, 0.1 equiv) were added sequentially and the reaction mixture was sparged with nitrogen for an additional 15 min. The reaction mixture was the stirred at 80° C. under nitrogen for 25 h, then cooled to room temperature. The reaction mixture was poured onto water and extracted with methyl t-butyl ether (3×60 mL). The combined organic layers were dried over sodium sulfate (30 g), filtered and concentrated under reduced pressure to give a brown oil. The crude product was absorbed onto silica gel (35 g) with dichloromethane (50 mL) and concentrated under reduced pressure. The material was purified on a Biotage automated chromatography system (100 g, 20 μm silica gel column), eluting with a gradient of 0 to 50% methyl t-butyl ether in heptanes to give the title compound (1.7 g, 71% yield) as a white solid. LCMS: m/z=512 [M+H]⁺.

### Example 17: (1S,3R)-1-(5-(Azetidin-3-ylmethyl)thiophen-2-yl)-2-(2-fluoro-2-methylpropyl)-3-methyl-2,3,4,9-tetrahydro-1H-pyrido[3,4-b]indole

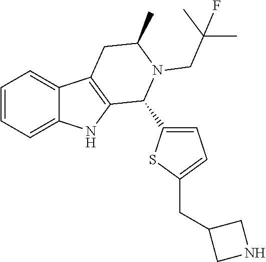 [SMILES: C[C@@H]1Cc2c([nH]c3ccccc23)[C@@H](c2ccc(CC3CNC3)s2)N1CC(C)(C)F]

A solution of tert-butyl-3-((5-((1S,3R)-2-(2-fluoro-2-methylpropyl)-3-methyl-2,3,4,9-tetrabydro-1H-pyrido[3,4-b]indol-1-yl)thiophen-2-yl)methyl)azetidine-1-carboxylate (600 mg, 1.2 mmol, 1.0 equiv) in dichloromethane (10 mL) was cooled to 0 to 5° C. with stirring. Trifluoroacetic acid (2.4 mL, 29.3 mmol, 25 equiv) was added in portions over 10 min. The deep purple reaction mixture was warmed to room temperature over 60 min, then diluted with dichloromethane (90 mL). The resulting solution was cooled to 0 to 5° C. Ice-cold saturated sodium carbonate (30 mL) was added over 10 min. The reaction mixture was warmed to room temperature over 30 min, the layers were separated, and the aqueous layer was extracted with dichloromethane (3×20 ml). The combined organic layers were dried over sodium sulfate (10 g), filtered and concentrated under reduced pressure to give the title compound (480 mg, 100% yield) as a yellow oil, which was used subsequently. LCMS: m/z=412 [M+H]⁺.

### Example 18: (1S,1R)-2-(2-Fluoro-2-methylpropyl)-3-methyl-1-(5-((1-propylazetidin-3-yl)methyl)thiophen-2-yl)-2,3,4,9-tetrahydro-1H-pyrido[3,4-b]indole

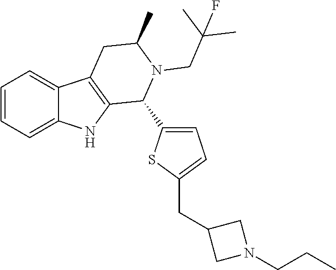 [SMILES: CCCN1CC(Cc2ccc([C@@H]3c4[nH]c5ccccc5c4C[C@@H](C)N3CC(C)(C)F)s2)C1]

1-Bromopropane (0.1 mL, 1.08 mmol, 0.9 equiv) and diisopropylethylamine (1.04 mL, 6.0 mmol, 5.0 equiv) was added to a solution of (1S,3R)-1-(5-(azetidin-3-ylmethyl)thiophen-2-yl)-2-(2-fluoro-2-methylpropyl)-3-methyl-2,3,4,9-tetrahydro-TH-pyrido[3,4-b]indole (0.49 g, 1.2 mmol, 1.0 equiv) in N,N-dimethylacetamide (12 mL) in a 40 mL vial and stirred at room temperature for 4 days. The reaction mixture was transferred to a separatory funnel with ethyl acetate (5 mL) then washed with saturated sodium bicarbonate (2×5 mL). The aqueous layer was extracted with ethyl acetate (2×5 mL). The combined organic layers were dried over sodium sulfate, filtered and concentrated under reduced pressure. The residue was divided into two equal batches and purified on an Interchim automated chromatography system using two Biotage KP-amino-d columns (28 g each, 50 μm), eluting each with a gradient from 0 to 50% dichloromethane in heptanes. The solid was dried under vacuum at 25° C. for 16 hours to give the title compound (205 mg, 65% yield) as a light tan foam. LCMS: m/z=454.3 [M+H]⁺; ¹H NMR (400 MHz, CDCl₃) δ=8.00 (br s, 1H), 7.53, 7.18 (dt, J=1.3, 7.5 Hz, 1H), 7.15-7.10 (m, 1H), 6.54 (br d, J=2.4 Hz, 1H), 6.50 (d, J=3.4 Hz, 1H), 5.29-5.18 (m, 1H), 3.44 (br t, J=7.2 Hz, 3H), 3.05-2.94 (m, 2H), 2.85 (q, J=6.7 Hz, 2H), 2.80-2.48 (m, 5H), 2.43-2.33 (m, 2H), 1.56-1.48 (m, 3H), 1.42-1.23 (m, 8H), 1.16 (d, J=6.7 Hz, 3H), 0.89 (t, J=7.4 Hz, 4H).

### Example 19: (1S,3R)-2-(2-Fluoro-2-methylpropyl)-1-(5-((1-(3-fluoropropyl)azetidin-3-yl)methyl)thiophen-2-yl)-3-methyl-2,3,4,9-tetrahydro-1H-pyrido[3,4-b]indole

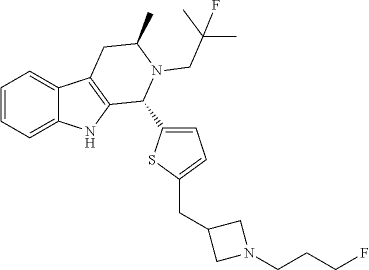 [SMILES: C[C@@H]1Cc2c([nH]c3ccccc23)[C@@H](c2ccc(CC3CN(CCCF)C3)s2)N1CC(C)(C)F]

1-Bromo-3-fluoropropane (0.27 mL, 1.9 mmol, 0.95 equiv) and diisopropylethylamine (1.73 mL, 10.0 mmol, 5.0 equiv) was added to a solution of (1S,3R)-1-(5-(azetidin-3-ylmethyl)thiophen-2-yl)-2-(2-fluoro-2-methylpropyl)-3-methyl-2,3,4,9-tetrahydro-1H-pyrido[3,4-b]indole (0.82 g, 2.0 mmol, 1.0 equiv) in N,N-dimethylacetamide (20 mL) in a 40 mL vial and stirred at room temperature for 4 days. The reaction mixture was transferred to a separatory funnel with ethyl acetate (10 mL) then washed with saturated sodium bicarbonate (2×10 mL). The aqueous layer was extracted with ethyl acetate (2×10 mL). The combined organic layers were dried over sodium sulfate, filtered and concentrated under reduced pressure. The residue was divided into two equal batches and purified on an Interchim automated chromatography system using two Biotage KP-amino-d columns (55 g each, 50 μm), eluting each with a gradient from 0 to 50% dichloromethane in heptanes. The solid was dried under vacuum at 25° C. for 16 hours to give the title compound (130 mg, 14% yield) as a white solid. LCMS: m/z=472.3 [M+H]⁺; ¹H NMR (400 MHz, CDCl₃) δ=8.27-7.96 (m, 1H), 7.58-7.47 (m, 1H), 7.36-7.28 (m, 1H), 7.18 (t, J=7.2 Hz, 1H), 7.14-7.09 (m, 1H), 6.68-6.41 (m, 2H), 5.32-5.12 (m, TH), 4.52 (t, J=6.0 Hz, 1H), 4.41 (t, J=6.0 Hz, 1H), 3.43 (br t, J=7.2 Hz, 3H), 2.98 (br d, 1 J=7.5 Hz, 2H), 2.86 (q, J=7.4 Hz, 2H), 2.80-2.47 (m, 7H), 1.81-1.65 (m, 2H), 1.60-1.45 (m, 3H), 1.39-1.29 (m, 3H), 1.27 (s, 1H), 1.16 (d, J=6.7 Hz, 3H).

### Example 20: (1S,3R)-1-(5-((1-(3,3-Difluoropropyl)azetidin-3-yl)methyl)thiophen-2-yl)-2-(2-fluoro-2-methylpropyl)-3-methyl-2,3,4,9-tetrahydro-1H-pyrido[3,4-b]indole

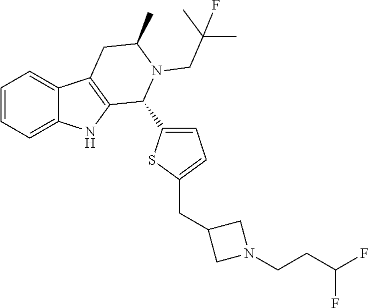 [SMILES: C[C@@H]1Cc2c([nH]c3ccccc23)[C@@H](c2ccc(CC3CN(CCC(F)F)C3)s2)N1CC(C)(C)F]

Diisopropylethylamine (0.27 g, 2.1 mmol, 5.0 equiv) was added to a solution of (1S,3R)-1-(5-(azetidin-3-ylmethyl)thiophen-2-yl)-2-(2-fluoro-2-methylpropyl)-3-methyl-2,3,4,9-tetrahydro-1H-pyrido[3,4-b]indole (170 mg, 0.41 mmol, 1.0 equiv) and 3,3-difluoropropyl tosylate (104 mg, 0.41 mmol, 1.0 equiv) in N,N-dimethylacetamide (4 mL). After stirring for 18 h, the reaction mixture was diluted with ethyl acetate (30 mL), washed with water (4×12 mL), dried over magnesium sulfate (10 g), filtered and concentrated under reduced pressure. The crude product was absorbed onto Celite® (2 g) with dichloromethane (10 mL) and solvent was removed under reduced pressure. The material was purified on a Biotage automated chromatography system (11 g, 60 μm silica gel column), eluting with a gradient of 0 to 100% ethyl acetate in heptanes to give crude title compound (30 mg, 16% yield). The crude product was treated with a 6 M sodium hydroxide solution (0.02 mL, 2 equiv) in methanol (1 mL) for 3 h. Solvent was removed under reduced pressure and the residue was diluted with methyl t-butyl ether (2 ml) and washed with water (2×1 mL), dried over magnesium sulfate (50 mg) and filtered. The filtrate was absorbed onto Celite® (1 g) and solvent was removed under reduced pressure. The material was purified on a Biotage automated chromatography system (Amino Duo column 11 g, 60 μm silica gel column), eluting with a gradient of 0 to 50% ethyl acetate in heptanes to give the title compound (11 mg, 5% yield) as a white solid. ¹H NMR (400 MHz, CDCl₃) δ=7.85 (br s, 11H), 7.53 (d, J=7.7 Hz, 1H), 7.36-7.31 (m, 1H), 7.21-7.10 (m, 2H), 6.67-6.49 (m, 2H), 5.89 (tt, J=4.5, 56.8 Hz, 1H), 5.31-5.18 (m, 1H), 3.45 (br s, 3H), 3.00 (d, J=7.7 Hz, 2H), 2.95-2.85 (m, 2H), 2.76 (td, J=6.8, 13.9 Hz, 1H), 2.70-2.48 (m, 6H), 1.88 (dtt, J=4.5, 7.2, 17.1 Hz, 2H), 1.56-1.47 (m, 2H), 1.38-1.31 (m, 3H), 1.16 (d, J=6.8 Hz, 3H); LCMS: m/z=490.3 [M+H]⁺.

### Example 21: (1S,3R)-2-(2-Fluoro-2-methylpropyl)-3-methyl-1-(5-((1-(3,3,3-trifluoropropyl)azetidin-3-yl)methyl)thiophen-2-yl)-2,3,4,9-tetrahydro-1H-pyrido[3,4-b]indole

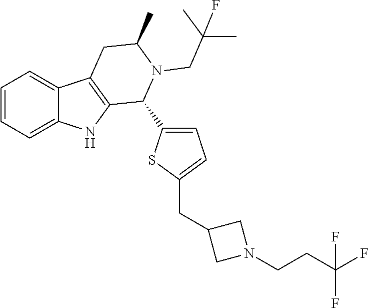 [SMILES: C[C@@H]1Cc2c([nH]c3ccccc23)[C@@H](c2ccc(CC3CN(CCC(F)(F)F)C3)s2)N1CC(C)(C)F]

Diisopropylethylamine (0.37 g, 2.9 mmol, 5.0 equiv) was added to a solution of (1S,3R)-1-(5-(azetidin-3-ylmethyl)thiophen-2-yl)-2-(2-fluoro-2-methylpropyl)-3-methyl-2,3,4,9-tetrahydro-1H-pyrido[3,4-b]indole (240 mg, 0.58 mmol, 1.0 equiv), 1-bromo-3,3,3-trifluoropropane (103 mg, 0.58 mmol, 1.0 equiv) in N,N-dimethylacetamide (5.5 mL). After stirring for 18 h, the reaction mixture was diluted with ethyl acetate (50 mL), washed with water (4×15 mL), dried over magnesium sulfate (10 g), filtered and concentrated under reduced pressure. The crude product was absorbed onto Celite® (2 g) with dichloromethane (5 mL) and solvent was removed under reduced pressure. The material was purified on a Biotage automated chromatography system (Amino Duo column 28 g, 60 μm silica gel column), eluting with a gradient of 0 to 100% ethyl acetate in heptanes to give the title compound (16 mg, 5% yield) as a white solid. ¹H NMR (400 MHz, CDCl₃) δ=7.82 (br s, 1H), 7.52 (d, J=7.8 Hz, 1H), 7.33 (d, J=7.9 Hz, 11H), 7.18 (dt, J=1.2, 7.5 Hz, 11H), 7.14-7.09 (m, 11H), 6.68-6.46 (m, 2H), 5.30-5.16 (m, 1H), 3.52-3.36 (m, 3H), 2.99 (d, J=7.6 Hz, 2H), 2.89 (q, 0.1=6.3 Hz, 2H), 2.81-2.47 (m, 7H), 2.20-2.05 (m, 2H), 1.57-1.45 (m, 3H), 1.37-1.30 (m, 3H), 1.15 (d, J=6.7 Hz, 3H); LCMS: m/z=508.3 [M+H]⁺.

### Example 22: (1S,3R)-1-(5-Bromothiophen-2-yl)-2-(3-((tert-butyldiphenylsilyl)oxy)-2,2-difluoropropyl)-3-methyl-2,3,4,9-tetrahydro-1H-pyrido[3,4-b]indole

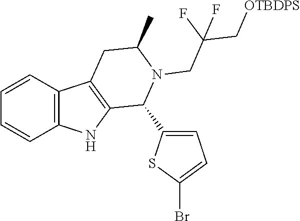

A mixture of 5-bromo-2-thiophenecarboxaldehyde (1.4 g, 7.32 mmol, 0.95 equiv), (R)—N-(1-(1H-indol-3-yl)propan-2-yl)-3-((tert-butyldiphenylsilyl)oxy)-2,2-difluoropropan-1-amine (3.9 g, 7.70 mmol, 1.0 equiv) and acetic acid (0.88 mL, 15.4 mmol, 2.0 equiv) in toluene (65 mL) was heated at 80° C. in a sealed bottle for 72 h. After cooling to room temperature, the mixture was diluted with ethyl acetate (200 mL) and washed with saturated sodium bicarbonate (80 mL). The aqueous layer was extracted with ethyl acetate (2×100 mL). The combined organic layers were washed with saturated brine (200 mL), dried over sodium sulfate, filtered and concentrated under reduced pressure. The residue was purified on an Interchim automated chromatography system (Sorbtech 330 g silica gel column), eluting with a gradient of 0 to 25% ethyl acetate in heptanes to give the title compound (3.56 g, 72% yield) as a white solid. LCMS: m/z=679.1 [M+H]⁺.

### Example 23: tert-Butyl 3-((5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiophen-2-yl)methyl)azetidine-1-carboxylate

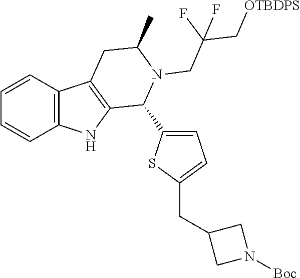

A suspension of (1S,3R)-1-(5-bromothiophen-2-yl)-2-(3-((tert-butyldiphenylsilyl)oxy)-2,2-difluoropropyl)-3-methyl-2,3,4,9-tetrahydro-1H-pyrido[3,4-b]indole (3.0 g, 4.41 mmol, 1.0 equiv), potassium (1-Boc-azetidin-3-yl)methyltrifluoroborate (1.47 g, 5.29 mmol, 1.2 equiv), potassium carbonate (1.83 g, 13.23 mmol, 3.0 equiv), palladium acetate (99 mg, 0.441 mmol, 0.1 equiv) and RuPhos (0.41 g, 0.882 mmol, 0.2 equiv) in toluene (30 mL) and water (15 mL) was sparged with nitrogen for 10 min. Then the suspension was heated at 80° C. for 24 h. After cooling to room temperature, the mixture was diluted with ethyl acetate (150 mL) and water (100 mL). The aqueous layer was separated and extracted with ethyl acetate (150 mL). The combined organic layers were washed with saturated brine (100 mL), dried over sodium sulfate, filtered and concentrated under reduced pressure. The residue was purified on an Interchim automated chromatography system (Sorbtech 330 g silica gel column), eluting with a gradient of 0 to 40% ethyl acetate in heptanes to give the title compound (3.0 g, 88% yield) as a yellow solid. LCMS: m/z=770.3 [M+H]⁺.

### Example 24: tert-Butyl (1S,3R)-1-(5-((1-(tert-butoxycarbonyl)azetidin-3-yl)methyl) thiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

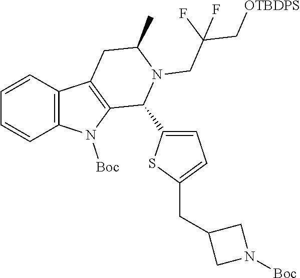

Di-tert-butyl dicarbonate (271 mg, 1.24 mmol, 1.8 equiv) was added to a solution of tert-butyl 3-((5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiophen-2-yl)methyl)azetidine-1-carboxylate (530 mg, 0.69 mmol, 1.0 equiv), trimethylamine (0.19 mL, 1.38 mmol, 2.0 equiv) and 4-dimethylaminopyridine (25 mg, 0.21 mmol, 0.3 equiv) in tetrahydrofuran (8 mL). The reaction mixture was stirred at room temperature for 16 h. The solvent was removed under reduced pressure and the residue was purified on an Interchim automated chromatography system (Biotage® Sfar Silica 50 g silica gel column), eluting with a gradient of 0 to 50% ethyl acetate in heptanes to give the title compound (600 mg, 99% yield) as a colorless oil. LCMS: m/z=870.2 [M+H]⁺.

### Example 25: tert-Butyl (1S,3R)-1-(5-((1-(tert-butoxycarbonyl)azetidin-3-yl)methyl) thiophen-2-yl)-2-(2,2-difluoro-3-hydroxypropyl)-3-methyl-1,2,3,4-tetrahydro-9H-pyrido[3,4-b]indole-9-carboxylate

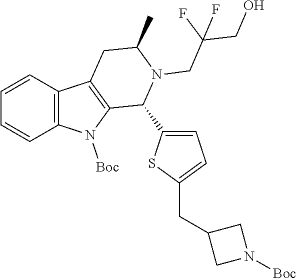 [SMILES: C[C@@H]1Cc2c(n(C(=O)OC(C)(C)C)c3ccccc23)[C@@H](c2ccc(CC3CN(C(=O)OC(C)(C)C)C3)s2)N1CC(F)(F)CO]

1.0 M Tetrabutylammonium fluoride solution in tetrahydrofuran (0.72 mL, 0.72 mmol, 1.05 equiv) was added to a solution of tert-butyl (1S,3R)-1-(5-((1-(tert-butoxycarbonyl)azetidin-3-yl)methyl) thiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (600 mg, 0.69 mmol, 1.0 equiv) in tetrahydrofuran (7 mL) at room temperature. The resulting mixture was stirred at room temperature for 1 h. The solvent was removed under reduced pressure and the residue was purified on an Interchim automated chromatography system (Biotage® Sfar Silica 50 g silica gel column), eluting with a gradient of 0 to 60% ethyl acetate in heptanes to give the compound (400 mg, 92% yield) as a colorless oil. LCMS: m/z=632.4 [M+H]⁺.

### Example 26: tert-Butyl (1S,3R)-1-(5-((1-(tert-butoxycarbonyl)azetidin-3-yl)methyl) thiophen-2-yl)-3-methyl-2-(2,2,3-trifluoropropyl)-1,2,3,4-tetrahydro-9H-pyrido[3,4-b]indole-9-carboxylate

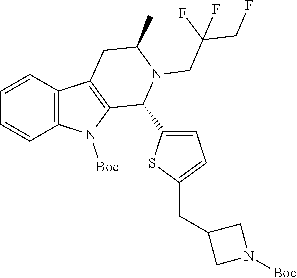 [SMILES: C[C@@H]1Cc2c(n(C(=O)OC(C)(C)C)c3ccccc23)[C@@H](c2ccc(CC3CN(C(=O)OC(C)(C)C)C3)s2)N1CC(F)(F)CF]

Trifluoromethanesulfonic anhydride (0.12 mL, 0.70 mmol, 1.1 equiv) was added to a solution of tert-butyl (1S,3R)-1-(5-((1-(tert-butoxycarbonyl)azetidin-3-yl)methyl) thiophen-2-yl)-2-(2,2-difluoro-3-hydroxypropyl)-3-methyl-1,2,3,4-tetrahydro-9H-pyrido[3,4-b]indole-9-carboxylate (400 mg, 0.63 equiv, 1.0 equiv) and 2,6-dimethylpyridine (0.22 mL, 1.9 mL, 3.0 equiv) in dichloromethane (3 mL) at 0° C. The reaction mixture was stirred at 0° C. for 1 h. The solvent was removed under reduced pressure. The residue was dissolved in tetrahydrofuran (3 mL) at room temperature. 1.0 M Tetrabutylammonium fluoride solution in tetrahydrofuran (3.2 mL, 3.2 mmol, 5.0 equiv) was added, and the resulting solution was stirred at room temperature for 1 h. The solvent was removed under reduced pressure and the residue was purified on an Interchim automated chromatography system (Biotage® Sfar Silica 50 g silica gel column), eluting with a gradient of 0 to 50% ethyl acetate in heptanes to give the compound (200 mg, 50% yield) as a colorless oil. LCMS: m/z=634.3 [M+H]⁺.

### Example 27: (1S,3R)-1-(5-(Azetidin-3-ylmethyl)thiophen-2-yl)-3-methyl-2-(2,2,3-trifluoropropyl)-2,3,4,9-tetrahydro-1H-pyrido[3,4-b]indole

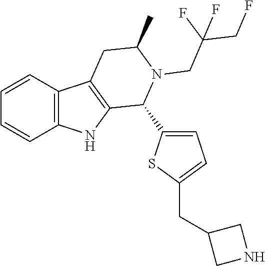 [SMILES: C[C@@H]1Cc2c([nH]c3ccccc23)[C@@H](c2ccc(CC3CNC3)s2)N1CC(F)(F)CF]

Trifluoroacetic acid (0.4 mL, 5.2 mmol, 17.4 equiv) was added to a solution of tert-butyl (1S,3R)-1-(5-((1-(tert-butoxycarbonyl)azetidin-3-yl)methyl) thiophen-2-yl)-3-methyl-2-(2,2,3-trifluoropropyl)-1,2,3,4-tetrahydro-9H-pyrido[3,4-b]indole-9-carboxylate (190 mg, 0.3 mmol, 1.0 equiv) in dichloromethane (1.6 mL) at 0° C. The reaction was warmed to room temperature, and stirred for 3.5 h, at which time LCMS analysis indicated the reaction was completed. The reaction was poured into a mixture of saturated sodium carbonate (30 mL) and dichloromethane (20 mL). The layers were separated, and the aqueous layer was extracted with dichloromethane (20 mL). The combined organic layers were washed with saturated brine (20 mL), dried over sodium sulfate and concentrated under reduced pressure to give the title compound (130 mg, 99% yield) as a grey solid. LCMS: m/z=434.2 [M+H]⁺.

### Example 28: (1S,3R)-1-(5-((1-(3-Fluoropropyl)azetidin-3-yl)methyl)thiophen-2-yl)-3-methyl-2-(2,2,3-trifluoropropyl)-2,3,4,9-tetrahydro-1H-pyrido[3,4-b]indole

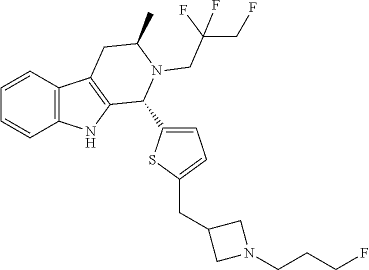 [SMILES: C[C@@H]1Cc2c([nH]c3ccccc23)[C@@H](c2ccc(CC3CN(CCCF)C3)s2)N1CC(F)(F)CF]

1-Bromo-3-fluoropropane (51 mg, 0.36 mmol, 1.2 equiv) and N,N-diisopropylethylamine (0.08 mL, 0.45 mmol, 1.5 equiv) were sequentially added to a solution of (1S,3R)-1-(5-(azetidin-3-ylmethyl)thiophen-2-yl)-3-methyl-2-(2,2,3-trifluoropropyl)-2,3,4,9-tetrahydro-1H-pyrido[3,4-b]indole (130 mg, 0.3 mmol, 1.0 equiv) in N,N-dimethylacetamide (2 mL) at room temperature. The resulting solution was stirred at room temperature for 2 days, at which time LCMS analysis indicated the reaction was completed. The reaction mixture was poured into a mixture of water (20 mL) and dichloromethane (20 mL). The layers were separated, and the aqueous layer was extracted with dichloromethane (2×15 mL). The combined organic layers were washed with saturated brine (30 mL), dried over sodium sulfate, filtered and concentrated under reduced pressure. The residue was purified on an Interchim automated chromatography system (28 g, Biotage® Sfar KP-Amino D), eluting with a gradient of 0 to 100% dichloromethane in heptanes to give the title compound (35 mg, 24% yield) as an off-white solid. LCMS: m/z=494.2 [M+H]⁺; ¹H NMR (400 MHz, CDCl₃) δ=7.96 (s, 1H), 7.52 (d, J=7.8 Hz, 1H), 7.36-7.30 (m, 1H), 7.20 (dt, J=1.2, 7.6 Hz, 1H), 7.16-7.10 (m, 1H), 6.62-6.43 (m, 2H), 5.15 (s, 1H), 4.95-4.55 (m, 2H), 4.53 (t, J=6.0 Hz, 1H), 4.41 (t, J=6.0 Hz, 1H), 3.52-3.36 (m, 3H), 3.24-3.10 (m, 1H), 3.05-2.94 (m, 2H), 2.91-2.79 (m, 3H), 2.79-2.66 (m, 2H), 2.61-2.48 (m, 3H), 1.82-1.65 (m, 3H), 1.19 (d, J=6.8 Hz, 3H).

### Example 29: tert-Butyl 3-((5-(methoxycarbonyl)thiophen-2-yl)methyl)azetidine-1-carboxylate

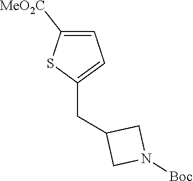 [SMILES: COC(=O)c1ccc(CC2CN(C(=O)OC(C)(C)C)C2)s1]

A mixture of methyl 5-bromothiophene-2-carboxylate (1.82 g, 8.21 mmol, 1 equiv), potassium (1-Boc-azetidin-3-yl)methyltrifluoroborate (2.73 g, 9.85 mmol, 1.2 equiv), RuPhos (0.67 g, 1.64 mmol, 0.2 equiv) and potassium carbonate (3.40 g, 24.63 mmol, 3 equiv) in 9 to 1 mixture of toluene and water (9:1, 100 mL) was sparged with nitrogen for 20 min. Palladium acetate (0.18 g, 0.82 mmol, 0.1 equiv) was added and the mixture was sparged with a stream of nitrogen for an additional 5 min. After heating at 100° C. for 9 h, the mixture was diluted with ethyl acetate (100 mL), washed with water (100 mL) and saturated brine (100 mL). The combined aqueous layers were extracted with ethyl acetate (2×100 mL). The combined organic layers were dried over anhydrous sodium sulfate, filtered and concentrated under reduced pressure. The residue was purified on an Interchim automated chromatography system (Sorbtech 120 g silica gel column), eluting with a gradient of 0 to 50% ethyl acetate in heptanes to give the title compound (1.9 g, 74% yield) as a yellow solid. LCMS: m/z=334.1 [M+Na]⁺; ¹H NMR (400 MHz, CDCl₃) δ=7.63 (d, 1H), 6.79 (d, 1H), 4.05 (m, 2H), 3.91 (m, 2H), 3.89 (s, 3H), 3.66 (m, 2H), 3.11 (d, 2H), 2.84 (m, 1H), 1.44 (s, 9H).

### Example 30: Methyl 5-(azetidin-3-ylmethyl)thiophene-2-carboxylate

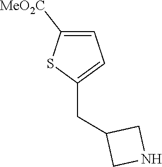 [SMILES: COC(=O)c1ccc(CC2CNC2)s1]

Trifluoroacetic acid (7.87 mL, 103 mmol, 20 equiv) was added to a solution of tert-butyl 3-((5-(methoxycarbonyl)thiophen-2-yl)methyl)azetidine-1-carboxylate (1.6 g, 5.14 mmol, 1 equiv) in dichloromethane (40 mL) at 0-5° C. After stirring at room temperature for 4 h, LCMS analysis indicated that the reaction was complete. The residue was concentrated under reduced pressure at 25° C., diluted with dichloromethane (100 mL) and treated with saturated sodium carbonate adjusting the pH to 9. The layers were separated, and the aqueous layer was extracted with dichloromethane (2×50 mL). The combined organic layers were dried over anhydrous sodium sulfate, filtered and concentrated under reduced pressure to give crude compound (1.05 g, 97% yield) as a yellow solid. LCMS: m/z=212.1 [M+H]⁺.

### Example 31: Methyl 5-((1-(3-fluoropropyl)azetidin-3-yl)methyl)thiophene-2-carboxylate

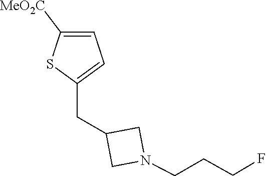 [SMILES: COC(=O)c1ccc(CC2CN(CCCF)C2)s1]

N,N-Diisopropylethylamine (4.33 mL, 24.9 mmol, 5 equiv) was added to a solution of methyl 5-(azetidin-3-ylmethyl)thiophene-2-carboxylate (1.05 g, 4.97 mmol, 1 equiv) in N,N-dimethylacetamide (20 mL) and the mixture was stirred at room temperature for 10 min. 1-Bromo-3-fluoropropane (0.7 g, 4.97 mmol, 1 equiv) was added, and the mixture was stirred at room temperature for 20 h. The mixture was diluted with ethyl acetate (100 mL) and washed with water (50 mL). The aqueous layer was extracted with ethyl acetate (2×50 mL). The combined organic layers were washed with saturated brine (100 mL), dried over anhydrous sodium sulfate, filtered and concentrated under reduced pressure. The residue was purified on an Interchim automated chromatography system (Redisep 100 g reverse phase C18 Gold column), eluting with a gradient of 0 to 100% acetonitrile in water to give the title compound (0.4 g, 30% yield) as a viscous yellow oil. LCMS: m/z=272.1 [M+H]⁺; ¹H NMR (400 MHz, CDCl₃) δ=7.63 (d, 1H), 6.77 (d, 1H), 4.50 (dd, 2H), 3.86 (s, 3H), 3.43 (m, 2H), 3.08 (d, 2H), 2.87 (m, 2H), 2.77 (m, 1H), 2.53 (m, 2H), 1.77-1.71 (m, 2H).

### Example 32: (5-((1-(3-Fluoropropyl)azetidin-3-yl)methyl)thiophen-2-yl)methanol

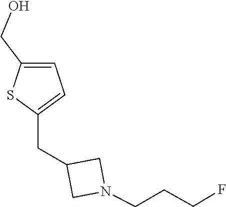 [SMILES: OCc1ccc(CC2CN(CCCF)C2)s1]

1.0 M Lithium aluminum hydride in THE (1.4 mL, 1.4 mmol, 1 equiv) was added dropwise to a solution of methyl 5-((1-(3-fluoropropyl)azetidin-3-yl)methyl)thiophene-2-carboxylate (370 mg, 1.36 mmol, 1 equiv) in anhydrous THF (10 mL) at 0 to 5° C. After stirring at room temperature for 3 h, LCMS analysis indicated that the reaction was complete. The mixture was cooled in an ice-bath, water (0.1 mL), 15% aqueous sodium hydroxide solution (0.1 mL), and water (0.3 mL) were sequentially added to quench the reaction. The mixture was extracted with ethyl acetate (3×40 mL) and the combined organic layers were washed with saturated brine (40 mL), dried over sodium sulfate, filtered and concentrated under reduced pressure to give the crude title compound (345 mg, quant) as a yellow oil. LCMS: m/z=244.2 [M+H]⁺; s ¹H NMR (400 MHz, CDCl₃) δ=6.78 (d, 1H), 6.59 (d, 1H), 4.73 (s, 2H), 4.52 (dd, 2H), 3.38 (m, 2H), 2.99 (d, 2H), 2.84 (m, 2H), 2.69 (m, 1H), 2.53 (m, 2H), 1.77-1.67 (m, 2H).

### Example 33: 5-((1-(3-Fluoropropyl)azetidin-3-yl)methyl)thiophene-2-carbaldehyde

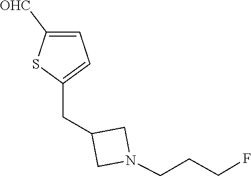 [SMILES: O=Cc1ccc(CC2CN(CCCF)C2)s1]

Activated manganese oxide (1.05 g, 12.1 mmol, 10 equiv) was added to a solution of (5-((1-(3-fluoropropyl)azetidin-3-yl)methyl)thiophen-2-yl)methanol (295 mg, 1.21 mmol, 1 equiv) in dichloromethane (15 mL) at room temperature. After stirring at room temperature for 20 h, the mixture was filtered through Celite®, which was washed with dichloromethane (3×10 mL). The filtrate was concentrated under reduced pressure to give the title compound (269 mg, 86% yield) as a yellow oil. LCMS: m/z=242.2 [M+H]⁺; ¹H NMR (400 MHz, CDCl₃) δ=9.82 (s, 1H), 7.61 (d, 1H), 6.89 (d, 1H), 4.45 (dd, 2H), 3.43 (m, 2H), 3.13 (d, 2H), 2.90 (m, 2H), 2.78 (m, 1H), 2.54 (t, 2H), 1.77-1.67 (m, 2H).

### Example 34: (1S,3R)-2-(3-((tert-Butyldiphenylsilyl)oxy)-2,2-difluoropropyl)-1-(5-((1-(3-fluoropropyl)azetidin-3-yl)methyl)thiophen-2-yl)-3-methyl-2,3,4,9-tetrahydro-1H-pyrido[3,4-b]indole

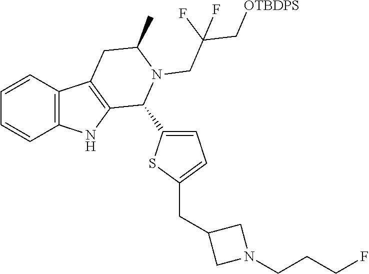

Acetic acid (0.046 mL, 0.82 mmol, 2.0 equiv) was added at room temperature to a solution of 5-((1-(3-fluoropropyl)azetidin-3-yl)methyl)thiophene-2-carbaldehyde (0.1 g, 0.414 mmol, 1 equiv) and (R)—N-(1-(1H-indol-3-yl)propan-2-yl)-3-((tert-butyldiphenylsilyl)oxy)-2,2-difluoropropan-1-amine (0.25 g, 0.497 mmol, 1.2 equiv) in toluene (2 mL). After heating at 80° C. for 16 h, additional acetic acid (0.07 mL, 1.24 mmol, 3 equiv) was added and the reaction was heated an additional 8 h at 80° C. The reaction was cooled to room temperature and diluted with ethyl acetate (10 mL) and saturated sodium bicarbonate (10 mL). The layers were separated, and the aqueous layer was extracted with ethyl acetate (3×10 mL). The combined organic layers were washed with saturated brine (10 mL), dried over sodium sulfate, filtered and concentrated under reduced pressure. The crude residue was purified on an Biotage automated chromatography system (Biotage 28 g KPNH), eluting with a gradient of 0 to 70% ethyl acetate in heptanes to give the title compound (146 mg, 49% yield) as white solid. LCMS: m/z=730.3 [M+H]⁺.

### Example 35: 2,2-Difluoro-3-((1S,3R)-1-(5-((1-(3-fluoropropyl)azetidin-3-yl)methyl)thiophen-2-yl)-3-methyl-1,3,4,9-tetrahydro-2H-pyrido[3,4-b]indol-2-yl)propan-1-ol

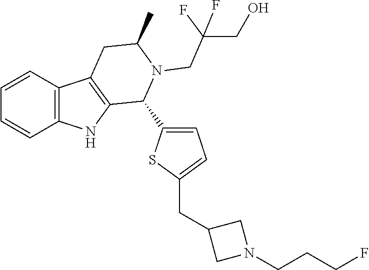 [SMILES: C[C@@H]1Cc2c([nH]c3ccccc23)[C@@H](c2ccc(CC3CN(CCCF)C3)s2)N1CC(F)(F)CO]

1 M Tetra-n-butylammonium fluoride in THE (0.22 mL, 0.215 mmol, 1.1 equiv) was added dropwise at room temperature to a solution of (1 S,3R)-2-(3-((tert-butyldiphenylsilyl)oxy)-2,2-difluoropropyl)-1-(5-((1-(3-fluoropropyl)azetidin-3-yl)methyl)thiophen-2-yl)-3-methyl-2,3,4,9-tetrahydro-1H-pyrido[3,4-b]indole (0.14 g, 0.196 mmol, 1 equiv) in THE (1.8 mL). After stirring for 1 h, the reaction was concentrated under reduced pressure. The residue was diluted with saturated ammonium chloride (5 mL). The suspension was extracted with ethyl acetate (3×5 mL). The combined organic layers were dried over sodium sulfate, filtered and concentrated under reduced pressure. The residue was purified on Biotage automated chromatography system (Biotage 11 g KPNH, eluting with gradient of 0 to 80% ethyl acetate in dichloromethane to give the title compound (52.8 mg, 55% yield) as a white solid. ¹H NMR (400 MHz, CDCl₃) δ=8.31 (br s, 1H), 7.53 (d, J=7.7 Hz, 1H), 7.30 (d, J=7.9 Hz, 1H), 7.21-7.10 (m, 2H), 6.59 (d, J=3.2 Hz, 1H), 6.51 (d, J=3.4 Hz, 1H), 5.22 (s, 1H), 4.50 (t, J=5.9 Hz, 1H), 4.39 (t, J=5.9 Hz, 1H), 4.13 (q, J=7.1 Hz, 1H), 4.00-3.90 (m, 2H), 3.58-3.48 (m, 1H), 3.40 (q, J=6.4 Hz, 2H), 3.23-3.10 (m, 1H), 2.99-2.82 (m, 5H), 2.79-2.66 (m, 2H), 2.60-2.48 (m, 3H), 1.78-1.64 (m, 2H), 1.26 (t, J=7.2 Hz, 1H), 1.23 (d, J=6.7 Hz, 3H); LCMS: m/z=492.2 [M+H]⁺.

### Example 36: (1S,3R)-2-(2,2-Difluoroethyl)-1-(5-((1-(3-fluoropropyl)azetidin-3-yl)methyl)thiophen-2-yl)-3-methyl-2,3,4,9-tetrahydro-1H-pyrido[3,4-b]indole

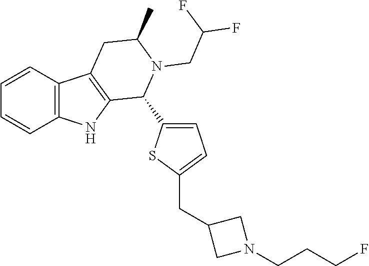 [SMILES: C[C@@H]1Cc2c([nH]c3ccccc23)[C@@H](c2ccc(CC3CN(CCCF)C3)s2)N1CC(F)F]

A mixture of (R)—N-(2,2-difluoroethyl)-1-(1H-indol-3-yl)propan-2-amine (128 mg, 0.539 mmol, 1.0 equiv), 5-((1-(3-fluoropropyl)azetidin-3-yl)methyl)thiophene-2-carbaldehyde (130 mg, 0.539 mmol, 1.2 equiv) and acetic acid (0.093 mL, 1.616 mmol, 3.0 equiv) in toluene (6 mL) was heated at 90° C. in a sealed bottle protected from light for 48 h. After cooling to room temperature, the mixture was diluted with ethyl acetate (100 mL) and washed with saturated sodium bicarbonate (40 mL). The aqueous layer was extracted with ethyl acetate (2×40 mL). The combined organic layers were washed with saturated brine (40 mL), dried over sodium sulfate, filtered and concentrated under reduced pressure. The residue was purified on an Interchim automated chromatography system (Redisep 50 g C18 Gold reverse phase column), eluting with a gradient of 0 to 100% acetonitrile in water to give the title compound (106 mg, 43% yield) as an off-white solid. LCMS: m/z=462.2 [M+H]⁺; ¹H NMR (400 MHz, CDCl₃) δ=7.90 (s, 1H), 7.52 (d, J=7.8 Hz, 1H), 7.32-7.28 (m, H), 7.18 (dt, J=1.3, 7.5 Hz, 1H), 7.14-7.09 (m, 1H), 6.67 (dd, J=0.7, 3.4 Hz, 1H), 6.54 (d, J=3.4 Hz, 1H), 5.78 (tt, J=4.4, 56.6 Hz, 1H), 5.07 (s, 1H), 4.52 (t, J=6.0 Hz, 1H), 4.40 (t, J=6.0 Hz, 1H), 3.52-3.39 (m, 3H), 3.09-2.95 (m, 3H), 2.88-2.64 (m, 6H), 2.60-2.50 (m, 3H), 1.79-1.65 (m, 2H), 1.18 (d, J=6.7 Hz, 3H); ¹⁹F NMR (376 MHz, CDCl₃) δ=−118.93-−119.37 (m, 2F), −219.30-−219.73 (m, 1F).

### Example 37: (1S,3R)-1-(5-((1-(3-Fluoropropyl)azetidin-3-yl)methyl)thiophen-2-yl)-3-methyl-2-(2,2,2-trifluoroethyl)-2,3,4,9-tetrahydro-1H-pyrido[3,4-b]indole

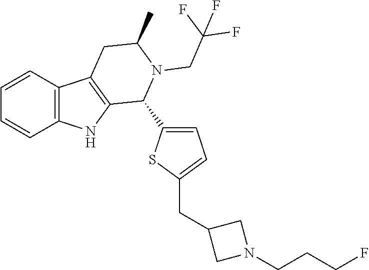 [SMILES: C[C@@H]1Cc2c([nH]c3ccccc23)[C@@H](c2ccc(CC3CN(CCCF)C3)s2)N1CC(F)(F)F]

A mixture of (R)-1-(1H-indol-3-yl)-N-(2,2,2-trifluoroethyl)propan-2-amine (149 mg, 0.539 mmol, 1.0 equiv), 5-((1-(3-fluoropropyl)azetidin-3-yl)methyl)thiophene-2-carbaldehyde (130 mg, 0.539 mmol, 1.0 equiv) and acetic acid (0.093 mL, 1.616 mmol, 3.0 equiv) in toluene (6 mL) was heated at 90° C. in a sealed bottle protected from light for 5 days. After cooling to room temperature, the mixture was diluted with ethyl acetate (100 mL) and washed with saturated sodium bicarbonate (40 mL). The aqueous layer was extracted with ethyl acetate (2×40 mL). The combined organic layers were washed with saturated brine (40 mL), dried over sodium sulfate, filtered and concentrated under reduced pressure. The residue was purified on an Interchim automated chromatography system (Redisep 50 g C18 Gold reverse phase column), eluting with a gradient of 0 to 100% acetonitrile in water to give the title compound (69 mg, 25% yield) as a yellow solid. This material was further purified by preparative HPLC to give pure title compound (2.0 mg) as an off-white solid. LCMS: m/z=480.2 [M+H]⁺; ¹H NMR (400 MHz, CDCl₃) δ=7.87 (s, 1H), 7.52 (d, J=7.6 Hz, 1H), 7.33 9d, J=8.1 Hz, 1H), 7.20 (dt, J=1.2, 7.5 Hz, 1H), 7.15-7.10 (m, 1H), 6.59 (dd, J=1.0, 3.5 Hz, 1H), 6.52 (d, J=3.5 Hz, 1H), 5.13 (s, 1H), 4.53 (t, J=6.0 Hz, 1H), 4.41 (t, J=6.1 Hz, 1H), 3.52-3.40 (m, 3H), 3.24 (qd, J=9.6, 15.6 Hz, 1H), 3.03-2.83 (m, 5H), 2.78-2.69 (m, 2H), 2.58-2.46 (m, 3H), 1.88-1.52 (m, 5H), 1.21 (d, J=6.7 Hz, 3H); ¹⁹F NMR (376 MHz, CDCl₃) δ=−71.09 (t, J=9.2 Hz, 3F), −219.38 (m, 1F).

### Example 38: (1S,3R)-1-(5-((1-(3-Fluoropropyl)azetidin-3-yl)methyl)thiophen-2-yl)-3-methyl-2-(2,2,3,3-tetrafluoropropyl)-2,3,4,9-tetrahydro-1H-pyrido[3,4-b]indole

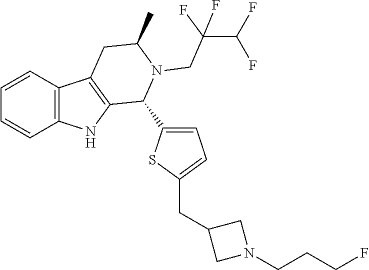 [SMILES: C[C@@H]1Cc2c([nH]c3ccccc23)[C@@H](c2ccc(CC3CN(CCCF)C3)s2)N1CC(F)(F)C(F)F]

Acetic acid (0.07 mL, 1.24 mmol, 3 equiv) was added at room temperature to a solution of 5-((1-(3-fluoropropyl)azetidin-3-yl)methyl)thiophene-2-carbaldehyde (0.1 g, 0.414 mmol, 1 equiv) and (R)—N-(1-(1H-indol-3-yl)propan-2-yl)-2,2,3,3-tetrafluoropropan-1-amine (0.143 g, 0.497 mmol, 1.2 equiv) in toluene (2 mL). After heating at 80° C. for 16 h, additional acetic acid (0.07 mL, 1.24 mmol, 3 equiv) was added and stirred the reaction was heated for additional 24 h at 80° C. The reaction was cooled to room temperature and diluted with ethyl acetate (10 mL) and saturated sodium bicarbonate (10 mL). The layers were separated, and the aqueous layer was extracted with ethyl acetate (3×10 mL). The combined organic layers were washed with saturated brine (10 mL), dried over sodium sulfate, filtered and concentrated under reduced pressure. The residue was purified on an Biotage automated chromatography system (Biotage 28 g KPNH), eluting with a gradient of 0 to 80% ethyl acetate in hexanes to give the title compound (32.5 mg, 15% yield) as a white solid. ¹H NMR (400 MHz, CDCl₃) δ=7.92 (s, 1H), 7.53 (d, J=7.7 Hz, 1H), 7.38-7.32 (m, 1H), 7.24-7.12 (m, 2H), 6.62-6.44 (m, 2H), 6.37-6.04 (m, 1H), 5.17 (s, 1H), 4.54 (t, J=6.0 Hz, 1H), 4.42 (t, J=6.0 Hz, 1H), 3.50-3.39 (m, 3H), 3.32-3.18 (m, 1H), 3.03-2.90 (m, 3H), 2.87 (q, J=6.4 Hz, 2H), 2.78-2.67 (m, 2H), 2.60-2.50 (m, 3H), 1.81-1.66 (m, 2H), 1.20 (d, J=6.8 Hz, 3H); LCMS: m/z=512.2 [M+H]⁺.

### Example 39: (1S,3R)-1-(5-((1-(3-Fluoropropyl)azetidin-3-yl)methyl)thiophen-2-yl)-3-methyl-2-(2,2,3,3,3-pentafluoropropyl)-2,3,4,9-tetrahydro-1H-pyrido[3,4-b]indole

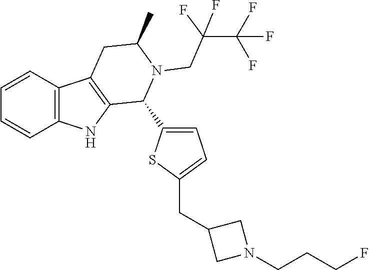 [SMILES: C[C@@H]1Cc2c([nH]c3ccccc23)[C@@H](c2ccc(CC3CN(CCCF)C3)s2)N1CC(F)(F)C(F)(F)F]

Acetic acid (0.07 mL, 1.24 mmol, 3 equiv) was added at room temperature to a solution of 5-((1-(3-fluoropropyl)azetidin-3-yl)methyl)thiophene-2-carbaldehyde (0.1 g, 0.411 mmol, 1 equiv) and compound (R)—N-(1-(1H-indol-3-yl)propan-2-yl)-2,2,3,3,3-pentafluoropropan-1-amine (0.25 g, 0.493 mmol, 1.2 equiv) in toluene (2 mL). After heating at 80° C. for 16 h, additional acetic acid (0.07 mL, 1.23 mmol, 3 equiv) was added and the reaction was heated for additional 24 h at 80° C. The reaction was cooled to room temperature and diluted with ethyl acetate (10 mL) and saturated sodium bicarbonate (10 mL). The layers were separated, and the aqueous layer was extracted with ethyl acetate (3×10 mL). The combined organic layers were washed with saturated brine (10 mL), dried over sodium sulfate, filtered and concentrated under reduced pressure. The residue was purified on an Biotage automated chromatography system (Biotage 28 g KPNH), eluting with a gradient of 0 to 80% ethyl acetate in hexanes to give the title compound (62.3 mg, 28% yield) as a white solid. ¹H NMR (400 MHz, CDCl₃-d) δ=7.93 (s, 1H), 7.53 (d, J=7.7 Hz, 1H), 7.35 (d, J=8.0 Hz, 1H), 7.24-7.12 (m, 2H), 6.64-6.46 (m, 2H), 5.14 (s, 1H), 4.54 (t, J=6.1 Hz, 1H), 4.42 (t, J=6.1 Hz, 1H), 3.52-3.40 (m, 3H), 3.36-3.23 (m, 1H), 3.08-2.95 (m, 3H), 2.86 (q, J=6.4 Hz, 2H), 2.79-2.69 (m, 2H), 2.59-2.48 (m, 3H), 1.80-1.67 (m, 2H), 1.21 (d, J=6.7 Hz, 3H); LCMS: m/z=530.2 [M+H]⁺.

### Example 40: (1S,3R)-2-(2,2-Difluoropropyl)-1-(5-((1-(3-fluoropropyl)azetidin-3-yl)methyl)thiophen-2-yl)-3-methyl-2,3,4,9-tetrahydro-1H-pyrido[3,4-b]indole

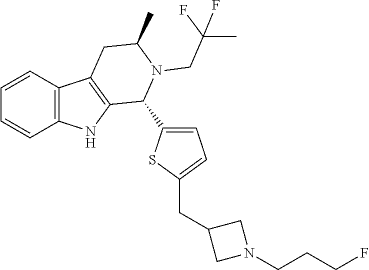 [SMILES: C[C@@H]1Cc2c([nH]c3ccccc23)[C@@H](c2ccc(CC3CN(CCCF)C3)s2)N1CC(C)(F)F]

Acetic acid (0.14 mL, 2.38 mmol, 2.0 equiv) was added at room temperature to a solution of 5-((1-(3-fluoropropyl)azetidin-3-yl)methyl)thiophene-2-carbaldehyde (0.29 g, 1.19 mmol, 1 equiv) and (R)—N-(1-(1H-indol-3-yl)propan-2-yl)-2,2-difluoropropan-1-amine) (0.36 g, 1.43 mmol, 1.2 equiv) in toluene (4 mL). After 16 h, the reaction was cooled to room temperature and diluted with ethyl acetate (20 mL) and saturated sodium bicarbonate (20 mL). The layers were separated, and the aqueous layer was extracted with ethyl acetate (3×20 mL). The combined organic layers were washed with saturated brine (20 mL), dried over sodium sulfate, filtered and concentrated under reduced pressure. The residue was purified on an Biotage automated chromatography system (Biotage 28 g KPNH), eluting with a gradient of 0 to 80% ethyl acetate in hexanes to give the title compound (234 mg, 41% yield) as white solid. ¹H NMR (400 MHz, CDCl₃) δ=8.14 (s, 1H), 7.52 (d, J=7.8 Hz, 1H), 7.32 (d, J=8.1 Hz, 1H), 7.21-7.10 (m, 2H), 6.55 (dd, J=0.9, 3.4 Hz, 1H), 6.50 (d, J=3.5 Hz, 1H), 5.15 (s, 1H), 4.52 (t, J=6.0 Hz, 1H), 4.41 (t, J=6.0 Hz, 1H), 3.50-3.40 (m, 3H), 3.03-2.67 (m, 8H), 2.54 (t, J=7.3 Hz, 3H), 1.81-1.67 (m, 5H), 1.19 (d, J=6.7 Hz, 3H); LCMS: m/z=476.2 [M+H]⁺.

### Example 41: (1S,3R)-1-(5-Bromo-3-methylthiophen-2-yl)-2-(2-fluoro-2-methylpropyl)-3-methyl-2,3,4,9-tetrahydro-1H-pyrido[3,4-b]indole

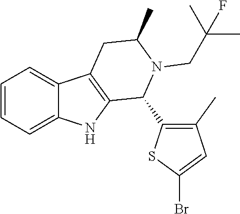 [SMILES: Cc1cc(Br)sc1[C@@H]1c2[nH]c3ccccc3c2C[C@@H](C)N1CC(C)(C)F]

5-Bromo-3-methylthiophene-2-carbaldehyde (0.810 g, 3.96 mmol, 1 equiv) and acetic acid (1.1 mL, 20 mmol, 4.5 equiv) were sequentially added to a solution of (R)—N-(1-(1H-indol-3-yl)propan-2-yl)-2-fluoro-2-methylpropan-1-amine (1.18 g, 4.75 mmol, 1.2 equiv) in toluene (20 mL) in a 40 mL vial. The reaction was heated at 80° C. for 3 days. The reaction was cooled to room temperature then transferred to a separatory funnel with ethyl acetate (30 mL) and washed with saturated sodium carbonate (20 mL). The aqueous layer was extracted with ethyl acetate (2×30 mL). The combined organic layers were washed with saturated brine (60 mL), dried over sodium sulfate, filtered and concentrated under reduced pressure onto silica (13 g). The residue was purified on a Buchi automated chromatography system (100 g, Biotage 20 μm silica gel column), eluting with a gradient of 0 to 5% methyl tert-butyl ether in heptanes. The solid was dried under vacuum at 40° C. for 16 h to give the title compound (1.21 g, 70% yield) as a white foam. LCMS: m/z=435.1 [M+H]⁺.

### Example 42: tert-Butyl 3-((5-((1S,3R)-2-(2-fluoro-2-methylpropyl)-3-methyl-2,3,4,9-tetrahydro-1H-pyrido[3,4-b]indol-1-yl)-4-methylthiophen-2-yl)methyl)azetidine-1-carboxylate

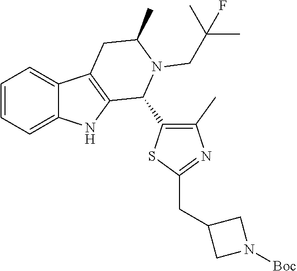 [SMILES: Cc1nc(CC2CN(C(=O)OC(C)(C)C)C2)sc1[C@@H]1c2[nH]c3ccccc3c2C[C@@H](C)N1CC(C)(C)F]

(1S,3R)-1-(5-Bromo-3-methylthiophen-2-yl)-2-(2-fluoro-2-methylpropyl)-3-methyl-2,3,4,9-tetrahydro-1H-pyrido[3,4-b]indole (1.00 g, 2.30 mmol, 1.0 equiv), palladium acetate (52 mg, 0.23 mmol, 10 mol %), RuPhos (0.215 g, 0.460 mmol, 20 mol %), potassium carbonate (0.954 g, 6.90 mmol, 3 equiv), and potassium (1-Boc-azetidin-3-yl)methyltrifluoroborate (0.770 g, 2.78 mmol, 1.2 equiv) were added to a 100 mL round bottom flask. Toluene (15.4 mL) and water (7.6 mL) was added to the reaction flask, which was sparged with nitrogen for 1-2 min. The reaction was heated at 80° C. for 24 h over which time it went from a dark orange solution to a black solution. The reaction was cooled to room temperature then transferred to a separatory funnel with dichloromethane (80 mL) and water (80 mL). The layers were separated, and the aqueous layer was extracted with dichloromethane (2×80 mL). The combined organic extracts were dried over sodium sulfate, filtered and concentrated under reduced pressure onto silica (15 g). The residue was purified on an Interchim automated chromatography system (100 g, Biotage 60 μm silica gel column), eluting with a gradient of 0 to 30% ethyl acetate in heptanes. The solid was dried under vacuum at 40° C. for 8 h to give the title compound (0.95 g, 79% yield) as a yellow/white solid. LCMS: m/z=526.3 [M+H]⁺.

### Example 43: (1S,3R)-1-(5-(Azetidin-3-ylmethyl)-3-methylthiophen-2-yl)-2-(2-fluoro-2-methylpropyl)-3-methyl-2,3,4,9-tetrahydro-1H-pyrido[3,4-b]indole

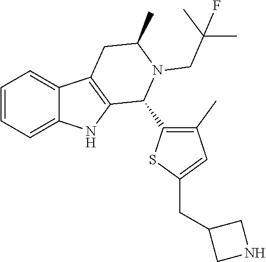 [SMILES: Cc1cc(CC2CNC2)sc1[C@@H]1c2[nH]c3ccccc3c2C[C@@H](C)N1CC(C)(C)F]

Trifluoroacetic acid (3.3 mL, 43 mmol, 24 equiv) was added to tert-butyl 3-((5-((1S,3R)-2-(2-fluoro-2-methylpropyl)-3-methyl-2,3,4,9-tetrahydro-1H-pyrido[3,4-b]indol-1-yl)-4-methylthiophen-2-yl)methyl)azetidine-1-carboxylate (0.93 g, 1.8 mmol, 1 equiv) in dichloromethane (14 mL) in a 40 mL vial. The reaction was stirred at room temperature for 30 min. The reaction mixture was concentrated under reduced pressure then transferred to a separatory funnel with dichloromethane (210 mL). The resulting organic layers were washed with saturated sodium carbonate (2×170 mL). The organic layers were dried over sodium sulfate, filtered and concentrated under reduced pressure to give the title compound (0.765 g, >95% yield) as a red foam, which was used subsequently. LCMS: m/z=426.2 [M+H]⁺.

### Example 44: (1S,3R)-2-(2-Fluoro-2-methylpropyl)-3-methyl-1-(3-methyl-5-((1-propylazetidin-3-yl)methyl)thiophen-2-yl)-2,3,4,9-tetrahydro-1H-pyrido[3,4-b]indole

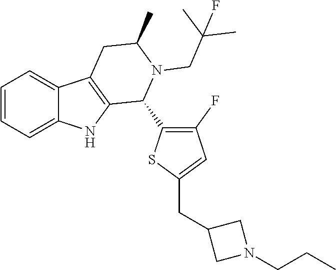 [SMILES: CCCN1CC(Cc2cc(F)c([C@@H]3c4[nH]c5ccccc5c4C[C@@H](C)N3CC(C)(C)F)s2)C1]

1-Bromopropane (0.05 mL, 0.6 mmol, 1 equiv) and diisopropylethylamine (0.51 mL, 2.9 mmol, 5 equiv) were sequentially added to (1S,3R)-1-(5-(azetidin-3-ylmethyl)-3-methylthiophen-2-yl)-2-(2-fluoro-2-methylpropyl)-3-methyl-2,3,4,9-tetrahydro-1H-pyrido[3,4-b]indole (0.250 g, 0.588 mmol, 1 equiv) in N,N-dimethylacetamide (5.9 mL) in a 40 mL vial. After stirring for 2 days at room temperature, the reaction mixture was transferred to a separatory funnel with ethyl acetate (30 mL) and sequentially washed with water (3×30 mL) and saturated brine (30 mL). The organic layer was dried over sodium sulfate, filtered and concentrated under reduced pressure. The residue was purified on a Buchi automated chromatography system (28 g, Biotage 50 μm KP-amino-d column), eluting with a gradient of 0 to 5% ethyl acetate in dichloromethane. The solid was dried under vacuum at 40° C. for 2 h to give the title compound (66 mg, 25% yield) as a white foam. ¹H NMR (400 MHz, CDCl₃) δ=7.68 (br s, 1H), 7.55-7.49 (m, 1H), 7.26-7.22 (m, 1H), 7.17-7.06 (m, 2H), 6.67 (s, 1H), 5.20 (br s, 1H), 3.84-3.63 (m, 1H), 3.53-3.40 (m, 2H), 2.93-2.70 (m, 6H), 2.70-2.51 (m, 3H), 2.43-2.31 (m, 2H), 2.16 (s, 3H), 1.44-1.31 (m, 5H), 1.31-1.25 (m, 3H), 1.10 (d, J=6.7 Hz, 3H), 0.89 (t, J=7.4 Hz, 3H); LCMS: m/z=468.3 [M+H]⁺.

### Example 45: (1S,3R)-2-(2-Fluoro-2-methylpropyl)-1-(5-((1-(3-fluoropropyl)azetidin-3-yl)methyl)-3-methylthiophen-2-yl)-3-methyl-2,3,4,9-tetrahydro-1H-pyrido[3,4-b]indole

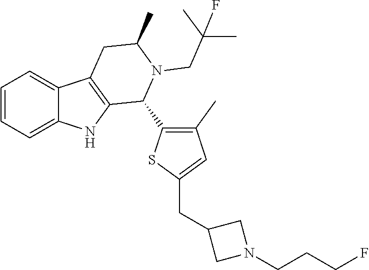 [SMILES: Cc1cc(CC2CN(CCCF)C2)sc1[C@@H]1c2[nH]c3ccccc3c2C[C@@H](C)N1CC(C)(C)F]

1-Bromo-3-fluoropropane (0.05 mL, 0.6 mmol, 1 equiv) and diisopropylethylamine (0.51 mL, 2.9 mmol, 5 equiv) were sequentially added to (1S,3R)-1-(5-(azetidin-3-ylmethyl)-3-methylthiophen-2-yl)-2-(2-fluoro-2-methylpropyl)-3-methyl-2,3,4,9-tetrahydro-1H-pyrido[3,4-b]indole (0.250 g, 0.588 mmol, 1 equiv) in N,N-dimethylacetamide (5.9 mL) in a 40 mL vial. After stirring for 2 days at room temperature, the reaction mixture was transferred to a separatory funnel with ethyl acetate (30 mL) and sequentially washed with water (3×30 mL) and saturated brine (30 mL). The organic layer dried over sodium sulfate, filtered and concentrated under reduced pressure. The residue was purified on a Buchi automated chromatography system (28 g, Biotage 50 μm KP-amino-d column), eluting with a gradient of 0 to 3% ethyl acetate in dichloromethane. The solid was dried under vacuum at 40° C. for 2 h to give the title compound (85 mg, 30% yield) as a white foam. ¹H NMR (400 MHz, CDCl₃) δ=7.62 (br s, 1H), 7.54-7.50 (m, 1H), 7.26-7.23 (m, 1H), 7.17-7.08 (m, 2H), 6.68 (s, 1H), 5.20 (br s, 1H), 4.53 (t, J=6.1 Hz, 1H), 4.42 (t, J=6.0 Hz, 1H), 3.79-3.67 (m, 1H), 3.47 (t, J=6.4 Hz, 2H), 2.93-2.64 (m, 7H), 2.63-2.51 (m, 4H), 2.17 (s, 3H), 1.80-1.66 (m, 2H), 1.37 (d, J=21.5 Hz, 3H), 1.28 (d, J=21.5 Hz, 3H), 1.10 (d, J=6.6 Hz, 3H); LCMS: m/z=486.3 [M+H]⁺.

### Example 46: (1S,3R)-1-(5-Bromo-4-methylthiophen-2-yl)-2-(2-fluoro-2-methylpropyl)-3-methyl-2,3,4,9-tetrahydro-1H-pyrido[3,4-b]indole

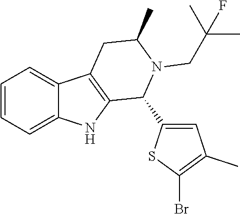 [SMILES: Cc1cc([C@@H]2c3[nH]c4ccccc4c3C[C@@H](C)N2CC(C)(C)F)sc1Br]

A mixture of 1-bromo-2-methylthiophene-5-carbaldehyde (0.9 g, 4.39 mmol, 1.0 equiv), (R)—N-(1-(1H-indol-3-yl)propan-2-yl)-2-fluoro-2-methylpropan-1-amine (1.31 g, 5.27 mmol, 1.2 equiv) and acetic acid (1 mL, 17.56 mmol, 4 equiv) in anhydrous toluene (22 mL) was heated at 80° C. for 19 h. After cooling to room temperature, the reaction was diluted with ethyl acetate (30 mL) and washed with saturated aqueous sodium bicarbonate (30 mL). The organic layer was dried over sodium sulfate, filtered, and concentrated under reduced pressure. The residue was pre-absorbed onto silica gel (5 g) and purified on an Interchim automated chromatography system (Sorbtech 80 g silica-gel column), eluting with a gradient of 0 to 30% ethyl acetate in heptanes to give the title compound (1.66 g, 87% yield) as yellowish foam. LCMS (ESI) m/z=436 [M+H]⁺.

### Example 47: tert-Butyl 3-((5-((1S,3R)-2-(2-fluoro-2-methylpropyl)-3-methyl-2,3,4,9-tetrahydro-1H-pyrido[3,4-b]indol-1-yl)-3-methylthiophen-2-yl)methyl)azetidine-1-carboxylate

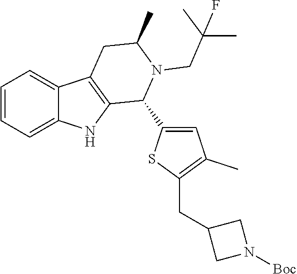 [SMILES: Cc1cc([C@@H]2c3[nH]c4ccccc4c3C[C@@H](C)N2CC(C)(C)F)sc1CC1CN(C(=O)OC(C)(C)C)C1]

A solution of (1S,3R)-1-(5-bromo-4-methylthiophen-2-yl)-2-(2-fluoro-2-methylpropyl)-3-methyl-2,3,4,9-tetrahydro-1H-pyrido[3,4-b]indole (1.63 g, 3.74 mmol, 1.0 equiv) and potassium (1-Boc-azetidin-3-yl)methyltrifluoroborate (1.24 g, 4.49 mmol, 1.2 equiv) in 2 to 1 mixture of toluene and water (36 mL) was sparged with nitrogen for 20 min. Potassium carbonate (1.55 g, 11.2 mmol, 3 equiv), palladium(II) acetate (84 mg, 0.374 mmol, 0.1 equiv) and RuPhos (0.35 g, 0.75 mmol, 0.2 equiv) were added. The reaction was sparged with nitrogen for 5 additional min. After heating at 80° C. for 19 h. The reaction was cooled to room temperature and diluted with dichloromethane (50 mL) and DI water (30 mL). The organic layer was separated, dried over sodium sulfate, filtered, and concentrated under reduced pressure. The residue was pre-absorbed onto silica gel (5 g) and purified on an Interchim automated chromatography system (Sorbtech 80 g silica-gel column), eluting with a gradient of 0 to 30% ethyl acetate in heptanes to give the title compound (1.66 g, 85% yield) as a white solid. LCMS (ESI) m/z=526 [M+H]⁺.

### Example 48: (1S,3R)-1-(5-(Azetidin-3-ylmethyl)-4-methylthiophen-2-yl)-2-(2-fluoro-2-methylpropyl)-3-methyl-2,3,4,9-tetrahydro-1H-pyrido[3,4-b]indole

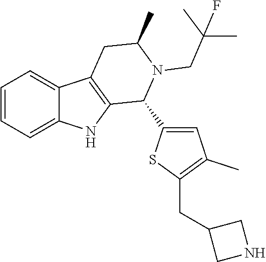 [SMILES: Cc1cc([C@@H]2c3[nH]c4ccccc4c3C[C@@H](C)N2CC(C)(C)F)sc1CC1CNC1]

Trifluoroacetic acid (0.73 mL, 9.52 mmol, 20 equiv) was added over 5 min to a stirred solution of tert-butyl 3-((5-((1S,3R)-2-(2-fluoro-2-methylpropyl)-3-methyl-2,3,4,9-tetrahydro-1H-pyrido[3,4-b]indol-1-yl)-3-methylthiophen-2-yl)methyl)azetidine-1-carboxylate (0.25 g, 0.476 mmol, 1 equiv) in anhydrous dichloromethane (4 mL) at 0° C. under a nitrogen atmosphere. The resulting mixture was stirred at 0° C. for 40 min. The reaction was diluted with cold dichloromethane (50 mL) and cold saturated sodium carbonate (70 mL). The organic layer was separated, dried over sodium sulfate, filtered, concentrated under reduced pressure to give crude title compound (186 mg, 92% yield) as a tan foam, which was used in subsequent syntheses. Crude title compound (25 mg) was purified on an Interchim automated chromatography system (Sorbtech 40 g silica gel column), eluting with a gradient of 0 to 5% methanol in dichloromethane to give the title compound (18 mg, 95.3% yield) as a white solid for assay. LCMS (ESI) m/z=426.2 [M+H]⁺; ¹H NMR (400 MHz, CDCl₃) δ=8.39 (br s, 1H), 7.52 (d, J=7.7 Hz, 1H), 7.33 (d, J=7.9 Hz, 1H), 7.17 (t, J=7.0 Hz, 1H), 7.14-7.09 (m, 1H), 6.41 (s, 1H), 5.23-5.14 (m, 1H), 3.75-3.63 (m, 2H), 3.51-3.36 (m, 3H), 3.01-2.72 (m, 4H), 2.68-2.51 (m, 4H), 2.07-1.97 (m, 3H), 1.58-1.46 (m, 3H), 1.39-1.29 (m, 3H), 1.29-1.18 (m, 1H), 1.18-1.09 (m, 3H).

### Example 49: (1S,3R)-2-(2-Fluoro-2-methylpropyl)-3-methyl-1-(4-methyl-5-((1-propylazetidin-3-yl)methyl)thiophen-2-yl)-2,3,4,9-tetrahydro-1H-pyrido[3,4-b]indole

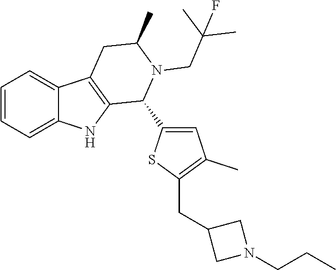 [SMILES: CCCN1CC(Cc2sc([C@@H]3c4[nH]c5ccccc5c4C[C@@H](C)N3CC(C)(C)F)cc2C)C1]

A solution of crude (1S,3R)-1-(5-(azetidin-3-ylmethyl)-4-methylthiophen-2-yl)-2-(2-fluoro-2-methylpropyl)-3-methyl-2,3,4,9-tetrahydro-1H-pyrido[3,4-b]indole (300 mg, 0.71 mmol, 1 equiv) in an anhydrous N,N-dimethylacetamide (5 mL) was sequentially treated with 1-bromopropane (35 uL, 0.385 mmol, 0.54 equiv) and NN-diisopropyl-N-ethylamine (0.5 mL, 2.87 mmol, 4 equiv) and stirred at room temperature for 72 h. The reaction was diluted with ethyl acetate (30 mL) and washed with saturated brine (50 mL). The organic layer was dried over sodium sulfate, filtered, and concentrated under reduced pressure. The residue was absorbed on Celite® (1 g) and purified on an Interchim automated chromatography system (KP-Amino Biotage 24 g silica gel column), eluting with a gradient of 0 to 100% dichloromethane in heptanes to give the title compound (17 mg, 5% yield) as yellow solid after lyophilization. LCMS (ESI) m/z=468 [M+H]⁺; ¹H NMR (400 MHz, CDCl₃) δ=8.12 (br s, 1H), 7.52 (d, J=7.7 Hz, 1H), 7.39-7.30 (m, 1H), 7.18 (t, J=7.1 Hz, 1H), 7.15-7.04 (m, 1H), 6.42 (s, 1H), 5.18 (br s, 1H), 3.54 (br t, J=7.2 Hz, 2H), 3.44 (br s, 1H), 3.03-2.75 (m, 5H), 2.72-2.61 (m, 2H), 2.61-2.41 (m, 4H), 2.08-1.95 (m, 3H), 1.57-1.46 (m, 3H), 1.46-1.38 (m, 2H), 1.37-1.30 (m, 3H), 1.15 (d, J=6.7 Hz, 3H), 0.90 (t, J=7.4 Hz, 3H).

### Example 50: (1S,3R)-2-(2-Fluoro-2-methylpropyl)-1-(5-((1-(3-fluoropropyl)azetidin-3-yl)methyl)-4-methylthiophen-2-yl)-3-methyl-2,3,4,9-tetrahydro-1H-pyrido[3,4-b]indole

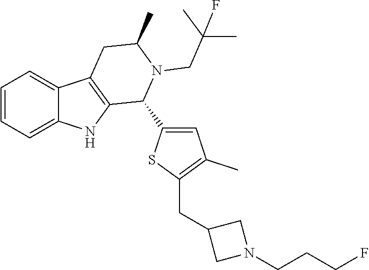 [SMILES: Cc1cc([C@@H]2c3[nH]c4ccccc4c3C[C@@H](C)N2CC(C)(C)F)sc1CC1CN(CCCF)C1]

1-Bromo-3-fluoropropane (35 uL, 0.379 mmol, 1.0 equiv) and N,N-diisopropyl-N-ethylamine (0.33 mL, 1.89 mmol, 5 equiv) were sequentially added to a solution of crude (1S,3R)-1-(5-(azetidin-3-ylmethyl)-4-methylthiophen-2-yl)-2-(2-fluoro-2-methylpropyl)-3-methyl-2,3,4,9-tetrahydro-1H-pyrido[3,4-b]indole (161 mg, 0.379 mmol, 1 equiv, 80% purity) in an anhydrous N,N-dimethylacetamide (5 mL) at room temperature. After stirring for 18 h, additional 1-bromo-3-fluoropropane (7 μL, 0.076 mmol, 0.2 equiv) was added and the reaction was stirred for 72 h. The reaction was diluted with ethyl acetate (30 mL) and washed with saturated brine (30 mL). The organic layer was dried over sodium sulfate, filtered, and concentrated under reduced pressure. The residue was absorbed on Celite® (2 g) and purified on an Interchim automated chromatography system (KP-Amino Biotage 24 g silicagel column), eluting with a gradient of 0 to 100% dichloromethane in heptanes to give the title compound (46 mg, 24% yield) as yellowish solid. LCMS (ESI) m/z=486.3 [M+H]⁺; ¹H NMR (400 MHz, CDCl₃) δ=8.07 (br s, 1H), 7.59-7.47 (m, 1H), 7.39-7.28 (m, 1H), 7.24-7.07 (m, 2H), 6.41 (s, 1H), 5.30-5.07 (m, 1H), 4.53 (t, J=6.0 Hz, 1H), 4.41 (t, J=6.0 Hz, 1H), 3.43 (br t, J=7.1 Hz, 3H), 2.96-2.79 (m, 4H), 2.77-2.49 (m, 7H), 2.09-1.97 (m, 3H), 1.86-1.59 (m, 3H), 1.58-1.46 (m, 3H), 1.45 (s, 1H), 1.37-1.30 (m, 3H), 1.28 (br s, 2H), 1.19-1.09 (m, 3H), 1.07-0.76 (m, 2H).

### Example 51: 5-Bromo-3-fluorothiophen-2-yl)methanol

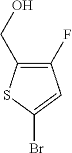

Methyl 5-bromo-3-fluorothiophene-2-carboxylate (1.0 g, 4.2 mmol, 1 equiv) was contacted with lithium borohydride (0.27 g, 8.4 mmol, 2 equiv) in diethyl ether (20 mL) at room temperature for 28 h. The reaction mixture was poured into saturated ammonium chloride (40 mL) and stirred for 90 min. The mixture was extracted with methyl t-butyl ether (3×20 mL). The combined organic layers were dried over magnesium sulfate (5 g), filtered and concentrated under reduced pressure to give the title compound (0.85 g, 96% yield) as a light-yellow oil. GCMS: m/z=210, 212 [M].

### Example 52: 5-Bromo-3-fluorothiophene-2-carbaldehyde

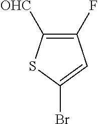

Manganese dioxide (8.7 g, 100 mmol, 25 equiv) was added to a solution of 5-bromo-3-fluorothiophen-2-yl)methanol (0.85 g, 4.0 mmol, 1 equiv) in dioxane (110 mL) at room temperature. After stirring for 5 h, GCMS analysis of a filtered aliquot indicated the reaction was complete. The reaction mixture was filtered through Celite® (20 g) and the filter pad was washed with dioxane (3×25 mL). The filtrate was concentrated under reduced pressure to give an oil. The oil was dissolved in dichloromethane (10 mL), absorbed onto silica gel (3 g) and concentrated under reduced pressure. Purification on a Biotage automated system (Biotage silica gel column 25 g, 20 μm), eluting with a gradient of 0 to 20% methyl t-butyl ether in heptanes, gave the title compound (0.46 g, 55% yield) as a pale-yellow oil. GCMS: m/z=208, 210 [M].

### Example 53: (1S,3R)-1-(S-Bromo-3-fluorothiophen-2-yl)-2-(2-fluoro-2-methylpropyl)-3-methyl-2,3,4,9-tetrahydro-1H-pyrido[3,4-b]indole

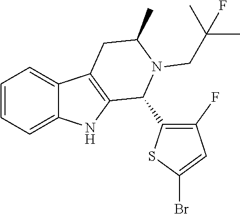 [SMILES: C[C@@H]1Cc2c([nH]c3ccccc23)[C@@H](c2sc(Br)cc2F)N1CC(C)(C)F]

A mixture of 5-bromo-3-fluorothiophene-2-carbaldehyde (0.46 g, 2.2 mmol, 1.0 equiv), (R)—N-(1-(1H-indol-3-yl)propan-2-yl)-2-fluoro-2-methylpropan-1-amine (0.66 g, 2.6 mmol, 1.2 equiv) and acetic acid 0.25 mL, 4.4 mmol, 2.0 equiv) in toluene (11 mL) was stirred at 80° C. for 21 h. The reaction mixture was cooled to room temperature, diluted with ethyl acetate (50 mL), washed with a saturated sodium carbonate solution (3×15 mL), dried over magnesium sulfate (10 g), filtered and concentrated under reduced pressure to give a red oil. This oil was absorbed onto silica gel (10 g) with dichloromethane (30 mL). Purification on a Biotage automated system (Biotage silica gel column (25 g, 20 μm), eluting with a gradient of 0 to 20% methyl t-butyl ether in heptanes, gave the title compound (0.5 g, 52% yield) as a pale-yellow foam. LCMS: m/z=439, 441 [M+H]⁺.

### Example 54: tert-Butyl 3-((4-fluoro-5-((1S,3R)-2-(2-fluoro-2-methylpropyl)-3-methyl-2,3,4,9-tetrahydro-1H-pyrido[3,4-b]indol-1-yl)thiophen-2-yl)methyl)azetidine-1-carboxylate

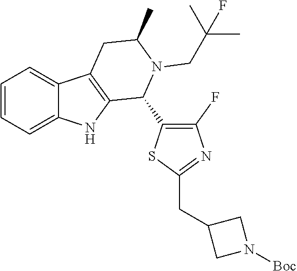 [SMILES: C[C@@H]1Cc2c([nH]c3ccccc23)[C@@H](c2sc(CC3CN(C(=O)OC(C)(C)C)C3)nc2F)N1CC(C)(C)F]

A mixture of (1S,3R)-1-(5-bromo-3-fluorothiophen-2-yl)-2-(2-fluoro-2-methylpropyl)-3-methyl-2,3,4,9-tetrahydro-1H-pyrido[3,4-b]indole (0.5 g, 1.1 mmol, 1.0 equiv), potassium (1-Boc-azetidin-3-yl)methyltrifluoroborate (0.38 g, 1.3 mmol, 1.2 equiv) in toluene (9 mL) and water (4.5 mL) was sparged with argon for 10 min. Potassium carbonate (0.47 g, 3.4 mmol, 3.0 equiv), XPhos (102 mg, 0.23 mmol, 0.2 equiv) and palladium acetate (26 mg, 0.12 mmol, 0.1 equiv) were added sequentially and the mixture was sparged with argon for 1 minute. The reaction mixture was heated in a sealed vial at 80° C. for 23 h. The reaction mixture was cooled to room temperature, diluted with water (9 mL) and extracted with methyl t-butyl ether (3×10 mL). The combined organic layers were dried over magnesium sulfate (5 g), filtered and concentrated under reduced pressure to give a black foam. This foam was absorbed onto silica gel (4 g) with dichloromethane (5 mL). Purification on a Biotage automated system (Biotage silica gel column (25 g, 20 μm)), eluting with a gradient of 0 to 50% methyl t-butyl ether-heptane, gave the title compound (0.28 g, 47% yield) as a tan foam. LCMS: m/z=530 [M+H]⁺.

### Example 55: (1S,3R)-1-(5-(Azetidin-3-ylmethyl)-3-fluorothiophen-2-yl)-2-(2-fluoro-2-methylpropyl)-3-methyl-2,3,4,9-tetrahydro-1H-pyrido[3,4-b]indole

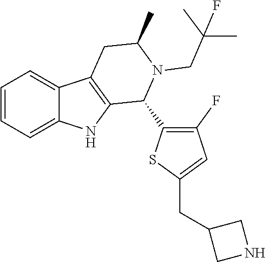 [SMILES: C[C@@H]1Cc2c([nH]c3ccccc23)[C@@H](c2sc(CC3CNC3)cc2F)N1CC(C)(C)F]

A solution of compound tert-butyl 3-((4-fluoro-5-((1S,3R)-2-(2-fluoro-2-methylpropyl)-3-methyl-2,3,4,9-tetrahydro-1H-pyrido[3,4-b]indol-1-yl)thiophen-2-yl)methyl)azetidine-1-carboxylate (254 mg, 0.48 mmol, 1.0 equiv) in dichloromethane (4 mL) was cooled to 0-5° C. under nitrogen. Trifluoroacetic acid (1 mL, 12.4 mol, 26 equiv) was added over 10 min. The deep purple reaction mixture was warmed to room temperature over 40 min, diluted with dichloromethane (36 mL), then cooled to 0-5° C. again. Cold saturated sodium carbonate (10 mL, 0-5° C.) was added over 10 min. The mixture was warmed to room temperature over 10 min; the layers were separated, and the organic layer was washed with saturated sodium carbonate (1×10 mL). The organic layer was dried over magnesium sulfate, filtered and concentrated under reduced pressure to give the title compound (180 mg, 81% yield) as a light-brown oil. LCMS: m/z=430 [M+H]⁺.

### Example 56: (1S,3R)-2-(2-Fluoro-2-methylpropyl)-1-(3-fluoro-5-((1-propylazetidin-3-yl)methyl)thiophen-2-yl)-3-methyl-2,3,4,9-tetrahydro-1-pyrido[3,4-b]indole

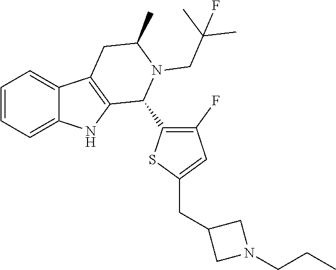 [SMILES: CCCN1CC(Cc2cc(F)c([C@@H]3c4[nH]c5ccccc5c4C[C@@H](C)N3CC(C)(C)F)s2)C1]

A solution of (1S,3R)-1-(5-(azetidin-3-ylmethyl)-3-fluorothiophen-2-yl)-2-(2-fluoro-2-methylpropyl)-3-methyl-2,3,4,9-tetrahydro-1H-pyrido[3,4-b]indole (90 mg, 0.21 mmol, 1.0 equiv) and 1-bromopropane (29 mg, 0.21 mmol, 1.0 equiv) in N,N-dimethylacetamide (2 mL) was stirred under nitrogen. Diisopropylethylamine (135 mg, 1.05 mmol, 5.0 equiv) was added and the reaction mixture was stirred for 37 h. The brown solution was diluted with ethyl acetate (10 mL), washed with water (4×5 mL) and saturated brine (1×5 mL), dried over magnesium sulfate (5 g), filtered and concentrated under reduced pressure to give a brown oil. Purification on a Biotage automated system (Biotage Amino Duo column 11 g, 50 μm), eluting with a gradient of 0 to 100% ethyl acetate in heptanes gave the title compound (17 mg, 17% yield) as a white foam.

¹H NMR (400 MHz, CDCl₃) δ=7.97 (br s, 1H), 7.51 (d, J=8, 1H), 7.34-7.28 (m, 1H), 7.17 (dt, J=1, 8, 1H), 7.13-7.08 (m, 1H), 6.44 (s, 1H), 5.27 (s, 1H), 3.69-3.60 (m, 1H), 3.39 (t, J=8, 2H), 2.88 (d, J=8, 2H), 2.83-2.61 (m, 5H), 2.59-2.53 (m, 2H), 2.39-2.31 (m, 2H), 2.01 (s, 2H), 1.48-1.39 (m, 3H), 1.37-1.29 (m, 5H), 1.19 (d, J=7, 3H), 0.88 (t, J=7, 3H); LCMS: m/z=472.2 [M+H]⁺.

### Example 57: (1S,3R)-2-(2-Fluoro-2-methylpropyl)-1-(3-fluoro-5-((1-(3-fluoropropyl)-azetidin-3-yl)methyl)thiophen-2-yl)-3-methyl-2,3,4,9-tetrahydro-1H-pyrido[3,4-b]indole

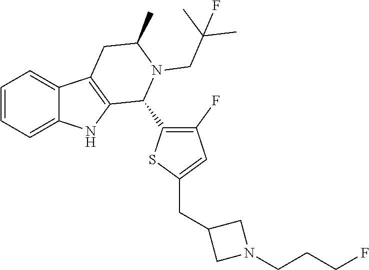 [SMILES: C[C@@H]1Cc2c([nH]c3ccccc23)[C@@H](c2sc(CC3CN(CCCF)C3)cc2F)N1CC(C)(C)F]

A solution of (1S,3R)-1-(5-(azetidin-3-ylmethyl)-3-fluorothiophen-2-yl)-2-(2-fluoro-2-methylpropyl)-3-methyl-2,3,4,9-tetrahydro-1H-pyrido[3,4-b]indole (90 mg, 0.21 mmol, 1.0 equiv) and 1-bromo-3-fluoropropane (30 mg, 0.21 mmol, 1.0 equiv) in N,N-dimethylacetamide (2 mL) was stirred under nitrogen. Diisopropylethylamine (135 mg, 1.05 mmol, 5.0 equiv) was added and the reaction mixture was stirred for 37 h. The brown solution was diluted with ethyl acetate (10 mL), washed with water (4×5 mL), then brine (1×5 mL), dried over magnesium sulfate (5 g), filtered and concentrated under reduced pressure to give a brown oil. Purification on a Biotage automated system (Biotage Amino Duo column 11 g, 50 μm), eluting with a gradient of 0 to 100% ethyl acetate in heptanes, gave the title compound (24 mg, 11% yield) as a yellow foam.

¹H NMR (400 MHz, CDCl₃) δ=7.91 (br s, 1H), 7.51 (d, J=8, 1H), 7.32 (d, J=8, 1H), 7.17 (dt, J=1, 8, 1H), 7.13-7.08 (m, 1H), 6.45 (s, 1H), 5.28 (s, 1H), 4.53 (t, J=6, 1H), 4.41 (t, J=6, 1H), 3.69-3.60 (m, 1H), 3.40 (t, J=8, 2H), 2.89 (d, J=8, 2H), 2.85-2.63 (m, 5H), 2.62-2.47 (m, 4H), 2.01 (s, 2H), 1.79-1.65 (in, 2H), 1.48-1.39 (m 3H), 1.36-1.29 (m, 3H), 1.19 (d, J=7, 3H); LCMS: m/z=490.3 [M+H]⁺.

### Example 58: (1S,3R)-1-(5-Bromo-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole

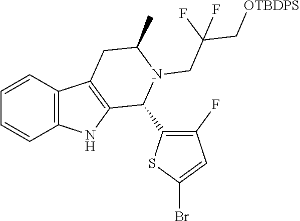

A solution of 5-bromo-3-fluorothiophene-2-carbaldehyde (1.0 g, 4.7 mmol, 1.0 equiv), (R)—N-(1-(1H-indol-3-yl)propan-2-yl)-3-((tert-butyldiphenylsilyl)oxy)-2,2-difluoropropan-1-amine (2.9 g, 5.7 mmol, 1.2 equiv) and acetic acid (1.3 mL, 23.7 mmol, 5.0 equiv) in toluene (25 mL) was heated at 80° C. under nitrogen for 20 hours. After cooling to room temperature, the reaction mixture was diluted with ethyl acetate (25 mL), washed with a saturated sodium carbonate (3×20 mL), dried over magnesium sulfate (5 g), filtered and concentrated under reduced pressure to give a red oil. The crude product was absorbed onto silica gel (40 g) with dichloromethane (100 mL) and then concentrated under reduced pressure. The material was purified on a Biotage automated chromatography system (200 g, 20 μm column), eluting with a gradient of 0 to 100% methyl t-butyl ether in heptanes, to give the title compound (2.9 g, 65% yield) as a yellow solid. LCMS: m/z=697, 699 [M+H]⁺.

### Example 59: tert-Butyl 3-((5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-4-fluorothiophen-2-yl)methyl)azetidine-1-carboxylate

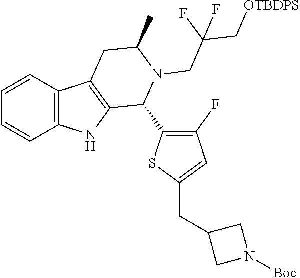

A mixture of (1S,3R)-1-(5-bromo-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole (0.7 g, 1.0 mmol, 1.0 equiv), tert-butyl 3-((trifluoro-14-boraneyl)methyl)azetidine-1-carboxylate, potassium salt (0.33 g, 1.2 mmol, 1.2 equiv), toluene (8 mL) and water (4 mL) was sparged with nitrogen for 30 minutes. Potassium carbonate (0.4 g, 3 mmol, 3.0 equiv), RuPhos (80 mg, 0.2 mmol, 0.2 equiv) and palladium acetate (22 mg, 0.1 mmol, 0.1 equiv) were added sequentially, then the reaction mixture was sparged with nitrogen for an additional 15 minutes. The reaction mixture was stirred at 85° C. under nitrogen for 22 hours, then cooled to room temperature. The reaction mixture was diluted with methyl t-butyl ether (40 mL) and washed with water (3×10 mL). The organic layer was dried over sodium sulfate (5 g), filtered and concentrated under reduced pressure to give a dark brown oil. The crude product was absorbed onto silica gel (30 g) with dichloromethane (50 mL) and then concentrated under reduced pressure. The material was purified on a Biotage automated chromatography system (50 g, 20 μm column), eluting with a gradient of 0 to 50% methyl t-butyl ether in heptanes, to give the title compound (0.49 g, 64% yield) as a tan oil. LCMS: m/z=789 [M+H]⁺.

### Example 60: (1S,3R)-1-(5-(Azetidin-3-ylmethyl)-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole

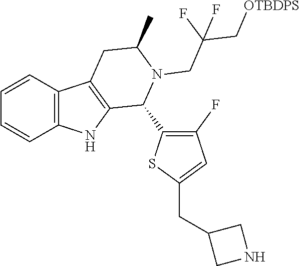

A solution of tert-butyl 3-((5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-4-fluorothiophen-2-yl)methyl)azetidine-1-carboxylate (490 mg, 0.6 mmol, 1.0 equiv), trifluoroacetic acid (1.2 mL, 15.6 mmol, 25 equiv) and dichloromethane (5 mL) was stirred at 0 to 5° C. for 1.5 hours, at which time thin layer chromatography and LCMS analysis indicated most of the starting material was consumed. The reaction mixture was added to a stirred saturated sodium carbonate solution (20 mL) such that the internal temperature did not exceed 5° C. The aqueous mixture (pH=10 by indicator paper) was extracted with dichloromethane (3×10 mL). The combined organic layers were dried over sodium sulfate, filtered and concentrated under reduced pressure to give the title compound (257 mg, 60% yield) as a tan foam, which was used subsequently. LCMS: m/z=689 [M+H]⁺.

### Example 61: (1S,3R)-2-(3-((tert-Butyldiphenylsilyl)oxy)-2,2-difluoropropyl)-1-(3-fluoro-5-((1-(3-fluoropropyl)azetidin-3-yl)methyl)thiophen-2-yl)-3-methyl-2,3,4,9-tetrahydro-1H-pyrido[3,4-b]indole

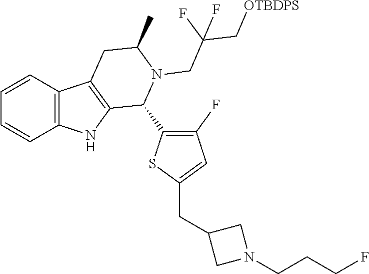

N,N-Diisopropylethylamine (0.24 g, 032 mL, 1.85 mmol, 5.0 equiv) was added to a solution of (1S,3R)-1-(5-(azetidin-3-ylmethyl)-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole (257 mg, 0.37 mmol, 1.0 equiv) and 1-bromo-3-fluoropropane (78 mg, 0.56 mmol, 1.5 equiv) in N,N-dimethylacetamide (3 mL) and the reaction mixture was stirred for 18 hours. The reaction mixture was diluted with ethyl acetate (10 mL), washed with water (4×5 mL), dried over magnesium sulfate (1 g), filtered and concentrated under reduced pressure. The crude product was absorbed onto Celite® (1 g) with dichloromethane (1 mL) and solvent was removed under reduced pressure. The material was purified on a Biotage automated chromatography system (Amino Duo column, 28 g, 50 pin), eluting with a gradient of 0 to 50% ethyl acetate in hexanes, to give the title compound (30 mg, 10% yield) as pale-yellow oil. LCMS: m/z=748 [M+H]⁺.

### Example 62: 2,2-Difluoro-3-((1S,3R)-1-(3-fluoro-5-((1-(3-fluoropropyl)azetidin-3-yl)methyl)thiophen-2-yl)-3-methyl-1,3,4,9-tetrahydro-2H-pyrido[3,4-b]indol-2-yl)propan-1-ol

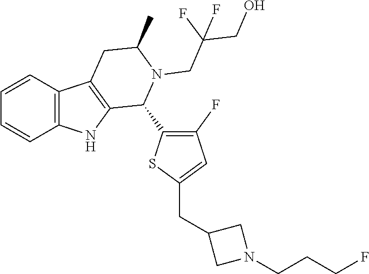 [SMILES: C[C@@H]1Cc2c([nH]c3ccccc23)[C@@H](c2sc(CC3CN(CCCF)C3)cc2F)N1CC(F)(F)CO]

1 M tetrabutylammonium fluoride in tetrahydrofuran (44 μL, 0.044 mmol, 1.1 equiv) was added to a solution of (1S,3R)-2-(3-((tert-butyldiphenylsilyl)oxy)-2,2-difluoropropyl)-1-(3-fluoro-5-((1-(3-fluoropropyl)azetidin-3-yl)methyl)thiophen-2-yl)-3-methyl-2,3,4,9-tetrahydro-1H-pyrido[3,4-b]indole (30 mg, 0.04 mmol, 1.0 equiv) in anhydrous tetrahydrofuran (0.3 mL). The reaction mixture was stirred for 1.3 hours, at which time thin layer chromatography and LCMS analysis indicated that the starting material was mostly consumed. The reaction was concentrated under reduced pressure. The residue was stirred with saturated ammonium chloride (2 mL) for 15 minutes, then extracted with ethyl acetate (3×1 mL). The combined organic layers were washed with a saturated ammonium chloride (3×1 mL), dried over magnesium sulfate, filtered and concentrated under reduced pressure. The material was purified on a Biotage automated chromatography system (Amino Duo column, 5 g, 50 m), eluting with a gradient of 0 to 100% ethyl acetate in hexanes to give the title compound (11 mg, 55% yield) as white solid after lyophilization. ¹H NMR (400 MHz, CDCl₃) δ=7.74 (s, 1H), 7.51 (d, J=7.7 Hz, 1H), 7.32-7.27 (m, 1H), 7.18 (dt, J=1.3, 7.5 Hz, 1H), 7.14-7.09 (m, 1H), 6.52-6.49 (m, 1H), 5.28 (s, 1H), 4.54-4.48 (m, 1H), 4.42-4.36 (m, 1H), 3.93-3.79 (m, 2H), 3.75-3.63 (m, 1H), 3.37 (t, J=7.5 Hz, 2H), 3.29-3.08 (m, 1H), 3.00-2.80 (m, 6H), 2.70-2.54 (m, 2H), 2.54-2.47 (m, 2H), 1.80-1.63 (m, 3H), 1.31-1.26 (m, 1H), 1.24 (d, J=6.6 Hz, 3H); LCMS: m/z=510.2 [M+H]⁺.

### Example 63: 5-Bromo-4-fluorothiophen-2-yl)methanol

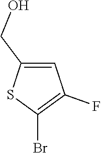

Methyl 5-bromo-4-fluorothiophene-2-carboxylate (0.8 g, 3.4 mmol, 1 equiv) was reacted with lithium borohydride (0.21 g, 6.7 mmol, 2 equiv) in diethyl ether (16 mL) at room temperature for 24 h. The reaction mixture was poured into saturated ammonium chloride (40 mL) and stirred for 90 min. The mixture was extracted with methyl t-butyl ether (3×20 mL). The combined organic layers were dried over magnesium sulfate (5 g), filtered and concentrated under reduced pressure to give the title compound (0.58 g, 83% yield) as a light-yellow oil. GCMS: m/z=210, 212 [M].

### Example 64: S-Bromo-4-fluorothiophene-2-carbaldehyde

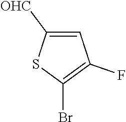

Manganese dioxide (4.8 g, 55 mmol, 27 equiv) was added to a solution of 5-bromo-4-fluorothiophen-2-yl)methanol (0.58 g, 2.8 mmol, 1 equiv) in dioxane (60 mL) at room temperature. After stirring for 5 h, GCMS analysis of a filtered aliquot indicated the reaction was complete. The reaction mixture was filtered through Celite® (10 g), and the filter pad was washed with dioxane (3×10 mL). The filtrate was concentrated under reduced pressure to give an oil. The oil was dissolved in dichloromethane (10 mL) and absorbed onto silica gel (3 g). Purification on a Biotage automated system (Biotage silica gel column 5 g, 20 μm), eluting with a gradient of 0 to 20% methyl t-butyl ether in heptanes, gave the title compound (0.36 g, 67% yield) as a pale-yellow oil. GCMS: m/z=208, 210 [M].

### Example 65: (1S,3R)-1-(5-Bromo-4-fluorothiophen-2-yl)-2-(2-fluoro-2-methylpropyl)-3-methyl-2,3,4,9-tetrahydro-1H-pyrido[3,4-b]indole

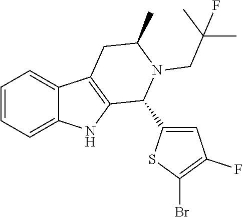 [SMILES: C[C@@H]1Cc2c([nH]c3ccccc23)[C@@H](c2cc(F)c(Br)s2)N1CC(C)(C)F]

A mixture of 5-bromo-4-fluorothiophene-2-carbaldehyde (0.36 g, 2.2 mmol, 1.0 equiv), (R)—N-(1-(1H-indol-3-yl)propan-2-yl)-2-fluoro-2-methylpropan-1-amine (0.51 g, 2.1 mmol, 1.2 equiv) and acetic acid (0.19 mL, 3.4 mmol, 2.0 equiv) in toluene (9 mL) was stirred at 80° C. for 24 h. The reaction mixture was cooled to room temperature, diluted with ethyl acetate (40 mL), washed with a saturated sodium carbonate solution (3×20 mL), dried over magnesium sulfate (10 g), filtered and concentrated under reduced pressure to give a red oil. This oil was absorbed onto silica gel (10 g) with dichloromethane (30 mL). Purification on a Biotage automated system (Biotage silica gel column 25 g, 20 μm), eluting with a gradient of 0 to 20% methyl t-butyl ether in heptanes gave the title compound (0.6 g, 80% yield) as a white foam. LCMS: m/z=439, 441 [M+H]⁻.

### Example 66: tert-Butyl 3-((3-fluoro-5-((1S,3R)-2-(2-fluoro-2-methylpropyl)-3-methyl-2,3,4,9-tetrahydro-1H-pyrido[3,4-b]indol-1-yl)thiophen-2-yl)methyl)azetidine-1-carboxylate

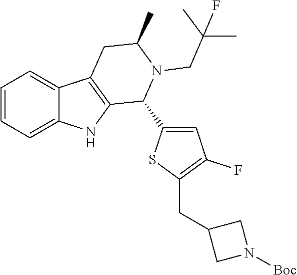 [SMILES: C[C@@H]1Cc2c([nH]c3ccccc23)[C@@H](c2cc(F)c(CC3CN(C(=O)OC(C)(C)C)C3)s2)N1CC(C)(C)F]

A mixture of (1S,3R)-1-(5-bromo-4-fluorothiophen-2-yl)-2-(2-fluoro-2-methylpropyl)-3-methyl-2,3,4,9-tetrahydro-1H-pyrido[3,4-b]indole (0.45 g, 1.0 mmol, 1.0 equiv) and potassium (1-Boc-azetidin-3-yl)methyltrifluoroborate (0.34 g, 1.3 mmol, 1.2 equiv) in toluene (7 mL) and water (3.5 mL) was sparged with argon for 10 min. Potassium carbonate (0.42 g, 3.1 mmol, 3.0 equiv), XPhos (97 mg, 0.20 mmol, 0.2 equiv) and palladium acetate (23 mg, 0.10 mmol, 0.1 equiv) were added sequentially and the mixture was sparged with argon for 1 minute. The reaction mixture was heated in a sealed vial at 80° C. for 23 h. The reaction mixture was cooled to room temperature, diluted with water (9 mL) and extracted with methyl t-butyl ether (3×10 mL). The combined organic layers were dried over magnesium sulfate (5 g), filtered and concentrated under reduced pressure to give a black foam. This foam was absorbed onto silica gel (4 g) with dichloromethane (5 mL). Purification on a Biotage automated system using a Biotage silica gel column (25 g, 20 μm) and eluting with a gradient of 0 to 50% methyl t-butyl ether in heptanes gave the title compound (0.28 g, 51% yield) as a tan foam. LCMS: m/z=530 [M+H]⁺.

### Example 67: (1S,3R)-1-(5-(Azetidin-3-ylmethyl)-4-fluorothiophen-2-yl)-2-(2-fluoro-2-methylpropyl)-3-methyl-2,3,4,9-tetrahydro-1H-pyrido[3,4-b]indole

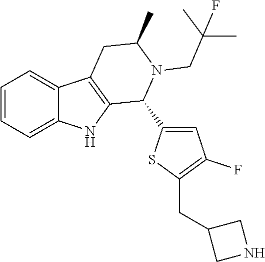 [SMILES: C[C@@H]1Cc2c([nH]c3ccccc23)[C@@H](c2cc(F)c(CC3CNC3)s2)N1CC(C)(C)F]

A solution of compound tert-butyl 3-((3-fluoro-5-((1S,3R)-2-(2-fluoro-2-methylpropyl)-3-methyl-2,3,4,9-tetrahydro-1H-pyrido[3,4-b]indol-1-yl)thiophen-2-yl)methyl)azetidine-1-carboxylate (280 mg, 0.52 mmol, 1.0 equiv) in dichloromethane (4 mL) was cooled to 0-5° C. under nitrogen. Trifluoroacetic acid (1 mL, 12.4 mol, 24 equiv) was added over 10 min. The deep purple reaction mixture was warmed to room temperature over 40 min, diluted with dichloromethane (36 mL), then cooled to 0-5° C. again. A cold saturated sodium carbonate solution (10 mL, 0-5° C.) was added over 10 min. The mixture was warmed to room temperature over 10 min; the layers were separated, and the organic layer was washed with saturated sodium carbonate solution (1×10 mL). The organic layer was dried over magnesium sulfate, filtered and concentrated under reduced pressure to give the title compound (180 mg, 81% yield) as a light brown oil which was used subsequently. LCMS: m/z=430 [M+H]⁺.

### Example 68: (1S,3R)-2-(2-Fluoro-2-methylpropyl)-1-(4-fluoro-5-((1-propylazetidin-3-yl)methyl)thiophen-2-yl)-3-methyl-2,3,4,9-tetrahydro-1H-pyrido[3,4-b]indole

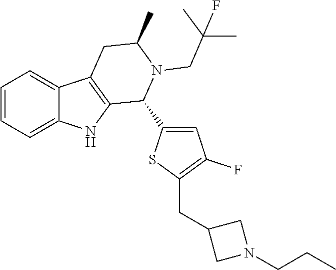 [SMILES: CCCN1CC(Cc2sc([C@@H]3c4[nH]c5ccccc5c4C[C@@H](C)N3CC(C)(C)F)cc2F)C1]

Diisopropylethylamine (0.2 mL, 1.05 mmol, 5.0 equiv) was added to a solution of (1S,3R)-1-(5-(azetidin-3-ylmethyl)-4-fluorothiophen-2-yl)-2-(2-fluoro-2-methylpropyl)-3-methyl-2,3,4,9-tetrahydro-1H-pyrido[3,4-b]indole (90 mg, 0.21 mmol, 1.0 equiv) and 1-bromopropane (29 mg, 0.21 mmol, 1.0 equiv) in N,N-dimethyl acetamide (2 mL) at room temperature under nitrogen. After stirring for 39 h, the reaction was diluted with ethyl acetate (20 mL), washed with water (4×10 mL) and saturated brine (10 mL), dried over magnesium sulfate, filtered and concentrated under reduced pressure to give a brown oil. Purification on a Biotage automated system (Biotage Amino Duo column (1 g, 50 μm), eluting with a gradient of 0 to 100% ethyl acetate in heptanes, gave the title compound (17 mg, 17% yield) as a white foam. ¹H NMR (400 MHz, CDCl₃) δ=8.07 (br s, 1H), 7.52 (d, J=8, 1H), 7.34 (d, J=8, 1H), 7.19 (dt, J=1, 8, 1H), 7.14-7.09 (m, 1H), 6.45 (s, 1H), 5.15 (br d, J=2, 1H), 3.46-3.35 (m, 3H), 3.22 (s, 1H), 2.95-2.79 (m, 4H), 2.75-2.48 (m, 5H), 2.42-2.29 (m, 2H), 1.56-1.48 (m, 3H), 1.41-1.26 (m, 6H), 1.19 (s, 2H), 1.17 (d, J=7, 3H), 0.88 (t, J=7, 3H); LCMS: m/z=472.3 [M+H]⁺.

### Example 69: (1S,3R)-2-(2-Fluoro-2-methylpropyl)-1-(4-fluoro-5-((1-(3-fluoropropyl) azetidin-3-yl)methyl)thiophen-2-yl)-3-methyl-2,3,4,9-tetrahydro-1H-pyrido[3,4-b]indole

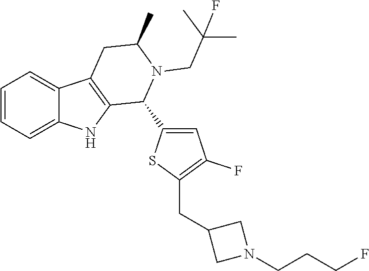 [SMILES: C[C@@H]1Cc2c([nH]c3ccccc23)[C@@H](c2cc(F)c(CC3CN(CCCF)C3)s2)N1CC(C)(C)F]

Diisopropylethylamine (0.2 mL, 1.05 mmol, 5.0 equiv) was added to a solution of (1S,3R)-1-(5-(azetidin-3-ylmethyl)-4-fluorothiophen-2-yl)-2-(2-fluoro-2-methylpropyl)-3-methyl-2,3,4,9-tetrahydro-1H-pyrido[3,4-b]indole (90 mg, 0.21 mmol, 1.0 equiv) and 1-bromo-3-fluoropropane (33 mg, 0.21 mmol, 1.0 equiv) in NN-dimethyl acetamide (2 mL) at room temperature under nitrogen. After stirring for 40 h, the reaction was diluted with ethyl acetate (20 mL), washed with water (4×10 mL) and saturated brine (1×10 mL), dried over magnesium sulfate, filtered and concentrated under reduced pressure to give a brown oil. Purification on a Biotage automated system (Biotage Amino Duo column 11 g, 50 μm), eluting with a gradient of 0 to 100% ethyl acetate in heptanes, gave partially purified title compound (24 mg, 23% yield) as a yellow oil. Further purification on a Teledyne Isco system equipped with a Waters T3 Atlantis column (5 μm, 19×250 mm), eluting with a gradient of 80 to 100% acetonitrile in water, gave the title compound (3 mg, 3% yield) as a white foam. ¹H NMR (400 MHz, CDCl₃) δ=7.86 (br s, 1H), 7.52 (d, J=8, 1H), 7.35 (d, J=8, 1H), 7.20 (dt, J=1, 8, 1H), 7.15-7.10 (m, 1H), 6.47 (s, 1H), 5.15 (br d, J=2, 1H), 4.53 (t, J=6, 1H), 4.41 (t, J=6, 1H), 3.49 (br s, 2H), 3.42-3.33 (m, 1H), 2.98-2.83 (m, 4H), 2.80-2.72 (m, 1H), 2.66-2.47 (m, 6H), 1.84-1.69 (m, 2H), 1.57-1.49 (m, 3H), 1.40-1.23 (m, 4H), 1.17 (d, J=7, 3H); LCMS: m/z=490.2 [M+H]⁺.

### Example 70: 5-Bromo-2-((1S,3R)-2-(2-fluoro-2-methylpropyl)-3-methyl-2,3,4,9-tetrahydro-1H-pyrido[3,4-b]indol-1-yl)thiazole

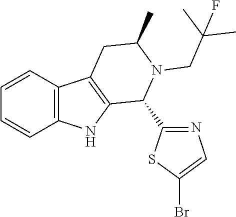 [SMILES: C[C@@H]1Cc2c([nH]c3ccccc23)[C@@H](c2ncc(Br)s2)N1CC(C)(C)F]

5-Bromothiazole-2-carbaldehyde (2.00 g, 10.4 mmol, 1 equiv) and acetic acid (1.2 mL, 21 mmol, 2.0 equiv) were sequentially added to a solution of (R)—N-(1-(1H-indol-3-yl)propan-2-yl)-2-fluoro-2-methylpropan-1-amine (3.10 g, 12.5 mmol, 1.2 equiv) in toluene (52 mL). After heating at 80° C. for 8 h, the reaction was cooled to room temperature and transferred to a separatory funnel and diluted with ethyl acetate (60 mL) and saturated sodium bicarbonate (40 mL). The layers were separated, and the aqueous layer was extracted with ethyl acetate (2×60 mL). The combined organic layers were dried over sodium sulfate, filtered and concentrated under reduced pressure onto silica (33 g). The product was purified on a Biotage automated chromatography system (200 g, Biotage 60 m silica gel column), eluting with a gradient of 0 to 30% methyl tert-butyl ether in heptanes. The solid was dried under vacuum at 35° C. for 16 h to give the title compound (3.68 g, 84% yield) as a brown/yellow solid. LCMS: m/z=478.1 [M+H]⁺.

### Example 71: tert-Butyl 3-((2-((1S,3R)-2-(2-fluoro-2-methylpropyl)-3-methyl-2,3,4,9-tetrahydro-1H-pyrido[3,4-b]indol-1-yl)thiazol-5-yl)methyl)azetidine-1-carboxylate

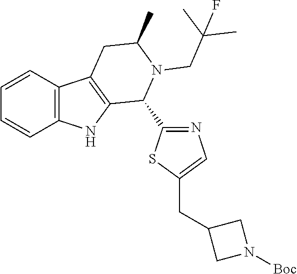 [SMILES: C[C@@H]1Cc2c([nH]c3ccccc23)[C@@H](c2ncc(CC3CN(C(=O)OC(C)(C)C)C3)s2)N1CC(C)(C)F]

5-Bromo-2-((1S,3R)-2-(2-fluoro-2-methylpropyl)-3-methyl-2,3,4,9-tetrahydro-1H-pyrido[3,4-b]indol-1-yl)thiazole (0.735 g, 1.74 mmol, 1.0 equiv), palladium acetate (66 mg, 0.30 mmol, 17 mol %), RuPhos (0.268 g, 0.574 mmol, 33 mol %), potassium carbonate (1.20 g, 8.70 mmol, 5 equiv) and potassium (1-Boc-azetidin-3-yl)methyltrifluoroborate (0.820 g, 2.96 mmol, 1.7 equiv) were added into a 40 mL vial. Then, toluene (15 mL) and water (7 mL) were added, and the mixture was sparged with nitrogen for 5 min. The reaction was sealed with a Teflon cap and was heated at 100° C. for 4 h over which time it went from a dark orange solution to a dark brown solution. The reaction was cooled to room temperature and diluted with dichloromethane (80 mL) and water (80 mL). The layers were separated, and the aqueous layer was extracted with dichloromethane (2×80 mL). The combined organic layers were dried over sodium sulfate, filtered and concentrated under reduced pressure onto Celite® (9 g). The residue was purified on a Biotage automated chromatography system (100 g, Biotage 60 μm silica gel column), eluting with a gradient of 0 to 40% ethyl acetate in heptanes. The solid was dried under vacuum at 40° C. for 1 h to give the title compound (0.437 g, 49% yield) as an off-white solid. LCMS: m/z=513.3 [M+H]⁺.

### Example 72: 5-(Azetidin-3-ylmethyl)-2-((1S,3R)-2-(2-fluoro-2-methylpropyl)-3-methyl-2,3,4,9-tetrahydro-1H-pyrido[3,4-b]indol-1-yl)thiazole

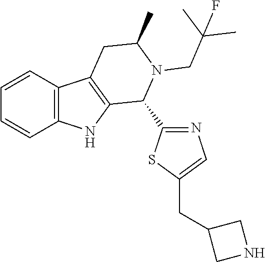 [SMILES: C[C@@H]1Cc2c([nH]c3ccccc23)[C@@H](c2ncc(CC3CNC3)s2)N1CC(C)(C)F]

Trifluoroacetic acid (1.6 mL, 21 mmol, 26 equiv) was added dropwise over ˜2 min to tert-butyl 3-((2-((1S,3R)-2-(2-fluoro-2-methylpropyl)-3-methyl-2,3,4,9-tetrahydro-1H-pyrido[3,4-b]indol-1-yl)thiazol-5-yl)methyl)azetidine-1-carboxylate (0.41 g, 0.80 mmol, 1 equiv) in dichloromethane (7.8 mL) in a 40 mL vial at 0° C. The reaction was stirred at 0° C. for 4 h. The reaction mixture was diluted with cold dichloromethane (160 mL) and transferred to a separatory funnel containing cold saturated sodium carbonate (100 mL). The layers were separated, and the aqueous layer was extracted with cold dichloromethane (2×120 mL). The combined organic layers were washed with saturated brine (200 mL), dried over sodium sulfate, filtered and concentrated under reduced pressure to give the title compound (0.330 g, >95% yield) as a brown solid. LCMS: m/z=413.2 [M+H]⁺.

### Example 73: (1S,1R)-2-(2-Fluoro-2-methylpropyl)-3-methyl-1-(5-((1-propylazetidin-3-yl)methyl)thiophen-2-yl)-2,3,4,9-tetrahydro-1H-pyrido[3,4-b]indole

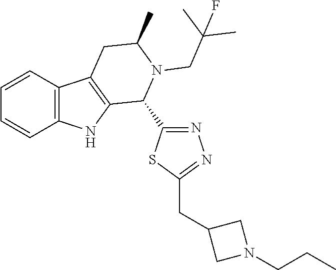 [SMILES: CCCN1CC(Cc2nnc([C@@H]3c4[nH]c5ccccc5c4C[C@@H](C)N3CC(C)(C)F)s2)C1]

Propanal (0.04 mL, 0.5 mmol, 1.5 equiv) and acetic acid (0.13 mL, 1.8 mmol, 5 equiv) were sequentially added to a solution of 5-(azetidin-3-ylmethyl)-2-((1S,3R)-2-(2-fluoro-2-methylpropyl)-3-methyl-2,3,4,9-tetrahydro-1H-pyrido[3,4-b]indol-1-yl)thiazole (0.150 g, 0.364 mmol, 1 equiv) in dichloromethane (7 mL) in a 40 mL vial. After stirring for 30 min at room temperature, sodium triacetoxyborohydride (0.309 g, 1.46 mmol, 4 equiv) was added portion wise over 5 min. The reaction was stirred at room temperature for 16 h. The reaction mixture was transferred to a separatory funnel with dichloromethane (20 mL) and washed with saturated sodium carbonate (20 mL). The aqueous layer was extracted with dichloromethane (3×20 mL). The combined organic layers were dried over sodium sulfate, filtered and concentrated under reduced pressure. The residue was purified on a Buchi automated chromatography system (28 g, Biotage 50 μm KP-amino-d column), eluting with a 100% dichloromethane. The solid was dried under vacuum at 35° C. for 2 h to give the title compound (4 mg, 2% yield) as a pale-brown foam. ¹H NMR (400 MHz, CDCl₃) δ=8.54 (br s, 1H), 7.48 (d, J=7.8 Hz, 1H), 7.36 (d, J=7.9 Hz, 1H), 7.31 (s, 1H), 7.15 (ddd, J=1.2, 7.1, 8.1 Hz, 1H), 7.10-7.05 (m, 1H), 5.26 (d, J=3.3 Hz, 1H), 3.48-3.30 (m, 3H), 3.01 (d, J=7.7 Hz, 2H), 2.89-2.79 (m, 2H), 2.77-2.66 (m, 3H), 2.60 (d, J=7.1 Hz, 2H), 2.43-2.30 (m, 2H), 1.54 (d, J=21.6 Hz, 3H), 1.39-1.32 (m, 5H), 1.28 (d, J=6.8 Hz, 3H), 0.88 (t, J=7.4 Hz, 3H); LCMS: m/z=455.3 [M+H]⁺.

### Example 74: 2-((1S,3R)-2-(2-Fluoro-2-methylpropyl)-3-methyl-2,3,4,9-tetrahydro-1H-pyrido[3,4-b]indol-1-yl)-5-((1-(3-fluoropropyl)azetidin-3-yl)methyl)thiazole

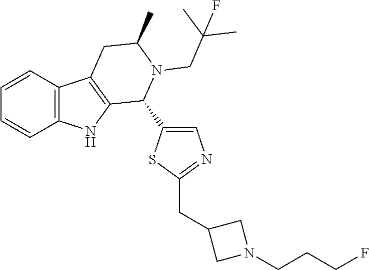 [SMILES: C[C@@H]1Cc2c([nH]c3ccccc23)[C@@H](c2cnc(CC3CN(CCCF)C3)s2)N1CC(C)(C)F]

1-Bromo-3-fluoropropane (0.03 mL, 0.4 mmol, 1 equiv) and diisopropylethylamine (0.32 mL, 1.8 mmol, 5 equiv) were added to 5-(azetidin-3-ylmethyl)-2-((1S,3R)-2-(2-fluoro-2-methylpropyl)-3-methyl-2,3,4,9-tetrahydro-1H-pyrido[3,4-b]indol-1-yl)thiazole (0.150 g, 0.364 mmol, 1 equiv) in N,N-dimethylacetamide (3.6 mL) in a 40 mL vial. After stirring room temperature for 3 days, the reaction mixture was transferred to a separatory funnel with ethyl acetate (20 mL) and washed with water (3×20 mL), then saturated brine (20 mL). The organic layer was dried over sodium sulfate, filtered and concentrated under reduced pressure onto Celite® (1.2 g). The residue was purified on a Buchi automated chromatography system (11 g, Biotage 50 μm KP-amino-d column), eluting with a gradient of 0 to 20% ethyl acetate in dichloromethane. The solid was dried under vacuum at 35° C. for 3 h to give the title compound (27 mg, 16% yield) as a yellow foam. ¹H NMR (400 MHz, CDCl₃) δ=8.54 (br s, 1H), 7.48 (d, J=7.7 Hz, 1H), 7.36 (d, J=8.1 Hz, 1H), 7.31 (s, 1H), 7.15 (ddd, J=1.2, 7.1, 8.1 Hz, 1H), 7.11-7.05 (m, 1H), 5.26 (d, J=3.3 Hz, 1H), 4.53 (t, J=6.0 Hz, 1H), 4.41 (t, J=6.0 Hz, 1H), 3.46-3.34 (m, 3H), 3.01 (d, J=7.7 Hz, 2H), 2.85 (dt, J=2.7, 6.7 Hz, 2H), 2.78-2.66 (m, 3H), 2.60 (d, J=7.0 Hz, 2H), 2.52 (t, J=7.2 Hz, 2H), 1.78-1.72 (m, 1H), 1.69 (t, J=7.1 Hz, 1H), 1.55 (d, J=21.8 Hz, 3H), 1.38-1.32 (m, 3H), 1.28 (d, J=6.7 Hz, 3H); LCMS: m/z=473.3 [M+H]⁺.

### Example 75: 5-Bromo-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole

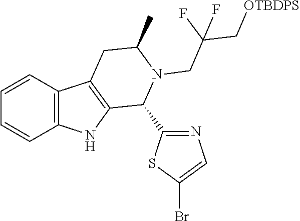

A solution of 5-bromothiazole-2-carbaldehyde (1.6 g, 8.6 mmol, 1.0 equiv), (R)—N-(1-(1H-indol-3-yl)propan-2-yl)-3-((tert-butyldiphenylsilyl)oxy)-2,2-difluoropropan-1-amine (5.2 g, 10.2 mmol, 1.2 equiv) and acetic acid (1.9 mL, 34.4 mmol, 4.0 equiv) in toluene (43 mL) was stirred at 100° C. under nitrogen for 10.5 h. After cooling to room temperature, the reaction mixture was diluted with ethyl acetate (36 mL), washed with saturated sodium carbonate (3×50 mL), dried over sodium sulfate (10 g), filtered and concentrated under reduced pressure. The crude product was absorbed onto silica gel (40 g) and purified on a Biotage automated chromatography system (220 g, 20 μm column), eluting with a gradient of 0 to 50% methyl t-butyl ether in heptanes, to give the title compound (4.1 g, 65% yield) as a gray foam. LCMS: m/z=680, 682 [M+H]⁺.

### Example 76: tert-Butyl 3-((2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazol-5-yl)methyl)azetidine-1-carboxylate

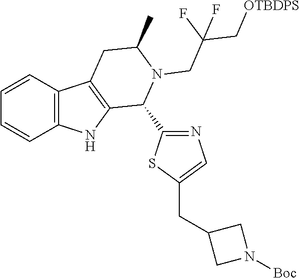

A mixture of 5-bromo-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole (3.1 g, 4.6 mmol, 1.0 equiv), potassium (1-Boc-azetidin-3-yl)methyltrifluoroborate (2.2 g, 7.8 mmol, 1.7 equiv), toluene (40 mL) and water (20 mL) was sparged with nitrogen for 30 min. Potassium carbonate (3.2 g, 23 mmol, 5.0 equiv), RuPhos (0.74 g, 1.6 mmol, 0.34 equiv) and palladium acetate (0.18 g, 0.78 mmol, 0.17 equiv) were added sequentially, then the reaction mixture was sparged with nitrogen for an additional 15 min. The reaction mixture was stirred at 100° C. under nitrogen for 7 h, then cooled to room temperature. The reaction mixture was diluted with methyl t-butyl ether (40 mL) and washed with water (3×10 mL). The organic layer was dried over sodium sulfate (5 g), filtered and concentrated under reduced pressure. The crude product was absorbed onto silica gel (30 g) under reduced pressure and purified on a Biotage automated chromatography system (200 g, 20 μm column), eluting with a gradient from 0 to 50% methyl t-butyl ether in heptanes, to give the title compound (2.1 g, 59% yield) as a tan oil. LCMS: m/z=771 [M+H]⁺.

### Example 77: 5-(Azetidin-3-ylmethyl)-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole

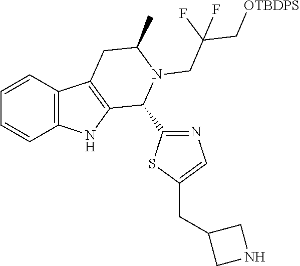

A solution of tert-butyl3-((2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazol-5-yl)methyl)azetidine-1-carboxylate (750 mg, 1.0 mmol, 1.0 equiv) in formic acid (9.2 g, 7.6 mL, 200 mmol, 200 equiv) was stirred at room temperature for 50 min, at which time thin layer chromatography and LCMS analysis indicated most of the starting material was consumed. The reaction mixture was added to a stirred saturated sodium carbonate (300 mL) such that the internal temperature did not exceed 5° C. The aqueous mixture (pH=10 by indicator paper) was extracted with methyl t-butyl ether (3×70 mL). The combined organic layers were dried over sodium sulfate, filtered and concentrated under reduced pressure to give the title compound (680 mg, quant) as a tan foam. LCMS: m/z=671 [M+H]⁺.

### Example 78: 2-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-5-((1-(3-fluoropropyl)azetidin-3-yl)methyl)thiazole

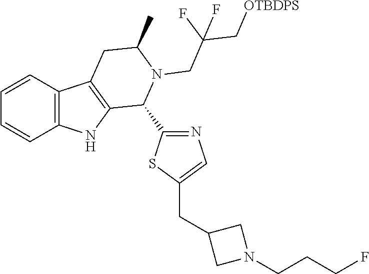

N,N-Diisopropylethylamine (0.67 g, 0.9 mL, 1.05 mmol, 5.0 equiv) was added to a solution of 5-(azetidin-3-ylmethyl)-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole (680 mg, 1.0 mmol, 1.0 equiv) and 1-bromo-3-fluoropropane (147 mg, 1.05 mmol, 1.0 equiv) in N,N-dimethylacetamide (9 mL). After stirring for 18 h, the reaction mixture was diluted with ethyl acetate (100 mL), washed with water (4×50 mL), dried over magnesium sulfate (10 g), filtered and concentrated under reduced pressure. The crude product was absorbed onto Celite® (2 g) under reduced pressure and purified on a Biotage automated chromatography system (Amino Duo column, 28 g, 50 μm), eluting with a gradient of 0 to 50% ethyl acetate in hexanes, to give the title compound (51 mg, 7% yield) as pale-yellow oil. LCMS: m/z=731 [M+H]⁺.

### Example 79: 2,2-Difluoro-3-((1S,3R)-1-(5-((1-(3-Fluoropropyl)azetidin-3-yl)methyl)thiazol-2-yl)-3-methyl-1,3,4,9-tetrahydro-2H-pyrido[3,4-b]indol-2-yl)propan-1-ol

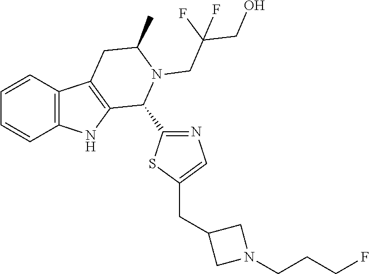 [SMILES: C[C@@H]1Cc2c([nH]c3ccccc23)[C@@H](c2ncc(CC3CN(CCCF)C3)s2)N1CC(F)(F)CO]

1 M Tetrabutylammonium fluoride in THF (70 μL, 0.07 mmol, 0.03 equiv) was added to a solution of 2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-5-((1-(3-fluoropropyl)azetidin-3-yl)methyl)thiazole (50 mg, 0.07 mmol, 1.0 equiv) in anhydrous tetrahydrofuran (0.5 mL) at 0 to 5° C. under an argon atmosphere. The reaction mixture was stirred for 3.5 h, at which time thin layer chromatography and LCMS analysis showed that starting material was mostly consumed. Ice-cold water (0.5 mL) was added. After stirring for 5 min, the reaction was extracted with dichloromethane (3×1 mL). The combined organic layers were dried over magnesium sulfate (0.5 g), filtered and concentrated under reduced pressure. The residue was purified on a Biotage automated chromatography system (Amino Duo column, 5 g, 50 pin), eluting with a gradient of 0 to 100% ethyl acetate in hexanes, followed by trituration with HPLC grade hexanes (3 mL) to give the title compound (11 mg, 37% yield) as pale-yellow solid. ¹H NMR (400 MHz, CDCl₃) δ=8.54 (s, 1H), 7.49 (d, J=7.8 Hz, 1H), 7.38-7.33 (m, 2H), 7.17 (dt, J=1.1, 7.6 Hz, 1H), 7.12-7.07 (m, 1H), 5.28 (s, 1H), 4.52 (t, J=5.9 Hz, 1H), 4.40 (t, J=5.9 Hz, 1H), 4.04-3.93 (m, 2H), 3.46-3.37 (m, 3H), 3.35-3.22 (m, 1H), 3.04-2.86 (m, 5H), 2.75-2.53 (m, 5H), 1.80-1.66 (m, 10H), 1.34 (d, J=6.8 Hz, 3H); LCMS: m/z=493.2 [M+H]⁺.

### Example 80: 2-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-5-((1-propylazetidin-3-yl)methyl)thiazole

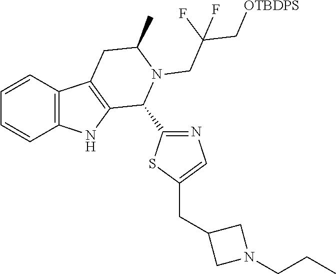

N,N-Diisopropylethylamine (0.67 g, 0.9 mL, 1.05 mmol, 5.0 equiv) was added to a solution of 5-(azetidin-3-ylmethyl)-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole (680 mg, 1.0 mmol, 1.0 equiv) and 1-bromopropane (128 mg, 1.05 mmol, 1.0 equiv) in N,N-dimethylacetamide (9 ml). After stirring for 18 h, the reaction mixture was diluted with ethyl acetate (100 mL), washed with water (4×50 mL), dried over magnesium sulfate (10 g), filtered and concentrated under reduced pressure. The crude product was absorbed onto Celite® (2 g) with dichloromethane (10 mL) under reduced pressure and purified on a Biotage automated chromatography system (Amino Duo column, 28 (11 g, 50 μm), eluting with a gradient from 0 to 100% ethyl acetate in hexanes to give the title compound (24 mg, 3% yield) as pale-yellow oil. LCMS: m/z=713 [M+H]⁺.

### Example 81: 2,2-Difluoro-3-((1S,3R)-3-methyl-1-(5-((1-propylazetidin-3-yl)methyl)thiazol-2-yl)-1,3,4,9-tetrahydro-2H-pyrido[3,4-b]indol-2-yl)propan-1-ol

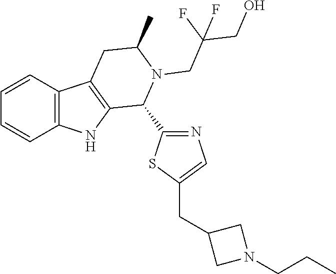 [SMILES: CCCN1CC(Cc2cnc([C@@H]3c4[nH]c5ccccc5c4C[C@@H](C)N3CC(F)(F)CO)s2)C1]

1 M Tetrabutylammonium fluoride in THF (33 μL, 0.033 mmol, 1.0 equiv) was added to a solution of 2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-5-((1-propylazetidin-3-yl)methyl)thiazole (24 mg, 0.033 mmol, 1.0 equiv) in anhydrous tetrahydrofuran (0.3 mL) at 0 to 5° C. under an argon atmosphere. The reaction mixture was stirred for 3.5 h, at which time thin layer chromatography and LCMS analysis showed that starting material was mostly consumed. Ice-cold water (0.5 mL) was added. After stirring for 5 min, the reaction was extracted with dichloromethane (3×1 mL). The combined organic layers were dried over magnesium sulfate (0.5 g), filtered and concentrated under reduced pressure. The residue was purified on a Biotage automated chromatography system (Amino Duo column, 5 g, 50 m), eluting with a gradient of 0 to 100% ethyl acetate in hexanes, followed by trituration with a 1 to 1 mixture of methyl t-butyl ether to hexanes (1 mL) to give the title compound (4 mg, 32% yield) as pale-yellow solid. ¹H NMR (400 MHz, acetone-d₆) δ=10.01 (br s, 1H), 7.46 (br d, J=7.8 Hz, 1H), 7.42 (br d, J=8.1 Hz, 1H), 7.35 (s, 11H), 7.10 (t, J=7.2 Hz, 11H), 7.04-6.97 (m, 11H), 5.32 (s, 1H), 4.05-3.88 (m, 2H), 3.42-3.23 (m, 4H), 3.05 (d, J=7.6 Hz, 2H), 2.94-2.77 (m, 4H), 2.67-2.57 (m, 3H), 2.30 (t, J=7.2 Hz, 2H), 1.38-1.25 (m, 6H), 0.84 (t, J=7.4 Hz, 3H); LCMS: m/z=474.2 [M+H]⁺.

### Example 82: 5-Bromo-2-((1S,3R)-3-methyl-2-(2,2,2-trifluoroethyl)-2,3,4,9-tetrahydro-1H-pyrido[3,4-b]indol-1-yl)thiazole

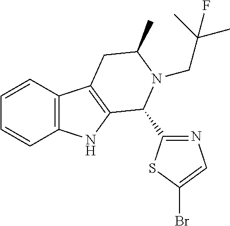 [SMILES: C[C@@H]1Cc2c([nH]c3ccccc23)[C@@H](c2ncc(Br)s2)N1CC(C)(C)F]

A solution of 5-bromothiazole-2-carbaldehyde (4.8 g, 25 mmol, 1.0 equiv), (R)-1-(1H-indol-3-yl)-N-(2,2,2-trifluoroethyl)propan-2-amine (7.7 g, 30 mmol, 1.2 equiv) and acetic acid (5.6 mL, 100 mmol, 4.0 equiv) in toluene (125 mL) was stirred at 100° C. under nitrogen for 7 h. After cooling to room temperature, the reaction mixture was diluted with ethyl acetate (200 mL), washed with saturated sodium carbonate (3×50 mL), dried over sodium sulfate (50 g), filtered and concentrated under reduced pressure. The crude product was absorbed onto silica gel (40 g) with dichloromethane (100 mL) under reduced pressure and purified on a Biotage automated chromatography system, (a silica gel column, 350 g, 20 μm), eluting with a gradient of 0 to 10% ethyl acetate in heptanes to give the title compound (7.0 g, 65% yield) as a gray-yellow solid. LCMS: m/z=430, 432 [M+H]⁺.

### Example 83: tert-Butyl3-((2-((1S,3R)-3-methyl-2-(2,2,2-trifluoroethyl)-2,3,4,9-tetrahydro-1H-pyrido[3,4-b]indol-1-yl)thiazol-5-yl)methyl)azetidine-1-carboxylate

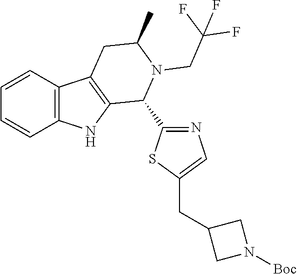 [SMILES: C[C@@H]1Cc2c([nH]c3ccccc23)[C@@H](c2ncc(CC3CN(C(=O)OC(C)(C)C)C3)s2)N1CC(F)(F)F]

A mixture of 5-bromo-2-((1S,3R)-3-methyl-2-(2,2,2-trifluoroethyl)-2,3,4,9-tetrahydro-1H-pyrido[3,4-b]indol-1-yl)thiazole (7.0 g, 16.3 mmol, 1.0 equiv), potassium (1-Boc-azetidin-3-yl)methyltrifluoroborate (7.7 g, 27.7 mmol, 1.7 equiv), toluene (138 mL) and water (69 mL) was sparged with nitrogen for 30 min. Potassium carbonate (11.3 g, 81.5 mmol, 5.0 equiv), RuPhos (2.6 g, 5.5 mmol, 0.34 equiv) and palladium acetate (0.62 g, 2.8 mmol, 0.17 equiv) were added sequentially and the reaction mixture was sparged with nitrogen for an additional 15 min. After stirring at 100° C. under nitrogen for 7 h, the reaction was cooled to room temperature, diluted with ethyl acetate (400 mL) and washed with water (3×100 mL). The organic layer was dried over sodium sulfate (30 g), filtered and concentrated under reduced pressure. The crude product was absorbed onto silica gel (35 g) with dichloromethane (50 mL) under reduced pressure and purified on a Biotage automated chromatography system, (silica gel column, 350 g, 20 μm), eluting with a gradient of 0 to 50% ethyl acetate in heptanes, to give the title compound (3.8 g, 45% yield) as a tan solid. LCMS: m/z=521 [M+H]⁺.

### Example 84: 5-(Azetidin-3-ylmethyl)-2-((1S,3R)-3-methyl-2-(2,2,2-trifluoroethyl)-2,3,4,9-tetrahydro-1H-pyrido[3,4-b]indol-1-yl)thiazole

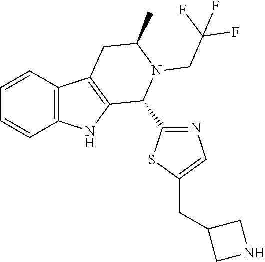 [SMILES: C[C@@H]1Cc2c([nH]c3ccccc23)[C@@H](c2ncc(CC3CNC3)s2)N1CC(F)(F)F]

A solution of tert-butyl3-((2-((1S,3R)-3-methyl-2-(2,2,2-trifluoroethyl)-2,3,4,9-tetrahydro-1H-pyrido[3,4-b]indol-1-yl)thiazol-5-yl)methyl)azetidine-1-carboxylate (700 mg, 1.4 mmol, 1.0 equiv) in formic acid (18.6 g, 15 mL, 403 mmol, 300 equiv) was stirred at room temperature for 50 min at which time thin layer chromatography and LCMS analysis indicated most of the starting material was consumed. The reaction mixture was added to a stirred saturated sodium carbonate solution (300 mL) such that the internal temperature did not exceed 5° C. The aqueous mixture (pH=10 by indicator paper) was extracted with methyl t-butyl ether (3×100 mL). The combined organic layers were dried over sodium sulfate, filtered and concentrated under reduced pressure to give the title compound (615 mg, 108% mass yield with 78% HPLC purity) as a tan solid. LCMS: m/z=421 [M+H]⁺.

### Example 85: 5-((1-(3-Fluoropropyl)azetidin-3-yl)methyl)-2-((1S,3R)-3-methyl-2-(2,2,2-trifluoroethyl)-2,3,4,9-tetrahydro-1H-pyrido[3,4-b]indol-1-yl)thiazole

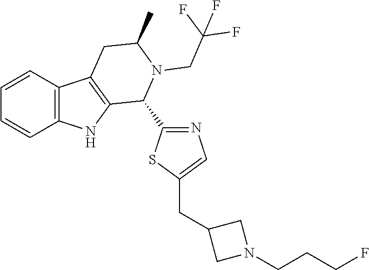 [SMILES: C[C@@H]1Cc2c([nH]c3ccccc23)[C@@H](c2ncc(CC3CN(CCCF)C3)s2)N1CC(F)(F)F]

N,N-Diisopropylethylamine (1.35 g 1.8 mL, 10.5 mmol, 5.0 equiv) was added to a solution of 5-(azetidin-3-ylmethyl)-2-((1S,3R)-3-methyl-2-(2,2,2-trifluoroethyl)-2,3,4,9-tetrahydro-1-pyrido[3,4-b]indol-1-yl)thiazole (840 mg, 2.1 mmol, 1.0 equiv) and 1-bromo-3-fluoropropane (295 mg, 2.1 mmcl, 1.0 equiv) in N,N-dimethylacetamide (17 mL). After stirring for 18 h, the reaction mixture was diluted with ethyl acetate (100 ml), washed with water (4×50 mL), dried over magnesium sulfate (10 g), filtered and concentrated under reduced pressure. The crude product was absorbed onto silica gel (2 g) with dichloromethane (10 mL) under reduced pressure and purified on a Biotage automated chromatography system (silica gel column, 10 g, 20 μm), eluting with a gradient of 0 to 10% methanol in dichloromethane to give the title compound (5 mg, 1% yield) as a white solid after lyophilization from acetonitrile-water. ¹H NMR (400 MHz, CDCl₃) δ=8.51 (s, 1H), 7.48 (d, J=7.9 Hz, 1H), 7.38 (d, J=8.1 Hz, 1H), 7.34 (s, 1H), 7.18 (dt, J=1.2, 7.6 Hz, 1H), 7.12-7.07 (m, 1H), 5.17 (s, 1H), 4.53 (t, J=6.0 Hz, 1H), 4.41 (t, J=6.0 Hz, 1H), 3.46-3.33 (m, 4H), 3.10-2.99 (m, 3H), 2.87 (br s, 2H), 2.76-2.66 (m, 2H), 2.63-2.50 (m, 3H), 1.79-1.66 (m, 2H), 1.35 (d, J=6.7 Hz, 3H); LCMS: m/z=481.2 [M+H]⁺.

### Example 86: 2-((1S,3R)-3-Methyl-2-(2,2,2-trifluoroethyl)-2,3,4,9-tetrahydro-1H-pyrido[3,4-b]indol-1-yl)-S-((1-propylazetidin-3-yl)methyl)thiazole

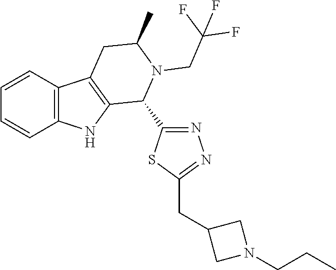 [SMILES: CCCN1CC(Cc2nnc([C@@H]3c4[nH]c5ccccc5c4C[C@@H](C)N3CC(F)(F)F)s2)C1]

N,N-Diisopropylethylamine (0.47 g 0.61 mL, 3.7 mmcl, 5.0 equiv) was added to a solution of 5-(azetidin-3-ylmethyl)-2-(1S,3R)-3-methyl-2-(2,2,2-trifluoroethyl)-2,3,4,9-tetrahydro-1H-pyrido[3,4-b]indol-1-yl)thiazole (308 mg, 0.73 mmol, 1.0 equiv) and 1-bromopropane (90 mg, 0.73 mmol, 1.0 equiv) in N,N-dimethylacetamide (5.5 mL). After stirring for 5 days, the reaction mixture was diluted with ethyl acetate (20 mL), washed with water (7×5 mL), dried over magnesium sulfate (10 g), filtered and concentrated under reduced pressure. The crude product was absorbed onto silica gel (2 g) with dichloromethane (5 mL) under reduced pressure and purified on a Biotage automated chromatography system (silica gel column, 10 g, 20 μm), eluting with a gradient of 0 to 10% methanol in dichloromethane to give partially purified title compound (32 mg, 9% yield) as a pale-yellow oil. This sample was absorbed onto Celite® (2 g) with dichloromethane (5 mL) under reduced pressure and purified on a Biotage automated chromatography system (Amino Duo column, 5 g, 60 μm), eluting with a gradient of 0 to 100% ethyl acetate in hexanes, to give the title compound (15 mg, 5% yield) as a pale-yellow solid. ¹H NMR (400 MHz, CDCl₃) δ=8.54 (s, 1H), 7.48 (d, J=7.9 Hz, 1H), 7.39-7.35 (m, 1H), 7.33 (s, 1H), 7.18 (dt, J=1.2, 7.6 Hz, 1H), 7.12-7.07 (m, 1H), 5.36-5.33 (m, 1H), 5.16 (s, 1H), 3.47-3.33 (m, 4H), 3.10-2.99 (m, 3H), 2.83 (dt, J=2.6, 6.7 Hz, 2H), 2.76-2.66 (m, 2H), 2.61-2.54 (m, 1H), 2.39-2.32 (m, 2H), 1.38-1.30 (m, 5H), 0.88 (t, J=7.4 Hz, 3H); LCMS: m/z=463.2 [M+H]⁺.

### Example 87: 5-((1-(3,3-Difluoropropyl)azetidin-3-yl)methyl)-2-((1S,3R)-3-methyl-2-(2,2,2-trifluoroethyl)-2,3,4,9-tetrahydro-1H-pyrido[3,4-b]indol-1-yl)thiazole

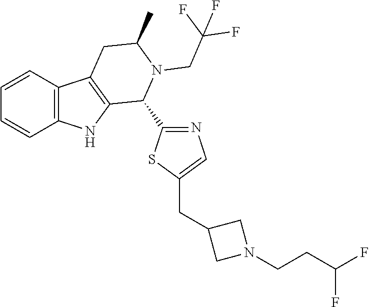 [SMILES: C[C@@H]1Cc2c([nH]c3ccccc23)[C@@H](c2ncc(CC3CN(CCC(F)F)C3)s2)N1CC(F)(F)F]

N,N-Diisopropylethylamine (0.47 g, 0.6 mL, 3.7 mmol, 5.0 equiv) was added to a solution of 5-(azetidin-3-ylmethyl)-2-((1S,3R)-3-methyl-2-(2,2,2-trifluoroethyl)-2,3,4,9-tetrahydro-1H-pyrido[3,4-b]indol-1-yl)thiazole (308 mg, 0.73 mmol, 1.0 equiv) and 2,2-difluoropropyl tosylate (183 mg, 0.73 mmol, 1.0 equiv) in N,N-dimethylacetamide (5.5 mL) and the reaction mixture was stirred for 4 days. Additional difluoropropyl tosylate (2×1.0 equiv) was added in two 24-hour increments. The reaction mixture was diluted with ethyl acetate (30 mL), washed with water (4×15 mL) and 10% lithium chloride. The organic layer was dried over magnesium sulfate (10 g), filtered and concentrated under reduced pressure. The sample was absorbed onto silica gel (5 g) with dichloromethane (10 mL) under reduced pressure and purified on a Biotage automated chromatography system (silica gel column, 10 g, 20 μm), eluting with a gradient of 0 to 10% methanol in dichloromethane to give partially purified title compound (22 mg, 6% yield) as a pale-yellow oil. This sample was absorbed onto Celite® (2 g) with dichloromethane (5 mL) under reduced pressure and purified on a Biotage automated chromatography system (Amino Duo column, 5 g, 60 μm), eluting with a gradient of 0 to 100% ethyl acetate in hexanes to give the title compound (9 mg, 2% yield) as a pale-yellow solid. ¹H NMR (400 MHz, CDCl₃) δ=8.54 (s, 1H), 7.48 (d, J=7.8 Hz, 1H), 7.37 (d, J=8.1 Hz, 1H), 7.33 (s, 1H), 7.18 (dt, J=1.2, 7.6 Hz, 1H), 7.13-7.07 (m, 1H), 5.87 (tt, J=4.7, 56.8 Hz, 1H), 5.35 (s, 1H), 5.16 (s, 1H), 3.47-3.35 (m, 4H), 3.10-2.99 (m, 3H), 2.86 (dt, J=2.4, 6.6 Hz, 2H), 2.75-2.66 (m, 2H), 2.62-2.51 (m, 3H), 1.85 (dtt, J=4.6, 7.2, 17.0 Hz, 2H), 1.35 (d, J=6.8 Hz, 3H); LCMS: m/z=499.2 [M+H]⁺.

### Example 88: 5-Bromo-2-((1S,3R)-2-(2,2-difluoroethyl)-3-methyl-2,3,4,9-tetrahydro-1H-pyrido[3,4-b]indol-1-yl)thiazole

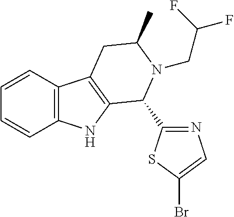 [SMILES: C[C@@H]1Cc2c([nH]c3ccccc23)[C@@H](c2ncc(Br)s2)N1CC(F)F]

5-Bromothiazole-2-carbaldehyde (2.00 g, 10.4 mmol, 1 equiv) and acetic acid (1.2 mL, 21 mmol, 2.0 equiv) were sequentially added to a solution of (R)—N-(2,2-difluoroethyl)-1-(1H-indol-3-yl)propan-2-amine (2.98 g, 12.5 mmol, 1.2 equiv) in toluene (52 mL). After heating at 80° C. for 1 h, the reaction was cooled to room temperature and diluted with ethyl acetate (60 mL) and saturated sodium bicarbonate (40 mL). The layers were separated, and the aqueous layer was extracted with ethyl acetate (2×60 mL). The combined organic layers were dried over sodium sulfate, filtered and concentrated under reduced pressure onto silica (30 g). The material was purified on a Biotage automated chromatography system (220 g, Sorbtech 40-75 μm silica gel column), eluting with a gradient of 0 to 30% methyl tert-butyl ether in heptanes. The solid was dried under vacuum at 40° C. for 1.5 h to give the title compound (2.94 g, 68% yield) as a yellow foam. LCMS: m/z=412.1 [M+H]⁺.

### Example 89: tert-Butyl 3-((2-((1S,3R)-2-(2,2-difluoroethyl)-3-methyl-2,3,4,9-tetrahydro-1H-pyrido[3,4-b]indol-1-yl)thiazol-5-yl)methyl)azetidine-1-carboxylate

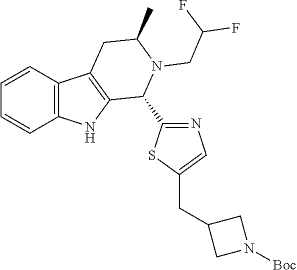 [SMILES: C[C@@H]1Cc2c([nH]c3ccccc23)[C@@H](c2ncc(CC3CN(C(=O)OC(C)(C)C)C3)s2)N1CC(F)F]

5-Bromo-2-((1S,3R)-2-(2-fluoro-2-methylpropyl)-3-methyl-2,3,4,9-tetrahydro-1H-pyrido[3,4-b]indol-1-yl)thiazole (1.50 g, 3.64 mmol, 1.0 equiv), palladium acetate (0.163 g, 0.728 mmol, 20 mol %), RuPhos (0.680 g, 1.46 mmol, 40 mol %), potassium carbonate (2.51 g, 18.2 mmol, 5 equiv), and potassium (1-Boc-azetidin-3-yl)methyltrifluoroborate (1.71 g, 6.19 mmol, 1.7 equiv) was added to a 100 mL round bottom flask. Toluene (24 mL) and water (12 mL) were added to the reaction flask, which was sparged with nitrogen for 10 min. After heating at 100° C. for 15 h, the reaction was cooled to room temperature and diluted with dichloromethane (80 mL) and water (80 mL). The layers were separated, and the aqueous layer was extracted with dichloromethane (2×80 mL). The combined organic layers were dried over sodium sulfate, filtered and concentrated under reduced pressure onto silica (22 g). The residue was purified on a Biotage automated chromatography system (120 g, Sorbtech 40-75 m silica gel column), eluting with a gradient of 0 to 40% ethyl acetate in heptanes. The solid was dried under vacuum at 30° C. for 3 h to give the title compound (1.04 g, 57% yield) as a tan foam. LCMS: m/z=503.3 [M+H]⁺.

### Example 90: 5-(Azetidin-3-ylmethyl)-2-((1S,3R)-2-(2,2-difluoroethyl)-3-methyl-2,3,4,9-tetrahydro-1H-pyrido[3,4-b]indol-1-yl)thiazole

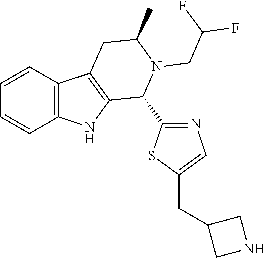 [SMILES: C[C@@H]1Cc2c([nH]c3ccccc23)[C@@H](c2ncc(CC3CNC3)s2)N1CC(F)F]

Trifluoroacetic acid (5.9 mL, 77 mmol, 39 equiv) was added to tert-butyl 3-((2-((1S,3R)-2-(2,2-difluoroethyl)-3-methyl-2,3,4,9-tetrahydro-1H-pyrido[3,4-b]indol-1-yl)thiazol-5-yl)methyl)azetidine-1-carboxylate (0.93 g, 1.8 mmol, 1 equiv) in dichloromethane (14 mL) at 0° C. in a 40 mL vial. The reaction was stirred at 0° C. for 45 min. The reaction mixture was transferred to an Erlenmeyer flask containing cold dichloromethane (200 mL). Saturated sodium carbonate (˜40 mL) was added to the mixture dropwise at 0° C. until pH was 8-9. The layers were separated, and the organic layer was washed sequentially with saturated sodium carbonate (170 mL), saturated brine (100 mL) and saturated sodium carbonate (170 mL). The organic layer was dried over sodium sulfate, filtered and concentrated under reduced pressure to give the title compound (0.249 g, 31% yield) as a yellow/brown semi-solid. LCMS: m/z=403.2 [M+H]⁺.

### Example 91: 2-((1S,3R)-2-(2,2-Difluoroethyl)-3-methyl-2,3,4,9-tetrahydro-1H-pyrido[3,4-b]indol-1-yl)-5-((1-(3-fluoropropyl)azetidin-3-yl)methyl)thiazole

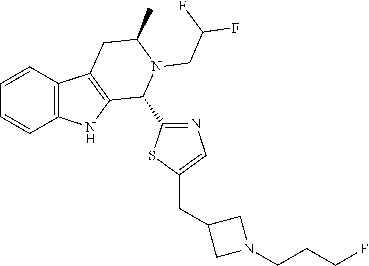 [SMILES: C[C@@H]1Cc2c([nH]c3ccccc23)[C@@H](c2ncc(CC3CN(CCCF)C3)s2)N1CC(F)F]

1-Bromo-3-fluoropropane (0.03 mL, 0.3 mmol, 1 equiv) and N,N-diisopropylethylamine (0.27 mL, 1.6 mmol, 5 equiv) were sequentially added to a solution of 5-(azetidin-3-ylmethyl)-2-((1S,3R)-2-(2,2-difluoroethyl)-3-methyl-2,3,4,9-tetrahydro-1H-pyrido[3,4-b]indol-1-yl)thiazole (0.125 g, 0.311 mmol, 1 equiv) in N,N-dimethylacetamide (3.1 mL) in a 40 mL vial. After stirring for 3 days at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite® (1.8 g). The residue was purified on a Biotage automated chromatography system (11 g, Biotage 50 μm KP-amino-d column), eluting with a gradient of 0 to 50% ethyl acetate in heptanes, to give partially purified title compound (30 mg). The material was absorbed onto Celite® (1.5 g). The material was purified on a Biotage automated chromatography system (5.5 g, Redigold C18 column), eluting with a gradient of 0 to 85% methanol in water. No separation was obtained, and the recovered material (26 mg) was absorbed onto Celite® (1 g). The material was purified on a Biotage automated chromatography system (4 g, Sorbtech 40-75 μm silica gel column) eluting with a gradient of 0 to 10% methanol in dichloromethane. The material was lyophilized from a mixture of acetonitrile and water to give the title compound (16 mg, 11% yield) as a yellow solid. ¹H NMR (400 MHz, CDCl₃) δ=8.46 (s, 1H), 7.49 (d, J=7.6 Hz, 1H), 7.35 (t, J=4.0 Hz, 2H), 7.17 (dt, J=1.2, 7.5 Hz, 1H), 7.12-7.07 (m, 1H), 5.89 (ddt, J=3.7, 4.9, 56.3 Hz, 1H), 5.14 (s, 1H), 4.53 (t, J=5.9 Hz, 1H), 4.42 (t, J=5.9 Hz, 1H), 3.51 (br s, 2H), 3.48-3.11 (m, 3H), 3.11-2.96 (m, 4H), 2.96-2.69 (m, 4H), 2.68-2.56 (m, 3H), 1.86-1.71 (m, 2H), 1.31 (d, J=6.7 Hz, 3H); LCMS: m/z=463.2 [M+H]⁺.

### Example 92: 2-((1S,3R)-2-(2,2-Difluoroethyl)-3-methyl-2,3,4,9-tetrahydro-1H-pyrido[3,4-b]indol-1-yl)-5-((1-propylazetidin-3-yl)methyl)thiazole

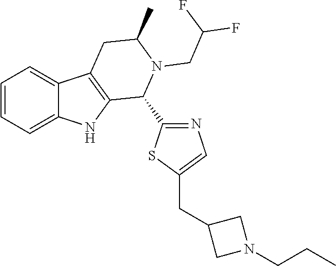 [SMILES: CCCN1CC(Cc2cnc([C@@H]3c4[nH]c5ccccc5c4C[C@@H](C)N3CC(F)F)s2)C1]

1-Bromopropane (0.03 mL, 0.3 mmol, 1 equiv) and N,N-diisopropylethylamine (0.27 mL, 1.6 mmol, 5 equiv) were sequentially added to a solution of 5-(azetidin-3-ylmethyl)-2-((1S,3R)-2-(2,2-difluoroethyl)-3-methyl-2,3,4,9-tetrahydro-1H-pyrido[3,4-b]indol-1-yl)thiazole (0.125 g, 0.311 mmol, 1 equiv) in N,N-dimethylacetamide (3.1 mL) in a 40 mL vial. After stirring for 6 days at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite® (1 g). The residue was purified on a Biotage automated chromatography system (11 g, Biotage 50 μm KP-amino-d column), eluting with a gradient of 0 to 50% ethyl acetate in heptanes. The material was lyophilized from a mixture of acetonitrile and water to give the title compound (15 mg, 11% yield) as a yellow solid. ¹H NMR (400 MHz, CDCl₃) δ=8.45 (s, 1H), 7.48 (d, J=7.7 Hz, 1H), 7.40-7.30 (m, 2H), 7.17 (dt, J=1.2, 7.6 Hz, 1H), 7.14-7.06 (m, 1H), 5.89 (ddt, J=3.9, 4.9, 56.5 Hz, 1H), 5.14 (s, 1H), 3.49-3.34 (m, 3H), 3.25-3.11 (m, 1H), 3.01 (d, J=7.7 Hz, 2H), 2.90-2.79 (m, 3H), 2.77-2.57 (m, 3H), 2.42-2.30 (m, 2H), 1.39-1.32 (m, 2H), 1.30 (d, J=6.7 Hz, 3H), 0.88 (t, J=7.4 Hz, 3H); LCMS: m/z=445.3 [M+H]⁺.

### Example 93: 2-((1S,3R)-2-(2,2-Difluoroethyl)-3-methyl-2,3,4,9-tetrahydro-1H-pyrido[3,4-b]indol-1-yl)-5-((1-(3,3-difluoropropyl)azetidin-3-yl)methyl)thiazole

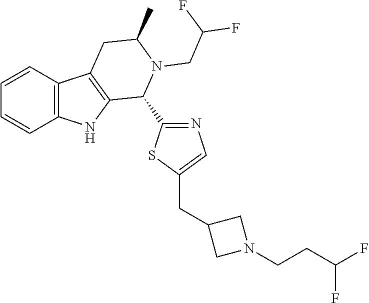 [SMILES: C[C@@H]1Cc2c([nH]c3ccccc23)[C@@H](c2ncc(CC3CN(CCC(F)F)C3)s2)N1CC(F)F]

3,3-Difluoropropyl 4-methylbenzenesulfonate (0.087 g, 0.311 mmol, 1 equiv) and N,N-diisopropylethylamine (0.30 mL, 1.74 mmol, 5 equiv) were sequentially added to a solution of 5-(azetidin-3-ylmethyl)-2-((1S,3R)-2-(2,2-difluoroethyl)-3-methyl-2,3,4,9-tetrahydro-1H-pyrido[3,4-b]indol-1-yl)thiazole (0.200 g, 0.348 mmol, 1 equiv, 70% purity) in N,N-dimethylacetamide (3.5 mL) in a 40 mL vial. After stirring for 14 h at room temperature, additional 3,3-difluoropropyl 4-methylbenzenesulfonate (0.188 g, 0.752 mmol, 2.2 equiv) was added. The reaction was stirred an additional 24 h at room temperature, then heated at 30° C. for 5 h. The reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite® (2.3 g). The residue was purified on a Biotage automated chromatography system (11 g, Biotage 50 m KP-amino-d column), eluting with a gradient of 50 to 100% dichloromethane in heptanes. The material was lyophilized from a 9 to 1 mixture of acetonitrile to give the title compound (20 mg, 12% yield) as a yellow solid. ¹H NMR (400 MHz, CDCl₃) δ=8.46 (s, 1H), 7.49 (d, J=7.7 Hz, 1H), 7.39-7.30 (m, 2H), 7.17 (dt, J=1.2, 7.6 Hz, 1H), 7.12-7.06 (m, 1H), 6.07-5.71 (m, 1H), 5.39-5.33 (m, 1H), 5.33-5.29 (m, 1H), 5.14 (s, 1H), 3.47-3.33 (m, 3H), 3.26-3.12 (m, 1H), 3.01 (d, J=7.7 Hz, 2H), 2.91-2.79 (m, 3H), 2.76-2.53 (m, 5H), 1.85 (dtt, J=4.6, 7.1, 17.0 Hz, 2H), 1.31 (d, J=6.7 Hz, 3H); LCMS: m/z=481.3 [M+H]⁺.

### Example 94: 2-Bromo-5-((1S,3R)-2-(2-fluoro-2-methylpropyl)-3-methyl-2,3,4,9-tetrahydro-1H-pyrido[3,4-b]indol-1-yl)thiazole

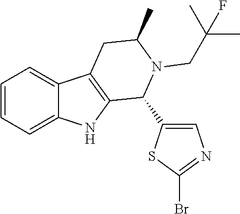 [SMILES: C[C@@H]1Cc2c([nH]c3ccccc23)[C@@H](c2cnc(Br)s2)N1CC(C)(C)F]

A mixture 2-bromothiazole-5-carbaldehyde (2 g, 10.4 mmol, 1 equiv), (R)—N-(1-(1H-indol-3-yl)propan-2-yl)-2-fluoro-2-methylpropan-1-amine (3.1 g, 12.5 mmol, 1.2 equiv) and acetic acid (1.2 mL, 20.8 mmol, 2.0 equiv) in toluene (35 mL) was heated at 80° C. in a 100 mL round bottom flask for 12 h. After cooling to room temperature, the mixture was diluted with ethyl acetate (50 mL) and washed with saturated sodium bicarbonate (100 mL). The aqueous layer was extracted with ethyl acetate (3×50 mL). The combined organic layers were washed with saturated brine (100 mL), dried over sodium sulfate, filtered and concentrated under reduced pressure. The residue was purified on an Biotage automated chromatography system (Biotage 100 g HC column), eluting with a gradient of 0 to 30% methyl-t-butyl ether in heptanes to give the title compound (3.65 g, 83% yield) as a white solid. LCMS: m/z=423.1 [M+H]⁺.

### Example 95: tert-Butyl 3-((5-((1S,3R)-2-(2-fluoro-2-methylpropyl)-3-methyl-2,3,4,9-tetrahydro-1H-pyrido[3,4-b]indol-1-yl)thiazol-2-yl)methyl)azetidine-1-carboxylate

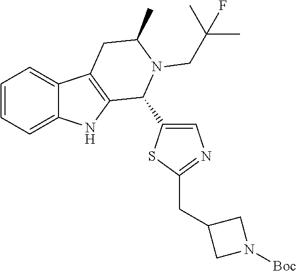 [SMILES: C[C@@H]1Cc2c([nH]c3ccccc23)[C@@H](c2cnc(CC3CN(C(=O)OC(C)(C)C)C3)s2)N1CC(C)(C)F]

To a 100 mL three-necked round bottom flask equipped with magnetic stirrer bar and thermocouple was added 2-bromo-5-((1S,3R)-2-(2-fluoro-2-methylpropyl)-3-methyl-2,3,4,9-tetrahydro-1H-pyrido[3,4-b]indol-1-yl)thiazole (940 mg, 2.22 mmol, 1.0 equiv), potassium (1-Boc-azetidin-3-yl)methyltrifluoroborate (924 mg, 3.34 mmol, 1.5 equiv), palladium(II) acetate (74.7 mg, 0.33 mmol, 0.15 equiv), RuPhos (308 mg, 0.66 mmol, 0.3 equiv), and potassium carbonate (1.53 g, 11.1 mmol, 5.0 equiv). A mixture of toluene (22 mL) and water (11 mL) was then added, and the mixture was sparged with nitrogen for 15 min. A condenser was attached, and the reaction was heated to 100° C. for 24 h. After cooling to room temperature, the mixture was filtered through a pad of Celite® (30 g), which was rinsed with ethyl acetate (3×30 mL). The filtrate was diluted with water (30 mL). The layers were separated, and the aqueous layer was extracted with ethyl acetate (3×20 mL). The combined organic layers were washed with saturated brine (30 mL), dried over sodium sulfate, filtered and concentrated under reduced pressure. The residue was purified on a Biotage automated chromatography system (Biotage® Sfar Silica HC 20 μm, 50 g), eluting with a gradient of 10 to 80% ethyl acetate in heptanes, to give the title compound (563 mg, 50% yield) as a brown solid. LCMS: m/z=513.3 [M+H]⁺.

### Example 96: 2-(Azetidin-3-ylmethyl)-5-((1S,3R)-2-(2-fluoro-2-methylpropyl)-3-methyl-2,3,4,9-tetrahydro-1H-pyrido[3,4-b]indol-1-yl)thiazole

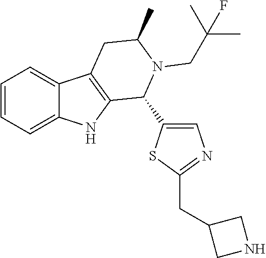 [SMILES: C[C@@H]1Cc2c([nH]c3ccccc23)[C@@H](c2cnc(CC3CNC3)s2)N1CC(C)(C)F]

Trifluoroacetic acid (2.5 mL, 33 mmol, 30 equiv) was added dropwise at room temperature to a solution of tert-butyl 3-((5-((1S,3R)-2-(2-fluoro-2-methylpropyl)-3-methyl-2,3,4,9-tetrahydro-1H-pyrido[3,4-b]indol-1-yl)thiazol-2-yl)methyl)azetidine-1-carboxylate (563 mg, 1.1 mmol, 1.0 equiv) in dichloromethane (10 mL). After stirring for 30 min at room temperature, the reaction was cooled to 0° C. and diluted with dichloromethane (30 mL). Saturated sodium carbonate solution was carefully added, and the resulting cloudy suspension was vigorously stirred for 5 min at room temperature. The layers were separated, and the aqueous layer was extracted with dichloromethane (3×10 mL). The combined organic layers were washed with 15% sodium hydroxide (2×20 mL) and saturated brine (20 mL), dried over sodium sulfate, filtered and concentrated under reduced pressure. The residue was dissolved in methanol (5 mL) and diluted with 6M sodium hydroxide to remove any trifluoroacetamide impurity. The mixture was stirred for 2 h at room temperature before the solvent was removed under reduced pressure. The residue was dissolved in dichloromethane (20 mL) and washed with water (10 mL) and saturated brine (10 mL), dried over sodium sulfate, filtered and concentrated under reduce pressure to give the title compound (428 mg, 95% yield) as a brown solid. LCMS: m/z=413.2 [M+H]⁺

### Example 97: 5-((1S,3R)-2-(2-Fluoro-2-methylpropyl)-3-methyl-2,3,4,9-tetrahydro-1H-pyrido[3,4-b]indol-1-yl)-2-((1-propylazetidin-3-yl)methyl)thiazole

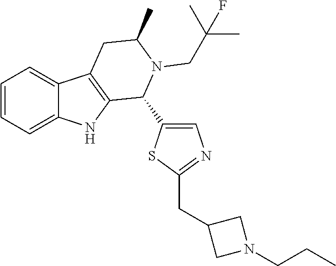 [SMILES: CCCN1CC(Cc2ncc([C@@H]3c4[nH]c5ccccc5c4C[C@@H](C)N3CC(C)(C)F)s2)C1]

Propanal (34 μL, 0.47 mmol, 1.5 equiv) and acetic acid (87 μL, 1.57 mmol, 5 equiv) were sequentially added to a solution of 2-(azetidin-3-ylmethyl)-5-((1S,3R)-2-(2-fluoro-2-methylpropyl)-3-methyl-2,3,4,9-tetrahydro-1H-pyrido[3,4-b]indol-1-yl)thiazole (0.13 g, 0.314 mmol, 1 equiv) in dichloromethane (6.5 mL). After stirring at room temperature for 30 min, sodium triacetoxyborohydride (0.2 g, 0.94 mmol, 3 equiv) was added portion-wise over 5 min and the resulting mixture was stirred at room temperature for 3.5 h. The reaction was quenched with saturated sodium bicarbonate (10 mL) and extracted with dichloromethane (2×15 mL). The combined organic layer was dried over sodium sulfate, filtered and concentrated under reduced pressure. The residue (70 mg) was purified on an Interchim automated chromatography system (Sorbtech 12 g silica-gel column, previously neutralized using 1% triethylamine in heptanes), eluting with a gradient of 0 to 100% ethyl acetate in dichloromethane, followed by a gradient of 5 to 10% methanol in dichloromethane.

The more polar product fractions were pooled and the solvents evaporated under reduced pressure to give product 5-((1S,3R)-2-(2-fluoro-2-methylpropyl)-3-methyl-2,3,4,9-tetrahydro-1H-pyrido[3,4-b]indol-1-yl)-2-((1-propylazetidin-3-yl)methyl)thiazole(19 mg, 13% yield) as a colorless oil. LCMS (ESI) m/z=455.3 (M+H)⁺; ¹H NMR (400 MHz, CDCl₃) δ=7.89 (br s, 1H), 7.52 (d, J=7.8 Hz, 1H), 7.34 (d, J=7.9 Hz, 1H), 7.23 (s, 1H), 7.20 (dt, J=1.2, 7.6 Hz, 1H), 7.16-7.09 (m, 1H), 5.27 (br s, 1H), 3.48 (br s, 2H), 3.39 (br s, 1H), 3.18 (d, J=6.2 Hz, 2H), 2.91 (br s, 3H), 2.71-2.56 (m, 3H), 2.56-2.49 (m, 1H), 2.38 (br t, J=7.5 Hz, 2H), 1.49 (d, J=21.8 Hz, 3H), 1.40-1.29 (m, 5H), 1.15 (d, J=6.8 Hz, 3H), 0.88 (t, J=7.4 Hz, 3H).

### Example 98: 5-((1S,3R)-2-(2-Fluoro-2-methylpropyl)-3-methyl-2,3,4,9-tetrahydro-1H-pyrido[3,4-b]indol-1-yl)-2-((1-(3-fluoropropyl)azetidin-3-yl)methyl)thiazole

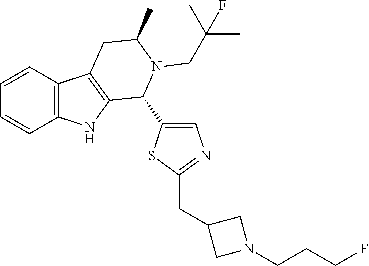 [SMILES: C[C@@H]1Cc2c([nH]c3ccccc23)[C@@H](c2cnc(CC3CN(CCCF)C3)s2)N1CC(C)(C)F]

N,N-Diisopropylethylamine (0.27 mL, 1.56 mmol, 1.5 equiv) and 1-bromo-3-fluoropropane (176 mg, 1.25 mmol, 1.2 equiv) were sequentially added to a solution of 2-(azetidin-3-ylmethyl)-5-((1S,3R)-2-(2-fluoro-2-methylpropyl)-3-methyl-2,3,4,9-tetrahydro-1H-pyrido[3,4-b]indol-1-yl)thiazole (428 mg, 1.04 mmol, 1.0 equiv) in N,N-dimethylacetamide (3.0 mL) at room temperature. After stirring for 16 h at room temperature, sodium bicarbonate (60 mL) was added. The layers were separated, and the aqueous layer was extracted with ethyl acetate (3×30 mL). The combined organic layers were washed with water (30 mL) and saturated brine (30 mL), dried over sodium sulfate, filtered and concentrated under reduced pressure. The residue was divided into two equal portions, and each was purified on a Biotage automated chromatography system (Biotage KP-amino-D column 25 g), eluting with a gradient of 0 to 100% ethyl acetate in dichloromethane, to give the title compound (38.1 mg, 15% yield) as a brown solid. ¹H NMR (400 MHz, CDCl₃) δ=8.11 (br s, 1H), 7.52 (d, J=7.8 Hz, 1H), 7.34 (d, J=8.3 Hz, 1H), 7.24-7.08 (m, 3H), 5.26 (br d, J=1.8 Hz, 1H), 4.52 (t, J=6.0 Hz, 1H), 4.40 (t, J=6.1 Hz, 1H), 3.53-3.43 (m, 2H), 3.38 (br s, 1H), 3.18 (d, J=6.7 Hz, 2H), 2.94-2.81 (m, 3H), 2.69-2.49 (m, 6H), 1.79-1.55 (m, 3H), 1.55-1.44 (m, 3H), 1.36-1.28 (m, 2H), 1.15 (d, J=6.8 Hz, 3H); LCMS: m/z=473.3 [M+H]⁺.

### Example 99: ((I-(tert-Butoxycarbonyl)azetidin-3-yl)methyl)zinc(II) iodide

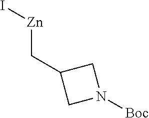

Zinc dust (2.86 g, 43.81 mmol, 1.3 equiv) was added at room temperature to a mixture of chlorotrimethylsilane (0.37 g, 0.428 ml, 3.37 mmol, 0.1 equiv) and dibromoethane (0.63 g, 0.29 mL, 3.37 mmol, 0.1 equiv) in N,N-dimethylacetamide (10 mL). After stirring for 20 minutes, a solution of tert-butyl 3-(iodomethyl)azetidine-1-carboxylate (10 g, 33.7 mmol, 1.0 equiv) in N,N-dimethylacetamide (14 mL) was slowly added at room temperature. The mixture was stirred for additional 24 hours at room temperature then used subsequently.

### Example 100: Methyl 2-((1-(tert-butoxycarbonyl)azetidin-3-yl)methyl)thiazole-S-carboxylate

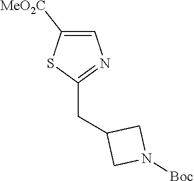 [SMILES: COC(=O)c1cnc(CC2CN(C(=O)OC(C)(C)C)C2)s1]

A mixture of methyl 2-bromothiazole-5-carboxylate (4.68 g, 21.06 mmol, 1.0 equiv), tetrakis(triphenylphosphine)palladium(O) (1.21 g, 1.05 mmol, 0.05 equiv) and copper iodide (0.46 g, 1.05 mmol, 0.1 equiv) in anhydrous N,N-dimethylacetamide (70 mL) was sparged with nitrogen for 20 minutes. A solution of ((1-(tert-butoxycarbonyl)azetidin-3-yl)methyl)zinc(II) iodide (prepared in previous step, 33.7 mmol, 1.6 equiv) was added and the reaction mixture was sparged for additional 5 minutes. After heating at 60° C. for 2 hours, the mixture was cooled to room temperature and diluted with water (100 mL). The resulting slurry was filtered through a pad of Celite®, and the solid was rinsed with ethyl acetate (3×50 mL). The filtrate was separated and the aqueous layer was extracted with ethyl acetate (3×70 mL). The combined organic layers were washed with water (100 mL), saturated brine (100 mL), dried over sodium sulfate, filtered and concentrated under reduced pressure. The residue was purified on a Biotage automated chromatography system (Biotage 200 g HC column), eluting with a gradient of 0 to 80% ethyl acetate in heptanes, to give the title compound (5.48 g, 83% yield) as a tan solid. LCMS: m/z=313.1 [M+H]⁺.

### Example 101: Methyl 2-(azetidin-3-ylmethyl)thiazole-5-carboxylate

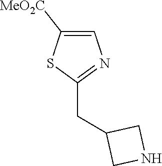 [SMILES: COC(=O)c1cnc(CC2CNC2)s1]

Trifluoroacetic acid (6.28 mL, 82.1 mmol, 10 equiv) was added at room temperature to a stirred solution of methyl 2-((1-(tert-butoxycarbonyl)azetidin-3-yl)methyl)thiazole-5-carboxylate (2.56 g, 8.21 mmol, 1 equiv) in dichloromethane (30 mL). The reaction was stirred at room temperature for 1 hour. The reaction mixture was concentrated under reduced pressure then suspended in methanol (20 mL). MP-carbonate resin (21.4 g, 3 equiv. in respective to loading capacity of trifluoroacetic acid) was added at 0° C. and the mixture was stirred for 1 hour. The suspension was then filtered and the solid was rinsed with dichloromethane (2×20 mL). The filtrate was concentrated under reduced pressure to give the title compound (2.67 g, >95% yield) as brown oil. LCMS: m/z=213.1 [M+H]⁺.

### Example 102: Methyl 2-((1-(3-fluoropropyl)azetidin-3-yl)methyl)thiazole-5-carboxylate

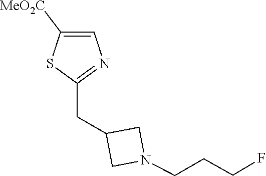 [SMILES: COC(=O)c1cnc(CC2CN(CCCF)C2)s1]

Potassium carbonate (2.8 g, 20.53 mmol, 2.5 equiv) and 1-bromo-3-fluoropropane (1.27 g, 9.03 mmol, 1.1 equiv) were sequentially added at room temperature to a solution of methyl 2-(azetidin-3-ylmethyl)thiazole-5-carboxylate (1.74 g, 8.21 mmol, 1.0 equiv) in acetonitrile (20 mL). After stirring for 16 hours at room temperature, reaction was concentrated under reduced pressure. The residue was diluted with water (50 mL) and ethyl acetate (50 mL). The layers were separated and the aqueous layer was extracted with ethyl acetate (3×30 mL). The combined organic layers were washed with water (30 mL) and saturated brine (30 mL), dried over sodium sulfate, filtered and concentrated under reduced pressure. The resulting residue was purified on Biotage automated chromatography system (Biotage 55 g KPNH), eluting with a gradient of 10 to 80% ethyl acetate in heptane, to give title compound (0.58 g, 26% yield) as yellow oil. LCMS: m/z=273.2 [M+H]⁺.

### Example 103: (2-((1-(3-Fluoropropyl)azetidin-3-yl)methyl)thiazol-5-yl)methanol

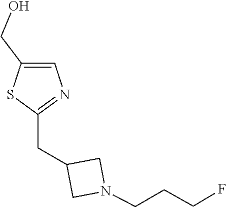 [SMILES: OCc1cnc(CC2CN(CCCF)C2)s1]

1M Lithium aluminum hydride in tetrahydrofuran (2.61 mL, 1 equiv) was slowly added at 0° C. to a solution of methyl 2-((1-(3-fluoropropyl)azetidin-3-yl)methyl)thiazole-5-carboxylate (0.710 g, 2.61 mmol, 1 equiv) in tetrahydrofuran (13 mL) under nitrogen. After stirring for 1 hour, water (0.1 mL), 15% sodium hydroxide solution (0.1 mL) and water (0.3 mL) were sequentially added at 0° C. The mixture was vigorously stirred for 15 minutes at 0° C., then allowed to warm to room temperature and stirred for additional 15 minutes. Magnesium sulfate was added to the resulting suspension which was stirred for an additional 10 minutes. The mixture was filtered and the solid was washed with ethyl acetate (2×10 mL). The filtrate was concentrated under reduced pressure to give the title compound (0.63 g, over 95% yield) as yellow oil. LCMS: m/z=245.1 [M+H]⁺.

### Example 104: 2-((1-(3-Fluoropropyl)azetidin-3-yl)methyl)thiazole-5-carbaldehyde

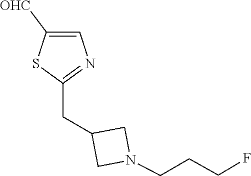 [SMILES: O=Cc1cnc(CC2CN(CCCF)C2)s1]

Activated manganese dioxide (2.24 g, 25.8 mmol, 10 equiv) was added at room temperature to a solution of (2-((1-(3-fluoropropyl)azetidin-3-yl)methyl)thiazol-5-yl)methanol (0.63 g, 2.58 mmol, 1 equiv) in dichloromethane (12 mL) and the mixture was stirred for 24 hours. The reaction mixture was filtered through Celite® (20 g) and the solid was washed with dichloromethane (3×25 mL). The filtrate was concentrated under reduced pressure to give the title compound (0.52 g, 84% yield) as yellow oil. LCMS: m/z=243.1 [M+H]⁺.

### Example 105: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-((1-(3-fluoropropyl)azetidin-3-yl)methyl)thiazole

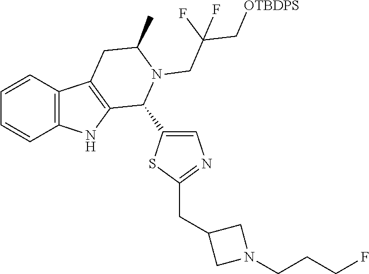

A mixture of 2-((1-(3-fluoropropyl)azetidin-3-yl)methyl)thiazole-5-carbaldehyde (0.2 g, 0.83 mmol, 1 equiv), (R)—N-(1-(1H-indol-3-yl)propan-2-yl)-3-((tert-butyldiphenylsilyl)oxy)-2,2-difluoropropan-1-amine (0.51 g, 0.996 mmol, 1.2 equiv) and acetic acid (0.09 mL, 1.66 mmol, 2.0 equiv) in toluene (0.8 mL) was heated at 80° C. in for 2 hours. After cooling to room temperature, the mixture was diluted with ethyl acetate (20 mL) and washed with saturated sodium bicarbonate (20 mL). The aqueous layer was extracted with ethyl acetate (3×10 mL). The combined organic layers were washed with saturated brine (10 mL), dried over sodium sulfate, filtered and concentrated under reduced pressure. The crude residue was purified on a Biotage automated chromatography system (Biotage 28 g KPNH), eluting with a gradient of 0 to 80% ethyl acetate in heptanes, to give the title compound (264 mg, 44% yield) as white solid. LCMS: m/z=731.3 [M+H]⁺.

### Example 106: 2,2-Difluoro-3-((1S,3R)-1-(2-((1-(3-fluoropropyl)azetidin-3-yl)methyl)thiazol-5-yl)-3-methyl-1,3,4,9-tetrahydro-2H-pyrido[3,4-b]indol-2-yl)propan-1-ol

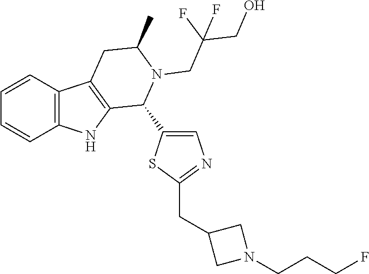 [SMILES: C[C@@H]1Cc2c([nH]c3ccccc23)[C@@H](c2cnc(CC3CN(CCCF)C3)s2)N1CC(F)(F)CO]

1M Tetra-N-butylammonium fluoride in THE (0.35 mL, 0.347 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-((1-(3-fluoropropyl)azetidin-3-yl)methyl)thiazole (0.23 g, 0.315 mmol, 1 equiv) in THF (2.9 mL). After stirring for 1 hour, the reaction was concentrated under reduced pressure. The residue was diluted with saturated ammonium chloride (5 mL). The suspension was extracted with ethyl acetate (3×5 mL). The combined organic layers were dried over sodium sulfate, filtered and concentrated under reduced pressure. The crude residue was purified on Biotage automated chromatography system (Biotage 11 g KPNH) eluting with a gradient of 5 to 100% ethyl acetate in dichloromethane to give the title compound (123.4 mg, 80% yield) as white solid. ¹H NMR (400 MHz, CDCl₃) δ=9.44 (br s, 1H), 7.51 (d, J=7.7 Hz, 1H), 7.33 (d, J=8.1 Hz, 1H), 7.19-7.08 (m, 3H), 5.42 (s, 1H), 5.23 (s, 1H), 4.53 (t, J=5.9 Hz, 1H), 4.41 (t, J=5.9 Hz, 1H), 4.02-3.91 (m, 1H), 3.88-3.78 (m, 1H), 3.49-3.39 (m, 2H), 3.39-3.18 (m, 3H), 3.16-3.06 (m, 2H), 3.03-2.83 (m, 4H), 2.72-2.64 (m, 1H), 2.63-2.52 (m, 3H), 1.80-1.67 (m, 2H), 1.26 (br t, J=7.2 Hz, 2H), 1.22 (d, J=6.8 Hz, 3H): LCMS: m/z=493.2 [M+H]⁺.

### Example 107: 5-((1S,3R)-2-(2,2-Difluoroethyl)-3-methyl-2,3,4,9-tetrahydro-1H-pyrido[3,4-b]indol-1-yl)-2-((1-(3-fluoropropyl)azetidin-3-yl)methyl)thiazole

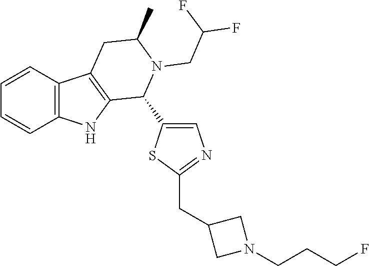 [SMILES: C[C@@H]1Cc2c([nH]c3ccccc23)[C@@H](c2cnc(CC3CN(CCCF)C3)s2)N1CC(F)F]

A mixture of 2-((1-(3-fluoropropyl)azetidin-3-yl)methyl)thiazole-5-carbaldehyde (0.05 g, 0.219 mmol, 1 equiv), (R)—N-(2,2-difluoroethyl)-1-(1H-indol-3-yl)propan-2-amine (0.06 g, 0.262 mmol, 1.2 equiv) and acetic acid (0.045 mL, 0.786 mmol, 3 equiv) in toluene (1 mL) was heated at 80° C. in for 2 hours. After cooling to room temperature, the mixture was diluted with ethyl acetate (20 mL) and washed with saturated sodium bicarbonate (20 mL). The aqueous layer was extracted with ethyl acetate (3×10 mL). The combined organic layers were washed with saturated brine (10 mL), dried over sodium sulfate, filtered and concentrated under reduced pressure. The residue was purified on an Biotage automated chromatography system (Biotage 11 g KPNH), eluting with a gradient of 0 to 80% ethyl acetate in hexanes to give the title compound (6.5 mg, 6% yield) as yellow solid. ¹H NMR (400 MHz, CDCl₃) δ=8.13-7.69 (m, 1H), 7.53 (d, J=7.7 Hz, 1H), 7.38 (d, J=1.0 Hz, 1H), 7.33 (d, J=7.9 Hz, 1H), 7.23-7.12 (m, 2H), 5.97-5.63 (m, 1H), 5.14 (s, 1H), 4.53 (t, J=6.1 Hz, 1H), 4.41 (t, J=6.1 Hz, 1H), 3.52-3.33 (m, 3H), 3.20 (d, J=7.3 Hz, 2H), 3.11-2.99 (m, 1H), 2.98-2.70 (m, 5H), 2.66-2.46 (m, 3H), 1.79-1.66 (m, 2H), 1.20 (d, J=6.7 Hz, 3H): LCMS: m/z=463.2 [M+H]⁺.

### Example 108: 2-((1-(3-Fluoropropyl)azetidin-3-yl)methyl)-5-((1S,3R)-3-methyl-2-(2,2,2-trifluoroethyl)-2,3,4,9-tetrahydro-1H-pyrido[3,4-b]indol-1-yl)thiazole

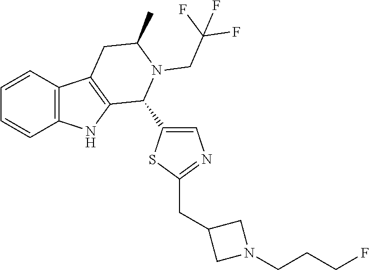 [SMILES: C[C@@H]1Cc2c([nH]c3ccccc23)[C@@H](c2cnc(CC3CN(CCCF)C3)s2)N1CC(F)(F)F]

A mixture of 2-((1-(3-fluoropropyl)azetidin-3-yl)methyl)thiazole-5-carbaldehyde (0.09 g, 0.372 mmol, 1 equiv), (R)-1-(1H-indol-3-yl)-N-(2,2,2-trifluoroethyl)propan-2-amine (0.11 g, 0.446 mmol, 1.2 equiv) and acetic acid (0.04 mL, 0.744 mmol, 2 equiv) in toluene (0.8 mL) was heated at 80° C. in for 2 hours. After cooling to room temperature, the mixture was diluted with ethyl acetate (10 mL) and washed with saturated sodium bicarbonate (10 mL). The aqueous layer was extracted with ethyl acetate (3×10 mL). The combined organic layers were washed with saturated brine (10 mL), dried over sodium sulfate, filtered and concentrated under reduced pressure. The residue was purified on a Biotage automated chromatography system (Biotage 11 g KPNH) eluting with a gradient of 0 to 80% ethyl acetate in hexanes, to give the title compound (39.7 mg, 22% yield) as white solid. ¹H NMR (400 MHz, CDCl₃-d)) δ=8.00 (s, 1H), 7.52 (d, J=7.7 Hz, 1H), 7.34 (d, J=8.1 Hz, 1H), 7.27 (d, J=1.1 Hz, 1H), 7.22 (dt, J=1.2, 7.6 Hz, 1H), 7.17-7.11 (m, 1H), 5.54 (s, 1H), 5.16 (s, 1H), 4.52 (t, J=6.0 Hz, 1H), 4.40 (t, J=6.0 Hz, 1H), 3.47-3.37 (m, 3H), 3.31-3.16 (m, 3H), 3.02-2.82 (m, 4H), 2.77 (dd, J=4.3, 16.1 Hz, 1H), 2.58-2.49 (m, 3H), 1.78-1.64 (m, 3H), 1.22 (d, J=6.7 Hz, 3H): LCMS: m/z=481.2 [M+H]⁺.

### Example 109: 5-((1S,3R)-2-(2,2-Difluoropropyl)-3-methyl-2,3,4,9-tetrahydro-1H-pyrido[3,4-b]indol-1-yl)-2-((1-(3-fluoropropyl)azetidin-3-yl)methyl)thiazole

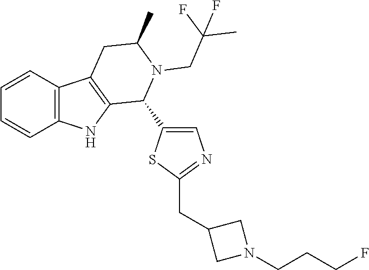 [SMILES: C[C@@H]1Cc2c([nH]c3ccccc23)[C@@H](c2cnc(CC3CN(CCCF)C3)s2)N1CC(C)(F)F]

A mixture of 2-((1-(3-fluoropropyl)azetidin-3-yl)methyl)thiazole-5-carbaldehyde (0.09 g, 0.372 mmol, 1 equiv), (R)—N-(1-(1H-indol-3-yl)propan-2-yl)-2,2-difluoropropan-1-amine (0.11 g, 0.446 mmol, 1.2 equiv) and acetic acid (0.04 mL, 0.744 mmol, 2 equiv) in toluene (1.3 mL) was heated at 80° C. in for 2 hours. After cooling to room temperature, the mixture was diluted with ethyl acetate (10 mL) and washed with saturated sodium bicarbonate (10 mL). The aqueous layer was extracted with ethyl acetate (3×10 mL). The combined organic layers were washed with saturated brine (10 mL), dried over sodium sulfate, filtered and concentrated under reduced pressure. The residue was purified on a Biotage automated chromatography system (Biotage 11 g KPNH), eluting with a gradient of 0 to 80% ethyl acetate in hexanes, to give the title compound (45.1 mg, 25% yield) as white solid. ¹H NMR (400 MHz, CDCl₃) δ=8.06 (s, 1H), 7.52 (d, J=7.7 Hz, 1H), 7.36-7.31 (m, 1H), 7.24 (d, J=1.1 Hz, 1H), 7.20 (dt, J=1.2, 7.6 Hz, 1H), 7.16-7.09 (m, 1H), 5.19 (s, 1H), 4.52 (t, J=6.0 Hz, 1H), 4.40 (t, J=6.0 Hz, 1H), 3.45 (t, J=6.9 Hz, 2H), 3.42-3.34 (m, 1H), 3.19 (d, J=6.8 Hz, 2H), 2.98-2.83 (m, 4H), 2.82-2.65 (m, 2H), 2.58-2.48 (m, 3H), 1.79-1.67 (m, 5H), 1.19 (d, J=6.7 Hz, 3H): LCMS: m/z=477.3 [M+H]⁺.

### Example 110: Methyl (R)-2-((1-(tert-butoxycarbonyl)pyrrolidin-3-yl)methyl)thiazole-5-carboxylate

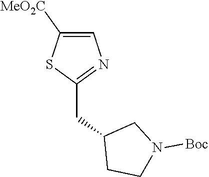 [SMILES: COC(=O)c1cnc(C[C@H]2CCN(C(=O)OC(C)(C)C)C2)s1]

A mixture of methyl 2-bromothiazole-5-carboxylate (2.5 g, 11.25 mmol, 1.0 equiv), tetrakis(triphenylphosphine)palladium(O) (0.66 g, 0.562 mmol, 0.05 equiv), and copper iodide (215 mg, 1.125 mmol, 0.1 equiv) in anhydrous dimethylacetamide (22.5 mL) was sparged with nitrogen for 10 min. A 0.5 M solution of (S)-((1-(tert-butoxycarbonyl)pyrrolidin-3-yl)methyl)zinc(II) iodide in tetrahydrofuran (36 mL, 18 mmol, 1.6 equiv) was added. After stirring at 65° C. for 18 h, the reaction mixture was cooled to room temperature and diluted with ethyl acetate (500 mL), and water (400 mL). The layers were separated, and the aqueous layer was extracted with ethyl acetate (2×800 mL). The combined organic layers were washed with saturated brine (400 mL), dried over sodium sulfate, filtered, and concentrated under reduced pressure. The residue was purified on an Interchim automated chromatography system (Sorbtech 220 g silica gel column), eluting with a gradient of 0 to 50% ethyl acetate in heptanes to give the title compound (1.63 g, 44% yield) as a yellow/orange semi-solid. LCMS (ESI) m/z=327.1 [M+H]⁺.

### Example 111: Methyl (R)-2-(pyrrolidin-3-ylmethyl)thiazole-5-carboxylate

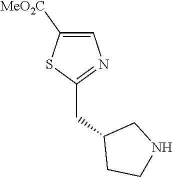 [SMILES: COC(=O)c1cnc(C[C@H]2CCNC2)s1]

Trifluoroacetic acid (6 mL, 75.9 mmol, 16.5 equiv) was added over 5 min to a stirred solution of methyl (R)-2-((1-(tert-butoxycarbonyl)pyrrolidin-3-yl)methyl)thiazole-5-carboxylate (1.5 g, 4.6 mmol, 1 equiv) in anhydrous dichloromethane (24 mL) at 0° C. The resulting mixture was stirred at 0° C. for 3.5 h. The volatiles were evaporated under reduced pressure. The residue was dissolved in methanol (10 mL) and cooled to 0° C. (ice bath). MP-carbonate resin (3 g, ˜2 equiv) was added at 0° C. The ice bath was removed, and the mixture was stirred at room temperature for 2 h. The resin was filtered off, and the filtrate was concentrated under reduced pressure. ¹H-NMR and ¹⁹F-NMR analysis of the residue indicated presence of trifluoroacetic acid. The residue was dissolved again in methanol (10 mL) and cooled to 0° C. (ice bath). MP-carbonate resin (3 g, ˜2 equiv) was added at 0° C. The ice bath was removed, and the mixture was stirred at room temperature for 2 h. The resin was filtered off, and the filtrate was concentrated under reduced pressure. ¹H-NMR and ¹⁹F-NMR analysis of the residue indicated that all trifluoroacetic acid was removed. The residue was dried under vacuum at room temperature overnight to give crude title compound (1.5 g) as a light tan foam, which was used subsequently. LCMS (ESI) m/z=227.1 [M+H]⁺.

### Example 112: Methyl (R)-2-((1-(3-fluoropropyl)pyrrolidin-3-yl)methyl)thiazole-5-carboxylate

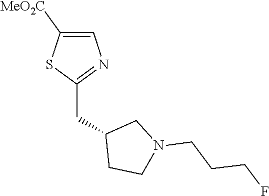 [SMILES: COC(=O)c1cnc(C[C@H]2CCN(CCCF)C2)s1]

1-Bromo-3-fluoropropane (0.93 g, 6.63 mmol, 1.5 equiv) and diisopropylethylamine (3.85 mL, 22.12 mmol, 5 equiv) were added to a solution of methyl (R)-2-(pyrrolidin-3-ylmethyl)thiazole-5-carboxylate (1.00 g, 4.42 mmol, 1 equiv) in N,N-dimethylacetamide (40 mL). The resulting mixture was stirred at room temperature for 48 h. The reaction mixture was diluted with ethyl acetate (100 mL) then washed with water (3×100 mL) and saturated brine (100 mL). The organic layer was dried over sodium sulfate, filtered and concentrated under reduced pressure to give the crude title compound (0.8 g, 61% yield over 2 steps) as a brown oil. LCMS (ESI) m/z=287.2 [M+H]⁺.

### Example 113: (R)-(2-((1-(3-Fluoropropyl)pyrrolidin-3-yl)methyl)thiazol-5-yl)methanol

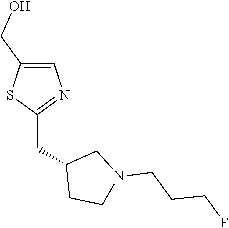 [SMILES: OCc1cnc(C[C@H]2CCN(CCCF)C2)s1]

1 M Lithium aluminum hydride in THF (4.2 mL, 4.2 mmol, 1.5 equiv) was added to a solution of methyl (R)-2-((1-(3-fluoropropyl)pyrrolidin-3-yl)methyl)thiazole-5-carboxylate (0.8 g, 2.78 mmol, 1 equiv) in anhydrous THF (8 mL) at 0° C. under nitrogen. Upon full addition of the lithium aluminum hydride solution, the reaction was warmed to room temperature and stirred for 1 h. The reaction was diluted with additional THF (80 mL) and cooled to 0° C. Sodium sulfate decahydrate (4 g) was cautiously added portion-wise. After stirring at room temperature for 15 min, the slurry was filtered off washing the solid with dichloromethane (2×50 mL). The filtrate was concentrated under reduced pressure to give the title compound (0.55 g, 79% yield) as a light-yellow oil. LCMS (ESI) m/z=259.2 [M+H]F.

### Example 114: (R)-2-((1-(3-Fluoropropyl)pyrrolidin-3-yl)methyl)thiazole-5-carbaldehyde

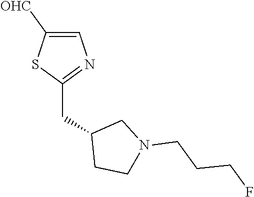 [SMILES: O=Cc1cnc(C[C@H]2CCN(CCCF)C2)s1]

Activated manganese dioxide (1.81 g, 10 equiv) was added to a solution of (R)-(2-((1-(3-fluoropropyl)pyrrolidin-3-yl)methyl)thiazol-5-yl)methanol (0.55 g, 2.12 mmol, 1 equiv) in dichloromethane (6.0 mL) and the resulting mixture was stirred at room temperature for 24 h. LCMS analysis at this time indicated 70% conversion to the desired product. Additional activated manganese dioxide (1.81 g, 10 equiv) was added, and the resulting mixture was stirred at room temperature for an additional 7 h. LCMS analysis at this time indicated full conversion to the desired product. The reaction mixture was passed through a Celite® pad (5 g), which was rinsed with dichloromethane (2×30 mL). The filtrate was concentrated under reduced pressure to give the title compound (0.38 g, 69% yield) as a brown oil. LCMS (ESI) m/z=257.2 [M+H]⁺.

### Example 115: 5-((1S,3R)-2-(2-Fluoro-2-methylpropyl)-3-methyl-2,3,4,9-tetrahydro-1H-pyrido[3,4-b]indol-1-yl)-2-(((R)-1-(3-fluoropropyl)pyrrolidin-3-yl)methyl) thiazole

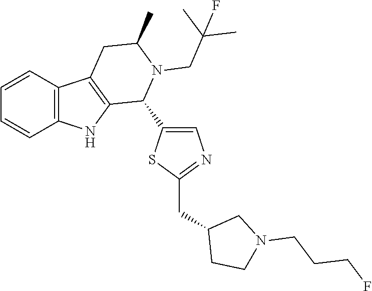 [SMILES: C[C@@H]1Cc2c([nH]c3ccccc23)[C@@H](c2cnc(C[C@H]3CCN(CCCF)C3)s2)N1CC(C)(C)F]

A mixture of (R)-2-((1-(3-fluoropropyl)pyrrolidin-3-yl)methyl)thiazole-5-carbaldehyde (100 mg, 0.39 mmol, 1 equiv), (R)—N-(1-(1H-indol-3-yl)propan-2-yl)-2-fluoro-2-methylpropan-1-amine (116 mg, 0.47 mmol, 1.2 equiv) and acetic acid (53 μL, 0.94 mmol, 2 equiv) in anhydrous toluene (0.75 mL) was heated at 80° C. for 15 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (60 mg, 39% yield) as a light-yellow solid. LCMS (ESI) m/z=487.3 [M+H]⁺; ¹H NMR (400 MHz, CDCl₃) δ=7.93 (br s, 1H), 7.53 (d, J=7.8 Hz, 1H), 7.36 (d, J=7.5 Hz, 1H), 7.27-7.17 (m, 2H), 7.17-7.09 (m, 1H), 5.28 (br s, 1H), 4.57 (t, J=6.1 Hz, 1H), 4.45 (t, J=6.1 Hz, 1H), 3.40 (br s, 1H), 3.09-2.95 (m, 2H), 2.81-2.75 (m, 1H), 2.74-2.45 (m, 9H), 2.29 (dd, J=6.4, 9.2 Hz, 1H), 2.11-2.01 (m, 1H), 1.95-1.81 (m, 2H), 1.62-1.54 (m, 3H), 1.54-1.44 (m, 3H), 1.36-1.30 (m, 3H), 1.16 (d, J=6.7 Hz, 3H).

### Example 116: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((R)-1-(3-fluoropropyl)pyrrolidin-3-yl)methyl)thiazole

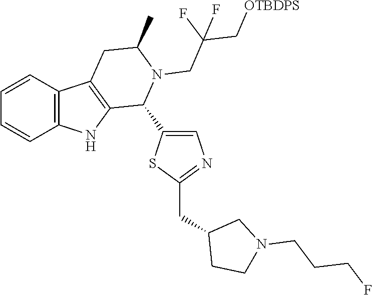

A mixture of (R)-2-((1-(3-fluoropropyl)pyrrolidin-3-yl)methyl)thiazole-5-carbaldehyde (93 mg, 0.36 mmol, 1 equiv), (R)—N-(1-(1H-indol-3-yl)propan-2-yl)-3-((tert-butyldiphenylsilyl)oxy)-2,2-difluoropropan-1-amine (218 mg, 0.43 mmol, 1.2 equiv) and acetic acid (41.5 μL, 0.72 mmol, 2 equiv) in anhydrous toluene (0.75 mL) was heated at 80° C. for 8 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (120 mg, 45% yield) as a yellow oil. LCMS (ESI) m/z=745.3 [M+H]⁺.

### Example 117: 2,2-Difluoro-3-((1S,3R)-1-(2-(((R)-1-(3-fluoropropyl)pyrrolidin-3-yl)methyl)thiazol-5-yl)-3-methyl-1,3,4,9-tetrahydro-2H-pyrido[3,4-b]indol-2-yl)propan-1-ol

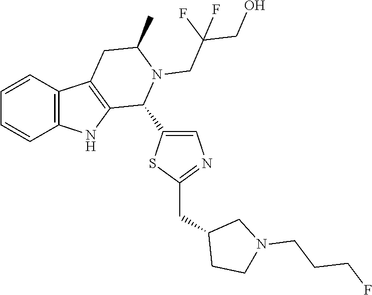 [SMILES: C[C@@H]1Cc2c([nH]c3ccccc23)[C@@H](c2cnc(C[C@H]3CCN(CCCF)C3)s2)N1CC(F)(F)CO]

1 M Tetrabutyl ammonium fluoride in THF (0.16 mmol, 1 equiv) was added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-1-(3-fluoropropyl)pyrrolidin-3-yl)methyl)thiazole (120 mg, 0.16 mmol) in THF (1.2 mL). The resulting mixture was stirred at room temperature for 4 h. The reaction was cooled down to 0° C. (ice bath) and water (5 mL) was added, followed by dichloromethane (5 mL). The layers were separated, and the aqueous layer was extracted with dichloromethane (2×5 mL). The combined organic layers were dried over sodium sulfate, filtered, and concentrated under reduced pressure. The residue was purified on an Interchim automated chromatography system (11 g Biotage 50 μm KP-amino-d column), eluting with a gradient of 0 to 100% ethyl acetate in heptanes to give the title compound (35 mg, 43% yield) as a light tan solid. LCMS (ESI) m/z=507.3 [M+H]⁺; ¹H NMR (400 MHz, CDCl₃) δ=8.61 (br s, 1H), 7.52 (d, J=7.8 Hz, 1H), 7.33 (d, J=7.9 Hz, 1H), 7.24-7.09 (m, 3H), 5.25 (s, 1H), 4.55 (t, J=6.0 Hz, 1H), 4.43 (t, J=6.0 Hz, 1H), 4.00-3.83 (m, 2H), 3.47-3.38 (m, 1H), 3.20-3.03 (m, 2H), 2.99-2.89 (m, 2H), 2.81-2.71 (m, 2H), 2.68-2.51 (m, 6H), 2.28 (dd, J=6.7, 9.2 Hz, 1H), 2.07-1.80 (m, 4H), 1.56 (tdd, J=6.5, 8.1, 12.9 Hz, 1H), 1.23 (d, J=6.7 Hz, 3H).

### Example 118: 5-((1S,3R)-2-(2,2-Difluoroethyl)-3-methyl-2,3,4,9-tetrahydro-1H-pyrido[3,4-b]indol-1-yl)-2-(((R)-1-(3-fluoropropyl)pyrrolidin-3-yl)methyl)thiazole

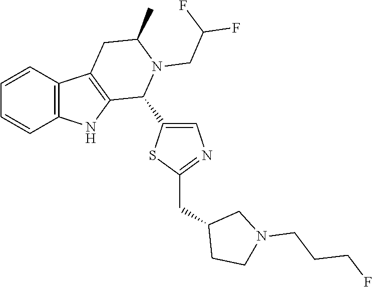 [SMILES: C[C@@H]1Cc2c([nH]c3ccccc23)[C@@H](c2cnc(C[C@H]3CCN(CCCF)C3)s2)N1CC(F)F]

A mixture of (R)-2-((1-(3-fluoropropyl)pyrrolidin-3-yl)methyl)thiazole-5-carbaldehyde (93 mg, 0.36 mmol, 1 equiv), (R)—N-(2,2-difluoroethyl)-1-(1H-indol-3-yl)propan-2-amine (116 mg, 0.43 mmol, 1.2 equiv) and acetic acid (41.5 μL, 0.72 mmol, 2 equiv) in anhydrous toluene (0.75 mL) was heated at 80° C. for 3 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (62 mg, 36% yield) as a yellow solid. LCMS (ESI) m/z=477.3 [M+H]⁺; ¹H NMR (400 MHz, CDCl₃) δ=7.94 (s, 1H), 7.52 (d, J=7.7 Hz, 1H), 7.40-7.27 (m, 2H), 7.20 (dt, J=1.2, 7.6 Hz, 1H), 7.16-7.10 (m, 1H), 5.80 (ddt, J=3.9, 4.9, 56.5 Hz, 1H), 5.13 (s, 1H), 4.55 (t, J=6.0 Hz, 1H), 4.43 (t, J=6.1 Hz, 1H), 3.50-3.34 (m, 1H), 3.11-2.97 (m, 3H), 2.86-2.72 (m, 3H), 2.66-2.48 (m, 6H), 2.28 (dd, J=6.3, 9.1 Hz, 1H), 2.09-2.00 (m, 1H), 1.93-1.80 (m, 2H), 1.55 (tdd, J=6.3, 8.1, 12.7 Hz, 1H), 1.19 (d, J=6.7 Hz, 3H).

### Example 119: 2-(((R)-1-(3-Fluoropropyl)pyrrolidin-3-yl)methyl)-5-((1S,3R)-3-methyl-2-(2,2,2-trifluoroethyl)-2,3,4,9-tetrahydro-1H-pyrido[3,4-b]indol-1-yl)thiazole

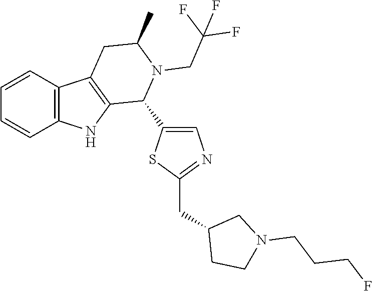 [SMILES: C[C@@H]1Cc2c([nH]c3ccccc23)[C@@H](c2cnc(C[C@H]3CCN(CCCF)C3)s2)N1CC(F)(F)F]

A mixture of (R)-2-((1-(3-fluoropropyl)pyrrolidin-3-yl)methyl)thiazole-5-carbaldehyde (93 mg, 0.36 mmol, 1 equiv), (R)—N-(2,2,2-trifluoroethyl)-1-(1H-indol-3-yl)propan-2-amine (110 mg, 0.43 mmol, 1.2 equiv) and acetic acid (41.5 μL, 0.72 mmol, 2 equiv) in anhydrous toluene (0.75 mL) was heated at 80° C. for 13 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (65 mg, 36% yield) as a yellow solid. LCMS (ESI) m/z=495.3 [M+H]⁺; ¹H NMR (400 MHz, CDCl₃) δ=8.01 (s, 1H), 7.53 (d, J=7.7 Hz, 1H), 7.34 (d, J=8.1 Hz, 1H), 7.27 (d, J=1.0 Hz, 1H), 7.24-7.19 (m, 1H), 7.17-7.12 (m, 1H), 5.54 (s, 1H), 5.16 (s, 1H), 4.55 (t, J=6.0 Hz, 1H), 4.44 (t, J=6.1 Hz, 1H), 3.47-3.38 (m, 1H), 3.25 (qd, J=9.5, 15.6 Hz, 1H), 3.05-2.91 (m, 3H), 2.80-2.73 (m, 2H), 2.67-2.50 (m, 6H), 2.29 (dd, J=6.4, 9.0 Hz, 1H), 2.10-2.00 (m, 1H), 1.94-1.80 (m, 2H), 1.60-1.51 (m, 1H), 1.22 (d, J=6.7 Hz, 3H).

### Example 120: Methyl (S)-2-((1-(tert-butoxycarbonyl)pyrrolidin-3-yl)methyl)thiazole-5-carboxylate

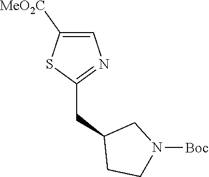 [SMILES: COC(=O)c1cnc(C[C@@H]2CCN(C(=O)OC(C)(C)C)C2)s1]

A mixture of methyl 2-bromothiazole-5-carboxylate (5.0 g, 22.5 mmol, 1.0 equiv), tetrakis(triphenylphosphine)palladium(O) (1.32 g, 1.125 mmol, 0.05 equiv), and copper iodide (0.43 g, 2.25 mmol, 0.1 equiv) in anhydrous dimethylacetamide (45 mL) was sparged with nitrogen for 10 min. A 0.5 M solution of (R)-((1-(tert-butoxycarbonyl)pyrrolidin-3-yl)methyl)zinc(II) iodide in tetrahydrofuran (36 mL, 18 mmol, 1.6 equiv) was added. After heating at 65° C. for 18 h, the reaction mixture was cooled to room temperature and diluted with ethyl acetate (1 L) and water (800 mL). The layers were separated, and the aqueous layer was extracted with ethyl acetate (2×1.5 L). The combined organic layers were washed with saturated brine (800 mL), dried over sodium sulfate, filtered and concentrated under reduced pressure. The residue was purified on an Interchim automated chromatography system (Sorbtech 330 g silica gel column), eluting with a gradient of 0 to 70% ethyl acetate in heptanes, to give the title compound (2.9 g, 39% yield) as a yellow/orange semi-solid. LCMS: m/z=327.1 [M+H]⁺.

### Example 121: Methyl (S)-2-(pyrrolidin-3-ylmethyl)thiazole-5-carboxylate

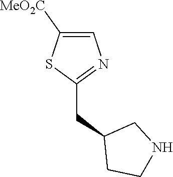 [SMILES: COC(=O)c1cnc(C[C@@H]2CCNC2)s1]

Trifluoroacetic acid (4 mL, 75.9 mmol, 16.5 equiv) was added over 5 min to a stirred solution of methyl (S)-2-((1-(tert-butoxycarbonyl)pyrrolidin-3-yl)methyl)thiazole-5-carboxylate (1.0 g, 4.6 mmol, 1 equiv) in anhydrous dichloromethane (16 mL) at 0° C. The resulting mixture was stirred at 0° C. for 2 h. The volatiles were evaporated under reduced pressure. The residue was dissolved in methanol (6 mL) and cooled to 0° C. (ice bath). MP-carbonate resin (2 g, ˜2 equiv) was added at 0° C., then the ice bath was removed, and the mixture was stirred at room temperature for 2 h. The resin was filtered off, and the filtrate was concentrated under reduced pressure. ¹H-NMR and ¹⁹F-NMR analysis of the residue indicated presence of trifluoroacetic acid. The residue was dissolved again in methanol (6 mL) and cooled to 0° C. (ice bath). MP-carbonate resin (2 g, ˜2 equiv) was added at 0° C., then the ice bath was removed, and the mixture was stirred at room temperature for 2 h. The resin was filtered off, and the filtrate was concentrated under reduced pressure. ¹H-NMR and ¹⁹F-NMR analysis of the residue indicated that all trifluoroacetic acid was removed. The residue was dried under vacuum at room temperature overnight to give the title compound (0.45 g, 65% yield) as a light-tan foam, which was used subsequently. LCMS: m/z=227.1 [M+H]⁺.

### Example 122: Methyl (S)-2-((1-propylpyrrolidin-3-yl)methyl)thiazole-5-carboxylate

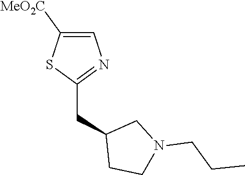 [SMILES: CCCN1CC[C@@H](Cc2ncc(C(=O)OC)s2)C1]

1-Bromopropane (0.27 g, 2.2 mmol, 1.1 equiv) and diisopropylethylamine (1.75 mL, 10.0 mmol, 5 equiv) were added to a solution of methyl (S)-2-((1-(tert-butoxycarbonyl)pyrrolidin-3-yl)methyl)thiazole-5-carboxylate (0.45 g, 2.0 mmol, 1 equiv) in N,N-dimethylacetamide (18 mL). The resulting mixture was stirred at room temperature for 90 h. The reaction mixture was diluted with ethyl acetate (50 mL) then washed with water (3×50 mL) and saturated brine (50 mL). The organic layer was dried over sodium sulfate, filtered and concentrated under reduced pressure to give the title compound (0.35 g, 65% yield) as an orange oil. LCMS: m/z=269.2 [M+H]⁺.

### Example 123: (S)-(2-((1-Propylpyrrolidin-3-yl)methyl)thiazol-5-yl)methanol

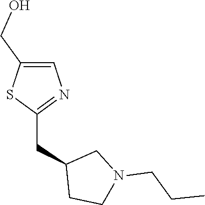 [SMILES: CCCN1CC[C@@H](Cc2ncc(CO)s2)C1]

1 M Lithium aluminum hydride in THF (1.95 mL, 1.95 mmol, 1.5 equiv) was added to a solution of methyl (S)-2-((1-propylpyrrolidin-3-yl)methyl)thiazole-5-carboxylate (0.35 g, 1.3 mmol, 1 equiv) in anhydrous THF (4.0 mL) at 0° C. under nitrogen. The reaction was stirred at 0° C. for 20 min, then warmed to room temperature and stirred for 2 h. The reaction was diluted with additional THF (30 mL) and cooled to 0° C. Sodium sulfate decahydrate (1.5 g) was cautiously added portionwise. The mixture was stirred at room temperature for 15 min, then the slurry was filtered off washing with dichloromethane (2×15 mL). The filtrate was concentrated under reduced pressure to give the title compound (0.23 g, 74% yield) as a light-yellow oil. LCMS: m/z=241.2 [M+H]⁺.

### Example 124: (S)-2-((1-Propylpyrrolidin-3-yl)methyl)thiazole-5-carbaldehyde

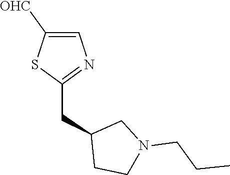 [SMILES: CCCN1CC[C@@H](Cc2ncc(C=O)s2)C1]

Activated manganese dioxide (1.59 g, 18.3 mmol, 20 equiv) was added to a solution of (S)-(2-((1-propylpyrrolidin-3-yl)methyl)thiazol-5-yl)methanol (0.22 g, 0.91 mmol, 1 equiv) in dichloromethane (6.0 mL) at room temperature. The resulting mixture was stirred at room temperature for 18 h. LCMS analysis at this time indicated full conversion to the desired product. The reaction mixture was passed through a Celite® pad (5 g), which was rinsed with dichloromethane (2×15 mL). The filtrate was concentrated under reduced pressure to give the title compound (0.16 g, 70% yield) as a brown oil. LCMS: m/z=239.2 [M+H]⁺.

### Example 125: 5-((1S,3R)-2-(2-Fluoro-2-methylpropyl)-3-methyl-2,3,4,9-tetrahydro-1H-pyrido[3,4-b]indol-1-yl)-2-(((S)-1-propylpyrrolidin-3-yl)methyl)thiazole

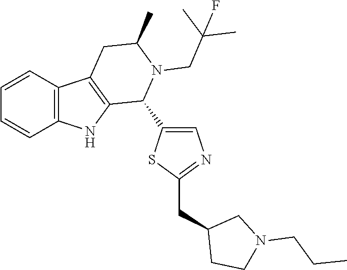 [SMILES: CCCN1CC[C@@H](Cc2ncc([C@@H]3c4[nH]c5ccccc5c4C[C@@H](C)N3CC(C)(C)F)s2)C1]

A mixture of (S)-2-((1-propylpyrrolidin-3-yl)methyl)thiazole-5-carbaldehyde (40 mg, 0.17 mmol, 1 equiv), (R)—N-(1-(1H-indol-3-yl)propan-2-yl)-2-fluoro-2-methylpropan-1-amine (50 mg, 0.2 mmol, 1.2 equiv) and acetic acid (19 μL, 0.34 mmol, 2 equiv) in anhydrous toluene (0.5 mL) was heated to 80° C. for 13 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (28 mg, 36% yield) as a tan solid. LCMS: m/z=469.3 [M+H]⁺; ¹H NMR (400 MHz, CDCl₃) δ=8.63 (br s, 1H), 7.52 (d, J=7.7 Hz, 1H), 7.33 (d, J=7.4 Hz, 1H), 7.24-7.04 (m, 3H), 5.25 (br s, 1H), 3.38 (br s, 1H), 3.00 (d, J=7.6 Hz, 2H), 2.83-2.76 (m, 1H), 2.69-2.31 (m, 9H), 2.23 (dd, J=6.8, 9.2 Hz, 1H), 2.09-1.98 (m, 1H), 1.58-1.45 (m, 6H), 1.36-1.29 (m, 3H), 1.15 (d, J=6.7 Hz, 3H), 0.90 (t, J=7.3 Hz, 3H).

### Example 126: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((S)-1-propylpyrrolidin-3-yl)methyl)thiazole

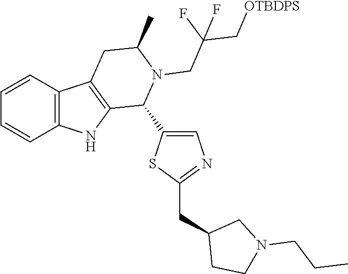

A mixture of (S)-2-((1-propylpyrrolidin-3-yl)methyl)thiazole-5-carbaldehyde (40 mg, 0.17 mmol, 1 equiv), (R)—N-(1-(1H-indol-3-yl)propan-2-yl)-3-((tert-butyldiphenylsilyl)oxy)-2,2-difluoropropan-1-amine (101 mg, 0.2 mmol, 1.2 equiv) and acetic acid (19 μL, 0.34 mmol, 2 equiv) in anhydrous toluene (0.5 mL) was heated to 80° C. for 13 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 m KP-amino-d column), eluting with a gradient of 0 to 100% ethyl acetate in heptanes, to give the title compound (51 mg, 42% yield) as a light-yellow oil. (LCMS: m/z=726.3 [M+H]⁺.

### Example 127: 2,2-Difluoro-3-((1S,3R)-3-methyl-1-(2-(((S)-1-propylpyrrolidin-3-yl)methyl)thiazol-5-yl)-1,3,4,9-tetrahydro-2H-pyrido[3,4-b]indol-2-yl)propan-1-ol

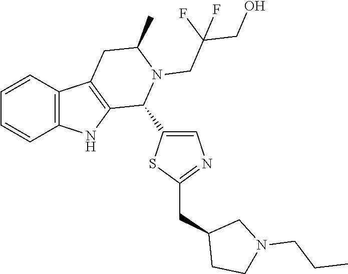 [SMILES: CCCN1CC[C@@H](Cc2ncc([C@@H]3c4[nH]c5ccccc5c4C[C@@H](C)N3CC(F)(F)CO)s2)C1]

1 M Tetrabutyl ammonium fluoride in THF (69 μL, 69 μmol, 1 equiv) was added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-1-propylpyrrolidin-3-yl)methyl)thiazole (50 mg, 69 μmol, 1 equiv) in THF (0.75 mL). After stirring at room temperature for 90 min, the reaction was cooled to 0° C. (ice bath) and diluted with water (5 mL) and dichloromethane (5 mL). The layers were separated, and the aqueous layer was extracted with dichloromethane (2×5 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (17 mg, 51% yield) as an off-white solid. LCMS: m/z=489.3 [M+H]⁺; ¹H NMR (400 MHz, CDCl₃) δ=9.93 (br s, 1H), 7.50 (d, J=7.7 Hz, 1H), 7.33 (d, J=7.9 Hz, 1H), 7.14 (dt, J=1.2, 7.5 Hz, 1H), 7.11-7.05 (m, 2H), 5.26 (s, 1H), 4.06-3.93 (m, 1H), 3.86-3.75 (m, 1H), 3.39-3.27 (m, 1H), 3.17 (dd, J=6.7, 14.6 Hz, 1H), 3.10-2.86 (m, 3H), 2.80-2.53 (m, 5H), 2.49-2.33 (m, 3H), 2.18-2.06 (m, 2H), 2.05 (s, 1H), 1.60-1.44 (m, 3H), 1.26 (t, J=7.2 Hz, 1H), 1.21 (d, J=6.7 Hz, 3H), 0.94 (t, J=7.3 Hz, 3H).

### Example 128: 5-((1S,3R)-2-(2,2-Difluoroethyl)-3-methyl-2,3,4,9-tetrahydro-1H-pyrido[3,4-b]indol-1-yl)-2-(((S)-1-propylpyrrolidin-3-yl)methyl)thiazole

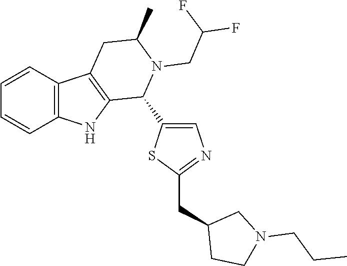 [SMILES: CCCN1CC[C@@H](Cc2ncc([C@@H]3c4[nH]c5ccccc5c4C[C@@H](C)N3CC(F)F)s2)C1]

A mixture of (S)-2-((1-propylpyrrolidin-3-yl)methyl)thiazole-5-carbaldehyde (40 mg, 0.17 mmol, 1 equiv), (R)—N-(2,2-difluoroethyl)-1-(1H-indol-3-yl)propan-2-amine (53 mg, 0.2 mmol, 1.2 equiv) and acetic acid (19 μL, 0.34 mmol, 2 equiv) in anhydrous toluene (0.5 mL) was heated to 80° C. for 3 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes to give the title compound (25 mg, 32% yield) as a yellow solid. LCMS: m/z=561.1 [M+H]⁺; ¹H NMR (400 MHz, CDCl₃) δ=8.28 (s, 1H), 7.52 (d, J=7.8 Hz, 1H), 7.35-7.26 (m, 2H), 7.19 (dt, J=1.3, 7.5 Hz, 1H), 7.15-7.10 (m, 1H), 5.96-5.55 (m, 1H), 5.11 (s, 1H), 3.46-3.29 (m, 1H), 3.13-2.96 (m, 3H), 2.87-2.71 (m, 3H), 2.66-2.46 (m, 4H), 2.44-2.30 (m, 2H), 2.27-2.21 (m, 1H), 2.09-1.99 (m, 1H), 1.57-1.45 (m, 3H), 1.19 (d, J=6.7 Hz, 3H), 0.89 (t, J=7.4 Hz, 3H).

### Example 129: 5-((1S,3R)-3-Methyl-2-(2,2,2-trifluoroethyl)-2,3,4,9-tetrahydro-1H-pyrido[3,4-b]indol-1-yl)-2-(((S)-1-propylpyrrolidin-3-yl)methyl)thiazole

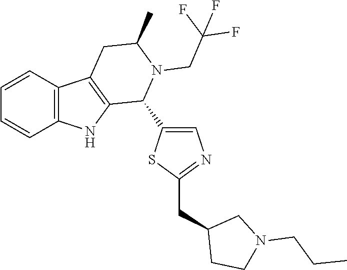 [SMILES: CCCN1CC[C@@H](Cc2ncc([C@@H]3c4[nH]c5ccccc5c4C[C@@H](C)N3CC(F)(F)F)s2)C1]

A mixture of (S)-2-((1-propylpyrrolidin-3-yl)methyl)thiazole-5-carbaldehyde (40 mg, 0.17 mmol, 1 equiv), (R)-1-(1H-indol-3-yl)-N-(2,2,2-trifluoroethyl)propan-2-amine (54 mg, 0.2 mmol, 1.2 equiv) and acetic acid (19 μL, 0.34 mmol, 2 equiv) in anhydrous toluene (0.5 mL) was heated to 80° C. for 14 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (25 mg, 31% yield) as a tan solid. LCMS: m/z=562.1 [M+H]⁺; ¹H NMR (400 MHz, CDCl₃) δ=8.68 (s, 1H), 7.52 (d, J=7.8 Hz, 1H), 7.33 (d, J=7.9 Hz, 1H), 7.24-7.17 (m, 2H), 7.16-7.10 (m, 1H), 5.53 (s, 1H), 5.13 (s, 1H), 3.46-3.37 (m, 1H), 3.33-3.19 (m, 1H), 3.03-2.90 (m, 3H), 2.81-2.72 (m, 2H), 2.69-2.45 (m, 4H), 2.37 (tdd, J=4.0, 7.7, 18.9 Hz, 2H), 2.24 (dd, J=6.6, 9.3 Hz, 1H), 2.09-1.98 (m, 1H), 1.57-1.45 (m, 3H), 1.22 (d, J=6.8 Hz, 3H), 0.89 (t, J=7.4 Hz, 3H).

### Example 130: Methyl (S)-2-((1-(3-fluoropropyl)pyrrolidin-3-yl)methyl)thiazole-5-carboxylate

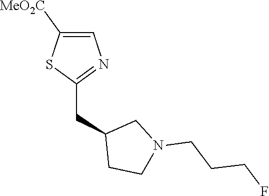 [SMILES: COC(=O)c1cnc(C[C@@H]2CCN(CCCF)C2)s1]

1-Bromo-3-fluoropropane (0.42 g, 3.0 mmol, 1.5 equiv) and diisopropylethylamine (1.75 mL, 10.0 mmol, 5 equiv) were added to a solution of methyl (S)-2-((1-(tert-butoxycarbonyl)pyrrolidin-3-yl)methyl)thiazole-5-carboxylate (0.45 g, 2.0 mmol, 1 equiv) in N,N-dimethylacetamide (18 mL). After stirring at room temperature for 48 h, the reaction mixture was diluted with ethyl acetate (50 mL) then washed with water (3×50 mL) and saturated brine (50 mL). The organic layer was dried over sodium sulfate, filtered and concentrated under reduced pressure to give the title compound (0.35 g, 61% yield) as a brown oil, which was used subsequently. LCMS: m/z=287.2 [M+H]⁺.

### Example 131: (S)-(2-((1-(3-Fluoropropyl)pyrrolidin-3-yl)methyl)thiazol-5-yl)methanol

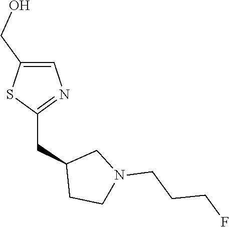 [SMILES: OCc1cnc(C[C@@H]2CCN(CCCF)C2)s1]

1 M Lithium aluminum hydride in THF (1.83 mL, 1.83 mmol, 1.5 equiv) was added to a solution of methyl (S)-2-((1-(3-fluoropropyl)pyrrolidin-3-yl)methyl)thiazole-5-carboxylate (0.35 g, 1.22 mmol, 1 equiv) in anhydrous THF (3.5 mL) at 0° C. under nitrogen. The reaction was warmed to room temperature and stirred for 1 h. The reaction was diluted with additional THE (40 mL) and cooled to 0° C. Sodium sulfate decahydrate (2 g) was cautiously added portionwise. The mixture was stirred at room temperature for 15 min, then the slurry was filtered off, washing with dichloromethane (2×25 mL). The filtrate was concentrated under reduced pressure to give the title compound (0.27 g, 87% yield) as a yellow oil. LCMS: m/z=259.2 [M+H]⁺.

### Example 132: (S)-2-((1-(3-Fluoropropyl)pyrrolidin-3-yl)methyl)thiazole-5-carbaldehyde

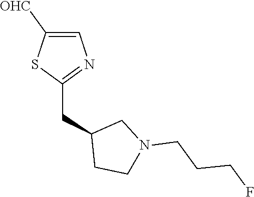 [SMILES: O=Cc1cnc(C[C@@H]2CCN(CCCF)C2)s1]

Activated manganese dioxide (1.81 g, 21.2 mmol, 20 equiv) was added to a solution of (S)-(2-((1-(3-fluoropropyl)pyrrolidin-3-yl)methyl)thiazol-5-yl)methanol (0.275 g, 1.06 mmol, 1 equiv) in dichloromethane (6.0 mL) at room temperature. After stirring at room temperature for 14 h, LCMS analysis at this time indicated full conversion to the desired product. The reaction mixture was passed through a Celite® pad (5 g), which was rinsed with dichloromethane (2×15 mL). The filtrate was concentrated under reduced pressure to give the title compound (0.2 g, 74% yield) as a brown oil. LCMS: m/z=257.1 [M+H]⁻.

### Example 133: 5-((1S,3R)-2-(2-Fluoro-2-methylpropyl)-3-methyl-2,3,4,9-tetrahydro-1H-pyrido[3,4-b]indol-1-yl)-2-(((S)-1-(3-fluoropropyl)pyrrolidin-3-yl)methyl)thiazole

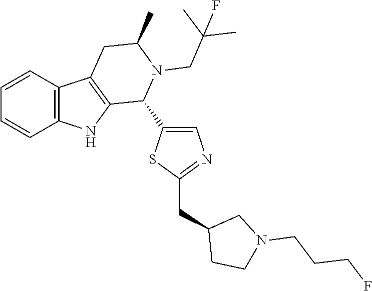 [SMILES: C[C@@H]1Cc2c([nH]c3ccccc23)[C@@H](c2cnc(C[C@@H]3CCN(CCCF)C3)s2)N1CC(C)(C)F]

A mixture of (S)-2-((1-(3-fluoropropyl)pyrrolidin-3-yl)methyl)thiazole-5-carbaldehyde (50 mg, 0.19 mmol, 1 equiv), (R)—N-(1-(1H-indol-3-yl)propan-2-yl)-2-fluoro-2-methylpropan-1-amine (58 mg, 0.23 mmol, 1.2 equiv) and acetic acid (22 μL, 0.39 mmol, 2 equiv) in anhydrous toluene (0.5 mL) was heated to 80° C. for 16 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (20 mg, 21% yield) as a light-yellow solid. LCMS: m/z=487.3 [M+H]⁺; ¹H NMR (400 MHz, CDCl₃) δ=8.39 (br s, 1H), 7.53 (d, J=7.8 Hz, 1H), 7.35 (d, J=7.9 Hz, 1H), 7.25-7.15 (m, 2H), 7.15-7.10 (m, 1H), 5.26 (br d, J=2.1 Hz, 1H), 4.56 (t, J=6.0 Hz, 1H), 4.44 (t, J=6.1 Hz, 1H), 3.48-3.30 (m, 1H), 3.01 (d, J=7.5 Hz, 2H), 2.78 (dd, J=7.6, 9.0 Hz, 1H), 2.71-2.49 (m, 9H), 2.29 (dd, J=6.4, 9.1 Hz, 1H), 2.10-2.01 (m, 1H), 1.94-1.87 (m, 1H), 1.87-1.81 (m, 1H), 1.61-1.54 (m, 1H), 1.54-1.48 (m, 3H), 1.36-1.30 (m, 3H), 1.16 (d, J=6.7 Hz, 3H).

### Example 134: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((S)-1-(3-fluoropropyl)pyrrolidin-3-yl)methyl)thiazole

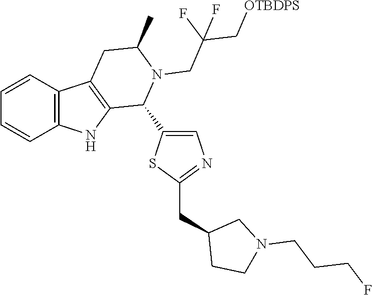

A mixture of(S)-2-((1-(3-fluoropropyl)pyrrolidin-3-yl)methyl)thiazole-5-carbaldehyde (55 mg, 0.21 mmol, 1 equiv), (R)—N-(1-(1H-indol-3-yl)propan-2-yl)-3-((tert-butyldiphenylsilyl)oxy)-2,2-difluoropropan-1-amine (130 mg, 0.25 mmol, 1.2 equiv) and acetic acid (24 μL, 0.42 mmol, 2 equiv) in anhydrous toluene (0.5 mL) was heated to 80° C. for 8 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (57 mg, 37% yield) as a yellow oil. LCMS: m/z=745.3 [M+H]⁺.

### Example 135: 2,2-Difluoro-3-((1S,3R)-1-(2-(((S)-1-(3-fluoropropyl)pyrrolidin-3-yl)methyl)thiazol-5-yl)-3-methyl-1,3,4,9-tetrahydro-2H-pyrido[3,4-b]indol-2-yl)propan-1-ol

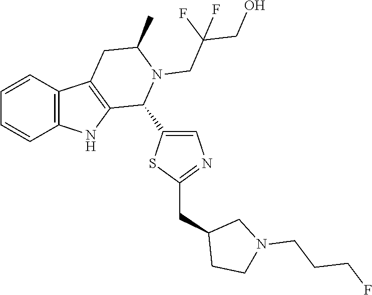 [SMILES: C[C@@H]1Cc2c([nH]c3ccccc23)[C@@H](c2cnc(C[C@@H]3CCN(CCCF)C3)s2)N1CC(F)(F)CO]

1M Tetrabutyl ammonium fluoride in THE (68 μL, 68 μmol, 1 equiv) was added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-1-(3-fluoropropyl)pyrrolidin-3-yl)methyl)thiazole (50 mg, 68 μmol, 1 equiv) in THF (0.75 mL). The resulting mixture was stirred at room temperature for 4 h. The reaction was cooled to 0° C. (ice bath) and diluted with water (5 mL) and dichloromethane (5 mL). The layers were separated, and the aqueous layer was extracted with dichloromethane (2×5 mL). The combined organic layers were dried over sodium sulfate, filtered, and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (35 mg, 43% yield) as a white solid. LCMS: m/z=507.3 [M+H]⁺; ¹H NMR (400 MHz, CDCl₃) δ=9.49 (br s, 1H), 7.51 (d, J=7.7 Hz, 1H), 7.33 (d, J=7.9 Hz, 1H), 7.20-7.07 (m, 3H), 5.24 (s, 1H), 4.59-4.51 (m, 1H), 4.48-4.39 (m, 1H), 4.02-3.92 (m, 1H), 3.85 (q, J=11.7 Hz, 1H), 3.41-3.30 (m, 1H), 3.16-3.02 (m, 2H), 2.99-2.87 (m, 2H), 2.77-2.48 (m, 8H), 2.25 (dd, J=6.7, 9.1 Hz, 1H), 2.13-2.04 (m, 1H), 1.96-1.82 (m, 2H), 1.58-1.48 (m, 1H), 1.22 (d, J=6.8 Hz, 3H).

### Example 136: 5-((1S,3R)-2-(2,2-Difluoroethyl)-3-methyl-2,3,4,9-tetrahydro-1H-pyrido[3,4-b]indol-1-yl)-2-(((S)-1-(3-fluoropropyl)pyrrolidin-3-yl)methyl)thiazole

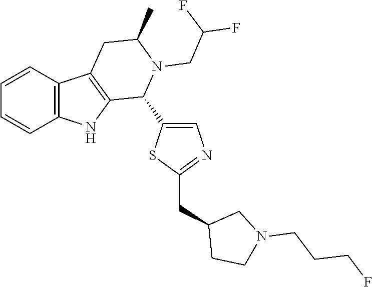 [SMILES: C[C@@H]1Cc2c([nH]c3ccccc23)[C@@H](c2cnc(C[C@@H]3CCN(CCCF)C3)s2)N1CC(F)F]

A mixture of (S)-2-((1-(3-fluoropropyl)pyrrolidin-3-yl)methyl)thiazole-5-carbaldehyde (50 mg, 0.19 mmol, 1 equiv), (R)—N-(2,2-difluoroethyl)-1-(1H-indol-3-yl)propan-2-amine (62 mg, 0.23 mmol, 1.2 equiv) and acetic acid (22 μL, 0.39 mmol, 2 equiv) in anhydrous toluene (0.5 mL) was heated to 80° C. for 3 h. After cooling to room temperature, the reaction mixture was diluted with dichloromethane (5 mL) and washed with saturated sodium carbonate (2 mL). The aqueous layer was extracted with dichloromethane (2×3 mL). The combined organic layers were dried over sodium sulfate, filtered, and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (62 mg, 36% yield) as a light-yellow solid. LCMS: m/z=477.2 [M+H]⁺; ¹H NMR (400 MHz, CDCl₃) δ=8.27 (s, 1H), 7.52 (d, J=7.7 Hz, 1H), 7.38-7.27 (m, 2H), 7.19 (dt, J=1.2, 7.5 Hz, 1H), 7.16-7.09 (m, 1H), 5.80 (ddt, J=3.8, 4.9, 56.6 Hz, 1H), 5.11 (s, 1H), 4.55 (t, J=6.0 Hz, 1H), 4.43 (t, J=6.0 Hz, 1H), 3.50-3.34 (m, 1H), 3.13-2.95 (m, 3H), 2.85-2.71 (m, 3H), 2.68-2.49 (m, 6H), 2.30 (dd, J=6.4, 9.2 Hz, 1H), 2.10-2.00 (m, 1H), 1.94-1.80 (m, 2H), 1.55 (tdd, J=6.3, 8.0, 12.7 Hz, 1H), 1.19 (d, J=6.7 Hz, 3H).

### Example 137: 2-(((S)-1-(3-Fluoropropyl)pyrrolidin-3-yl)methyl)-5-((1S,3R)-3-methyl-2-(2,2,2-trifluoroethyl)-2,3,4,9-tetrahydro-1H-pyrido[3,4-b]indol-1-yl)thiazole

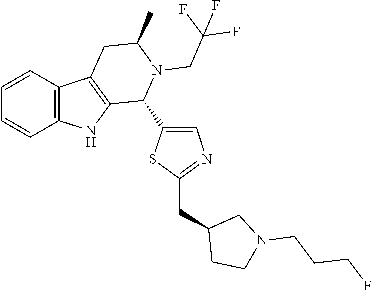 [SMILES: C[C@@H]1Cc2c([nH]c3ccccc23)[C@@H](c2cnc(C[C@@H]3CCN(CCCF)C3)s2)N1CC(F)(F)F]

A mixture of (S)-2-((1-(3-fluoropropyl)pyrrolidin-3-yl)methyl)thiazole-5-carbaldehyde (50 mg, 0.19 mmol, 1 equiv), (R)-1-(1H-indol-3-yl)-N-(2,2,2-trifluoroethyl)propan-2-amine (60 mg, 0.23 mmol, 1.2 equiv) and acetic acid (22 μL, 0.39 mmol, 2 equiv) in anhydrous toluene (0.5 mL) was heated to 80° C. for 16 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give the title compound (30 mg, 31% yield) as a tan solid. LCMS: m/z=495.2 [M+H]⁺; ¹H NMR (400 MHz, CDCl₃) δ=8.56 (s, 1H), 7.52 (d, J=7.8 Hz, 1H), 7.33 (d, J=8.1 Hz, 1H), 7.25-7.08 (m, 3H), 5.53 (s, 1H), 5.13 (s, 1H), 4.55 (t, J=6.0 Hz, 1H), 4.43 (t, J=6.1 Hz, 1H), 3.48-3.36 (m, 1H), 3.33-3.18 (m, 1H), 3.06-2.91 (m, 3H), 2.79-2.72 (m, 2H), 2.68-2.48 (m, 6H), 2.29 (dd, J=6.3, 9.1 Hz, 1H), 2.14-2.00 (m, 1H), 1.94-1.80 (m, 2H), 1.67-1.67 (m, 1H), 1.54 (tdd, J=6.3, 7.9, 12.7 Hz, 1H), 1.37-1.25 (m, 1H), 1.22 (d, J=6.7 Hz, 3H), 1.01-0.79 (m, 1H).

### Example 138: Methyl (S)-2-((1-(3,3-difluoropropyl)pyrrolidin-3-yl)methyl)thiazole-5-carboxylate

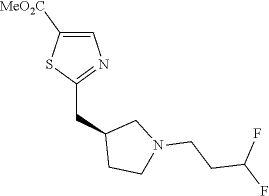 [SMILES: COC(=O)c1cnc(C[C@@H]2CCN(CCC(F)F)C2)s1]

3,3-Difluoropropyl tosylate (0.5 g, 2.0 mmol, 1.0 equiv) and diisopropylethylamine (1.75 mL, 10.0 mmol, 5 equiv) were sequentially added to a solution of methyl (S)-2-((1-(tert-butoxycarbonyl)pyrrolidin-3-yl)methyl)thiazole-5-carboxylate (0.45 g, 2.0 mmol, 1 equiv) in N,N-dimethylacetamide (18 mL). The resulting mixture was stirred at room temperature for 72 h. Additional difluoropropyl tosylate (2×0.5 equiv) was added in two 24-hour increments. The reaction mixture was diluted with ethyl acetate (50 mL) then washed with water (3×50 mL) and saturated brine (50 mL). The organic layer was dried over sodium sulfate, filtered and concentrated under reduced pressure to give crude compound (0.67 g) as a colorless oil. LCMS: m/z=305.1 [M+H]⁺.

### Example 139: (S)-(2-((1-(3,3-Difluoropropyl)pyrrolidin-3-yl)methyl)thiazol-5-yl)methanol

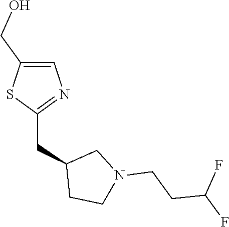 [SMILES: OCc1cnc(C[C@@H]2CCN(CCC(F)F)C2)s1]

1 M Lithium aluminum hydride in THF (3.3 mL, 3.3 mmol, 1.5 equiv) was added to a solution of crude methyl (S)-2-((1-(3,3-difluoropropyl)pyrrolidin-3-yl)methyl)thiazole-5-carboxylate (0.67 g, 2.2 mmol, 1 equiv) in anhydrous THE (6 mL) at 0° C. under nitrogen. The reaction was warmed to room temperature and stirred for 1 h. The reaction was diluted with additional THE (60 mL) and cooled to 0° C. Sodium sulfate decahydrate (3 g) was cautiously added portion-wise. The mixture was stirred at room temperature for 15 min, then the slurry was filtered off washing with dichloromethane (2×50 mL). The filtrate was concentrated under reduced pressure to give crude compound (0.26 g) as a yellow oil. LCMS: m/z=277.1 [M+H]⁺.

### Example 140: (S)-2-((1-(3,3-Difluoropropyl)pyrrolidin-3-yl)methyl)thiazole-5-carbaldehyde

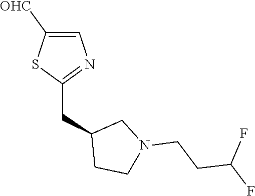 [SMILES: O=Cc1cnc(C[C@@H]2CCN(CCC(F)F)C2)s1]

Activated manganese dioxide (1.63 g, 20 equiv) was added to a solution of crude (S)-(2-((1-(3,3-difluoropropyl)pyrrolidin-3-yl)methyl)thiazol-5-yl)methanol (0.26 g, 0.93 mmol, 1 equiv) in dichloromethane (7.0 mL) and the resulting mixture was stirred at room temperature for 20 h. The reaction mixture was passed through a Celite® pad (5 g), which was rinsed with dichloromethane (2×30 mL). The filtrate was concentrated under reduced pressure to give crude compound (0.15 g) as a brown oil, which was used subsequently. LCMS: m/z=275.1 [M+H]⁺.

### Example 141: 2-(((S)-1-(3,3-Difluoropropyl)pyrrolidin-3-yl)methyl)-5-((1S,3R)-2-(2-fluoro-2-methylpropyl)-3-methyl-2,3,4,9-tetrahydro-1H-pyrido[3,4-b]indol-1-yl)thiazole

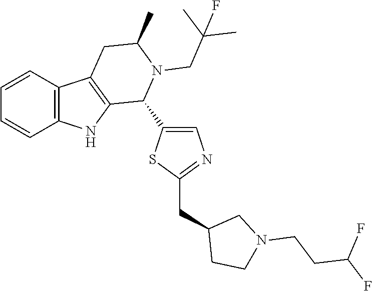 [SMILES: C[C@@H]1Cc2c([nH]c3ccccc23)[C@@H](c2cnc(C[C@@H]3CCN(CCC(F)F)C3)s2)N1CC(C)(C)F]

A mixture of crude (S)-2-((1-(3,3-difluoropropyl)pyrrolidin-3-yl)methyl)thiazole-5-carbaldehyde (150 mg, 0.36 mmol, 1 equiv), (R)—N-(1-(1H-indol-3-yl)propan-2-yl)-2-fluoro-2-methylpropan-1-amine (104 mg, 0.42 mmol, 1.2 equiv) and acetic acid (42 μL, 0.72 mmol, 2 equiv) in anhydrous toluene (0.5 mL) was heated to 80° C. for 15 h. After cooling to room temperature, the reaction was diluted with dichloromethane (5 mL) and washed with saturated sodium carbonate (2 mL). The aqueous layer was extracted with dichloromethane (2×3 mL). The combined organic layers were dried over sodium sulfate, filtered and concentrated under reduced pressure. The residue was purified on an Interchim automated chromatography system (11 g Biotage 50 μm KP-amino-d column), eluting with a gradient of 0 to 100% ethyl acetate in heptanes, to give partially purified title compound (20 mg). The semi-solid product was washed with pentane (3×5 mL) and the washings were discarded. The solid residue which remained after washing was lyophilized from acetonitrile-water, to give the title compound (1.5 mg, 0.2% yield over four steps) as a light-tan solid. LCMS (ESI) m/z=505.3 [M+H]⁺; ¹H NMR (400 MHz, CDCl₃) δ=7.87 (br s, 1H), 7.52 (d, J=7.6 Hz, 1H), 7.36-7.32 (m, 1H), 7.25-7.17 (m, 2H), 7.15-7.11 (m, 1H), 6.06-5.74 (m, 1H), 5.28 (br s, 1H), 3.39 (br s, 1H), 3.01 (dd, J=1.7, 7.5 Hz, 2H), 2.79-2.73 (m, 1H), 2.69-2.49 (m, 11H), 2.34-2.29 (m, 1H), 2.13-1.91 (m, 5H), 1.53-1.47 (m, 3H), 1.35-1.29 (m, 3H), 1.15 (d, J=6.8 Hz, 3H).

### Example 142: 5-bromo-1,3,4-thiadiazole-2-carbaldehyde

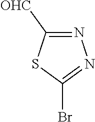

To ethyl 5-bromo-1,3,4-thiadiazole-2-carboxylate (1.00 equiv, 1.07 g, 4.52 mmol) in toluene (15 mL) at −76° C. under an atmosphere of nitrogen was slowly added the diisobutylaluminum hydride (1.20 equiv, 5.4 mL, 5.43 mmol) and stirred at −76° C. for 2 h. The reaction mixture was quenched with 2 mL of EtOAc, warmed to 0° C. and then treated with 50 mL of saturated Rochele's salt solution and 50 mL of EtOAc. The reaction mixture was stirred vigorously for 30 mins and the layers separated. The EtOAc layer was washed with brine, dried over sodium sulfate, filtered and concentrated. The crude was purified by silica gel chromatography using 0-40% EtOAc in hexane to give the title compound (380 mg, 1.97 mmol, 43.52% yield). LCMS: m/z=193.1 [M+H]⁺.

### Example 143: 2-bromo-5-[(1S,3R)-2-(2-fluoro-2-methyl-propyl)-3-methyl-1,3,4,9-tetrahydropyrido[3,4-b]indol-1-yl]-1,3,4-thiadiazole

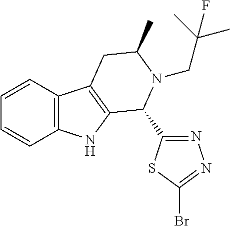 [SMILES: C[C@@H]1Cc2c([nH]c3ccccc23)[C@@H](c2nnc(Br)s2)N1CC(C)(C)F]

To 5-bromo-1,3,4-thiadiazole-2-carbaldehyde (1.00 equiv, 350 mg, 1.81 mmol) and 2-fluoro-N-[(1R)-2-(1H-indol-3-yl)-1-methyl-ethyl]-2-methyl-propan-1-amine (1.00 equiv, 450 mg, 1.81 mmol) in DCE (4 mL) was added the acetic acid (2.00 equiv, 0.21 mL, 3.63 mmol). The reaction was heated in microwave at 110° C. for 3 h. The reaction mixture was extracted with DCM, washed with saturated sodium bicarbonate, dried over sodium sulfate, filtered and concentrated. The crude was purified by silica gel chromatography using 0-50% EtOAc in hexane to give the title compound (600 mg, 1.42 mmol, 78.20% yield). LCMS: m/z=423.3 [M+H]⁺.

### Example 144: tert-butyl 3-[[5-[(1S,3R)-2-(2-fluoro-2-methyl-propyl)-3-methyl-1,3,4,9-tetrahydropyrido[3,4-b]indol-1-yl]-1,3,4-thiadiazol-2-yl]methyl]azetidine-1-carboxylate

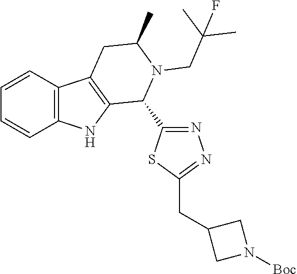 [SMILES: C[C@@H]1Cc2c([nH]c3ccccc23)[C@@H](c2nnc(CC3CN(C(=O)OC(C)(C)C)C3)s2)N1CC(C)(C)F]

To 2-bromo-5-[(1S,3R)-2-(2-fluoro-2-methyl-propyl)-3-methyl-1,3,4,9-tetrahydropyrido[3,4-b]indol-1-yl]-1,3,4-thiadiazole (1.0 equiv, 400 mg, 0.945 mmol) in DMA (6 mL) under nitrogen was added ((1-(tert-butoxycarbonyl)azetidin-3-yl)methyl)zinc(II) iodide (3.18 equiv, 6.0 mL, 3.00 mmol), copper(I) iodide (0.10 equiv, 18 mg, 0.0945 mmol) and tetrakis(triphenylphosphine)palladium(O) (0.10 equiv, 109 mg, 0.0945 mmol). The reaction mixture was flushed with nitrogen for 10 mins, capped and heated at 60° C. for 2 days. The reaction was filtered through Celite©, washing with ethyl acetate. The filtrate was washed with water, brine, dried over sodium sulfate, filtered and concentrated. The crude was purified by silica gel chromatography using 0-50% EtOAc in hexane to give the title compound (80 mg, 0.156 mmol, 16.5% yield). LCMS: m/z=514.9 [M+H]⁺.

### Example 145: 2-(azetidin-3-ylmethyl)-5-[(1S,3R)-2-(2-fluoro-2-methyl-propyl)-3-methyl-1,3,4,9-tetrahydropyrido[3,4-b]indol-1-yl]-1,3,4-thiadiazole

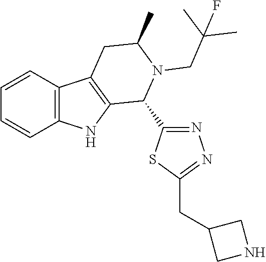 [SMILES: C[C@@H]1Cc2c([nH]c3ccccc23)[C@@H](c2nnc(CC3CNC3)s2)N1CC(C)(C)F]

To tert-butyl 3-[[5-[(1S,3R)-2-(2-fluoro-2-methyl-propyl)-3-methyl-1,3,4,9-tetrahydropyrido[3,4-b]indol-1-yl]-1,3,4-thiadiazol-2-yl]methyl]azetidine-1-carboxylate (1.00 equiv, 80 mg, 0.156 mmol) in DCM (2 mL) cooled to 0° C. was added the trifluoroacetic acid (41.2 equiv, 0.49 mL, 6.41 mmol) dropwise and stirred at 0° C. for 4 h. The reaction was diluted with 5 mL of toluene and concentrated under vacuum to remove the excess TFA. The crude mixture was extracted with EtOAc, cooled to 0° C. and neutralized with saturated sodium bicarbonate slowly. The organic layer was separated, washed with brine, dried over sodium sulfate, filtered and concentrated to give the title compound (60 mg, 0.145 mmol, 93.16% yield). LCMS: m/z=414.2 [M+H]⁺.

### Example 146: 2-[(1S,3R)-2-(2-fluoro-2-methyl-propyl)-3-methyl-1,3,4,9-tetrahydropyrido[3,4-b]indol-1-yl]-5-[[1-(3-fluoropropyl)azetidin-3-yl]methyl]-1,3,4-thiadiazole

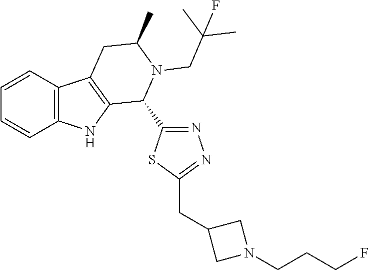 [SMILES: C[C@@H]1Cc2c([nH]c3ccccc23)[C@@H](c2nnc(CC3CN(CCCF)C3)s2)N1CC(C)(C)F]

To 2-(azetidin-3-ylmethyl)-5-[(1S,3R)-2-(2-fluoro-2-methyl-propyl)-3-methyl-1,3,4,9-tetrahydropyrido[3,4-b]indol-1-yl]-1,3,4-thiadiazole (1.00 equiv, 60 mg, 0.145 mmol) in DMA (1 mL) at 0° C. was added the N,N-diisopropylethylamine (2.0 equiv, 0.051 mL, 0.290 mmol) and 1-bromo-3-fluoro-propane (1.0 equiv, 0.15 mL, 0.145 mmol). The reaction was slowly warmed to room temperature and stirred for 18 h. The reaction mixture was purified by C-18 reverse phase chromatography using 10-50% ACN in water with 0.1% formic acid as modifier to give the title compound (6.0 mg, 0.0127 mmol, 8.73% yield). LCMS: m/z=474.2 [M+H]⁺.

### Example 147: tert-Butyl 3-((5-formylthiophen-2-yl)thio)azetidine-1-carboxylate

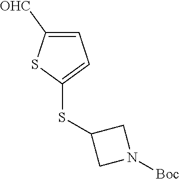 [SMILES: CC(C)(C)OC(=O)N1CC(Sc2ccc(C=O)s2)C1]

tert-Butyl 3-mercaptoazetidine-1-carboxylate (2.0 g, 10.6 mmol, 1.2 equiv) was added at room temperature to a solution of potassium hydroxide (1.76 g, 26.4 mmol, 3.0 equiv) in ethanol (68 mL). After stirring for 10 min, 5-bromothiophene-2-carbaldehyde (1.68 g, 8.8 mmol, 1.0 equiv) was added. After heating at 60° C. for 4 h, the resulting red solution was cooled to room temperature, and the solvent was removed under reduced pressure. The residue was diluted with dichloromethane (100 mL) and washed with saturated sodium bicarbonate (100 mL), water (100 mL) and saturated brine (100 mL). The combined aqueous layers were extracted with dichloromethane (2×50 mL). The combined organic layers were dried over sodium sulfate, filtered and concentrated under reduced pressure. The crude residue was purified on an Interchim automated chromatography system (Biotage 100 g HC), eluting with a gradient of 0 to 80% ethyl acetate in heptanes, to give the title compound as brown oil (2.37 g, 90% yield). LCMS: m/z=300.0 [M+H]⁺.

### Example 148: tert-Butyl 3-((5-((1S,3R)-2-(2-fluoro-2-methylpropyl)-3-methyl-2,3,4,9-tetrahydro-1H-pyrido[3,4-b]indol-1-yl)thiophen-2-yl)thio)azetidine-1-carboxylate

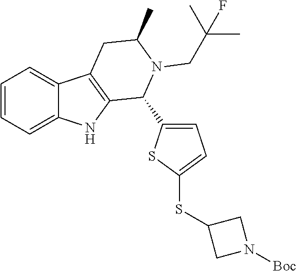 [SMILES: C[C@@H]1Cc2c([nH]c3ccccc23)[C@@H](c2ccc(SC3CN(C(=O)OC(C)(C)C)C3)s2)N1CC(C)(C)F]

Acetic acid (0.9 mL, 15.8 mmol, 2.0 equiv) was added at room temperature to a solution of tert-butyl 3-((5-formylthiophen-2-yl)thio)azetidine-1-carboxylate (2.37 g, 7.9 mmol, 1.0 equiv) and (R)—N-(1-(1H-indol-3-yl)propan-2-yl)-2-fluoro-2-methylpropan-1-amine (2.36 g, 9.5 mmol, 1.2 equiv) in toluene (23 mL). After heating at 80° C. for 16 h, the reaction mixture was cooled to room temperature. The mixture was diluted with ethyl acetate (50 mL) and saturated sodium bicarbonate (50 mL). The layers were separated, and the aqueous layer was extracted with ethyl acetate (3×40 mL). The combined organic layers were washed with saturated brine (50 mL), dried over sodium sulfate, filtered and concentrated under reduced pressure. The crude residue was purified on an Interchim automated chromatography system (Biotage 50 g HC), eluting with a gradient of 0 to 40% ethyl acetate in heptanes, to give the title compound (3.23 g, 77% yield) as a brown solid. LCMS: m/z=530.2 [M+H]⁺.

### Example 149: (1S,3R)-1-(5-(Azetidin-3-ylthio)thiophen-2-yl)-2-(2-fluoro-2-methylpropyl)-3-methyl-2,3,4,9-tetrahydro-1H-pyrido[3,4-b]indole

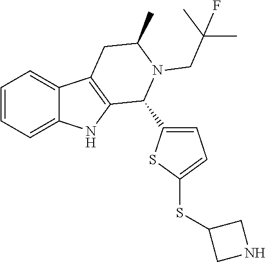 [SMILES: C[C@@H]1Cc2c([nH]c3ccccc23)[C@@H](c2ccc(SC3CNC3)s2)N1CC(C)(C)F]

Trifluoroacetic acid (2.6 mL, 34.0 mmol, 30 equiv) was added at room temperature to a solution of tert-butyl 3-((5-((1S,3R)-2-(2-fluoro-2-methylpropyl)-3-methyl-2,3,4,9-tetrahydro-1H-pyrido[3,4-b]indol-1-yl)thiophen-2-yl)thio)azetidine-1-carboxylate (600 mg, 1.13 mmol, 1.0 equiv) in dichloromethane (10 mL). After stirring for 30 min, the reaction was cooled to 0° C., and diluted with dichloromethane (30 mL). Saturated sodium carbonate (30 mL) was carefully added, and the resulting cloudy suspension was vigorously stirred for 5 min at room temperature. The layers were separated, and the aqueous layer was extracted with dichloromethane (3×20 mL). Combined organic layers were washed with 15% sodium hydroxide (2×20 mL) and saturated brine (30 mL), dried over sodium sulfate, filtered and concentrated under reduced pressure to give the title compound (410.6 mg, 84% yield) as a yellow solid. LCMS: m/z=430.1 [M+H]⁺.

### Example 150: (1S,3R)-2-(2-Fluoro-2-methylpropyl)-3-methyl-1-(5-((1-propylazetidin-3-yl)thio)thiophen-2-yl)-2,3,4,9-tetrahydro-1H-pyrido[3,4-b]indole

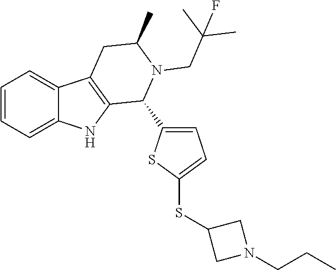 [SMILES: CCCN1CC(Sc2ccc([C@@H]3c4[nH]c5ccccc5c4C[C@@H](C)N3CC(C)(C)F)s2)C1]

N,N-Diisopropylethylamine (0.25 mL, 1.43 mmol, 1.5 equiv) and 1-bromopropane (129 mg, 1.05 mmol, 1.1 equiv) were sequentially added to a solution of (1S,3R)-1-(5-(azetidin-3-ylthio)thiophen-2-yl)-2-(2-fluoro-2-methylpropyl)-3-methyl-2,3,4,9-tetrahydro-1H-pyrido[3,4-b]indole (410 mg, 0.95 mmol, 1.0 equiv) in N,N-dimethylacetamide (2.7 mL). After stirring for 16 h at room temperature, saturated sodium bicarbonate (50 mL) was added. The layers were separated, and the aqueous layer was extracted with ethyl acetate (3×25 mL). The combined organic layers were washed with water (30 mL) and saturated brine (30 mL), dried over sodium sulfate, filtered and concentrated under reduced pressure. The resulting residue was divided into two equal portions and purified on an Interchim automated chromatography system (Biotage KP-amino-D column 25 g), eluting with a gradient of 0 to 100% ethyl acetate in dichloromethane, to give the title compound (101.5 mg, 23% yield) as a white solid. ¹H NMR (400 MHz, CDCl₃) δ=8.36-8.11 (m, 1H), 7.59-7.51 (m, 1H), 7.36-7.30 (m, 1H), 7.19 (dt, J=1.2, 7.5 Hz, 1H), 7.15-7.10 (m, 1H), 6.86 (d, J=4.3 Hz, 1H), 6.63 (d, J=2.9 Hz, 1H), 5.30-5.19 (m, 1H), 3.77-3.66 (m, 1H), 3.66-3.56 (m, 2H), 3.46-3.34 (m, 1H), 3.03 (td, J=7.4, 15.1 Hz, 2H), 2.71-2.44 (m, 4H), 2.34-2.25 (m, 2H), 2.05 (s, 1H), 1.60-1.50 (m, 3H), 1.39-1.24 (m, 6H), 1.20-1.06 (m, 3H), 0.88-0.82 (m, 3H); LCMS: m/z=472.2 [M+H]⁺.

### Example 151: (1S,3R)-2-(2-Fluoro-2-methylpropyl)-1-(5-((1-(3-fluoropropyl)azetidin-3-yl)thio)thiophen-2-yl)-3-methyl-2,3,4,9-tetrahydro-1H-pyrido[3,4-b]indole

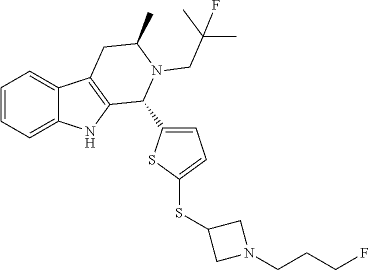 [SMILES: C[C@@H]1Cc2c([nH]c3ccccc23)[C@@H](c2ccc(SC3CN(CCCF)C3)s2)N1CC(C)(C)F]

N,N-Diisopropylethylamine (0.85 mL, 4.95 mmol, 5.0 equiv) and 1-bromo-3-fluoropropane (168 mg, 1.19 mmol, 1.2 equiv) were sequentially added to a solution of (1S,3R)-1-(5-(azetidin-3-ylthio)thiophen-2-yl)-2-(2-fluoro-2-methylpropyl)-3-methyl-2,3,4,9-tetrahydro-1H-pyrido[3,4-b]indole (424 mg, 0.99 mmol, 1.0 equiv) in N,N-dimethylacetamide (3.3 mL). Saturated sodium bicarbonate (50 mL) was added. The layers were separated, and the aqueous layer was extracted with ethyl acetate (3×25 mL). The combined organic layers were washed with water (30 mL) and saturated brine (30 mL), dried over sodium sulfate, filtered and concentrated under reduced pressure. The resulting residue was divided into two equal portions. Each portion was purified on an Interchim automated chromatography system (Biotage 25 g HC), eluting with a gradient of 0 to 10% methanol in dichloromethane, to give the title compound (65.9 mg, 14% yield) as a yellow solid. ¹H NMR (400 MHz, CDCl₃) δ=8.21 (br s, 1H), 7.53 (d, J=7.7 Hz, 1H), 7.34 (d, J=7.6 Hz, 1H), 7.19 (dt, J=1.3, 7.5 Hz, 1H), 7.15-7.10 (m, 1H), 6.88 (d, J=3.7 Hz, 1H), 6.65 (d, J=2.9 Hz, 1H), 5.45-5.23 (m, 1H), 4.49 (t, J=5.9 Hz, 1H), 4.37 (t, J=5.9 Hz, 1H), 3.77-3.56 (m, 3H), 3.48-3.33 (m, 1H), 3.15-2.92 (m, 3H), 2.68-2.43 (m, 6H), 1.75-1.60 (m, 3H), 1.60-1.50 (m, 3H), 1.38-1.31 (m, 3H), 1.28 (br s, 1H), 1.18 (d, J=6.7 Hz, 3H); LCMS: m/z=490.2 [M+H]⁺.

### Example 152: tert-Butyl (1S,3R)-1-(2-((1-(tert-butoxycarbonyl)azetidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

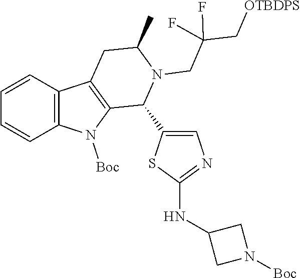

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (500 mg, 0.64 mmol, 1 equiv), tert-butyl 3-aminoazetidine-1-carboxylate (220 mg, 1.28 mmol, 2 equiv), sodium tert-butoxide (92.2 mg, 0.96 mmol, 1.5 equiv), 2,2′-bis(diphenylphosphino)-1,1′-binaphthyl (80 mg, 0.128 mmol, 0.2 equiv), tris(dibenzylideneacetone)dipalladium(O) (59 mg, 0.064 mmol, 0.1 equiv) and toluene (8 mL). The mixture was sparged with nitrogen for 10 minutes, sealed and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue which was purified on a Biotage automated chromatography system (55 g Biotage KPNH column), eluting with a gradient of 0 to 50% ethyl acetate in hexanes to give the title compound (119 mg, 21% yield) as a yellow foam. LCMS: m/z=873.3 [M+H]⁺.

### Example 153: N-(Azetidin-3-yl)-5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazol-2-amine

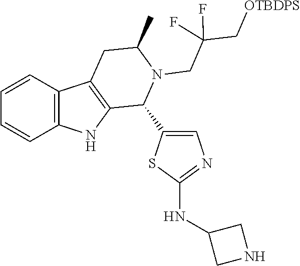

Trifluoroacetic acid (0.63 mL, 8.2 mmol, 60 equiv) was added to tert-butyl (1S,3R)-1-(2-((1-(tert-butoxycarbonyl)azetidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (119 mg, 0.136 mmol, 1 equiv) in dichloromethane (1.2 mL) at 0° C., and the reaction was stirred at 0° C. for 24 hours. The reaction was diluted with cold dichloromethane (50 mL) and adjusted to pH 10 with saturated sodium carbonate (40 mL). The layers were separated and the organic layer was washed with saturated sodium carbonate (60 mL), dried over sodium sulfate, filtered and concentrated under reduced pressure to give the title compound (103 mg, >99% yield) as an orange solid. LCMS: m/z=673.3 [M+H]⁺.

### Example 154: 5-((J S, 3)-2-(3-((tert-Butyldiphenylsilyl)oxy)-2,2-difluoropropyl)-3-methyl-2,3,4,9-tetrahydro-1H-pyrido[3,4-b]indol-1-yl)-N-(1-(3-fluoropropyl)azetidin-3-yl)thiazol-2-amine

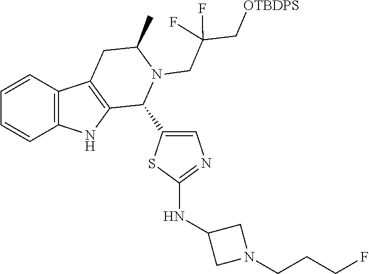

1-Bromo-3-fluoropropane (19 mg, 0.163 mmol, 1.0 equiv) and N,N-diisopropylethylamine (0.035 mL 0.204 mmol, 1.5 equiv) were sequentially added to a solution of N-(Azetidin-3-yl)-5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazol-2-amine (103 mg, 0.136 mmol, 1 equiv) in N,N-dimethylacetamide (0.5 mL). After stirring for 20 hours at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite®. The residue was purified on a Biotage automated chromatography system (11 g, Biotage 50 μm KPNH column), eluting with a gradient of 1 to 100% ethyl acetate in dichloromethane to give the title compound (54 mg, 84% yield) as a white solid. LCMS: m/z=733.3 [M+H]⁻.

### Example 155: 2,2-difluoro-3-((1S,3R)-1-(2-((1-(3-fluoropropyl)azetidin-3-yl)amino)thiazol-5-yl)-3-methyl-1,3,4,9-tetrahydro-2H-pyrido[3,4-b]indol-2-yl)propan-1-ol

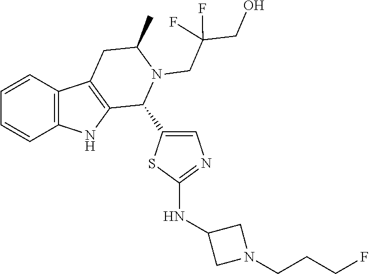 [SMILES: C[C@@H]1Cc2c([nH]c3ccccc23)[C@@H](c2cnc(NC3CN(CCCF)C3)s2)N1CC(F)(F)CO]

1M Tetra-n-butylammonium fluoride in THF (0.081 mL, 0.081 mmol, 1.1 equiv) was added dropwise at room temperature to a solution of 5-((1S,3)-2-(3-((tert-Butyldiphenylsilyl)oxy)-2,2-difluoropropyl)-3-methyl-2,3,4,9-tetrahydro-1H-pyrido[3,4-b]indol-1-yl)-N-(1-(3-fluoropropyl)azetidin-3-yl)thiazol-2-amine (54 mg, 0.074 mmol, 1 equiv) in THF (1.5 mL). After stirring for 1 hour, the reaction was concentrated under reduced pressure and the residue was diluted with saturated ammonium chloride (5 mL). The suspension was extracted with ethyl acetate (3×5 mL). The combined organic layers were dried over sodium sulfate, filtered and concentrated under reduced pressure. The residue was purified on a Biotage automated chromatography system (Biotage 11 g KPNH), eluting with gradient of 0 to 10% methanol in dichloromethane to give the title compound (16.7 mg, 48% yield) as a tan solid. ¹H-NMR (400 MHz, CDCl₃) δ=8.27 (br s, 1H), 7.51 (d, J=7.9 Hz, 1H), 7.31 (d, J=7.7 Hz, 1H), 7.18 (dt, J=1.2, 7.6 Hz, 1H), 7.14-7.09 (m, 1H), 6.71 (s, 1H), 5.60 (br s, 1H), 5.10 (s, 1H), 4.53 (t, J=5.9 Hz, 1H), 4.41 (t, J=5.9 Hz, 1H), 4.16 (br s, 1H), 4.01-3.82 (m, 2H), 3.74-3.46 (m, 4H), 3.28-3.06 (m, 1H), 3.03-2.89 (m, 3H), 2.77 (dd, J=4.5, 16.1 Hz, 1H), 2.62-2.52 (m, 3H), 1.81-1.65 (m, 4H), 1.22 (d, J=6.7 Hz, 3H); LCMS: m/z=494.2 [M+H]⁺.

### Example 156: tert-Butyl (1S,3R)-1-(2-(((R)-1-(tert-butoxycarbonyl)pyrrolidin-3-yl)amino) thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

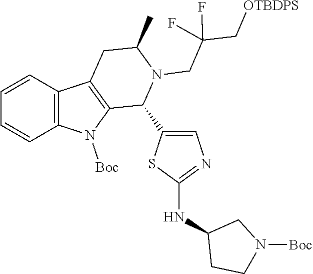

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (300 mg, 0.385 mmol, 1 equiv), tert-butyl (R)-3-aminopyrrolidine-1-carboxylate (144 mg, 0.77 mmol, 2 equiv), sodium tert-butoxide (55.7 mg, 0.58 mmol, 1.5 equiv), 2,2′-bis(diphenylphosphino)-1,1′-binaphthyl (48 mg, 0.077 mmol, 0.2 equiv), tris(dibenzylideneacetone) dipalladium(O) (36 mg, 0.039 mmol, 0.1 equiv) and toluene (5 mL). The mixture was sparged with nitrogen for 10 minutes, sealed and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue which was purified on a Biotage automated chromatography system (55 g Biotage KPNH column), eluting with a gradient of 5 to 60% ethyl acetate in hexanes to give the title compound (132 mg, 39% yield) as a yellow foam. LCMS: m/z=887.3 [M+H]⁺.

### Example 157: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N—((R)-pyrrolidin-3-yl)thiazol-2-amine

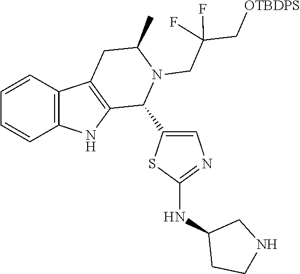

Trifluoroacetic acid (0.68 mL, 8.94 mmol, 60 equiv) was added to tert-butyl (1S,3R)-1-(2-(((R)-1-(tert-butoxycarbonyl)pyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (132 mg, 0.149 mmol, 1 equiv) in dichloromethane (1.7 mL) at 0° C. and the reaction was stirred at 0° C. for 24 hours. The reaction was diluted with cold dichloromethane (50 mL) and adjusted to pH 10 with saturated sodium carbonate (40 mL). The layers were separated, and the organic layer was washed with saturated sodium carbonate (60 mL). The organic layer was dried over sodium sulfate, filtered and concentrated under reduced pressure to give title compound (100 mg, >99% yield) as an orange solid. LCMS: m/z=686.3 [M+H]⁺.

### Example 158: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((R)-1-(3-fluoropropyl) pyrrolidin-3-yl)thiazol-2-amine

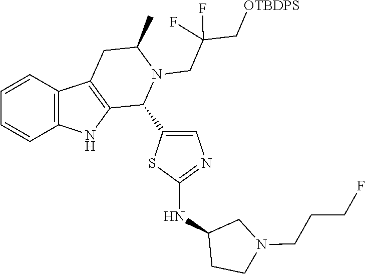

1-Bromo-3-fluoropropane (21 mg, 0.148 mmol, 1.0 equiv) and N,N-diisopropylethylamine (0.038 mL 0.222 mmol, 1.5 equiv) were sequentially added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((R)-pyrrolidin-3-yl)thiazol-2-amine (100 mg, 0.148 mmol, 1 equiv) in N,N-dimethylacetamide (0.4 mL). After stirring for 20 hours at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite®. The residue was purified on a Biotage automated chromatography system (11 g, Biotage 50 m KPNH column), eluting with a gradient of 1 to 100% ethyl acetate in dichloromethane to give the title compound (45 mg, 41% yield) as a white solid. LCMS: m/z=747.4 [M+H]⁺.

### Example 159: 2,2-Difluoro-3-((1S,3R)-1-(2-(((R)-1-(3-fluoropropyl)pyrrolidin-3-yl)amino)thiazol-5-yl)-3-methyl-1,3,4,9-tetrahydro-2H-pyrido[3,4-b]indol-2-yl)propan-1-ol

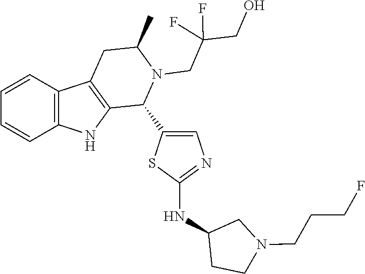 [SMILES: C[C@@H]1Cc2c([nH]c3ccccc23)[C@@H](c2cnc(N[C@@H]3CCN(CCCF)C3)s2)N1CC(F)(F)CO]

1M Tetra-n-butylammonium fluoride in THF (0.066 mL, 0.066 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((R)-1-(3-fluoropropyl)pyrrolidin-3-yl)thiazol-2-amine (45 mg, 0.06 mmol, 1 equiv) in THF (3 mL). After stirring for 1 hour, the reaction was concentrated under reduced pressure and the residue was diluted with saturated ammonium chloride (5 mL). The suspension was extracted with ethyl acetate (3×5 mL). The combined organic layers were dried over sodium sulfate, filtered and concentrated under reduced pressure. The residue was purified on a Biotage automated chromatography system (Biotage 11 g KPNH), eluting with gradient of 0 to 10% methanol in dichloromethane to give the title compound (17.7 mg, 59% yield) as a white solid. ¹H-NMR (400 MHz, CDCl₃) δ=8.44 (br s, 1H), 7.50 (d, J=7.8 Hz, 1H), 7.31 (d, J=8.1 Hz, 1H), 7.17 (dt, J=1.3, 7.5 Hz, 1H), 7.13-7.08 (m, 1H), 6.69 (s, 1H), 5.47 (br d, 0.1=7.9 Hz, 1H), 5.09 (s, 1H), 4.56 (t, J=5.9 Hz, 1H), 4.44 (t, J=5.9 Hz, 1H), 4.06 (br s, 1H), 4.02-3.76 (m, 3H), 3.70-3.44 (m, 1H), 3.34-3.05 (m, 1H), 3.01-2.84 (m, 2H), 2.81-2.69 (m, 2H), 2.64-2.52 (m, 4H), 2.36-2.24 (m, 2H), 1.92-1.80 (m, 2H), 1.76-1.66 (m, 1H), 1.22 (d, J=6.7 Hz, 3H); LCMS: m/z=508.2 [M+H]⁺.

### Example 160: tert-Butyl (1S,3R)-1-(2-(((3S,4R)-1-(tert-butoxycarbonyl)-4-fluoropyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

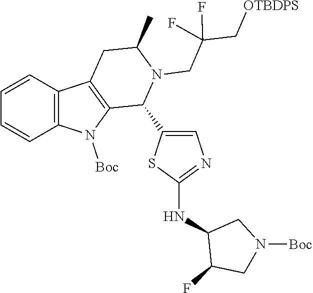

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (500 mg, 0.64 mmol, 1 equiv), tert-butyl (3S,4R)-3-amino-4-fluoropyrrolidine-1-carboxylate (261 mg, 1.28 mmol, 2 equiv), sodium tert-butoxide (92.2 mg, 0.96 mmol, 1.5 equiv), 2,2′-bis(diphenylphosphino)-1,1′-binaphthyl (80 mg, 0.128 mmol, 0.2 equiv), tris(dibenzylideneacetone)dipalladium(O) (59 mg, 0.064 mmol, 0.1 equiv) and toluene (8 mL). The mixture was sparged with nitrogen for 10 minutes, sealed, and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue which was purified on a Biotage automated chromatography system (55 g Biotage KPNH column), eluting with a gradient of 0 to 50% ethyl acetate in hexanes to give the title compound (318 mg, 55% yield) as a yellow foam. LCMS: m/z=905.3 [M+H]⁺.

### Example 161: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4R)-4-fluoropyrrolidin-3-yl)thiazol-2-amine

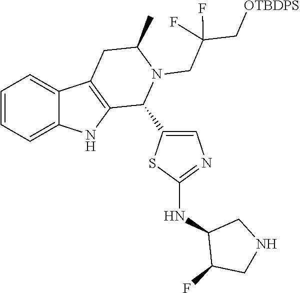

Trifluoroacetic acid (1.6 mL, 21.1 mmol, 60 equiv) was added to compound tert-butyl (1S,3R)-1-(2-(((3S,4R)-1-(tert-butoxycarbonyl)-4-fluoropyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (318 mg, 0.35 mmol, 1 equiv) in dichloromethane (3.0 mL) at 0° C. in a 40 mL vial. After stirring at 0° C. for 24 hours, the reaction was diluted with cold dichloromethane (50 mL) and adjusted to pH 10 with saturated sodium carbonate (40 mL). The layers were separated and the organic layer was washed with saturated sodium carbonate (60 mL). The organic layer was dried over sodium sulfate, filtered and concentrated under reduced pressure to give title compound (272 mg, >99% yield) as an orange solid. LCMS: m/z=704.3 [M+H]⁺.

### Example 162: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4R)-4-fluoro-1-(3-fluoropropyl)pyrrolidin-3-yl)thiazol-2-amine

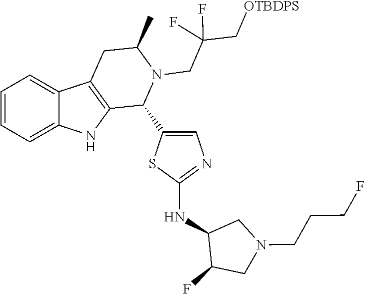

1-Bromo-3-fluoropropane (59 mg, 0.42 mmol, 1.2 equiv) and N,N-diisopropylethylamine (0.09 mL, 0.53 mmol, 1.5 equiv) were sequentially added to a solution of 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4R)-4-fluoropyrrolidin-3-yl)thiazol-2-amine (272 mg, 0.35 mmol, 1 equiv) in N,N-dimethylacetamide (0.8 mL). After stirring for 20 hours at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite® (1 g). The residue was purified on a Biotage automated chromatography system (28 g, Biotage KPNH column), eluting with a gradient of 5 to 100% ethyl acetate in dichloromethane to give the title compound (81 mg, 30% yield) as a yellow solid. LCMS: m/z=765.2 [M+H]⁺.

### Example 163: 2,2-Difluoro-3-(1S,3R)-1-(2-(((3S,4R)-4-fluoro-1-(3-fluoropropyl)pyrrolidin-3-yl)amino)thiazol-5-yl)-3-methyl-1,3,4,9-tetrahydro-2H-pyrido[3,4-b]indol-2-yl)propan-1-ol

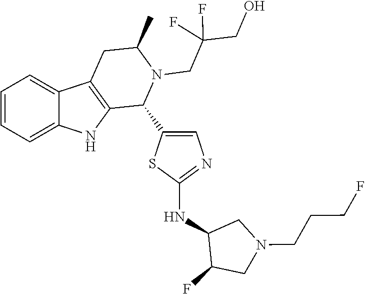 [SMILES: C[C@@H]1Cc2c([nH]c3ccccc23)[C@@H](c2cnc(N[C@H]3CN(CCCF)C[C@H]3F)s2)N1CC(F)(F)CO]

1M Tetra-n-butylammonium fluoride in THF (0.12 mL, 0.12 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N-((3S,4R)-4-fluoro-1-(3-fluoropropyl)pyrrolidin-3-yl)thiazol-2-amine (0.81 mg, 0.106 mmol, 1 equiv) in THF (1.8 mL). After stirring for 1 hour, the reaction was concentrated under reduced pressure. The residue was diluted with saturated ammonium chloride (5 mL) and the suspension was extracted with ethyl acetate (3×5 mL). The combined organic layers were dried over sodium sulfate, filtered and concentrated under reduced pressure. The residue was purified on Biotage automated chromatography system (Biotage 11 g KPNH, eluting with gradient of 10 to 100% ethyl acetate in dichloromethane to give title compound (45 mg, 80% yield) as a white solid. ¹H-NMR (400 MHz, CDCl₃) δ=8.28 (br s, 1H), 7.51 (d, J=7.7 Hz, 1H), 7.31 (d, J=7.9 Hz, 1H), 7.21-7.09 (m, 2H), 6.70 (s, 1H), 5.55 (br d, J=8.3 Hz, 1H), 5.20-4.96 (m, 2H), 4.54 (t, J=5.9 Hz, 1H), 4.42 (t, J=5.9 Hz, 1H), 4.30-4.18 (m, 1H), 3.92 (br t, J=12.2 Hz, 2H), 3.66-3.47 (m, 2H), 3.23-2.83 (m, 4H), 2.83-2.73 (m, 1H), 2.70-2.52 (m, 4H), 1.91-1.78 (m, 2H), 1.75-1.49 (m, 1H), 1.23 (d, J=6.7 Hz, 3H); LCMS: m/z=526.3 [M+H]⁺.

### Example 164: tert-Butyl (1S,3R)-1-(2-(((3S,4S)-1-(tert-butoxycarbonyl)-4-fluoropyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

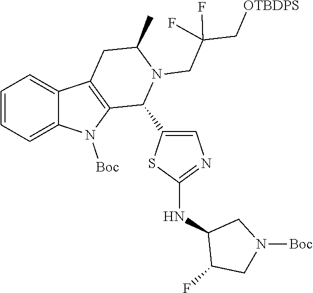

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (500 mg, 0.64 mmol, 1 equiv), tert-butyl (3S,4S)-3-amino-4-fluoropyrrolidine-1-carboxylate (261 mg, 1.28 mmol, 2 equiv), sodium tert-butoxide (92.2 mg, 0.96 mmol, 1.5 equiv), 2,2′-bis(diphenylphosphino)-1,1′-binaphthyl (80 mg, 0.128 mmol, 0.2 equiv), tris(dibenzylideneacetone)dipalladium(O) (59 mg, 0.064 mmol, 0.1 equiv) and toluene (8 mL). The mixture was sparged with nitrogen for 10 minutes, sealed, and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue which was purified on a Biotage automated chromatography system (55 g Biotage KPNH column), eluting with a gradient of 0 to 50% ethyl acetate in hexanes to give the title compound (168 mg, 29% yield) as a yellow foam. LCMS: m/z=905.3 [M+H]⁺.

### Example 165: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4S)-4-fluoropyrrolidin-3-yl)thiazol-2-amine

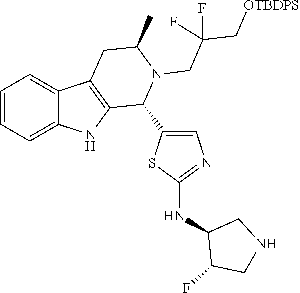

Trifluoroacetic acid (1.6 mL, 21.1 mmol, 60 equiv) was added to tert-butyl (1S,3R)-1-(2-(((3S,4S)-1-(tert-butoxycarbonyl)-4-fluoropyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (168 mg, 0.186 mmol, 1 equiv) in dichloromethane (1.7 mL) at 0° C. in a 40 mL vial. After stirring at 0° C. for 24 hours, the reaction was diluted with cold dichloromethane (50 mL) and adjusted to pH 10 with saturated sodium carbonate (40 mL). The layers were separated and the organic layer was washed with saturated sodium carbonate (60 mL). The organic layer was dried over sodium sulfate, filtered and concentrated under reduced pressure to give the title compound (142 mg, >99% yield) as an orange solid. LCMS: m/z=704.1 [M+H]⁺.

### Example 166: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4S)-4-fluoro-1-(3-fluoropropyl)pyrrolidin-3-yl)thiazol-2-amine

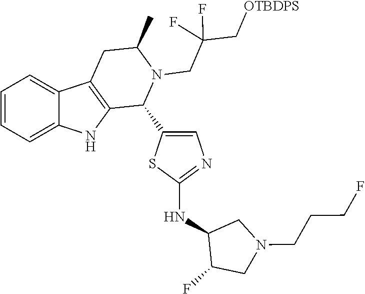

1-Bromo-3-fluoropropane (31 mg, 0.223 mmol, 1.2 equiv) and N,N-diisopropylethylamine (0.048 mL 0.28 mmol, 1.5 equiv) were sequentially added to a solution of 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4S)-4-fluoropyrrolidin-3-yl)thiazol-2-amine (142 mg, 0.186 mmol, 1 equiv) in N,N-dimethylacetamide (0.6 mL). After stirring for 20 hours at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite®. The residue was purified on a Biotage automated chromatography system (11 g, Biotage 50 m KPNH column), eluting with a gradient of 5 to 100% ethyl acetate in dichloromethane to give the title compound (68 mg, 48% yield) as a yellow solid. LCMS: m/z=765.2 [M+H]⁺.

### Example 167: 2,2-Difluoro-3-((1S,3R)-1-(2-(((3S,4S)-4-fluoro-1-(3-fluoropropyl)pyrrolidin-3-yl)amino)thiazol-5-yl)-3-methyl-1,3,4,9-tetrahydro-2H-pyrido[3,4-b]indol-2-yl)propan-1-ol

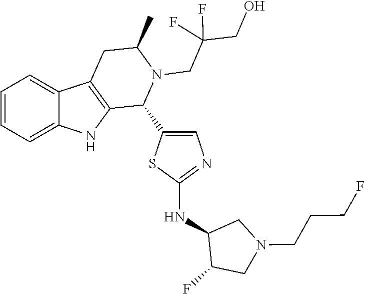 [SMILES: C[C@@H]1Cc2c([nH]c3ccccc23)[C@@H](c2cnc(N[C@H]3CN(CCCF)C[C@@H]3F)s2)N1CC(F)(F)CO]

1M Tetra-n-butylammonium fluoride in THF (0.098 mL, 0.098 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N-((3S,4R)-4-fluoro-1-(3-fluoropropyl)pyrrolidin-3-yl)thiazol-2-amine (68 mg, 0.09 mmol, 1 equiv) in THF (1.5 mL). After stirring for 1 hour, the reaction was concentrated under reduced pressure and the residue was diluted with saturated ammonium chloride (5 mL). The suspension was extracted with ethyl acetate (3×5 mL). The combined organic layers were dried over sodium sulfate, filtered and concentrated under reduced pressure. The residue was purified on Biotage automated chromatography system (Biotage 11 g KPNH, eluting with gradient of 10 to 100% ethyl acetate in dichloromethane to give the title compound (15 mg, 32% yield) as a white solid. ¹H NMR (400 MHz, CDCl₃) δ=8.32 (br s, 1H), 8.21 (s, 1H), 7.51 (d, J=7.5 Hz, 1H), 7.34-7.28 (m, 1H), 7.21-7.09 (m, 2H), 6.75 (s, 1H), 5.50 (br d, J=5.5 Hz, 1H), 5.15-5.07 (m, 1H), 5.03-4.85 (m, 1H), 4.55 (t, J=5.9 Hz, 1H), 4.47-4.41 (m, 1H), 4.33-4.07 (m, 1H), 3.93 (br t, J=12.3 Hz, 2H), 3.70-3.44 (m, 2H), 3.44-3.26 (m, 1H), 3.23-3.09 (m, 2H), 3.00-2.87 (m, 2H), 2.82-2.71 (m, 1H), 2.67-2.40 (m, 5H), 1.92-1.64 (m, 2H), 1.31-1.16 (m, 3H); LCMS: m/z=526.2 [M+H]⁺.

### Example 168: tert-Butyl (1S,3R)-1-(2-(((S)-1-(tert-butoxycarbonyl)piperidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

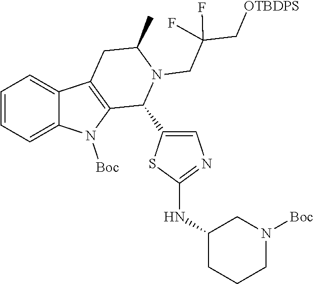

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (600 mg, 0.768 mmol, 1 equiv), tert-butyl (S)-3-aminopiperidine-1-carboxylate (308 mg, 1.54 mmol, 2 equiv), sodium tert-butoxide (111 mg, 1.15 mmol, 1.5 equiv), 2,2′-bis(diphenylphosphino)-1,1′-binaphthyl (96 mg, 0.154 mmol, 0.2 equiv), tris(dibenzylideneacetone)dipalladium(O) (71 mg, 0.077 mmol, 0.1 equiv) and toluene (10 mL). The mixture was sparged with nitrogen for 10 minutes, sealed and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue which was purified on a Biotage automated chromatography system (55 g Biotage KPNH column), eluting with a gradient of 0 to 50% ethyl acetate in hexanes to give the title compound (530 mg, 76% yield) as a yellow solid. LCMS: m/z=901.4 [M+H]⁺.

### Example 169: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N—((S)-piperidin-3-yl)thiazol-2-amine

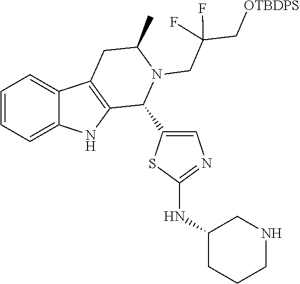

Trifluoroacetic acid (2.7 mL, 35.3 mmol, 60 equiv) was added to tert-butyl (1S,3R)-1-(2-(((S)-1-(tert-butoxycarbonyl)piperidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (530 mg, 0.589 mmol, 1 equiv) in dichloromethane (5.4 mL) at 0° C. After stirring at 0° C. for 24 hours, the reaction was diluted with cold dichloromethane (50 mL) and adjusted to pH 10 with saturated sodium carbonate (40 mL). The layers were separated and the organic layer was washed with saturated sodium carbonate (60 mL). The organic layer was dried over sodium sulfate, filtered and concentrated under reduced pressure to give title compound (224 mg, 54% yield) as an orange solid. LCMS: m/z=700.3 [M+H]⁺.

### Example 170: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N—((S)-1-(3-fluoropropyl) piperidin-3-yl)thiazol-2-amine

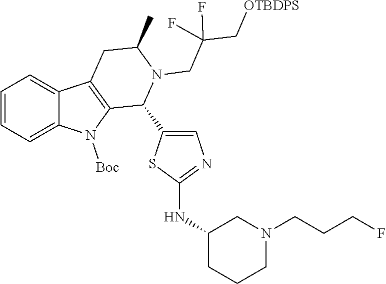

1-Bromo-3-fluoropropane (54 mg, 0.38 mmol, 1.2 equiv) and N,N-diisopropylethylamine (0.083 mL 0.48 mmol, 1.5 equiv) were sequentially added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((S)-piperidin-3-yl)thiazol-2-amine (224 mg, 0.32 mmol, 1 equiv) in N,N-dimethylacetamide (1.0 mL). After stirring for 20 hours at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite®. The residue was purified on a Biotage automated chromatography system (11 g, Biotage 50 μm KPNH column), eluting with a gradient of 1 to 100% ethyl acetate in dichloromethane to give the title compound (102 mg, 42% yield) as a white solid. LCMS: m/z=761.2 [M+H]⁻.

### Example 171: 2,2-Difluoro-3-((1S,3R)-1-(2-(((S)-1-(3-fluoropropyl)piperidin-3-yl)amino) thiazol-5-yl)-3-methyl-1,3,4,9-tetrahydro-2H-pyrido[3,4-b]indol-2-yl)propan-1-ol

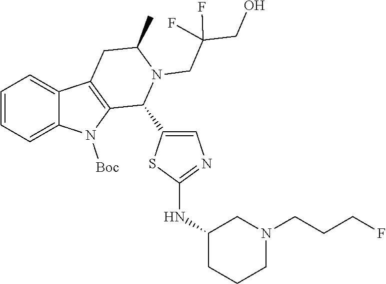 [SMILES: C[C@@H]1Cc2c(n(C(=O)OC(C)(C)C)c3ccccc23)[C@@H](c2cnc(N[C@H]3CCCN(CCCF)C3)s2)N1CC(F)(F)CO]

1M Tetra-n-butylammonium fluoride in THF (0.148 mL, 0.148 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((S)-1-(3-fluoropropyl) piperidin-3-yl)thiazol-2-amine (102 mg, 0.134 mmol, 1 equiv) in THF (1.5 mL). After stirring for 1 hour, the reaction was concentrated under reduced pressure and the residue was diluted with saturated ammonium chloride (5 mL). The suspension was extracted with ethyl acetate (3×5 mL). The combined organic layers were dried over sodium sulfate, filtered and concentrated under reduced pressure. The residue was purified on a Biotage automated chromatography system (Biotage 11 g KPNH, eluting with gradient of 0 to 10% methanol in dichloromethane to give the title compound (32 mg, 46% yield) as an off-white solid. ¹H-NMR (400 MHz, CDCl₃) δ=8.11 (br s, 1H), 7.51 (d, J=7.2 Hz, 1H), 7.32 (d, J=7.9 Hz, 1H), 7.18 (dt, J=1.3, 7.5 Hz, 1H), 7.14-7.09 (m, 1H), 6.72 (s, 1H), 5.67 (br s, 1H), 5.08 (s, 1H), 4.53 (t, J=5.9 Hz, 1H), 4.41 (t, J=5.9 Hz, 1H), 3.97-3.86 (m, 2H), 3.72-3.45 (m, 3H), 3.29-3.10 (m, 1H), 3.05-2.88 (m, 1H), 2.80 (dd, J=4.4, 16.0 Hz, 1H), 2.62-2.37 (m, 6H), 2.21 (br s, 1H), 1.90-1.73 (m, 3H), 1.66-1.43 (m, 3H), 1.28-1.21 (m, 3H); LCMS: m/z=522.3 [M+H]⁺.

### Example 172: tert-Butyl (1S,3R)-1-(2-(((R)-1-(tert-butoxycarbonyl)piperidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

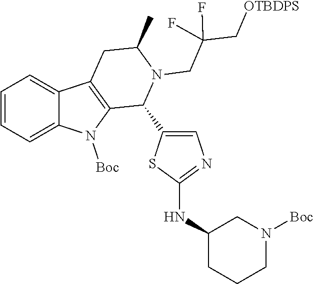

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (600 mg, 0.768 mmol, 1 equiv), tert-butyl (R)-3-aminopiperidine-1-carboxylate (308 mg, 1.54 mmol, 2 equiv), sodium tert-butoxide (111 mg, 1.15 mmol, 1.5 equiv), 2,2′-bis(diphenylphosphino)-1,1′-binaphthyl (96 mg, 0.154 mmol, 0.2 equiv), tris(dibenzylideneacetone)dipalladium(O) (71 mg, 0.077 mmol, 0.1 equiv) and toluene (10 mL). The mixture was sparged with nitrogen for 10 minutes, sealed, and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue was purified on a Biotage automated chromatography system (55 g Biotage KPNH column), eluting with a gradient of 0 to 50% ethyl acetate in dichloromethane to give the title compound (181 mg, 29% yield) as a yellow solid. LCMS: m/z=901.4 [M+H]⁺.

### Example 173: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N—((R)-piperidin-3-yl)thiazol-2-amine

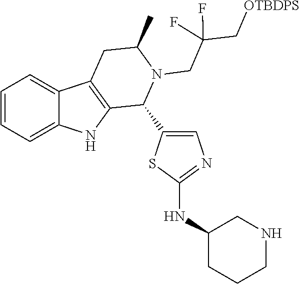

Trifluoroacetic acid (1.0 mL, 12.1 mmol, 60 equiv) was added to tert-butyl (1S,3R)-1-(2-(((R)-1-(tert-butoxycarbonyl)piperidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (181 mg, 0.2 mmol, 1 equiv) in dichloromethane (1.0 mL) at 0° C. After stirring at 0° C. for 24 hours, the reaction was diluted with cold dichloromethane (10 mL) and adjusted to pH 10 with saturated sodium carbonate solution (4 mL). The layers were separated and the organic layer was washed with saturated sodium carbonate (60 mL). The organic layer was dried over sodium sulfate, filtered and concentrated under reduced pressure to give the title compound (160 mg, 114% yield) as an orange solid. LCMS: m/z=700.3 [M+H]⁺.

### Example 174: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N—((R)-1-(3-fluoropropyl) piperidin-3-yl)thiazol-2-amine

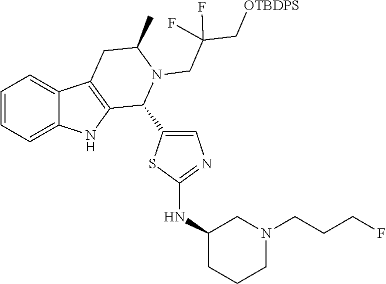

1.0 M 1-Bromo-3-fluoropropane in N,N-dimethylacetamide (0.25 mL, 0.25 mmol, 1.1 equiv) and N,N-diisopropylethylamine (0.06 mL 0.35 mmol, 1.5 equiv) were sequentially added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((R)-piperidin-3-yl)thiazol-2-amine (160 mg, 0.23 mmol, 1 equiv) in N,N-dimethylacetamide (0.75 mL). After stirring for 20 hours at room temperature, the reaction mixture was diluted with ethyl acetate (5 mL) and sequentially washed with water (3×2 mL) and saturated brine (2 mL). The organic layer was dried over sodium sulfate, filtered and concentrated under reduced pressure onto Celite®. The residue was purified on a Biotage automated chromatography system (11 g, Biotage 50 μm KPNH column), eluting with a gradient of 1 to 70% ethyl acetate in hexane to give the title compound (40 mg, 23% yield) as a white solid. LCMS: m/z=761.2 [M+H]⁺.

### Example 175: 2,2-Difluoro-3-((1S,3R)-1-(2-(((R)-1-(3-fluoropropyl)piperidin-3-yl)amino) thiazol-5-yl)-3-methyl-1,3,4,9-tetrahydro-2H-pyrido[3,4-b]indol-2-yl)propan-1-ol

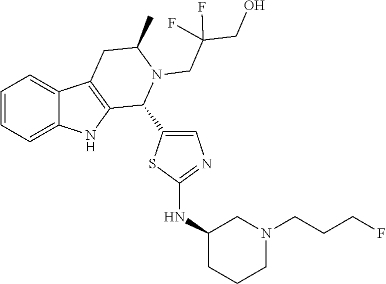 [SMILES: C[C@@H]1Cc2c([nH]c3ccccc23)[C@@H](c2cnc(N[C@@H]3CCCN(CCCF)C3)s2)N1CC(F)(F)CO]

1M Tetra-n-butylammonium fluoride in THF (0.06 mL, 0.057 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N—((R)-1-(3-fluoropropyl) piperidin-3-yl)thiazol-2-amine (40 mg, 0.052 mmol, 1 equiv) in THF (1.5 mL). After stirring for 1 hour, the reaction was concentrated under reduced pressure and the residue was diluted with saturated ammonium chloride (5 mL). The suspension was extracted with ethyl acetate (3×5 mL). The combined organic layers were dried over sodium sulfate, filtered and concentrated under reduced pressure. The residue was purified on Biotage automated chromatography system (Biotage 5 g KPNH, eluting with gradient of 0 to 10% methanol in dichloromethane to give the title compound (7 mg, 26% yield) as an off-white solid. ¹H-NMR (400 MHz, CDCl₃) δ=8.00 (br s, 1H), 7.51 (d, J=7.8 Hz, 1H), 7.31 (d, J=7.8 Hz, 1H), 7.18 (dt, J=1.3, 7.5 Hz, 1H), 7.14-7.09 (m, 1H), 6.72 (s, 1H), 5.67 (br s, 1H), 5.07 (s, 1H), 4.54 (t, J=5.9 Hz, 1H), 4.42 (t, J=6.0 Hz, 1H), 3.92 (br t, J=12.2, 2H), 3.70-3.46 (m, 3H), 3.25-3.10 (m, 1H), 3.05-2.90 (m, 1H), 2.81 (dd, J=4.4, 15.9 Hz, 1H), 2.61-2.38 (m, 6H), 2.24 (br s, 1H), 1.90-1.77 (m, 3H), 1.75-1.59 (m, 3H), 1.57-1.45 (m, 1H), 1.22 (d, 0.1=6.7, 3H); LCMS: m/z=522.2 [M+H]⁺.

### Example 176: tert-Butyl 4-(5-formylthiophen-2-yl)-3,6-dihydropyridine-1(2H)-carboxylate

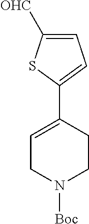 [SMILES: CC(C)(C)OC(=O)N1CC=C(c2ccc(C=O)s2)CC1]

5-Bromothiophene-2-carbaldehyde (5 g, 26.2 mmol, 1.0 equiv) and 3,6-dihydro-2H-pyridine-1-N-Boc-4-boronic acid pinacol ester (9.7 g, 31.4 mmol, 1.2 equiv) were stirred at room temperature in a 3 to 1 mixture of 1,4-dioxane and saturated sodium bicarbonate (130 mL). The reaction mixture was sparged with a stream of nitrogen for 15 minutes. Tetrakis(triphenylphosphine)palladium(O) (1.51 g, 1.31 mmol, 0.05 equiv) was then added and the reaction mixture was sparged with a stream of nitrogen for another 15 minutes. After heating at 90° C. for 18 hours. The reaction mixture was cooled to room temperature and transferred to a separatory funnel with dichloromethane (50 mL) and water (50 mL). The layers were separated and the aqueous phase was extracted with dichloromethane (2×30 mL). The combined organic layers were washed with saturated brine (50 mL), dried over sodium sulfate, filtered and concentrated under reduced pressure onto silica gel (40 g). The residue was purified on a Biotage automated chromatography system (200 g, Biotage® Sfar Silica HC High Capacity 20 μm), eluting with a gradient of 0 to 20% ethyl acetate in heptanes. The solid was dried under vacuum at 45° C. for 16 hours to give the title compound (4.60 g, 60% yield) as a white solid. LCMS: m/z=294.3 [M+H]⁺.

### Example 177: tert-Butyl 4-(5-(hydroxymethyl)thiophen-2-yl)piperidine-1-carboxylate

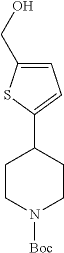 [SMILES: CC(C)(C)OC(=O)N1CCC(c2ccc(CO)s2)CC1]

A mixture of tert-butyl 4-(5-formylthiophen-2-yl)-3,6-dihydropyridine-1(2H)-carboxylate (5 g, 17.0 mmol, 1.0 equiv) and 10% Pd/C (2.5 g, 50% wet) in methanol (40 mL) was hydrogenated @25 psi at room temperature for 18 hours. After the reaction was completed as judged by LC-MS, the mixture was filtered through a pad of Celite® (10 g) under an atmosphere of nitrogen. The pad was washed with methanol (2×30 mL). The filtrate was concentrated under reduced pressure onto silica gel (20 g). The residue was purified on a Biotage automated chromatography system (100 g, Biotage® Sfar Silica HC High Capacity 60 μm), eluting with a gradient of 0 to 30% ethyl acetate in hexanes. The solid was dried under vacuum at 45° C. for 16 hours to give the title compound (2.34 g, 46% yield) as a white solid. LCMS: m/z=298.3 [M+H]⁺.

### Example 178: tert-Butyl 4-(5-formylthiophen-2-yl)piperidine-1-carboxylate

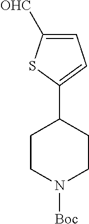 [SMILES: CC(C)(C)OC(=O)N1CCC(c2ccc(C=O)s2)CC1]

tert-Butyl 4-(5-(hydroxymethyl)thiophen-2-yl)piperidine-1-carboxylate (2.34 g, 7.9 mmol, 1.0 equiv) was treated with activated manganese dioxide (10.3 g, 118 mmol, 15.0 equiv) in dichloromethane at room temperature for 18 hours. After the reaction was completed as judged by LC-MS, the mixture was filtered through a pad of Celite® (10 g) under an atmosphere of nitrogen. The pad was washed with dichloromethane (3×20 mL). The filtrate was concentrated under reduced pressure and dried under vacuum at 45° C. for 16 hours to give the title compound (2.31 g, 99% yield) as a white solid, which was used subsequently. LCMS: m/z=296.3 [M+H]⁺

### Example 179: tert-Butyl 4-(5-((1S,3R)-2-(2-fluoro-2-methylpropyl)-3-methyl-2,3,4,9-tetrahydro-1H-pyrido[3,4-b]indol-1-yl)thiophen-2-yl)piperidine-1-carboxylate

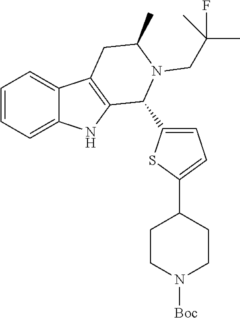 [SMILES: C[C@@H]1Cc2c([nH]c3ccccc23)[C@@H](c2ccc(C3CCN(C(=O)OC(C)(C)C)CC3)s2)N1CC(C)(C)F]

tert-Butyl 4-(5-formylthiophen-2-yl)piperidine-1-carboxylate (0.5 g, 1.69 mmol, 1 equiv) and acetic acid (0.5 mL, 8.5 mmol, 5.0 equiv) were sequentially added to a solution of (R)—N-(1-(1H-indol-3-yl)propan-2-yl)-2-fluoro-2-methylpropan-1-amine (0.51 g, 2.03 mmol, 1.2 equiv) in toluene (10 mL) in a 50 mL round bottom flask. After heating at 80° C. for 21 hours, the reaction was cooled to room temperature and transferred to a separatory funnel with ethyl acetate (30 mL) and washed with saturated sodium bicarbonate (3×10 mL). The organic layer was dried over sodium sulfate, filtered and concentrated under reduced pressure onto silica gel (10 g). The residue was purified on a Biotage automated chromatography system (50 g, Biotage® Sfar Silica HC High Capacity 60 m), eluting with a gradient of 0 to 20% ethyl acetate in hexanes. The solid was dried under vacuum at 40° C. for 16 hours to give the title compound (0.72 g, 81% yield) as a yellow solid. LCMS: m/z=526.3 [M+H]⁺.

### Example 180: (1S,3R)-2-(2-Fluoro-2-methylpropyl)-3-methyl-1-(5-(piperidin-4-yl)thiophen-2-yl)-2,3,4,9-tetrahydro-1H-pyrido[3,4-b]indole

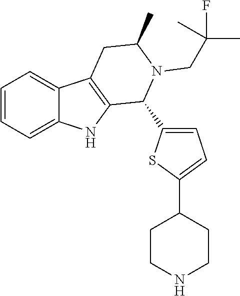 [SMILES: C[C@@H]1Cc2c([nH]c3ccccc23)[C@@H](c2ccc(C3CCNCC3)s2)N1CC(C)(C)F]

Trifluoroacetic acid (3.2 mL, 41.1 mmol, 30 equiv) was added dropwise over −2 minutes to tert-butyl 4-(5-((1S,3R)-2-(2-fluoro-2-methylpropyl)-3-methyl-2,3,4,9-tetrahydro-1H-pyrido[3,4-b]indol-1-yl)thiophen-2-yl)piperidine-1-carboxylate (0.72 g, 1.37 mmol, 1.0 equiv) in dichloromethane (3.2 mL) in a 40 mL vial at 0° C. The reaction was stirred at 0° C. for 4 hours. Cold saturated sodium carbonate was added to the mixture adjusting the pH to 10. The solution was transferred to a separatory funnel and was extracted with dichloromethane (3×15 mL). The combined organic layers were washed with saturated brine (1×5 mL), dried over sodium sulfate, filtered and concentrated under reduced pressure. The product was dried under vacuum at 25° C. for 1.5 hours to give the title compound (0.58 g, 100% yield) as a yellow solid, which was used subsequently. LCMS: m/z=426.3 [M+H]⁺.

### Example 181: (1S,3R)-2-(2-Fluoro-2-methylpropyl)-1-(5-(1-(2-fluoroethyl)piperidin-4-yl)thiophen-2-yl)-3-methyl-2,3,4,9-tetrahydro-1H-pyrido[3,4-b]indole

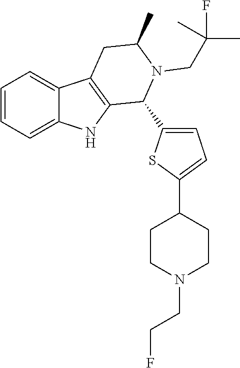 [SMILES: C[C@@H]1Cc2c([nH]c3ccccc23)[C@@H](c2ccc(C3CCN(CCF)CC3)s2)N1CC(C)(C)F]

1-Fluoro-2-iodoethane (36 μL, 0.44 mmol, 1.5 equiv) and N,N-diisopropylethylamine (0.25 mL, 1.5 mmol, 5.0 equiv) were added to (1S,3R)-2-(2-fluoro-2-methylpropyl)-3-methyl-1-(5-(piperidin-4-yl)thiophen-2-yl)-2,3,4,9-tetrahydro-1H-pyrido[3,4-b]indole (123 mg, 0.29 mmol, 1.0 equiv) in N,N-dimethylacetamide (2.0 mL) in a 20 mL vial and stirred at room temperature for 3 days. The reaction mixture was transferred to a separatory funnel with ethyl acetate (20 mL) and washed with saturated brine (2×15 mL). The organic layer was dried over sodium sulfate, filtered and concentrated under reduced pressure onto Celite® (2 g). The material was purified on a Biotage automated chromatography system (28 g, Biotage 50 m KP-amino-d column), eluting with a gradient of 0 to 100% ethyl acetate in hexanes. The solid was dried under vacuum at 25° C. for 16 hours to give the title compound (70 mg, 51% yield) as a cream solid. ¹H NMR (400 MHz, CDCl₃) δ=7.83 (br s, 1H), 7.51 (d, J=7.7 Hz, 1H), 7.32 (d, J=7.9 Hz, 1H), 7.17 (dt, J=1.3, 7.5 Hz, 1H), 7.13-7.08 (m, 1H), 6.55 (s, 2H), 5.22 (br s, 1H), 4.63 (t, J=5.0 Hz, 1H), 4.51 (t, J=5.0 Hz, 1H), 3.45 (br s, 1H), 3.06-2.99 (m, 2H), 2.88-2.48 (m, 7H), 2.17 (dt, J=2.2, 11.7 Hz, 2H), 1.98 (br d, J=13.2 Hz, 2H), 1.86-1.69 (m, 2H), 1.55-1.27 (m, 6H), 1.15 (d, J=6.8 Hz, 3H); LCMS: m/z=472.3 [M+H]⁺.

### Example 182: (1S,3R)-1-(5-(1-Ethylpiperidin-4-yl)thiophen-2-yl)-2-(2-fluoro-2-methylpropyl)-3-methyl-2,3,4,9-tetrahydro-1H-pyrido[3,4-b]indole

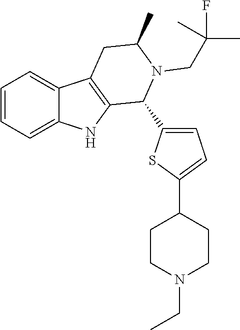 [SMILES: CCN1CCC(c2ccc([C@@H]3c4[nH]c5ccccc5c4C[C@@H](C)N3CC(C)(C)F)s2)CC1]

2.4 M Acetaldehyde in dichloromethane (95 μL, 0.23 mmol, 1.2 equiv) and acetic acid (28 μL, 0.48 mmol, 2.5 equiv) were sequentially added to a solution of (1S,3R)-2-(2-fluoro-2-methylpropyl)-3-methyl-1-(5-(piperidin-4-yl)thiophen-2-yl)-2,3,4,9-tetrahydro-1H-pyrido[3,4-b]indole (80 mg, 0.19 mmol, 1 equiv) in methanol (2 mL) in a 20 mL vial and stirred for 30 minutes. Sodium triacetoxyborohydride (81 mg, 0.38 mmol, 2.0 equiv) was added portion wise over 5 minutes. After stirring at room temperature for 24 hours, LC-MS analysis indicated the reaction was complete. Methanol was concentrated under reduced pressure and the reaction mixture was transferred to a separatory funnel with dichloromethane (20 mL) and washed with saturated sodium carbonate (20 mL). The aqueous layer was extracted with dichloromethane (3×20 mL). The combined organic layers were dried over sodium sulfate, filtered and concentrated under reduced pressure onto Celite® (2 g). The residue was purified on a Biotage automated chromatography system (28 g, Biotage 50 μm KP-amino-d column), eluting with a gradient of 0 to 100% ethyl acetate in hexanes to give the title compound (30 mg, ˜₇₀% LC purity). This material was absorbed onto Celite® (1 g) and was purified on a Biotage automated chromatography system (15.5 g, Redigold C18 column), eluting with a gradient of 0 to 100% acetonitrile in water to give the title compound (12 mg, 14% yield) as a white solid. ¹H NMR (400 MHz, CDCl₃) δ=7.86 (br s, 1H), 7.51 (d, J=7.7 Hz, 1H), 7.32 (d, J=8.1 Hz, 1H), 7.17 (dt, J=1.3, 7.5 Hz, 1H), 7.13-7.08 (m, 1H), 6.55 (s, 2H), 5.22 (br s, 1H), 3.45 (br s, 1H), 3.06-2.97 (m, 2H), 2.76-2.67 (m, 1H), 2.67-2.61 (m, 2H), 2.61-2.57 (m, 1H), 2.57-2.39 (m, 3H), 2.04-1.93 (m, 4H), 1.85-1.68 (m, 3H), 1.54-1.46 (m, 3H), 1.36 (s, 2H), 1.17-1.07 (m, 6H), LCMS (ESI) m/z=454.3 [M+H]⁺.

### Example 183: (1S,3R)-2-(2-Fluoro-2-methylpropyl)-3-methyl-1-(5-(1-propylpiperidin-4-yl)thiophen-2-yl)-2,3,4,9-tetrahydro-1H-pyrido[3,4-b]indole

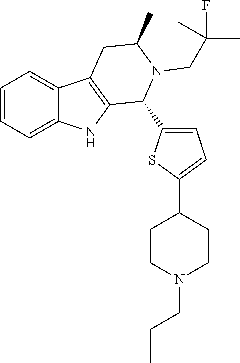 [SMILES: CCCN1CCC(c2ccc([C@@H]3c4[nH]c5ccccc5c4C[C@@H](C)N3CC(C)(C)F)s2)CC1]

Propionaldehyde (20 μL, 0.28 mmol, 1.2 equiv) and acetic acid (33 μL, 0.58 mmol, 2.5 equiv) were sequentially added to a solution of (1S,3R)-2-(2-fluoro-2-methylpropyl)-3-methyl-1-(5-(piperidin-4-yl)thiophen-2-yl)-2,3,4,9-tetrahydro-1H-pyrido[3,4-b]indole (100 mg, 0.24 mmol, 1 equiv) in methanol (2 mL) in a 20 mL vial and stirred at room temperature for 30 minutes. Sodium triacetoxyborohydride (98 mg, 0.5 mmol, 2.0 equiv) was added portion wise over 5 minutes. After stirring at room temperature for 3 hours, LC-MS analysis indicated the reaction was complete. Methanol was concentrated under reduced pressure and the reaction mixture was transferred to a separatory funnel with dichloromethane (20 mL) and washed with saturated sodium carbonate (20 mL). The aqueous layer was extracted with dichloromethane (3×20 mL). The combined organic layers were dried over sodium sulfate, filtered and concentrated under reduced pressure onto Celite® (2 g). The residue was purified on a Biotage automated chromatography system (28 g, Biotage 50 μm KP-amino-d column), eluting with a gradient of 0 to 100% ethyl acetate in hexanes. The solid was dried under vacuum at 25° C. for 16 hours to give the title compound (30 mg, 27% yield) as a white solid. ¹H NMR (400 MHz, CDCl₃) δ=7.83 (br s, 1H), 7.51 (d, J=7.8 Hz, 1H), 7.32 (d, J=7.9 Hz, 1H), 7.17 (dt, J=1.3, 7.5 Hz, 1H), 7.13-7.08 (m, 1H), 6.55 (s, 2H), 5.22 (br s, 1H), 3.45 (br s, 1H), 3.00 (br d, J=11.5 Hz, 2H), 2.75-2.48 (m, 5H), 2.33-2.27 (m, 2H), 2.05-1.92 (m, 4H), 1.87-1.68 (m, 2H), 1.56-1.46 (m, 5H), 1.37-1.29 (m, 3H), 1.15 (d, J=6.7 Hz, 3H), 0.90 (t, J=7.4 Hz, 3H); LCMS (ESI) m/z=468.3 [M+H]⁺.

### Example 184: (1S,3R)-2-(2-Fluoro-2-methylpropyl)-1-(5-(1-(3-fluoropropyl)piperidin-4-yl)thiophen-2-yl)-3-methyl-2,3,4,9-tetrahydro-1H-pyrido[3,4-b]indole

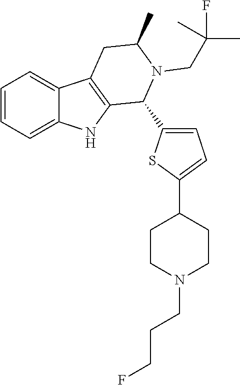 [SMILES: C[C@@H]1Cc2c([nH]c3ccccc23)[C@@H](c2ccc(C3CCN(CCCF)CC3)s2)N1CC(C)(C)F]

1-Bromo-3-fluoropropane (32 μL, 0.34 mmol, 1.5 equiv) and N,N-diisopropylethylamine (0.2 mL, 1.5 mmol, 5.0 equiv) was added to (1S,3R)-2-(2-fluoro-2-methylpropyl)-3-methyl-1-(5-(piperidin-4-yl)thiophen-2-yl)-2,3,4,9-tetrahydro-1H-pyrido[3,4-b]indole (97 mg, 0.23 mmol, 1.0 equiv) in N,N-dimethylacetamide (2.0 mL) in a 20 mL vial and stirred at room temperature for 3 days. The reaction mixture was transferred to a separatory funnel with ethyl acetate (20 mL) and washed with saturated brine (2×15 mL). The organic layer was dried over sodium sulfate, filtered and concentrated under reduced pressure onto Celite® (2 g). The material was purified on a Buchi automated chromatography system (28 g, Biotage 50 μm KP-amino-d column), eluting with a gradient of 0 to 100% ethyl acetate in hexanes. The solid was dried under vacuum at 25° C. for 16 hours to give the title compound (33 mg, 30% yield) as a cream solid. ¹H NMR (400 MHz, CDCl₃) δ=7.81 (br s, 1H), 7.51 (d, J=7.7 Hz, 1H), 7.32 (d, J=7.7 Hz, 1H), 7.17 (dt, J=1.3, 7.5 Hz, 1H), 7.13-7.08 (m, 1H), 6.55 (s, 2H), 5.22 (br s, 1H), 4.57 (t, J=6.0 Hz, 1H), 4.45 (t, J=6.0 Hz, 1H), 3.45 (br s, 1H), 2.98 (br d, J=11.6 Hz, 2H), 2.77-2.58 (m, 4H), 2.55-2.46 (m, 3H), 2.09-1.84 (m, 6H), 1.82-1.72 (m, 2H), 1.52-1.45 (m, 2H), 1.36 (s, 2H), 1.31 (s, 2H), 1.15 (d, J=6.7 Hz, 3H); LCMS: m/z=486.3 [M+H]⁺.

### Example 185: tert-Butyl (1S,3R)-1-(2-((3S,4S)-1-(tert-butoxycarbonyl)-4-fluoropyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

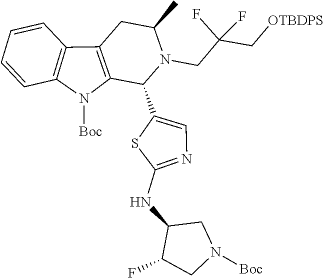

A 40 mL vial was charged with tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (500 mg, 0.64 mmol, 1 equiv), tert-butyl (3S,4S)-3-amino-4-fluoropyrrolidine-1-carboxylate (261 mg, 1.28 mmol, 2 equiv), sodium tert-butoxide (92.2 mg, 0.96 mmol, 1.5 equiv), 2,2′-bis(diphenylphosphino)-1,1′-binaphthyl (80 mg, 0.128 mmol, 0.2 equiv), tris(dibenzylideneacetone)dipalladium(O) (59 mg, 0.064 mmol, 0.1 equiv) and toluene (8 mL). The mixture was sparged with nitrogen for 10 minutes, sealed, and heated in a CEM microwave at 110° C. for 1 hour (constant power method, 150 watts). After cooling to room temperature, water (10 mL) was added and the mixture was extracted with ethyl acetate (3×20 mL). The combined organic layers were dried over sodium sulfate, filtered and concentrated under reduced pressure. The residue was purified on a Biotage automated chromatography system (55 g Biotage KPNH column), eluting with a gradient of 0 to 50% ethyl acetate in hexanes to give the title compound (168 mg, 29% yield) as a yellow foam. LCMS: m/z=905.3 [M+H]⁺.

### Example 186: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-yl)-N-((3S,4S)-4-fluoropyrrolidin-3-yl)thiazol-2-amine

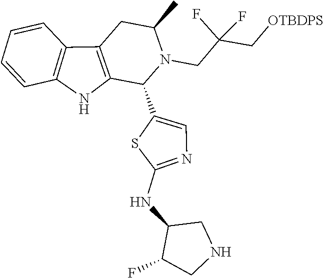

Trifluoroacetic acid (1.6 mL, 21.1 mmol, 60 equiv) was added to tert-butyl (1S,3R)-1-(2-(((3S,4S)-1-(tert-butoxycarbonyl)-4-fluoropyrrolidin-3-yl)amino)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (168 mg, 0.186 mmol, 1 equiv) in dichloromethane (1.7 mL) at 0° C. in a 40 mL vial. After stirring at 0° C. for 24 hours, the reaction was diluted with cold dichloromethane (50 mL) and adjusted to pH 10 with saturated sodium carbonate (40 mL). The layers were separated and the organic layer was washed with saturated sodium carbonate (60 mL). The organic layer was dried over sodium sulfate, filtered and concentrated under reduced pressure to give the title compound (142 mg, >99% yield) as an orange solid. LCMS: m/z=704.1 [M+H]⁺.

### Example 187: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-N-((3S,4S)-4-fluoro-1-(3-fluoropropyl)pyrrolidin-3-yl)thiazol-2-amine

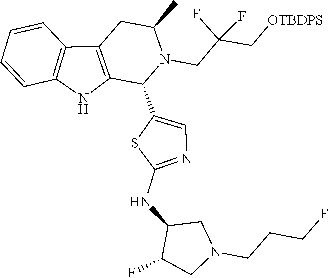

1-Bromo-3-fluoropropane (31 mg, 0.223 mmol, 1.2 equiv) and N,N-diisopropylethylamine (0.048 mL, 0.28 mmol, 1.5 equiv) were sequentially added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N-((3S,4S)-4-fluoropyrrolidin-3-yl)thiazol-2-amine (142 mg, 0.186 mmol, 1 equiv) in N,N-dimethylacetamide (0.6 mL). After stirring for 20 hours at room temperature, the reaction mixture was diluted with ethyl acetate (20 mL) and sequentially washed with water (3×20 mL) and saturated brine (20 mL). The organic layer was dried over sodium sulfate, filtered and concentrated under reduced pressure onto Celite®. The residue was purified on a Biotage automated chromatography system (11 g, Biotage 50 μm KPNH column), eluting with a gradient of 5 to 100% ethyl acetate in dichloromethane to give the title compound (68 mg, 48% yield) as a yellow solid. LCMS: m/z=765.2 [M+H]⁺.

### Example 188: 2,2-Difluoro-3-((1S,3R)-1-(2-(((3S,4S)-4-fluoro-1-(3-fluoropropyl)pyrrolidin-3-yl)amino)thiazol-5-yl)-3-methyl-1,3,4,9-tetrahydro-2H-pyrido[3,4-b]indol-2-yl)propan-1-ol

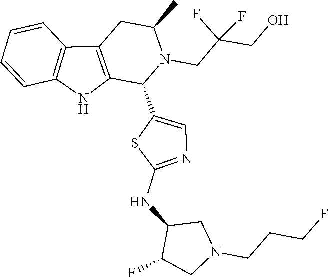 [SMILES: C[C@@H]1Cc2c([nH]c3ccccc23)[C@@H](c2cnc(N[C@H]3CN(CCCF)C[C@@H]3F)s2)N1CC(F)(F)CO]

1M Tetra-n-butylammonium fluoride in THE (0.098 mL, 0.098 mmol, 1.1 equiv) was added dropwise at room temperature to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-N-((3S,4R)-4-fluoro-1-(3-fluoropropyl)pyrrolidin-3-yl)thiazol-2-amine (68 mg, 0.09 mmol, 1 equiv) in THF (1.5 mL). After stirring for 1 hour, the reaction was concentrated under reduced pressure and the residue was diluted with saturated ammonium chloride (5 mL). The suspension was extracted with ethyl acetate (3×5 mL). The combined organic layers were dried over sodium sulfate, filtered and concentrated under reduced pressure. The residue was purified on a Biotage automated chromatography system (Biotage 11 g KPNH), eluting with gradient of 10 to 100% ethyl acetate in dichloromethane to give the title compound (15 mg, 32% yield) as a white solid. ¹H NMR (400 MHz, CDCl₃) δ=8.32 (br s, 1H), 8.21 (s, 1H), 7.51 (d, J=7.5 Hz, 1H), 7.34-7.28 (m, 1H), 7.21-7.09 (m, 2H), 6.75 (s, 1H), 5.50 (br d, J=5.5 Hz, 1H), 5.15-5.07 (m, 1H), 5.03-4.85 (m, 1H), 4.55 (t, J=5.9 Hz, 1H), 4.47-4.41 (m, 1H), 4.33-4.07 (m, 1H), 3.93 (br t, J=12.3 Hz, 2H), 3.70-3.44 (m, 2H), 3.44-3.26 (m, 1H), 3.23-3.09 (m, 2H), 3.00-2.87 (m, 2H), 2.82-2.71 (m, 1H), 2.67-2.40 (m, 5H), 1.92-1.64 (m, 2H), 1.31-1.16 (m, 3H); LCMS: m/z=526.2 [M+H]⁺.

### Example 189: Biological Evaluation

ECC-1 cells were trypsinized and resuspended in hormone-depleted media and plated at a density of 15 k cells per well into a 96-well plate for at least 4 hours. Cells were treated with test compounds in the absence of E2 (for agonist mode) or in the presence of 500 pM E2 (for antagonist mode) for 3 days and plates were subsequently frozen at −80° C. Thawed plates were incubated with a chromogenic substrate of AP, p-nitrophenyl phosphate (Thermo Fisher Scientific), for 40 minutes at 42° C., and absorbances were read at 405 nm. AP activity was normalized to the activity of 500 pM E2 alone. This assay was shown to correlate with the in vivo studies comparing uterine wet weight in ovariectomized rats following treatment with a number of antiestrogens.

Table 6 shows estrogen receptor modulation (e.g., agonism and antagonism) of certain compounds of the present disclosure. The compound numbers correspond to the compound numbers of Table 1 and 2. Compounds having an activity designated as “+” provided estrogen receptor agonism having (i) at least 80% increase in the E2-normalized signal in the AP assay (agonist mode) and (ii) no more than 80% reduction in the E2-normalized signal in the AP assay (antagonist mode). Compounds having an activity designated as “++” provided estrogen receptor antagonism having (i) between 10% and 80% increase in the E2-normalized signal in the AP assay (agonist mode) and (ii) between 10% and 80% reduction in the E2-normalized signal in the AP assay (antagonist mode). Compounds having an activity designated as “+++” provided estrogen receptor antagonism (i) with pIC₅₀ greater than 7.5 and at least a 10% reduction in the E2-normalized signal in the AP assay (antagonist mode); and no more than 10% increase in E2-normalized signal in the AP assay (agonist mode).

In some embodiments, particularly useful modulators of the estrogen receptor are compounds having greater than “+” activity in Table 6. In some embodiments, particularly useful modulators of the estrogen receptor are compounds having greater than “++” activity in Table 6. In some embodiments, particularly useful modulators of the estrogen receptor are compounds having “+++” activity in Table 6.

TABLE 6

| Compound No. | Activity |
|---|---|
| I-1 | ++ |
| I-2 | ++ |
| I-3 | + |
| I-4 | + |
| I-5 | ++ |
| I-6 | ++ |
| I-7 | ++ |
| I-8 | ++ |
| I-9 | ++ |
| I-10 | ++ |
| I-11 | ++ |
| I-12 | ++ |
| I-13 | ++ |
| I-14 | ++ |
| I-15 | ++ |
| I-16 | +++ |
| I-17 | +++ |
| I-18 | ++ |
| I-19 | ++ |
| I-20 | ++ |
| I-21 | +++ |
| I-22 | ++ |
| I-23 | ++ |
| I-24 | ++ |
| I-25 | ++ |
| I-26 | ++ |
| I-27 | ++ |
| I-28 | ++ |
| I-29 | +++ |
| I-30 | ++ |
| I-31 | ++ |
| I-32 | ++ |
| I-33 | ++ |
| I-34 | ++ |
| 1-35 | ++ |
| I-36 | + |
| I-37 | ++ |
| I-38 | ++ |
| I-39 | ++ |
| I-40 | +++ |
| I-41 | ++ |
| I-42 | ++ |
| I-43 | ++ |
| I-44 | ++ |
| I-45 | + |
| I-46 | ++ |
| I-47 | + |
| I-48 | ++ |
| I-49 | + |
| I-50 | ++ |
| I-51 | + |
| I-52 | ++ |
| 1-53 | ++ |
| I-54 | + |
| I-55 | +++ |
| I-56 | ++ |
| I-57 | ++ |
| I-58 | + |
| I-59 | + |
| I-60 | ++ |
| I-61 | ++ |
| I-62 | ++ |
| I-63 | ++ |
| I-64 | ++ |
| I-65 | + |
| I-66 | ++ |
| I-67 | + |
| I-68 | ++ |
| I-69 | ++ |
| I-70 | + |
| I-71 | + |
| I-72 | ++ |
| I-73 | ++ |
| I-74 | + |

The embodiments of the disclosure described above are intended to be merely exemplary, numerous variations and modifications will be apparent to those skilled in the art. All such variations and modifications are intended to be within the scope of the present invention as defined in any appended claims.

1. A compound of Formula I: 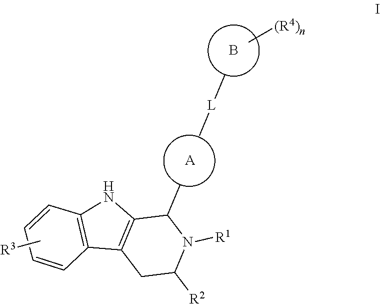
or a pharmaceutically acceptable salt thereof, wherein:
A is an optionally substituted 5-membered heteroaryl comprising 1-3 heteroatoms selected from N, O, and S, wherein at least 1 heteroatom is S or O;
L is a covalent bond or an optionally substituted bivalent group selected from C₁-C₆ aliphatic, -L^{a}-C₀-C₅ aliphatic-, and —C₁-C₅ aliphatic-L^{a}-, wherein L^{a} is selected from —S—, —SO—, —SO₂—, and —N(R^{a})—;
B is selected from 3- to 12-membered heterocyclyl comprising 1-3 heteroatoms selected from N, O, and S and C₃-C₆ cycloaliphatic;
R¹ is selected from hydrogen and optionally substituted C₁-C₆ aliphatic;
R² is selected from hydrogen and optionally substituted C₁-C₆ aliphatic;
R³ is selected from hydrogen, halogen, —CN, —OR^{a}, —C(O)R^{a}, —C(O)OR^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)OR^{a}, —N(R^{a})S(O)₂R^{a}, —SR a, —S(O)₂R^{a}, —S(O)N(R^{a})₂, —S(O)₂N(R^{a})₂, and an optionally substituted C₁₋₆ aliphatic group;
each R⁴ is independently oxo, halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S;
each R^{a} is independently selected from hydrogen and optionally substituted C₁-C₆ aliphatic; and
n is 0 to 5.

2. The compound of claim 1, wherein R¹ is optionally substituted C₁-C₆ aliphatic.

3. The compound of claim 1 or 2, wherein R¹ is C₁-C₆ aliphatic optionally substituted with one or more halogen or —OH.

4. The compound of claim 3, wherein R¹ is C₁-C₆ aliphatic optionally substituted with one or more fluoro or —OH.

5. The compound of any one of claims 1-4, wherein R¹ is selected from:

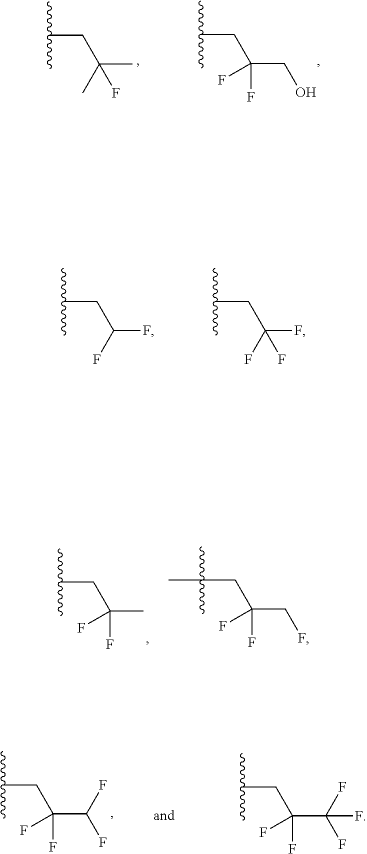

6. The compound of any one of claims 1-5, wherein R² is optionally substituted C₁-C₆ aliphatic.

7. The compound of any one of claims 1-6, wherein R² is methyl.

8. The compound of any one of claims 1-7, wherein R³ is hydrogen.

9. The compound of any one of claims 1-8, wherein A is optionally substituted 5-membered heteroaryl comprising 1-3 heteroatoms selected from N and S, and wherein at least 1 heteroatom is S.

10. The compound of any one of claims 1-9, wherein A is optionally substituted thiophenyl or optionally substituted thiazolyl.

11. The compound of any one of claims 1-10, wherein A is optionally substituted with halogen or C₁-C₆ aliphatic.

12. The compound of any one of claims 1-10, wherein A is selected from: 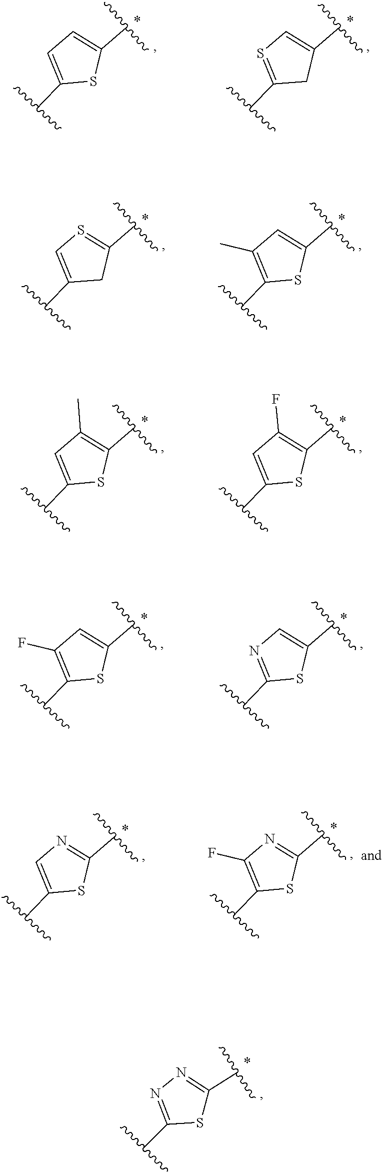
wherein * represents a point of attachment to moiety L.

13. The compound of any one of claims 1-12, wherein B is 3- to 6-membered heterocyclyl comprising 1-3 heteroatoms selected from N, O, and S.

14. The compound of any one of claims 1-13, wherein B is 4- to 5-membered heterocyclyl comprising 1-3 heteroatoms selected from N, O, and S.

15. The compound of any one of claims 1-14, wherein B is azetidinyl or pyrrolidinyl.

16. The compound of any one of claims 1-15,
wherein: 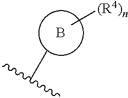
is a moiety selected from:

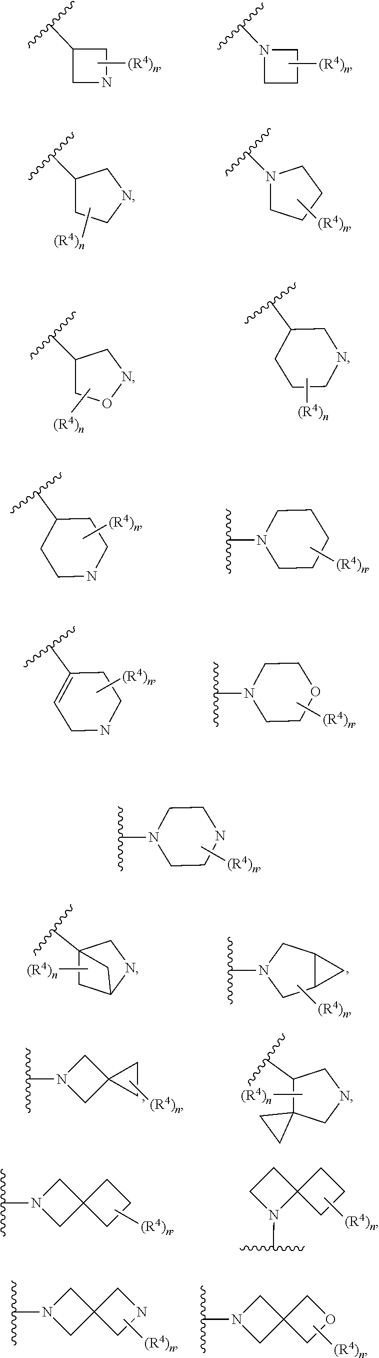

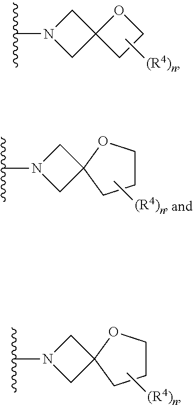

17. The compound of any one of claims 1-16, wherein 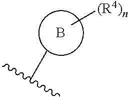
is a moiety selected from:

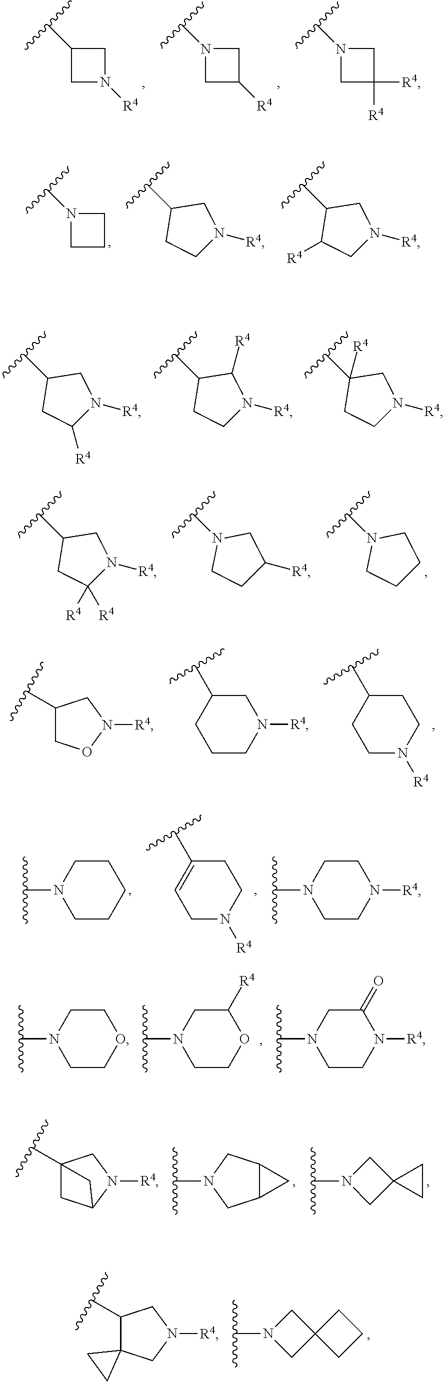

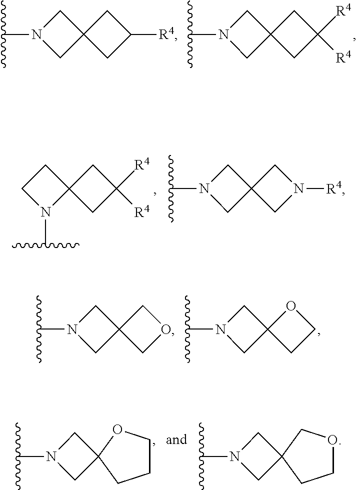

18. The compound of any one of claims 1-12, wherein B is C₃-C₆ cycloaliphatic.

19. The compound of claim 18, wherein B is selected from cyclopropyl, cyclobutyl, cyclopentyl, and cyclohexyl.

20. The compound of any one of claims 1-19, wherein L is selected from optionally substituted C₁-C₆ aliphatic, —S—C₀-C₅ aliphatic-, and —N(H)—C₀-C₅ aliphatic-.

21. The compound of any one of claims 1-20, wherein L is optionally substituted C₁-C₆ aliphatic.

22. The compound of any one of claims 1-21, wherein L is —CH₂—.

23. The compound of any one of claims 1-20, wherein L is selected from —CH₂—, —CH₂—CH₂—, —CH₂—CH₂—CH₂—, —S—, and —N(H)—.

24. The compound of any one of claims 1-16 or 18-23, wherein n is 1.

25. The compound of any one of claims 1-16 or 18-23, wherein n is 0.

26. The compound of any one of claims 1-24, wherein each R⁴ is independently selected from halogen and an optionally substituted C₁-C₆ aliphatic group.

27. The compound of any one of claims 1-24 or 26, wherein each R⁴ is an independently selected optionally substituted C₁-C₆ aliphatic group.

28. The compound of any one of claims 1-24 or 26-27, wherein R⁴ is —(CH₂)₂CH₃.

29. The compound of any one of claims 1-24 or 26-27, wherein R⁴ is —(CH₂)₃F.

30. The compound of any one of claims 1-24, wherein each R⁴ is independently selected from fluoro, —CH₃, —CH₂CH₃, —(CH₂)₂CH₃, —CH₂F, —(CH₂)₃F, —(CH₂)₂F, —(CH₂)₂CF₃, —(CH₂)₂CHF₂, —(CH₂)₃—O—CH₃, —CH₂C≡CH, and —CH₂CH₂CN.

31. The compound of any one of claims 1-30, wherein the compound is of Formula II: 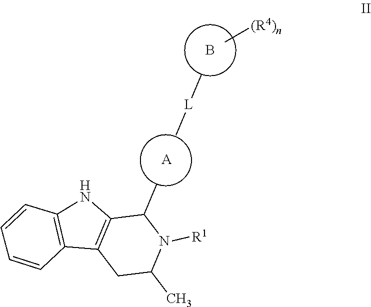
or a pharmaceutically acceptable salt thereof.

32. The compound of claim 31, wherein the compound is of Formula II-a: 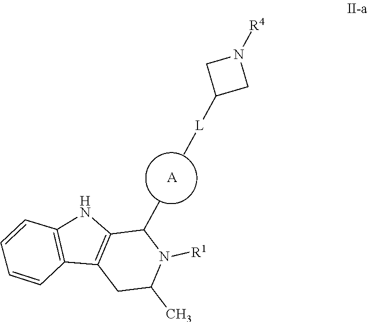
or a pharmaceutically acceptable salt thereof.

33. The compound of claim 31, wherein the compound is of Formula II-b: 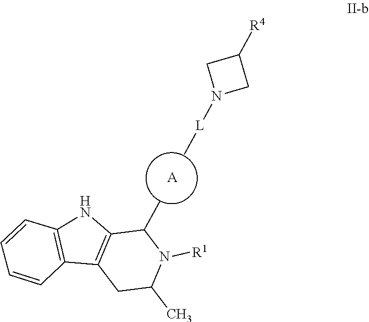
or a pharmaceutically acceptable salt thereof.

34. The compound of claim 31, wherein the compound is of Formula II-c: 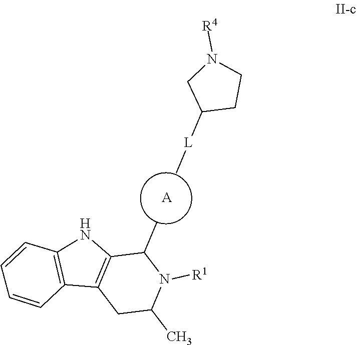
or a pharmaceutically acceptable salt thereof.

35. The compound of claim 31, wherein the compound is of Formula II-d: 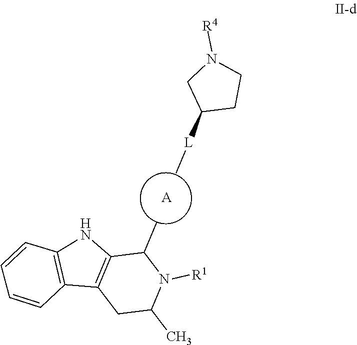
or a pharmaceutically acceptable salt thereof.

36. The compound of claim 31, wherein the compound is of Formula II-e: 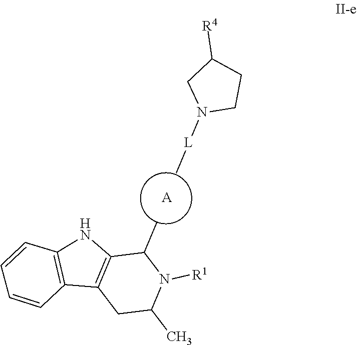
or a pharmaceutically acceptable salt thereof.

37. The compound of claim 31, wherein the compound is of Formula II-f: 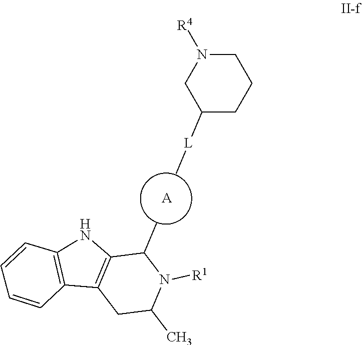
or a pharmaceutically acceptable salt thereof.

38. The compound of claim 31, wherein the compound is of Formula II-g: 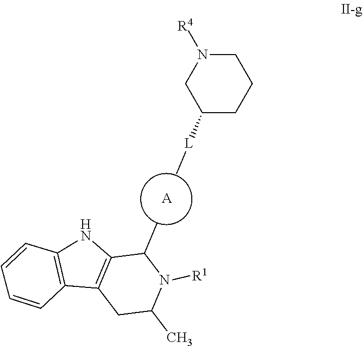
or a pharmaceutically acceptable salt thereof.

39. The compound of claim 31, wherein the compound is of Formula II-h: 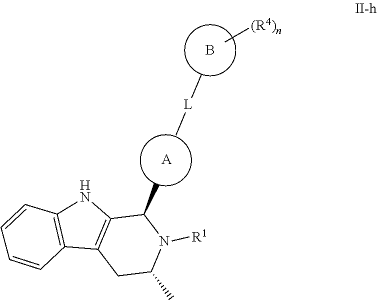
or a pharmaceutically acceptable salt thereof.

40. The compound of any one of claims 1-30, wherein the compound is of Formula III: 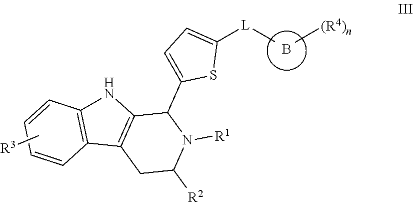
or a pharmaceutically acceptable salt thereof.

41. The compound of any one of claims 1-30, wherein the compound is of Formula IV: 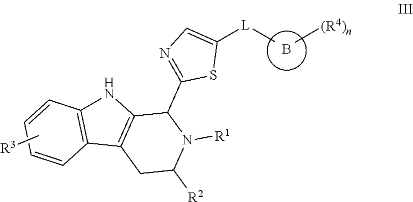
or a pharmaceutically acceptable salt thereof.

42. The compound of any one of claims 1-30, wherein the compound is of Formula V: 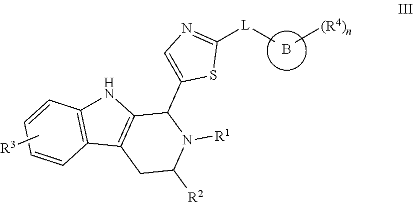
or a pharmaceutically acceptable salt thereof.

43. The compound of any one of claims 1-30, wherein the compound is of Formula VI: 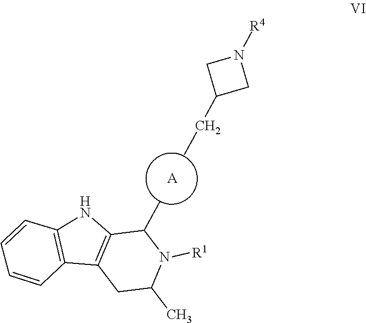
or a pharmaceutically acceptable salt thereof.

44. A compound selected from Table 1, or a pharmaceutically acceptable salt thereof.

45. A compound selected from Table 3, or a pharmaceutically acceptable salt thereof.

46. A compound selected from Table 4, or a pharmaceutically acceptable salt thereof.

47. A compound selected from Table 5, or a pharmaceutically acceptable salt thereof.

48. A pharmaceutical composition comprising a compound of any one of claims 1-47, or a pharmaceutically acceptable salt thereof, and a pharmaceutically acceptable carrier or excipient.

49. A method for treating a disorder mediated by an estrogen receptor in a subject, comprising administering to the subject a therapeutically effective amount of the compound of any one of claims 1-47, or a pharmaceutically acceptable salt thereof, or the composition of claim 48.

50. The method of claim 49, wherein the disorder is selected from the group consisting of breast cancer, ovarian cancer, endometrial cancer, vaginal cancer, lung cancer, bone cancer, uterine cancer, and endometriosis.

51. The method of claim 49 or 50, further comprising administering the compound or composition in combination or alternation with an anti-cancer agent.

52. The method of claim 51, wherein the anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

53. The method of claim 49 or 50, further comprising administering the compound or composition in combination or alternation with an estrogen receptor antagonist or a partial estrogen receptor antagonist.

54. The method of any one of claims 49-53, wherein the disorder is breast cancer.

55. A method of treating a subject suffering from a cancer comprising administering the compound of any one of claims 1-47, or a pharmaceutically acceptable salt thereof, or the composition of claim 48, in combination with an anti-cancer agent.

56. The method of claim 55, wherein the anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

57. The method of claim 55, wherein the anti-cancer agent is an mTOR inhibitor.

58. The method of claim 57, wherein the mTOR inhibitor is selected from everolimus, sirolimus, temsirolimus, and LY3023414.

59. The method of claim 55, wherein the anti-cancer agent is a CDK4/6 inhibitor.

60. The method of claim 59, wherein the CDK4/6 inhibitor is selected from palbociclib, abemaciclib, ribociclib, lerociclib, trilaciclib, and SHR6390.

61. The method of claim 55, wherein the anti-cancer agent is an antibody to or inhibitor of PD-1, PD-L1 or CTLA-4.

62. The method of claim 55, wherein the anti-cancer agent is an antibody to or inhibitor of EGFR, PGFR, or IGFR.

63. The method of claim 55, wherein the anti-cancer agent is a HER2 inhibitor.

64. The method of claim 63, wherein the HER2 inhibitor is selected from tucatinib, trastuzumab, pertuzumab, ado-trastuzumab, trastuzumab emtansine, ado-trastuzumab emtansine, trastuzumab deruxtecan pertuzumab, lapatinib, and neratinib.

65. The method of claim 55, wherein the anti-cancer agent is a PI3 kinase inhibitor.

66. The method of claim 65, wherein the PI3 kinase inhibitor is selected from perifosine, CAL101, BEZ235, XL147, XL765, GDC-0941, and IPI-145.

67. The method of claim 55, wherein the anti-cancer agent is a PIK3CA inhibitor.

68. The method of claim 67, wherein the PIK3CA inhibitor is selected from alpelisib, taselisib, and LY3023414.

69. The method of claim 55, wherein the anti-cancer agent is an aromatase inhibitor.

70. The method of claim 69, wherein the aromatase inhibitor is selected from aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione.

71. The method of claim 69, wherein the aromatase inhibitor is selected from anastrozole, letrozole, and exemestane.

72. A method of preventing recurrence of a cancer in a subject comprising administering to the subject the compound of any one of claims 1-47, or a pharmaceutically acceptable salt thereof, or the composition of claim 48.

73. The method of claim 72, wherein the cancer is selected from breast cancer, ovarian cancer, endometrial cancer, vaginal cancer, lung cancer, bone cancer, and uterine cancer.

74. The method of claim 72 or 73, wherein the compound or composition is administered as an adjunctive therapy after or instead of chemotherapy, radiation, or surgery.

75. The method of any one of claims 72-74, wherein the compound or composition is administered after surgery.

76. The method of claim 72 or 73, wherein the compound or composition is administered prior to surgery.

77. The method of any one of claims 72-76, wherein the cancer is breast cancer.

78. The method of claim 77, wherein the breast cancer has progressed in the presence of endocrine or aromatase therapy.
